(19) United States
(12) Patent Application Publication
(10) Pub. No.: US 2022/0282304 A1
(43) Pub. Date: Sep. 8, 2022

(54) SAME-SAMPLE ANTIBIOTIC SUSCEPTIBILITY TEST AND RELATED COMPOSITIONS, METHODS AND SYSTEMS

(71) Applicant: CALIFORNIA INSTITUTE OF TECHNOLOGY, Pasadena, CA (US)

(72) Inventors: Rustem F. ISMAGILOV, Pasadena, CA (US); Eric LIAW, Pasadena, CA (US); Anna E. ROMANO, Pasadena, CA (US)

(21) Appl. No.: 17/683,246

(22) Filed: Feb. 28, 2022

Related U.S. Application Data
(60) Provisional application No. 63/154,642, filed on Feb. 26, 2021.

(51) Int. Cl. C12Q 1/689; C12Q 1/18

(52) U.S. Cl. CPC C12Q 1/689; C12Q 1/18; C12Q 1/6806

(57) ABSTRACT

Provided herein is an antibiotic susceptibility and related compositions, methods and systems based on nucleic acid detection based on detected intracellular and extracellular nucleic acid from a same sample, which allows determination of antibiotic susceptibility of microorganisms as well as the diagnosis and/or treatment of related infections in individuals.

### CROSS-REFERENCE TO RELATED APPLICATION

The present application claims priority to U.S. Provisional Application No. 63/154,642, entitled “Same Sample Antibiotic Susceptibility Test and Related Compositions Methods and Systems” filed on Feb. 26, 2021, with docket number P2569-USP, the contents of which is incorporated by reference in its entirety. The present application may also be related to U.S. application Ser. No. 16/218,633 filed on Oct. 11, 2018 and published on Jun. 27, 2019 with publication No. US2019/0194726, to U.S. application Ser. No. 17/164,674 filed on Feb. 1, 2021 and published on Sep. 30, 2021 with publication No. US2021/0301326, and to International Application PCT/US2018/055501 filed on Oct. 11, 2018 and published on Apr. 18, 2019 with publication No. WO2019/075624, the content on each of which is also incorporated herein by reference in its entirety.

### STATEMENT OF GOVERNMENT GRANT

This invention was made with U.S. Government support under Agreement No. W15QKN-16-9-1002 awarded by the ACC-NJ to the MCDC. The Government has certain rights in the invention.

### FIELD

The present disclosure relates to microorganisms and related biology as well as to diagnosis and treatment of related conditions in individuals. In particular, the present disclosure relates to antibiotic susceptibility of microorganisms and related markers, compositions, methods and systems. More particularly, the present disclosure relates to a same-sample antibiotic susceptibility test (AST) and related compositions, methods and systems.

### BACKGROUND

Antibiotic susceptibility is an important feature of the biology of various microorganisms, which can be used in identifying approaches to treat or prevent bacterial infections.

Ideal antibiotic therapy is based on determination of the etiological agent for a particular condition and determination of the antibiotic sensitivity of the identified agent. In particular, the effectiveness of individual antibiotics varies with various factors including the ability of the microorganism to resist or inactivate the antibiotic.

Despite progress in identifying methods and systems to test antibiotic susceptibility for various microorganisms, as well as the identification of related markers, determination of antibiotic susceptibility can still be challenging, in particular when determination of antibiotic susceptibility performed with rapid and yet accurate detection is desired.

### SUMMARY

Provided herein is a same-sample antibiotic susceptibility test (AST) and related compositions, methods and systems which allow a rapid AST determination with an improved accuracy with respect to existing nucleic acid accessibility AST, by detecting extracellular/accessible nucleic acid and intracellular/inaccessible nucleic acid from a same sample subjected to the testing.

In particular, according to a first aspect, embodiments of a same-sample AST compositions methods and systems herein described are based on the detection of intracellular and extracellular nucleic acid from cellular and extracellular components (herein also indicated as fractions) of a same sample respectively, and the use of an intra/extra NA proportion value of the same sample obtained therefrom for live and death determination and/or the AST determination.

Accordingly, methods according to the first aspect comprises a method to detect a nucleic acid of a microorganism in a sample. The method according to the first aspect comprises contacting the sample with an antibiotic to provide an antibiotic-treated sample and separating the antibiotic-treated sample into an antibiotic-treated extracellular component and an antibiotic-treated cellular component.

The method according to the first aspect further comprises detecting a nucleic acid concentration of the antibiotic-treated extracellular component to obtain an antibiotic-treated extracellular nucleic acid concentration value and detecting a nucleic acid concentration of the antibiotic-treated cellular component to obtain an antibiotic-treated intracellular nucleic acid concentration value.

Determination of an intra/extra NA proportion value of the sample, determination of live and dead cells and/or AST determination can be performed based on the antibiotic-treated extracellular nucleic acid concentration value and an antibiotic-treated intracellular nucleic acid concentration value, as will be understood by a skilled person upon reading of the present disclosure. Determination of an intra/extra NA proportion value of the sample allows determination of live and dead cells in the sample and/or determination of susceptibility or resistance of the microorganism to the antibiotic, in absence and without the need, of an additional detection (in particular marker detection) in the same sample and/or in a separate sample.

In particular, in some embodiments the method comprises
determining an intra/extra proportion value by providing a value corresponding to a proportion of the antibiotic-treated extracellular nucleic acid concentration value and the antibiotic-treated intracellular nucleic acid concentration value
determining a proportionality of dead and live microorganism cells in the sample caused by and/or or as a function of, the antibiotic by determining an intra/extra proportion value of the sample to provide a dead/live proportion value of the microorganism cells in the sample and/or
determining a susceptibility or resistance of the microorganism in the by determining intra/extra proportion value of the antibiotic-treated sample and comparing the intra/extra proportion value of the antibiotic-treated sample with a reference value to provide a dead/live proportion value of the microorganism cells in the sample caused by the antibiotic,
   as will be understood by a skilled person upon reading of the present disclosure.

The systems according to the first aspect comprise at least means and/or reagents for performing exposure, separation of a same sample into extracellular fraction and intracellular fraction, and reagents for detecting an intracellular nucleic acid concentration value and an extracellular nucleic acid concentration value in a same sample according to methods herein described. The system can further comprise a look-up table and/or software to determine the intra/extra NA proportion value, determine live and dead cells and/or resistance, determine susceptibility or resistance of the microorganism to the antibiotic, according to methods of the first aspect herein described.

According to a second aspect, same-sample AST, and related compositions methods and systems herein described replace the need for a control with the use of one or more thresholds from experiments and/or literature search to account for background events of the sample unrelated to antibiotic susceptibility of the microorganism in the sample, which however affect intracellular and/or extracellular nucleic acid concentration of the microorganism in the same sample. The one or more thresholds can replace or be added in various combinations to performance of reference experiments such as control experiments as will be understood by a skilled person upon reading of the present disclosure.

Accordingly, methods according to the second aspect comprise
comparing an antibiotic treated intracellular/extracellular nucleic acid proportion value of a sample with a reference value indicative of an intracellular/extracellular nucleic acid proportion in the sample in absence of antibiotic treatment,
the comparing performed to obtain a treated-reference nucleic acid comparison outcome of the sample, wherein the reference value comprises or consists of one or more thresholds.

The treated-reference nucleic acid comparison outcome can then be used to perform a live/dead determination and/or an AST determination in absence, and without the need, of an additional detection (in particular marker detection) in the same sample and/or in a separate sample according to methods herein described as will be understood by a skilled person.

The systems according to the second aspect comprise a look up table and/or software to obtain a treated-reference nucleic acid comparison outcome of a sample in combination with an antibiotic treated intracellular/extracellular nucleic acid proportion value of the sample in accordance with methods herein described.

According to a third aspect, same-sample AST compositions methods and systems herein described can be configured to perform detection of an intra/extra NA proportion value of a same sample after repeated antibiotic exposures of the same sample in time (herein also indicated as time series).

In particular, a method according to the third aspect, a same sample is subjected to n cycles (time series) of antibiotic exposure separation of the same sample in extracellular and cellular fractions, detection of an extracellular nucleic acid in the extracellular fraction of the same sample, reconstitution of a sample from the cellular fraction of the same sample to provide a reconstituted sample. The n cycles are then followed by detecting an intracellular nucleic acid. in the cellular fraction of the nth reconstituted sample.

In the method of the third aspect, each n-reconstituted sample is obtained by adding culture medium to a cellular fraction of the same sample or of a previous, reconstituted sample of the n-reconstituted samples.

In particular, in the method according to the third aspect, the method comprises n-cycles of
antibiotic exposure of the sample to obtain a treated sample after the antibiotic exposure;
separation of the treated sample to obtain an extracellular component and a cellular component of the treated sample,
detection of an extracellular nucleic acid concentration value in the extracellular fraction of the sample following the exposure, and
combination the antibiotic treated cellular fraction of the sample with culture media to reconstitute the sample;
   to obtain an nth reconstituted sample, n being an integer equal or higher than 1.

In the method according to third aspect, an n+1 cycle is performed comprising the antibiotic exposure, separation and detection of the extracellular nucleic acid concentration value, followed by detection of an intracellular nucleic acid concentration value in the cellular fraction of the nth reconstituted sample.

The method further comprises performing n intracellular nucleic acid calculations based on n extracellular nucleic acid detection and the intracellular and extracellular nucleic acid detection of the finally treated samples, to provide an intra/extra NA proportion value of each measurement and performed live and dead cells determination and/or the susceptibility or resistance determination for the microorganism in absence, and without the need, of an additional detection (in particular marker detection) in the same sample and/or in a separate sample.

In particular, determination of live and dead microorganism cells and/or determination of susceptibility or resistance of the microorganism to the antibiotic, can be performed in the sample in combination with thresholds and/or reference measurements performed a different times, to account for the lag time of the nucleic acid release in the same sample due to the antibiotic administration and/or for variation in time additional biological events interfering with the AST determination.

The systems according to the third aspect comprise at least means and/or reagents for performing separation of a same sample into extracellular fraction and intracellular fraction, reagents for detecting an intracellular nucleic acid concentration value and an extracellular nucleic acid concentration value in a same sample according to methods herein described as well as culture medium. The system can further comprise a look-up table and/or software to determine the intra/extra NA proportion value according to methods according to the third aspect herein described.

According to a fourth aspect, same-sample AST compositions methods and systems herein described can be configured to perform AST in samples obtained by partitioning a specimen in a plurality of samples (herein specimen partitions). Also, a sample can be partitioned to obtain a plurality of sample partitions (herein sample partitions or sub-samples).

In methods according to the fourth aspect any one of the same-sample methods according to the first aspect, second aspect and/or third aspect can be performed on each partition of a plurality of specimen or sample partitions to determine intra/extra NA proportion value of the each partition. Determination of the intra/extra NA proportion value of the each partition can be followed by calculation directed to determine the live and death determination and/or determination of susceptibility or resistance of the microorganism to the antibiotic in the each partition of the plurality of partitions of the specimen or sample in absence, and without the need, of an additional detection (in particular marker detection) in the same sample and/or in a separate sample. Embodiments of method according to the fourth aspect allow performing parallel multiplexed determination of live and dead microorganism cells and/or susceptibility/resistance determination in an array of partitions each subjected to different experimental conditions, thus providing a profile of the specimen or sample.

The system according to the fourth aspect comprise components of the systems according to the first aspect, second aspect and/or third aspect configured for exposure, separation, sample reconstitution and/or extraction and nucleic acid detection, in specimen and/or sample partitions.

According to a fifth aspect, same-sample AST compositions methods and systems herein described according to the first aspect, second aspect and/or third aspect can be configured to perform AST wherein the sample is partitioned before antibiotic exposure to provide a plurality of partitions (herein also sub-samples) and performing the antibiotic exposure under at least one same experimental condition, in a corresponding set of partitions. In particular, the in methods according to the fifth aspect antibiotic exposure is performed under at least one same test condition in a corresponding at least one set of test partitions. In some embodiments antibiotic exposure can be performed also under at least one same reference condition in a corresponding at least one set of reference partitions.

In the method according to the fifth aspect, separation and detection of intracellular and extracellular nucleic acid in each partition is performed to determine intra/extra NA proportion value of the each partition of the at least one set of partitions subjected to the at least one same experimental conditions. Determination of the intra/extra NA proportion value of the each partition can be followed by calculation directed to determine the live and death status of microorganism cells inside of the each partition and of the at least one set of partitions, and/or by determination of susceptibility or resistance of microorganism in the sample based on the intra/extra NA proportion value of the at least one set of partitions.

The system according to the fifth aspect comprise components of the systems according to the first aspect, second aspect and/or third aspect configured for exposure, separation, nucleic acid extraction, nucleic acid detection and/or sample reconstitution, in specimen and/or sample partitions.

Same-sample AST performed in specimen partitions and/or sample partitions according to the fourth or fifth aspects, enables performing in parallel multiplexing of same-sample methods of the disclosure in the partitions as will be understood by a skilled person upon reading of the present disclosure. The method of the fifth aspect also allow performance of in series multiplexing and digital embodiments of the same-sample method of the disclosure as will be understood by a skilled person.

According to a sixth aspect, a system is described for performing at least one of the methods herein described to detect a nucleic acid of a microorganism in a sample, to detect antibiotic susceptibility of a microorganism, to perform an antibiotic susceptibility test for the microorganism, and/or to diagnose and/or treat a microorganism infection in an individual. The system comprises an antibiotic, at least a probe specific for a nucleic acid of the microorganism or for a polynucleotide complementary thereto, and reagents for detecting the at least one probe. The system can optionally comprise reagents to perform a lysis treatment, a separation treatment and/or mechanical separation of the sample for concurrent sequential or combined use in any one of the methods of the disclosure. The system can also comprise one or more of the high-throughput instrumentations herein described, such as filter plate, microfluidic devices and laboratory automation systems.

Additional features of the same-sample antibiotic susceptibility tests and related compositions methods and systems are indicated in other portions of the description and in the enclosed claims.

The same-sample AST and related compositions methods and systems herein described can be advantageously used in connection with performing AST in specimens (such as clinical specimens) including a low number of cells. In particular, same-sample AST of this disclosure and related compositions, methods and systems can be configured to target high copy number nucleic acids to increase detection of nucleic acids from samples with low numbers or densities of cells of interest as will be understood by a skilled person. Provided herein is an exemplary specific protocol for amplifying ribosomal RNAs.

The same-sample AST and related compositions methods and systems herein described can be advantageously used to perform rapid AST (within 60 mins or less with an increased accuracy of the related determination.

In particular, same-sample AST herein described and related compositions, methods and systems, can be configured to be performed in parallelized and multiplexed fashions.

Parallelization of assays increases throughput and reduces random noise. It also provides for methods of performing multiple ASTs in parallel, including ASTs on multiple clinical specimens, ASTs with multiple antibiotics, ASTs with multiple antibiotic concentrations. It can be performed in multi-well plates and other standard laboratory automation techniques. It can use, for example, plate-based filtration and plate-based centrifugation as exemplary methods of separating intracellular and extracellular nucleic acids.

Multiplexing of nucleic acid amplification decreases the number of assay runs needed to yield the same information, decreases the amount of sample required to yield the same information, and increases assay sensitivity. The same-sample AST and related compositions methods and systems herein described can also be performed with various enhancing treatments that increase the assays' discrimination of susceptible and resistant strains.

The same-sample AST and related compositions methods and systems herein described can be performed in connection with partitioning of a specimen in digital specimen partitions or partitioning of a sample in digital sample partitions (herein indicate also as “digital sample partitioning”) to achieve increased accuracy in detection and determination through “digital loading” of the samples which can be used to obtain additional information about the sample's microorganism of interest. In particular, when digital partition of a sample is performed, both extracellular and intracellular subsets are recovered by filtration and quantified from each digitally partitioned sample to perform the live or dead cells determination and/or the same-sample AST of the disclosure.

The same-sample AST and related compositions methods and systems herein described can be performed with samples or sample partitions containing 10 cells or less, 5 cells or less, and with single cell sample allowing detection of the antibiotic effect at a cellular level which cannot be performed with existing ASTs.

The same-sample AST and related compositions methods and systems herein described can be performed using the “relative difference index”, a summary statistic that can be calculated from the results of accessibility ASTs. Relative difference index can be calculated for accessibility AST methods.

The same-sample AST and related compositions methods and systems herein described can be configured to have one or more clinically useful properties, such as speed, simplicity, cost, robustness to sample matrix, accuracy, coverage of pathogens, and interpretability.

The same-sample AST and related compositions methods and systems herein described allows separating the intracellular and extracellular subsets of the sample's nucleic acids without necessarily losing or ignoring certain nucleic acids. Lossless recovery increases the number of nucleic acid of a cell which are detected, as same-sample AST can theoretically enable the measurement of all nucleic acids present in a sample, which other accessibility AST modalities cannot.

The same-sample AST and related compositions methods and systems herein described allow optional addition of digital sample partitioning to all accessibility AST methods. In partitioning embodiments of a same-sample AST, a given specimen or a given sample is split into multiple partitions before or after the antibiotic exposure takes place. The specimen or sample is partitioned such that the number of cells in each partition can be estimated by the number of partitions that are occupied by cells of the microorganism of interest. When such a partitioning is performed, the specimen or sample is said to have been partitioned “digitally”, and the partitioning is deemed in “the digital range”. Accessibility AST methods that include digital partitioning yield additional information than when digital partitioning is not performed, namely the total number or density of cells in the sample and the responses of individual cells (or low numbers of individual cells) to the antimicrobial agent and additional information identifiable by a skilled person.

The same-sample antibiotic susceptibility test and related compositions, methods and systems herein described can be used in connection with various applications wherein live or death determination and/or detection of antibiotic susceptibility for a microorganism is desired. For example, the same-sample antibiotic susceptibility test and related compositions, methods and systems herein described can be used in drug research and to develop diagnostic and therapeutic approaches and tools to counteract infections, and to enable development and commercialization of narrow-spectrum antimicrobial therapeutics, such as antimicrobial therapeutics with a narrower spectrum than the therapeutic that would have been prescribed in the absence of the test. Additional exemplary applications include uses of the same-sample antibiotic susceptibility test and related compositions, methods and systems herein described in several fields including basic biology research, applied biology, bio-engineering, etiology, medical research, medical diagnostics, therapeutics, and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIG. 1 shows a schematic representation of the nucleic acid accessibility as a marker of changes in the integrity of the cell membrane.

FIG. 2 shows a schematic diagram of an exemplary workflow of the same-sample AST, schematically illustrating 6 stages f the workflow which can but not necessarily have to be included to perform detection and AST testing as will be understood by a skilled person upon reading of the disclosure.

FIG. 3 shows a chart illustrating the results of three same-sample AST on a sample comprising a susceptible E. coli isolate performed as a proof of principle. The chart nucleic acid accessibility as percentage extracellular nucleic acid (y axis) detected under different experimental conditions (x-axis) and in particular test condition (exposure for 30 mins to 1 ug/ml Ertapenem, black circle) and control conditions (exposure for 30 mins to culture medium without Ertapenem, white diamond).

FIG. 4 shows a chart illustrating a same-sample AST performed as a proof of principle on sample comprising a susceptible E. coli strain, with multiple replicate treated conditions and multiple concurrent reference conditions. The chart illustrates nucleic acid accessibility as percentage extracellular amplicons (y axis) detected in 32 filtrates and lysates respectively having different pairs of extracellular and intracellular concentrations (x-axis) under test condition (exposure for 60 mins to 1 ug/ml Ertapenem, solid line) and control conditions (exposure for 30 mins to culture medium without Ertapenem, dotted line). The illustration of FIG. 4 ignores the 95% Poisson confidence intervals.

FIG. 5 shows a chart illustrating a same-sample AST performed as a proof of principle on sample comprising a resistant E. coli strain, with multiple replicate treated conditions and multiple concurrent reference conditions. The chart illustrates nucleic acid accessibility as percentage extracellular amplicons (y axis) detected in 32 filtrates and lysates respectively having different pairs of extracellular and intracellular concentrations (x-axis) under test condition (exposure for 60 mins to 1 ug/ml ertapenem, solid line) and control conditions (exposure for 30 mins to culture medium without ertapenem, dotted line). The illustration of FIG. 5 ignores the 95% Poisson confidence intervals.

FIG. 6 shows a chart illustrating the results of a cluster analysis of extracellular and intracellular nucleic acid concentrations detected by qPCR of 23S rRNA in a digitally partitioned sample exposed for 70 minutes to 1 ug/ml ertapenem (test condition, black symbols) or culture medium (reference condition, white symbols), with an exemplary same-sample testing according to the present disclosure. In particular, the chart shows the intracellular nucleic acid threshold cycles (Cq) (y-axis) which reflect intracellular nucleic acid concentration, of the lysate at both testing and control conditions, and the extracellular nucleic acid threshold cycles (Cq) (x-axis), which reflect extracellular nucleic acid concentration, of the filtrate at both testing and control conditions. The results also show the corresponding inferred cell status shown by the different shapes of the symbols (lysed squares, intact diamonds and empty circles).

FIG. 7 shows a chart illustrating the results of a cluster analysis of extracellular and intracellular nucleic acid concentrations detected by ddPCR of 23S rRNA in a digitally partitioned sample exposed for 70 minutes to 1 ug/ml Ertapenem (test condition, black symbols) or culture medium (control, white symbols), with an exemplary same-sample testing according to the present disclosure. In particular, the chart shows the lysate copies/ul (y-axis) which reflect intracellular nucleic acid concentrations at both testing and control conditions, and the filtrate copies/ul (x-axis), which reflect extracellular nucleic acid concentrations of the filtrate at both testing and control conditions. The results also show the corresponding inferred cell status shown by the different shapes of the symbols (lysed squares, intact diamonds and empty circles).

FIG. 8 shows a chart illustrating the results of a cluster analysis of extracellular and intracellular nucleic acid concentrations detected by ddPCR of 23S rRNA in a digitally partitioned sample exposed for 40 minutes to 1 ug/ml Ertapenem (test condition, black symbols) or culture medium (reference conditions, white symbols), with an exemplary same-sample testing according to the present disclosure. In particular, the chart shows the lysate copies/ul (y-axis) which reflect intracellular nucleic acid concentrations at both testing and control conditions, and the filtrate copies/ul (x-axis), which reflect extracellular nucleic acid concentrations of the filtrate at both testing and reference conditions. The results also show the corresponding inferred cell status in each partition (live cells square, dead cells triangle, and live and dead cells square including triangle).

FIG. 9 shows a chart illustrating the results of a cluster analysis of extracellular and intracellular nucleic acid concentrations detected by qPCR of 23SRNA in a digitally partitioned sample having a cell density of 0, 0.5, 1, and 2, following exposure for 40 minutes to 1 ug/ml Ertapenem (test condition black symbols) or culture medium (reference condition, white symbols), with an exemplary same-sample testing according to the present disclosure. In particular, the chart shows the intracellular nucleic acid threshold cycles (Cq) (y-axis) which reflect intracellular nucleic acid concentration, of the lysate at both testing and reference conditions, and the extracellular nucleic acid threshold cycles (Cq) (x-axis), which reflect extracellular nucleic acid concentration, of the filtrate at both testing and control conditions. The results also show the corresponding inferred cell status using different shape of the symbols (lysed squares, intact diamonds, and empty circles).

FIG. 10 shows a chart illustrating the results of a statistical analysis performed based on the Extracellular Intracellular Nucleic Acid Proportion Value (EINAPV) of the same-sample AST illustrated in FIG. 9. In the graph of FIG. 10 the false positive rate (where a “susceptible” call is considered positive) (y-axis) is show as a function of an A Priori Threshold Value (APTV) (x-axis) and three cell densities (500 cells/ml narrow dotted line, 1000 cells/ml long dotted line and 4000 cells/ml solid line).

FIG. 11A shows a chart illustrating the results of a cluster analysis of extracellular and intracellular nucleic acid concentrations detected by qPCR of 23SRNA in a digitally partitioned sample having exposure duration of 0, 30, 60, and 120 min to 1 ug/ml Ertapenem (test condition, black symbols) or culture medium (reference condition, white symbols), with an exemplary same-sample testing according to the present disclosure. In particular, the chart shows the intracellular nucleic acid threshold cycles (Cq) (y-axis) which reflect intracellular nucleic acid concentration, of the lysate at both testing and reference conditions, and the extracellular nucleic acid threshold cycles (Cq) (x-axis), which reflect extracellular nucleic acid concentration, of the filtrate at both testing and control conditions. The results also show the corresponding inferred cell status using different shape of the symbols (lysed squares, intact diamonds and empty circles).

FIG. 11B shows the results of a digitally-loaded same-sample AST run containing 1 control condition and 2 test conditions. The strain examined was E. coli K12 MG1655. The test conditions were a 0.25 μg/mL ertapenem exposure and a 2.0 μg/mL ertapenem exposure, both lasting 20 minutes. Each test condition comprised 32 sample partitions. Each panel shows 32 extracellular and 32 intracellular nucleic acid concentration values in the form of qPCR threshold cycles, some of which were recorded as “infinity”. The results of the well loading status algorithm are depicted by the different point shapes. The extracellular/intracellular nucleic acid proportion value (EINAPV) from each condition is not printed but calculated in the description accompanying the figure.

FIG. 12 shows changes in extracellular and total genomic DNA over time seen in replicates of bulk accessibility AST. These phenomena are expected to occur during same-sample AST exposures.

FIG. 13 shows how antibiotic concentration affects antibiotic killing, using replicates of bulk accessibility AST. The information in the graph can be used to construct the strain's dose-response curve at each duration exposure.

FIG. 14 shows an example of a compartment model of in vitro antibiotic exposure.

FIG. 15 shows example population trajectories allowed by the compartment model.

FIG. 16 shows an example of choice of function to link cell population to nucleic acid quantity.

FIG. 17 shows an example of hierarchical Bayesian statistical error modelling that corrects for batch effects.

FIG. 18A shows a schematic, with simulated data, of digitally-loaded same-sample AST with a categorical well status loading algorithm with a rate of lysis parameter being twice the growth rate.

FIG. 18B shows a schematic, with simulated data, of digitally-loaded same-sample AST with a categorical well status loading algorithm with a rate of lysis parameter being three times the growth rate.

FIG. 19 shows an example of a derivation of a mathematical expression which is the likelihood of observing the observed tally of well loading statuses given values of parameters, and assuming that the population behaves according to a Markov birth-death process.

FIG. 20 shows example values for parameters [resulting from fitting algorithms and] used in future inferences.

### DETAILED DESCRIPTION

Provided herein is a same sample antibiotic susceptibility test (AST) and related compositions, methods and systems which allow a rapid AST determination with an improved accuracy with respect to existing nucleic acid accessibility AST, by detecting extracellular/accessible nucleic acid and intracellular/inaccessible nucleic acid from a same sample subjected to the testing.

In particular, the methods of the present disclosure are methods for measuring susceptibility of microorganisms to antimicrobial drugs (a.k.a. antibiotics) that use nucleic acid as a marker of antibiotic susceptibility of microorganisms. These methods herein also named “accessibility AST” are ASTs based on a determination of accessibility of nucleic acids of a microorganism to detection reagents, as a marker event of susceptibility/resistance of the microorganism to one or more antibiotics.

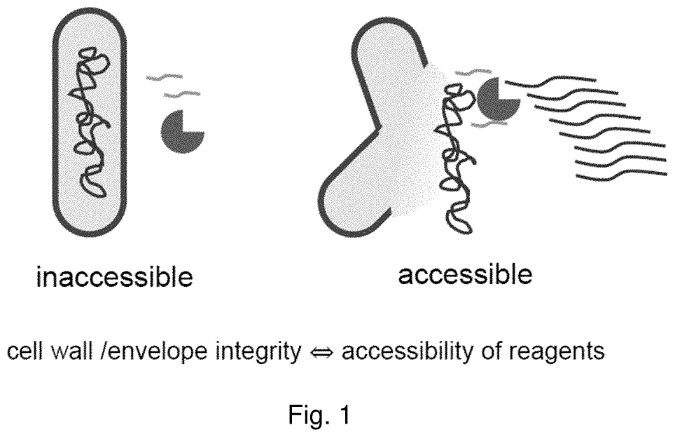

This class of tests is based on the observation that any breach in the cell envelope's continuity very rapidly changes the accessibility of nucleic acid to detection reagents, since the intracellular nucleic acids contained within the cell become topologically equivalent to extracellular nucleic acids that can diffuse to, contact, and interact with the detection reagents, as schematically represented in FIG. 1. Thus, the amount or rate of cell-wall targeting antibiotic activity promotes an increase in extracellular nucleic acid and a decrease in intracellular nucleic acid in a sample containing the microorganism. The increase in extracellular nucleic acid or the decrease in intracellular can be performed based on detection of any type of nucleic acids alone or in combination independently from the specific of any detected nucleic acid (such as quantity and/or timing of expression for mRNA).

Accordingly, a change in accessibility of nucleic acid is a biological event fundamentally distinct from other events such as synthesis of new biomass by the living population of microorganisms or changes in the transcriptional regulation or turnover of messenger RNAs (Ref. US2019/0194726, US2021/0301326, and WO2019/075624). In particular, since lysis can occur early after exposure to antibiotics, accessibility ASTs allow a rapid AST determination with antibiotic contacting times which results in a larger initial signal than one measured the change in total biomass of the microorganism or the expression of many genes. The larger early signal of accessibility provides a rapid and accurate susceptibility/resistance determination compared with AST based on determination of different biological events [1]-[3].

A description of accessibility ASTs is provided in US2019/0194726, US2021/0301326, and WO2019/075624 incorporated herein by reference in their entirety [1]-[3].

In this disclosure, an additional class of accessibility ASTs, is described called “same-sample AST” which changes the operation of existing ASTs by recovering and quantifying both accessible/extracellular and inaccessible/intracellular nucleic acids from cellular and extracellular components (herein also fractions) of a same given sample.

In particular, in same-sample ASTs, changing one's operations to obtain both intracellular and extracellular nucleic acid concentrations from a same given sample enables analysis of the AST sample as a system in which biological events interfering with nucleic acid accessibility are considered and characterized as confounding sources of physical or biological stochasticity.

Accordingly, in a same-sample AST, intracellular and extracellular nucleic acid detected from a same sample are used in mathematical elaboration and statistical modeling which takes into account biological events interfering with nucleic acid accessibility as confounding variables/phenomena of the sample system which impact determination of susceptibility or resistance. The results of this mathematical elaboration yield key information that improves the accuracy of the AST because it allows one of skill to determine the impact on the susceptibility determination of phenomenon interfering with the detected markers of susceptibility. Events such as number of cells in the sample or loaded into partitions of the sample, background lysis, cell growth, as well as modifications of these features in time, impact the external/accessible nucleic acid or internal/inaccessible nucleic acid of the sample as will be understood by a skilled person.

Accordingly, quantitatively detecting both accessible/extracellular and inaccessible/intracellular nucleic acids from separated components (herein also fractions) of a same given sample allows an improved AST determination with respect to exiting ASTs at least because it reduces and even minimizes the impact on the susceptible/resistant determination of these biological events by considering them as confounding variables of the biologically stochastic sample system.

In particular, methods and systems of the same-sample AST of the disclosure are based on detection of and determination an intracellular/extracellular nucleic proportion value of the same sample which allows, in addition to analysis of the sample as a biologically stochastic system, to determine a dead and live proportion of microorganism caused by the antibiotic and/or determine antibiotic susceptibility while minimizing the impact on these determinations of the number of cells present in the sample.

Minimization of the impact of this confounding variable on the AST determination further allows fundamentally new operations of accessibility AST (such as digital same-sample AST and time series same-sample AST) that in turn address other confounding variables of the of the biologically stochastic sample system as will be understood by a skilled person upon reading of the present disclosure.

Accordingly, in particular, in some embodiments, the same-sample AST herein described reduces and even minimizes the impact on the determination of dead/live cell proportion and/or antibiotic susceptibility of at least three confounding variables of an AST.

The first confounding variable impacting the AST determination is the number of cells in a sample. In same-sample AST of the disclosure, the impact of such confounding variable is addressed by
using an intracellular/extracellular nucleic proportion value of the same sample established by comparing intracellular nucleic acid detected in a cellular fraction of the same sample and extracellular nucleic acid detected in an extracellular fraction of the same sample.

The use of an intracellular/extracellular nucleic proportion value of a same sample thus allows one of skill to minimize the impact of variability in the nucleic acid detection of the sample due to the unknown number of cells as will be understood by a skilled person.

The second confounding variable impacting the AST determination is the background lysis in a sample: in the same-sample AST compositions methods and systems of the disclosure, the impact of such confounding variable is addressed by
comparing an intracellular/extracellular nucleic proportion value of the same sample obtained by comparing intracellular nucleic acid detected in a cellular fraction of the sample and extracellular nucleic acid detected in an extracellular fraction of the same sample with a corresponding (comparable) intracellular/extracellular nucleic proportion value of a reference sample (such as a control sample) or a corresponding (comparable) intracellular/extracellular nucleic acid proportion value of a reference measurement (if multiple measurements on a same sample are performed in time according to embodiments herein described) and/or
establishing one or more thresholds based on standard deviations of distributions derived from experiments and/or literature data and accounting for background events unrelated to antibiotic susceptibility, and comparing an intracellular/extracellular nucleic proportion value of the same sample with the established thresholds to determine antibiotic susceptibility.

The third confounding variable impacting the AST determination is the lag time of the nucleic acid release in a sample due to the antibiotic administration: addressed by the same-sample AST of the present disclosure by performing multiple nucleic acid detection of the same sample in time and
performing multiple measurements and comparing the treated and/or control intra/extra nucleic acid proportion value of each measurement; and/or
establishing a base lag time (e.g., derived from experiments and/or literature data) between the contacting and the detecting, to give enough time to the cell to release the nucleic acid.

Additional biological events/confounding variables addressed by the same-sample herein described comprise cell growth, batch effects, as well as variation in time of the biological events/confounding variables herein described. In embodiments wherein time series are performed a proportion of dead and live cells caused by the antibiotic and/or susceptibility of the microorganism to the antibiotic can also be determined minimizing the impact of heteroresistance and/or presence of persister cells as will be understood by a skilled person upon reading of the present disclosure.

All these phenomena can be addressed by determining intra/extra nucleic acid proportion value of samples under experimental conditions selected to test an antibiotic treatment and by comparing determined intra/extra nucleic acid proportion value with a reference value indicative of an intracellular/extracellular nucleic acid proportion in the sample in absence of the tested antibiotic treatment. Accordingly, same-sample methods and systems herein described allow determination of live and death status of cells and/or susceptibility or resistance of a microorganism to one or more antibiotic in absence and without the need of additional detection, in the same sample and/or more remarkably in a separate sample as will be understood by a skilled person upon reading of the present disclosure.

Accordingly, provided herein is an antibiotic susceptibility test (sometimes abbreviated as AST) and related compositions, methods and systems based on nucleic acid detection performed on fractions of a same sample typically from a specimen, which in several embodiments allows determination of antibiotic susceptibility of microorganisms as well as the diagnosis and/or treatment of related infections in individuals based on extracellular/accessible and intracellular/inaccessible concentrations value detected in a same sample typically from a specimen or an isolate.

The term “individual” as used herein when referred to a noun, in the context of treatment refers to a single biological organism, including but not limited to, animals and in particular higher animals and in particular vertebrates such as mammals and in particular human beings.

The word “specimen” as used herein indicates a portion of matter from an environment for use in testing, examination, or study. The environment can comprise individuals and, in particular, human beings. In these instances, a specimen can include a portion of tissues, organs or other biological material from the living being such as urethra, urine, cervix, vagina, rectum, oropharynges, conjunctiva, or any body fluids. A specimen for analysis of living organisms within the specimen, is also indicated as a “biological specimen”. Examples include specimens taken from environments or from patients. A specimen for a medical or veterinary diagnosis, such as from a human patient, from an animal, or from a hospital surface, is also indicated as a “clinical specimen”. Exemplary clinical specimens comprise the following: whole venous and arterial blood, blood plasma, blood serum, dried blood spots, cerebrospinal fluid, lumbar punctures, nasal secretions, sinus washings, tears, corneal scrapings, saliva, sputum or expectorate, bronchoscopy secretions, transtracheal aspirate, endotracheal aspirations, bronchoalveolar lavage, vomit, endoscopic biopsies, colonoscopic biopsies, bile, vaginal fluids and secretions, endometrial fluids and secretions, urethral fluids and secretions, mucosal secretions, synovial fluid, ascitic fluid, peritoneal washes, tympanic membrane aspirate, urine, clean-catch midstream urine, catheterized urine, suprapubic aspirate, kidney stones, prostatic secretions, feces, mucus, pus, wound draining, skin scrapings, skin snips and skin biopsies, hair, nail clippings, cheek tissue, bone marrow biopsy, solid organ biopsies, surgical specimens, solid organ tissue, cadavers, or tumor cells, among others identifiable by a skilled person. Biological specimens can be obtained using sterile techniques or non-sterile techniques, as appropriate for the specimen type, as identifiable by persons skilled in the art. Some clinical specimens can be obtained by contacting a swab with a surface on a human body and removing some material from said surface, examples include throat swab, nasal swab, nasopharyngeal swab, oropharyngeal swab, cheek or buccal swab, urethral swab, vaginal swab, cervical swab, genital swab, anal swab, rectal swab, conjunctival swab, skin swab, and any wound swab. Depending on the type of biological sample and the intended analysis, clinical specimens can be used freshly for sample preparation and analysis or can be fixed using fixative. Preferably, in methods and systems herein described, the specimen contains live target microorganisms.

A specimen in the sense of the disclosure usually represents a single biological datum that the practitioner believes will differ from other datum in connection with a query from a practitioner with respect to the environment. Accordingly, a specimen is a portion of matter that is typically collected at a certain location (e.g. individual, anatomical location, tissue type), at a certain time, and in a certain manner.

In some embodiments herein described a specimen can undergo processing after initial collection from the patient or environment. Example processing techniques that result in a processed specimen include a brief (for example, 3 hour) incubation with media, enrichment of microorganisms from blood, removal of host (for example, human) cells, or isolation to pure culture of the microorganism using standard microbiological techniques. Thus, a specimen inputted to a same-sample AST can be a processed or an unprocessed specimen, and exemplary inputs to same-sample AST include bodily fluids, processed bodily fluids, or a culture of microorganisms obtained from bodily fluid which can be used in a same-sample AST.

The term “isolate” as used herein indicates a portion of matter resulting from a separation of a strain of a microorganism from a natural, usually mixed population of living microbes, as present in a natural or experimental environment, for example in water or soil flora, or from living beings with skin flora, oral flora or gut flora. Isolates can be used in a same-sample AST as will be understood by a skilled person.

The term “sample” as used herein indicates a limited quantity of something that is indicative of a larger quantity of that something, including but not limited to fluids from an isolate or a specimen such as biological environment, cultures, tissues, commercial recombinant proteins, synthetic compounds or portions thereof. In particular, biological sample can comprise one or more cells of any biological lineage, as being representative of the total population of similar cells in the sampled individual. In methods and systems herein described a sample can be split in two or more parts (also indicated as sub-samples, aliquots or sample partitions) each including a smaller quantity of the original sample, and thus providing a sample of the original sample, as will be understood by a skilled person. Partitioning can be performed for example by volumetric transfer of some but not all of the original specimen/sample into a new vessel and by additional approaches identifiable by a skilled person.

In several embodiments of same-sample methods and system herein described, a sample can be the portion of matter which is intended by the practitioner to be analyzed by a given assay. in particular, in some embodiments, a specimen can be split into multiple samples of it, with each sample being inputted into different assays to yield different answers.

The term “partition” or “split” as used herein indicate a physical subdivision of a reference quantity in two or more parts each including a smaller quantity of the original reference quantity. In some embodiments the reference quantity is a specimen in some embodiments the reference quantity is a sample. Accordingly, a specimen can be partitioned or split in a plurality of samples which can then be used for different assay. Additionally, a sample can be split in two or more parts (also indicated as sub-samples, aliquots, or sample partitions) each including a smaller quantity of the original sample, and thus providing a sample of the original sample which can be used to run an assay for example in a digital setting and/or under different experimental conditions in a multiple detection, as will be understood by a skilled person.

In particular, in some exemplary embodiments, one can create multiple antibiotic exposures from the same sample by partitioning the specimen to provide a plurality of sample partitions and performing the sample-sample AST on each of the plurality of sample partitions. In those embodiments one can test multiple antibiotics, multiple antibiotic concentrations, multiple dilutions of the same sample and in general multiple experimental conditions as will be understood by a skilled person upon reading of the present disclosure.

In accordance with embodiments herein described an “experimental condition” an experimental procedure selected based on is a specific choice of an independent variable that is manipulated by the researcher in order to assess the effect on a dependent variable. In particular, in same-sample AST since the rapid changes in the accessibility of nucleic acid to detection reagents are consequent to changes in the continuity of the cell envelope, the main independent variable is addition of an antibiotic at a specific concentration and the main dependent variable is the lysis of the cell.

The wording “lysis,” “lyse,” and “lysing” as used herein indicates disruption of the cell membranes and release of intracellular contents which results in death of the cell. As will be understood by a skilled person, cell death or cell viability can be measured according to one or more measurement methods such as serial dilution on solid growth media to quantify CFU/mL most probable number (MPN) assays, LIVE/DEAD flow cytometry (such as kits available through ThermoFisher scientific), Live/Dead viability staining assays cytometry (such as kits available through ThermoFisher scientific), and automated cell counters (such as the QUANTOM Tx Microbial Cell Counter from Logos Biosystems), metabolic assays and metabolic stains and additional methods identifiable by a skilled person.

A skilled person will understand that cells of different organisms can undergo lysis under different conditions, and that lysis conditions for mammalian cells can be different that lysis conditions of the microorganism cells. Accordingly, a treatment directed to lyse one or more cell in a sample can be set up based on the type of cells targeted (e.g., bacterial or mammalian) and the composition of the reference mixture as well as reaction conditions such as pH temperature and osmolarity of the reaction mixture. Lysis in the sense of the disclosure can occur by mechanisms including natural cell death, as well lytic agents produced by cells or added exogenously, or environmental stresses.

The term “antibiotic” sometimes abbreviated as ABX, as used herein refers to a type of antimicrobial used in the treatment and prevention of bacterial infection. Some antibiotics can either kill or inhibit the growth of bacteria. Others can be effective against fungi and protozoans.

Accordingly, the term “antibiotic” in the sense of the present disclosure is used interchangeable with the term “antimicrobial” and can be used to refer to any substance used against microorganisms. Antibiotics are classified based on their mechanism of action, chemical structure, or spectrum of activity. Most antibiotics target bacterial functions or growth processes. Antibiotics having bactericidal activities target the bacterial cell wall, such as penicillin and cephalosporins, or target the cell membrane, such as polymyxins, or interfere with essential bacterial enzymes, such as rifamycins, lipiarmycins, quinolones and sulfonamides. Antibiotics having bacteriostatic properties target protein synthesis, such as macrolides, lincosamides and tetracyclines. Antibiotics can be further categorized based on their target specificity. “Narrow-spectrum” antibacterial antibiotics target specific types of bacteria, such as Gram-negative or Gram-positive bacteria or a specific genus of bacteria. “Broad-spectrum” antibiotics affect a wide range of bacteria. Antibiotics can also be used in combinations with each other or with adjuvant substances (such as cilastatin or beta-lactamase inhibitors) that enhance their antimicrobial activity. These combinations are often approved by the Food and Drug Administration as distinct drug names as will be understood by a skilled person.

In a same-sample AST can be performed with additional experimental conditions and in particular with independent variables additional to the presence of antibiotic are typically antibiotic related such as timing of exposure and antibiotic concentrations as well as other variables identifiable by a skilled person. Additional dependent variables are typically lysis related such as rate of lysis, probability of lysis and additional dependent variables identifiable by a skilled person.

In general, in embodiments of accessibility AST, one of the antibiotic exposures performed is intended to examine the independent variable of a non-zero concentration of an antibiotic of interest. This antibiotic exposure is called a “test condition”. If one creates antibiotic exposures for multiple non-zero concentrations, possibly of multiple antibiotics, then all are considered test conditions. The results of the test condition (detected nucleic acid concentration values and/or related intra/extra proportion value) are compared with a “reference value” which is a value indicative of results of the experiments in the sample in absence of the independent variable of the antibiotic treatment. Specific examples of reference values comprise reference conditions and thresholds.

In particular, reference conditions are experimental conditions providing a standard for comparison against an antibiotic treated sample where the factor being tested (here antibiotic treatment) is applied during a testing procedure. For example, an antibiotic exposure can be performed in which the independent variable of no antibiotic was included. Such conditions are called “control conditions”. Each control condition corresponds to one or more test conditions such that the only intentional and/or relevant difference between the control condition and the corresponding test conditions is the absence of antibiotic. Control conditions are a specific example of “reference conditions”. Additional reference conditions can be provided by conditions where other differences/independent variables are intentionally included with respect to the test condition in alternative or in addition to the antibiotic concentration, which can be antibiotic related (such as timing of antibiotic exposure) and/or related to other features of the experiments (such as number of cells of a sample).

“Thresholds” in the sense of the disclosure are reference value derived from experiments and/or literature search to account for background events of the sample affecting intracellular and/or extracellular nucleic acid concentration of a same sample unrelated to antibiotic susceptibility as will be also understood by a skilled person.

In embodiments herein described a same-sample intra/extra proportion value determined under test conditions can be compared with a reference value for determination of AST.

In particular, in accordance with embodiments herein described, wherein same-sample AST methods and systems are performed on sample partitions, an experimental condition or condition applies to a grouping of one or more partitions wherein a same independent variable is modified to determine a same dependent variable depending on the practitioner's query of the AST. For example, a query can be “what is the rate of lysis of this patient's bacteria (dependent variable) when the concentration of ceftriaxone is 2.0 μg/mL (independent variable)”, and all sample partitions that contain 2.0 μg/mL of ceftriaxone used to answer that query would constitute one test condition.

Accordingly, embodiments herein described, wherein same-sample AST methods and systems are performed on partitions, the use of partitions allows multiplexing test conditions and/or reference conditions which can then be used alone or in various combination with thresholds can be used for the AST as will be understood by a skilled person upon reading of the disclosure. Multiplexed experimental conditions comprise testing multiple antibiotics or, multiple antibiotic concentrations, multiple dilutions of the same sample, multiple timing of exposure, multiple number of cells, as well as multiple additional experimental conditions such as multiple reference conditions as will be understood by a skilled person.

In preferred embodiments, this method is used to analyze susceptibility and resistant antibiotics that directly or indirectly interact with cell envelope, structure and function, and integrity. Thus, use of this invention is applicable to any pairing of antibiotic and microorganism in which the antibiotic is expected to cause an increased amount, rate, proportion, or probability of lysis of a susceptible strain of microorganism versus the amount, rate, proportion, or probability of lysis of a resistant strain of the same microorganism.

Exemplary antibiotics that cause lysis in all affected microorganisms include the beta-lactam antibiotics. The beta-lactam antibiotics comprise a group of antibiotic agents that contain a beta-lactam ring in their molecular structures. The beta-lactam antibiotics include penicillin derivatives (penams), cephalosporins (cephems), monobactams, and carbapenems. Penams include narrow-spectrum penams such as, benzathine penicillin (benzathine & benzylpenicillin), benzylpenicillin (penicillin G), phenoxymethylpenicillin (penicillin V), Procaine penicillin (procaine & benzylpenicillin), and Pheneticillin. Broad spectrum penams include amoxicillin and ampicillin. Extended spectrum penems include mecillinam, nafcillin, oxacillin, dicloxacillin, carboxypenicillins (including carbenicillin and ticarcillin), and ueidopenicillins (including azlocillin, mezlocillin, and piperacillin). Cephems include first, second, third, fourth, and fifth generation cephalosporins; including cefazolin, cephalexin, cephalosporin C, cephalothin, cefaclor, cefamandole, cefuroxime, cefotetan, cefoxitin, cefixime, cefdinir, cefoperazone, cefotaxime, cefpodoxime, ceftazidime, ceftriaxone, cefepime, cefpirome, and ceftaroline. Carbapenems include biapenem, doripenem, ertapenem, faropenem, imipenem, meropenem, panipenem, razupenem, tebipenem, and thienamycin. Monobactams include aztreonam, tigemonam, nocardicin A, and tabtoxinine b-lactam. Exemplary combinations of antibiotics and adjuvant substances include ampicillin/sulbactam, amoxicillin/clavulanate (clavulanate is also known as clavulanic acid), ticarcillin/clavulanate, piperacillin/tazobactam, ceftazidime/avibactam, ceftazidime/clavulanate, ceftolozane/tazobactam, cefotaxime/clavulanate, imipenem/cilastatin, and meropenem/vaborbactam.

Other antibiotics that can impact the cell envelope directly or indirectly include polymixin B, colistin, depolarizing antibiotics such as daptomycin, antibiotics that hydrolyze NAM-NAG, tyrothricin (Gramicidin or Tyrocidine), isoniazid, and teixobactin. Antibiotics that inhibit peptidoglycan chain elongation including vancomycin (Oritavancin Telavancin), teicoplanin (Dalbavancin), and ramoplanin. Antibiotics that inhibit peptidoglycan subunit synthesis and transport include NAM synthesis inhibition (fosfomycin), DADAL/AR inhibitors (Cycloserine), and bactoprenol inhibitors (bacitracin). The three classes of antibiotics just mentioned are all expected to induce some amount of cell lysis in all affected cells.

The same-sample AST methods in this disclosure can be applied to all combinations of an antimicrobial and a target microorganism, so long as the antimicrobial is known to cause cell lysis in that target microorganism. The antibiotics can cause cell death by cell lysis, or cell lysis can be a highly frequent consequence of other mechanisms of antibiotic action. Examples of such antimicrobials currently in clinical use include the beta-lactam antibiotics (the penicillins, cephalosporins, monobactams, and carbapenems), daptomycin, vancomycin, streptogramins, azole antifungals, allylamine antifungals, echinocandins, and polyene antifungals. Example target microorganisms include all peptidoglycan-producing bacteria (Gram-positive and Gram-negative bacteria), unicellular fungi, and unicellular protozoan parasites. The majority of antimicrobials currently in clinical use are small chemical compounds, but susceptibility to other types of antimicrobials, such as macromolecular (e.g., antimicrobial peptides and proteins), nanoparticle-based, or organismal (e.g. bacteriophages, predatory bacteria) antimicrobial agents can also be measured by our method, so long as the antimicrobial agent causes cell lysis in the target microorganism.

Some antibiotics can cause cell lysis even though their target molecule or cellular process is not traditionally considered part of the cell wall or the cell envelope. So long as cell lysis of a microorganism is expected to be caused by a particular antibiotic, then same-sample accessibility AST can be used to assess that microorganism's susceptibility to that particular antibiotic. For example, cells of Neisseria gonorrhoeae may undergo autolysis, a biologically driven cell lysis, when they are stressed. Fluoroquinolone antibiotics cause DNA strand breakage in Neisseria gonorrhoeae, and the subsequent detection of DNA damage by intracellular signaling pathways triggers autolysis. Thus, same-sample accessibility AST can be used to assess fluoroquinolone activity in Neisseria gonorrhoeae even though fluoroquinolones are not considered cell wall-targeting antibiotics.

The wording “antibiotic susceptibility” or “antibiotic sensitivity” as used herein indicates the susceptibility of bacteria to antibiotics and the antibiotic susceptibility can vary within a species. Antibiotic susceptibility testing (AST) can be carried out to predict the clinical response to treatment and guide the selection of antibiotics as will be understood by a person skilled in the art. In some embodiments, AST categorizes organisms as susceptible, resistant, or intermediate to a certain antibiotic.

Microorganisms can be classified as susceptible (sensitive), intermediate or resistant based on breakpoint minimum inhibitory concentration (MIC) values that are arbitrarily defined and reflect the achievable levels of the antibiotic, the distribution of MICs for the organism and their correlation with clinical outcome. MIC value of a microorganism is the lowest concentration of an antibiotic that will inhibit its growth. Methods that can be used to measure the MIC of a microorganism comprise broth macrodilution, broth microdilution, agar dilution and gradient diffusion (the ‘E test’), where twofold serial dilutions of antibiotic are incorporated into tubes of broth, agar plates or on a paper strip, respectively, as will be understood by a person skilled in the art. The disk diffusion method defines an organism as susceptible or resistant based on the extent of its growth around an antibiotic-containing disk. MIC values are influenced by several laboratory factors. Laboratories follow standard for parameters such as incubation temperature, incubation environment, growth media, as well as inoculum and quality control parameters. In the U.S., standards for performing AST as well as breakpoint MIC values for various bacteria can be found in Clinical & Laboratory Standards Institute (CLSI) publications [4] as will be understood by the skilled person. In Europe, standards for performing AST as well as breakpoint MIC values for bacteria can be found in European Committee on Antimicrobial Susceptibility Testing (EUCAST, see www.eucast.org/clinical_breakpoints/ at the time of filing of the instant disclosure) [5] as will be understood by the skilled person.

The term “microorganism”, or “microbe” as used herein indicates a microscopic living organism, which may exist in its single-celled form or in a colony of cells, such as prokaryotes and in particular bacteria, and including fungi (yeast and molds), and protozoal parasites. Microorganisms include human and animal pathogens. Microorganisms can comprise one or more prokaryotes or individual genera or species of prokaryotes.

The term “prokaryotic” is used herein interchangeably with the terms “cell” and refers to a microbial species which contains no nucleus or other membrane-bound organelles in the cell. Exemplary prokaryotic cells include bacteria and archaea.

The term “bacteria” or “bacterial cell”, used herein interchangeably with the term “cell” when discussing bacteria indicates a large domain of prokaryotic microorganisms. Typically a few micrometers in length, bacteria have a number of shapes, ranging from spheres to rods and spirals, and are present in most habitats on Earth, such as terrestrial habitats like deserts, tundra, Arctic and Antarctic deserts, forests, savannah, chaparral, shrublands, grasslands, mountains, plains, caves, islands, and the soil, detritus, and sediments present in said terrestrial habitats; freshwater habitats such as streams, springs, rivers, lakes, ponds, ephemeral pools, marshes, salt marshes, bogs, peat bogs, underground rivers and lakes, geothermal hot springs, sub-glacial lakes, and wetlands; marine habitats such as ocean water, marine detritus and sediments, flotsam and insoluble particles, geothermal vents and reefs; man-made habitats such as sites of human habitation, human dwellings, man-made buildings and parts of human-made structures, plumbing systems, sewage systems, water towers, cooling towers, cooling systems, air-conditioning systems, water systems, farms, agricultural fields, ranchlands, livestock feedlots, hospitals, outpatient clinics, health-care facilities, operating rooms, hospital equipment, long-term care facilities, nursing homes, hospice care, clinical laboratories, research laboratories, waste, landfills, radioactive waste; and the deep portions of Earth's crust, as well as in symbiotic and parasitic relationships with plants, animals, fungi, algae, humans, livestock, and other macroscopic life forms. Bacteria in the sense of the disclosure refers to several prokaryotic microbial species which comprise Gram-negative bacteria, Gram-positive bacteria, Proteobacteria, Cyanobacteria, Spirochetes and related species, Planctomyces, Bacteroides, Flavobacteria, Chlamydia, Green sulfur bacteria, Green non-sulfur bacteria including anaerobic phototrophs, Radioresistant micrococci and related species, Thermotoga and Thermosipho thermophiles as would be understood by a skilled person. Taxonomic names of bacteria that have been accepted as valid by the International Committee of Systematic Bacteriology are published in issues of the International Journal of Systematic and Evolutionary Microbiology. More specifically, the wording “Gram positive bacteria” refers to cocci, nonsporulating rods and sporulating rods that stain positive on Gram stain, such as, for example, Actinomyces, Bacillus, Clostridium, Corynebacterium, Cutibacterium (previously Propionibacterium), Erysipelothrix, Lactobacillus, Listeria, Mycobacterium, Nocardia, Staphylococcus, Streptococcus, Enterococcus, Peptostreptococcus, and Streptomyces. Bacteria in the sense of the disclosure refers also to the species within the genera Clostridium, Sarcina, Lachnospira, Peptostreptococcus, Peptoniphilus, Helcococcus, Eubacterium, Peptococcus, Acidaminococcus, Veillonella, Mycoplasma, Ureaplasma, Erysipelothrix, Holdemania, Bacillus, Amphibacillus, Exiguobacterium, Gracilibacillus, Halobacillus, Saccharococcus, Salibacillus, Virgibacillus, Planococcus, Kurthia, Caryophanon, Listeria, Brochothrix, Staphylococcus, Gemella, Macrococcus, Salinococcus, Sporolactobacillus, Marinococcus, Paenibacillus, Aneurinibacillus, Brevibacillus, Alicyclobacillus, Lactobacillus, Pediococus, Aerococcus, Abiotrophia, Dolosicoccus, Eremococcus, Facklamia, Globicatella, Ignavigranum, Carnobacterium, Alloiococcus, Dolosigranulum, Enterococcus, Melissococcus, Tetragenococcus, Vagococcus, Leuconostoc, Oenococcus, Weissella, Streptococcus, Lactococcus, Actinomyces, Arachnia, Actinobaculum, Arcanobacterium, Mobiluncus, Micrococcus, Arthrobacter, Kocuria, Nesterenkonia, Rothia, Stomatococcus, Brevibacterium, Cellulomonas, Oerskovia, Dermabacter, Brachybacterium, Dermatophilus, Dermacoccus, Kytococcus, Sanguibacter, Jonesia, Microbacteirum, Agrococcus, Agromyces, Aureobacterium, Cryobacterium, Corynebacterium, Dietzia, Gordonia, Skermania, Mycobacterium, Nocardia, Rhodococcus, Tsukamurella, Micromonospora, Propioniferax, Nocardioides, Streptomyces, Nocardiopsis, Thermomonospora, Actinomadura, Bifidobacterium, Gardnerella, Turicella, Chlamydia, Chlamydophila, Borrelia, Treponema, Serpulina, Leptospira, Bacteroides, Porphyromonas, Prevotella, Flavobacterium, Elizabethkingia, Bergeyella, Capnocytophaga, Chryseobacterium, Weeksella, Myroides, Tannerella, Sphingobacterium, Flexibacter, Fusobacterium, Streptobacillus, Wolbachia, Bradyrhizobium, Tropheryma, Megasphera, Anaeroglobus.

The term “proteobacteria” as used herein refers to a major phylum of Gram-negative bacteria. Many move about using flagella, but some are nonmotile or rely on bacterial gliding. As understood by skilled persons, taxonomic classification as proteobacteria is determined primarily in terms of ribosomal RNA (rRNA) sequences. The Proteobacteria are divided into six classes, referred to by the Greek letters alpha through epsilon and the Acidithiobacillia and Oligoflexia, including the alphaproteobacteria, betaproteobacteria and gammaproteobacteria as will be understood by a skilled person. Proteobacteria comprise the following genera: in the Alphaproteobacteria, Rickettsia, Ehrlichia, Anaplasma, Sphingomonas, Brevundimonas, Agrobacterium, Bartonella, Brucella, Ochrobactrum, Afipia, Methylobacterium, and Roseomonas; in the Betaproteobacteria, Burkholderia, Ralsonia, Alcaligenes, Achromobacter, Chromobacterium, Bordetella, Taylorella, Comamonas, Neisseria, Alysiella, Eikenella, Kingella, and Spirillum; in the Gammaproteobacteria, Xanthomonas, Stenotrophomonas, Cardiobacterium, Suttonella, Francisella, Legionella, Coxiella, Ricketsiella, Pseudomonas, Chryseomonas, Flavimonas, Oligella, Moraxella (Branhamella), Acinetobacter, Psychrobacter, Shewanella, Vibrio, Photobacterium, Aeromonas, Succinivibrio, Anaerobiospirillum, Ruminobacter, Succinimonas, Enterobacter, Brenneria, Budvicia, Buttiauxella, Calymmatobacterium, Cedeceae, Citrobacter, Edwardsiella, Erwinia, Escherichia, Ewingella, Hafnia, Klebsiella, Kluyvera, Leclercia, Leminorella, Moellerella, Morganella, Obesumbacterium, Pantoea, Plesiomonas, Proteus, Providencia, Rahnella, Salmonella, Serratia, Shigella, Tatumella, Trabulsiella, Yersinia, Yokenella, Pasteurella, Actinobacillus (Aggregatibacter), Haemophilus, and Mannheimia; in the Deltaproteobacteria, Desulfovibrio and Biophila; in the Epsilonproteobacteria, Campylobacter, Arcobacter, Helicobacter, and Wolinella [6]. The Proteobacteria also comprise the species which are classified within the aforementioned genera. Within the Proteobacteria are the species Neisseria gonorrhoeae and Neisseria meningitidis within the class Betaproteobacteria, the order Neisseriales the family Neisseriaceae, and the genus Neisseria. It will be understood by the skilled practitioner that the classification and nomenclature of formal bacterial species is subject to revision as new scientific knowledge is discovered. Changes in name are performed according to rules in the International Code of Nomenclature of Bacteria [7], and future name changes can be found by consulting the International Journal of Systematic and Evolutionary Microbiology.

The term “Enterobacteriaceae” in the sense of the disclosure refers to members of the Proteobacteria that fall within the family Enterobacteriaceae, Class Gammaproteobacteria, as defined by the International Committee of Systematic Bacteriology. These bacteria are Gram-negative rods that can inhabit the gastrointestinal tracts of animals as well as environmental surfaces. Many species are pathogenic in humans and other animals. Many species are commensals that become pathogenic when their hosts immune barriers are breached. Enterobacteriaceae are frequently encountered in clinical specimens [6]. Enterobacteriaceae include the following taxa and clinical entities: Escherichia coli (E. coli), uropathogenic E. coli, enterotoxigenic E. coli, enteroaggregative E. coli, enteropathogenic E. coli, enteroinvasive E. coli, enterohemorrhagic E. coli, Shiga toxin-producing E. coli, diffusely adherent E. coli, Klebsiella pneumoniae subsp. ozaenae, Klebsiella pneumoniae subsp. pneumoniae, Klebsiella pneumoniae subsp. rhinoscleromatis, Klebsiella oxytoca, Enterobacter aerogenes, Enterobacter cloacae, Citrobacter freundii, Citrobacter koseri (Citrobacter diversus), Salmonella enterica subsp. enterica and its serovars, Salmonella enterica Typhi, Salmonella enterica Paratyphi, Salmonella bongori, Shigella dysenteria, Shigella flexneri, Shigella boydii, Shigella sonnei, Proteus mirabilis, Proteus vulgaris, Serratia marcescens, Yersinia pestis, Yersinia enterocolitica, Yersinia pseudotuberculosis, Providencia stuartii, Edwardsiella hoshinae, Raoultella ornithinolytica, Raoultella planticola, Raoultella terrigena, Arizona hinshawii, Budvicia aquatica, Buttiauxella agrestis, Buttiauxella brennerae, Buttiauxella ferragutiae, Buttiauxella gaviniae, Buttiauxella izardii, Buttiauxella noackiae, Buttiauxella warmboldiae, Cedecea davisae, Cedecea lapagei, Cedecea neteri, Cedecea species 3, Cedecea species 5, Citrobacter amalonaticus, Citrobacter braakii, Citrobacter farmer, Citrobacter gillenii, Citrobacter murliniae, Citrobacter rodentium, Citrobacter sedlakii, Citrobacter werkmanii, Citrobacter youngae, Edwardsiella ictaluri, Edwardsiella tarda, Edwardsiella tarda biogroup 1, Enterobacter amnigenus, Enterobacter asburiae, Enterobacter cancero genus (Enterobacter taylorae), Enterobacter cowanii, Enterobacter dissolvens, Enterobacter gergoviae, Enterobacter hormaechei, Enterobacter intermedius, Enterobacter kobei, Enterobacter nimipressuralis, Enterobacter pyrinus, Enterobacter sakazakii, Erwinia spp., Ewingella americana, Hafnia alvei, Kluyvera ascorbate, Kuyvera cryocrescens, Kluyvera georgiana, Leclercia adecarboxylata, Leminorella grimontii, richardii, Moellerella wisconsensis, Morganella morganii, Obesumbacterium proteus, Pantoea agglomerans, Pantoea dispersa, Photorhabdus luminescens, Photorhabdus asymbiotica, Pragia fontium, Proteus hauseri, Proteus myxofaciens, Proteus penneri, Providencia alcalifaciens, Providencia heimbachae, Providencia rettgeri, Providencia rustigianii, Rahnella aquatilis, Serratia entomophilia, Serratia ficaria, “Serratia fonticola”, Serratia liquifaciens group, Serratia odorifera, Serratia plymuthica, Serratia rubidea, Tatumella ptyseos, Trabulsiella guamensis, Xenorhabdus nematophilus, Yersinia aldovae, Yersinia bercoviera, Yersinia frederiksenii, Yersinia intermedia, Yersinia kristensenii, Yersinia mollaretii, Yersinia rohdei, “Yersinia ruckeri”, Yokenella regensburgei.

The term “carbapenem-resistant Enterobacteriaceae” in the sense of this disclosure refers to any member of the family Enterobacteriaceae, defined earlier, that exhibit resistance to at least one member of the carbapenem class of antibiotics, defined earlier. The term carbapenem-resistant Enterobacteriaceae can be abbreviated as “CRE”. CRE isolates are frequently resistant to classes of beta-lactam antibiotics besides the carbapenems, namely the penicillins, cephalosporins, and monobactams. CRE isolates also frequently carry resistance toward other classes of antibiotics. Some CRE isolates are susceptible very few antibiotics, and some CRE isolates have been found to be resistant to all antibiotics available for use in humans in the USA or Europe. CRE achieve antibiotic resistance through a variety of resistance mechanisms, including the expression of enzymes that degrade beta-lactam antibiotics (carbapenemases, extended-spectrum beta-lactamases, and beta-lactamases), alterations in expression of their porin genes, and by unknown mechanisms. CRE prevalence has increased worldwide and in the USA in the past three decades. CRE cause a significant fraction of health-care associated infections. CRE infections have an estimated 50% mortality rate in the USA [8].

In particular, in AST methods herein described, antibiotic susceptibility is determined based on detected nucleic acid concentration in extracellular and cellular fraction to following an accessibility approach applied to a same sample.

Calculating the probability of susceptibility is a primary purpose of the accessibility AST methods described herein. Accessibility AST methods measure susceptibility of microorganisms to antimicrobials which cause cell lysis, and they do so by detecting the extracellular or intracellular location (which determines the “accessibility”) of nucleic acids produced by the cells of interest.

One can define the “intracellular subset”, “cellular subset”, “intracellular fraction”, or “cellular fraction” of a sample's nucleic acids to be all nucleic acids which are contained within any of the intact cells present in the sample at a given time. If there are multiple cells, all of their nucleic acids are in the intracellular subset. If there are no intact cells, no nucleic acids are in the intracellular subset. Likewise, one can define the “extracellular subset” or “extracellular fraction” of a sample's nucleic acids to be all nucleic acids that are not contained within any of the intact cells present in the sample at a given time. If one or multiple cells lyse, all of the nucleic acids formerly contained within them now reside in the extracellular subset. Nucleic acids in our sample either reside in the intracellular subset or in the extracellular subset.

To begin a same-sample AST protocol, a sample (such as clinical sample derived by partitioning a specimen from an individual) is typically mixed with growth media and a known amount of antibiotic, at minimum. The contacting of a volume of growth media, bacteria, and antibiotic constitutes an “antibiotic exposure.” The exact volumes making up the antibiotic exposure can vary. The bacteria and antibiotics remain in contact for a chosen duration of time, during which some bacteria lyse if the bacteria are susceptible to the antibiotic.

In some embodiments, of the methods of the instant disclosure, the time period of contacting the sample with an antibiotic can be up to 5 minutes, up to 10 minutes, up to 15 minutes, up to 20 minutes, 25 minutes, 30 minutes, up to 45 minutes, up to 60 up to 90 up to 120 up to 360 or higher, inclusive of any value therebetween or fraction thereof. In some embodiments of the methods of the instant disclosure, the time period of contacting the sample with an antibiotic is shorter than the doubling time of the target organism. For example, the time of contacting could be less than 1× doubling time, less than 0.75× doubling time, less than 0.5 doubling time, less than 0.35 doubling time, less than 0.25 doubling time, less than 0.2 doubling time, less than 0.15 doubling time, less than 0.1 doubling time, less than 0.075 doubling time, less than 0.05 doubling time. In one example of this disclosure, (see e.g. Example 7), antibiotic exposure times greater than the doubling time, or many doubling times can be used, and longer exposure times correlates with a reduced probability of a lag in antibiotic killing or other false positive result for susceptibility preferably for a time up to 120 minutes.

Typically, microbiological medias used in the methods support metabolism of the microorganism and do not interfere significantly with the antibiotic action. The terms “microbiological media”, “growth media”, and “microbiological growth media” are all used interchangeably herein to refer to substances or mixtures of substances in a liquid or solid form that form a suitable habitat for microorganism growth. Different microbiological medias are used or are designed to serve different functions, both clinical and non-clinical, including collection/transport, selective cultivation and isolation, differentiation, cultivation, and maintenance of cultures. Many medias can serve multiple purposes, either as a preferred option by current practitioners or as a less optimal but still suitable choice. Some media are used to isolate bacteria from a clinical specimen that may contain many types of bacteria, most of which are not the causative pathogen and could be contamination introduced merely during specimen collection. Some medias are used to detect and/or differentiate certain taxa, such as by their metabolic abilities, and often are used as an aid in identifying bacterial taxa, including for clinical purposes. Some medias are used to cultivate bacteria at high growth rates and fast division times (e.g., for biotechnology and manufacturing).

Additionally, some medias are used to support growth of microorganisms during phenotypic AST. These media are typically chosen for enabling relatively fast growth rates in diverse pathogenic microorganisms and with minimal antibiotic-specific in vitro artifacts. Examples of an antibiotic-specific artifact phenomenon is the variation of aminoglycoside activity with cation composition, the binding of antibiotic to media components like proteins, or the reaction or degradation of the antibiotic with media compounds. Typically the media can be non-viscous liquid in particular in embodiments where separation is performed by filtration. A list of popular growth medias for the handling of clinical specimens during the diagnostic workflow can be found in the clinical microbiology literature, such as the American Society for Microbiology's Manual of Clinical Microbiology [9]. The compositions of these growth medias can also be found in the academic literature or in product specification manuals published by major media manufacturers, except that some commercial medias have proprietary compositions. Common ingredients include yeast extract, beef extract, casein digest, soybean digest (soy trypticase), peptone, tryptone, other vegetable or animal tissue digests, casamino acids (amino acids), animal blood, animal plasma, animal serum, albumin, gelatin, starch, sugars and similar compounds (glucose, pyruvate, succinate), vitamins, hemin, and various salts. Many microbiological media can be used in a liquid broth form or as a solid, gelatinous medium. The most common method for producing a solid media is to add agar polymer to the media composition. Many medias contain additives with specific functions ranging from selection to differentiation to protection of certain bacterial species.

Many medias designed for isolating a target taxon, suspected by clinicians and other assay users to be present in the clinical specimen based on clinical history and presentation, contain antimicrobials or other substances that preferentially inhibit the growth of other contaminating organisms. Some antimicrobials are not necessary to include in the media once a pure culture has been obtained. Some microbiological medias contain colorimetric indicators to detect general or taxon-specific bacterial metabolic reactions. Many automated blood culture systems or bottles contain and employ custom medias [10]. Some commonly-used microbiological media for cultivation of a broader range of taxa include brain heart infusion (BHI), Barbour-Stoenner-Kelly medium, Brucella agar, Brucella agar base with blood and selective supplement, Brucella blood culture broth, Brucella broth (Brucella Albimi broth), CDC anaerobe 5% sheep blood agar, chocolate agar, MacConkey agar, malt agar, chopped meat broth, cooked meat medium, Columbia broth, Columbia blood agar, cystine tryptic agar, Eugonic agar, heart infusion agar, liver infusion agar, tryptic soy blood agar (tryptose blood agar, TSA blood agar), trypticase soy agar (tryptic soy agar, soybean-casein digest medium), trypticase soy agar with sheep's blood, trypticase soy broth with or without sucrose (also known as tryptic soy broth), soybean-casein digest broth with or without resins, soybean-casein digest/Columbia broth, soybean-casein digest thioglycollate broth, Schaedler broth, VersaTREK® REDOX 1 aerobic media (Thermo Fisher Scientific), VersaTREK® REDOX 1 anaerobic media (Thermo Fisher Scientific), VersaTREK® REDOX 2 aerobic media (Thermo Fisher Scientific), VersaTREK® REDOX 2 anaerobic media (Thermo Fisher Scientific), BD BACTEC® Standard aerobic media (BD, formerly Becton Dickinson), BD BACTEC® Standard anaerobic media (BD), BD BACTEC® Plus aerobic media (BD), BD BACTEC® Plus anaerobic media (BD), BD BACTEC® Lytic anaerobic media (BD), BD BACTEC® Plus aerobic media (BD), BD BACTEC® Mycosis-IC/F® lytic medium, BD BACTEC® Myco/F® lytic medium, bioMerieux BacT/Alert® standard aerobic culture media, bioMerieux BacT/Alert® standard anaerobic culture media, bioMerieux Fastidious Antimicrobial Neutralization Plus® aerobic media, bioMerieux Fastidious Antimicrobial Neutralization Plus® anaerobic media, and bioMerieux Fastidious Antimicrobial Neutralization Plus® pediatric media. Many other medias for cultivation of specific taxa, fastidious taxa, or slow-growing taxa are reported in the literature as well [9], [10] and are thus identifiable by a skilled person.

It is possible to perform phenotypic AST, including the methods herein, with any growth media that enables cells to be viable and to replicate. It is preferred however, that the media maintains fast growth of one or more microorganisms of interest and that the media components do not significantly alter the efficacy of the antimicrobial. A number of standards-setting organizations such as the Clinical and Laboratory Standards Institute (CLSI)[4], The European Committee on Antimicrobial Susceptibility Testing (EUCAST)[11], the British Society for Antimicrobial Chemotherapy (BSAC), and the International Organization for Standardization (ISO, Geneva, Switzerland) have published widely-accepted guidelines on the recommended standard media to choose for growth-based phenotypic AST for almost every human pathogen for which growth-based phenotypic AST is performed for clinical use today. Cation-adjusted Mueller-Hinton broth is the media recommended by most standards-setting organizations for most microorganisms [4], [9]. Other media include Haemophilus test medium (for H. influenzae); Middlebrook 7H8, 7H9, and 7H10 medias (for Mycobacterium spp.); Mueller-Hinton agar; Mueller-Hinton chocolate agar (for Neisseria); Mueller-Hinton II agar (for H. influenzae, Streptococcus pneumoniae, and for the Kirby-Bauer test); Wilkins-Chalgren anaerobe broth (for anaerobic bacteria) and others identifiable to a person skilled in the art. Additional media can found in Clinical & Laboratory Standards Institute (CLSI), European Committee on Antimicrobial Susceptibility Testing (EUCAST) and other public resources as will be understood by a skilled person.

In preferred embodiments, the methods herein described before proceeding to contacting the sample with an antibiotic performing an antibiotic exposure, same-sample AST methods herein described further comprises an enriching step. Enriching a sample with the target microorganisms can be performed between sample collection from a specimen (and optionally elution from a collection tool such as a swab) and antibiotic exposure.

In particular enriching a sample with target microorganisms can be performed by capturing the target microorganism using a solid support (e.g. a membrane, a filtration membrane, an affinity membrane, an affinity column) or a suspension of a solid reagent (e.g. microspheres, beads). Capture of a target microorganism can improve the assay and the response to antibiotic. Capture can be used to enrich/concentrate low-concentration samples. Capture followed by washing can be used to remove inhibitors or components that may interfere with the method described here. Capture followed by washing may be used to remove inhibitors of nucleic acid amplification or inhibitors of other quantitative detection assays. Enrichment can also be performed using lysis-filtration techniques to lyse host cells and dissolve protein and/or salt precipitates while maintaining bacterial cell integrity then capturing target bacteria on filters (e.g. mixed cellulose ester membranes, polypropylene and polysulfone membranes). Enrichment can also be performed by binding target bacteria to membranes of microspheres, optionally coated with an affinity reagent (e.g. an antibody, an aptamer) specific to the target bacteria's cell envelope. When microspheres or beads are used for capture, they can be filtered, centrifuged, or collected using a magnet to enrich bacteria. AST in the format described here can then be performed directly on captured bacteria, or the bacteria can be released before performing the method.

In same-sample AST methods herein described, the sample is contacted with an antibiotic in an antibiotic exposure. An antibiotic exposure can be performed in the presence of ambient levels of oxygen (aerobic) without the presence of oxygen (anaerobic), or in the presence of other controlled levels of oxygen, such as to create microoxic conditions. Levels of other gases, such as CO₂, can be controlled.

In some embodiments, the antibiotic exposure can be performed in combination with an enhancement treatment.

An “enhancement treatment” or an “enhancing treatment” in the sense of the disclosure refers to a concurrent combined or sequential administration of lytic agents and or stressors that results in in lysis of <90%, preferably ≤60%, more preferably ≤30%, and even more preferable ≤35%, more preferably ≤15% most preferably ≤5% target cells in a control sample [12]. An enhancing treatment used together with the antibiotic exposure in the antibiotic treated sample is directed to preserve the viability of at least 10% of microorganism in a sample.

A “lytic agent” in the sense of the disclosure indicates any substance or energy that that results in lysis of a target cell if applied to the target.

Lytic agents in the sense of the disclosure comprise chemical lytic agent such as detergents and/or enzyme capable of catalyzing disassembly of cell walls, mechanical methods capable of disrupting the cell wall or membrane such as sonication at Covaris M220 sonication parameters 75 W peak incident power, >15% duty cycle, >200 cycles per burst, and >30 minutes in a volume of 50 uL such as high pH or high temperature (see Examples 3). Examples of chemical lytic agents suitable to perform a lysis of the disclosure Triton X-100, Tween-20, SDS, NP-40, and lysozyme. Examples of mechanical lytic agents suitable to perform a lysis of the disclosure include sonication.

Exemplary enhancing treatment in the sense of the disclosure for most target microorganism comprises pH above optimal physiological conditions for the cell, such as pHs greater or equal to 7.5 and lower than 9, or equal or less than 6.5 and greater than 5 for 30 minutes or less, high temperatures such as >38 C and <80 C for 30 minutes or less depending on the temperature selected, or high or low osmolarity values deviating from the physiological osmolarity by up to 250 mOsmol for 30 minutes or less, in some embodiments applied in a form of osmotic shock, in some embodiments approaching zero osmolarity. A skilled person will be able to identify the correct conditions for a lysis treatment depending on the taxonomy of the target cell. For example, lytic treatment of Gram-positive cells can be performed with additional enzymatic treatment of the cell wall in combination or in parallel with the above listed conditions. Lytic treatment of a Gram-negative like N. gonorrhoeae can be performed by any one of the conditions above.

In some embodiments, sterile techniques can be used to minimize contamination of the samples during antibiotic exposure, including the use of sterile equipment, sterile disposable plasticware, sterile media and antibiotic solutions, and environmental controls, such as HEPA filters and biological safety cabinets (BSCs).

In embodiments of same-sample AST, before antibiotic exposure the sample can optionally be partitioned as will be understood by a skilled person upon reading of the present disclosure. The sample and/or related partitions are then separated in an extracellular and intracellular fraction which are further analyzed according to methods herein described as will be understood by a skilled person.

The wording “separate” or “separation” as used herein indicates an action performed on a sample such that two desired components of the sample (such as nucleic acid and cells of a target microorganism) are no longer able to come into molecular contact. Separation in the sense of the disclosure can be performed mechanically by filtrating and/or centrifugating the sample and recovering a filtrate and a retentate. The filtrate comprises the extracellular nucleic acid of any microorganism present and other compounds and molecules of the sample located outside any cell present therein (extracellular fraction). The retentate comprises the cell of the sample retained in the separation. Filtration and/or centrifugation can be set up to select the microorganism as part of the retentate. For example, in an exemplary embodiment, the separation include filtration through a filter with a pore size (such as 0.2 um) such that cells are removed from surrounding liquid and any components of the surrounding liquid smaller than the pore size of the filter to obtain retention of microorganism cells as will be understood by a skilled person.

Additional methods to perform separation and provide extracellular and cellular fraction of a same sample able to recover all fractions of nucleic acids from a given sample or sample partition are identifiable by a skilled person upon reading of the present disclosure.

In some embodiments, wherein separation comprise filtrating the sample, the separation can be followed by a washing to remove any extracellular nucleic acid from the filter to improve the efficacy of the separation as will be understood by a skilled person upon reading of the present disclosure (see e.g. Examples 1, 2, 3, 6, 7, 8 and 13).

In some embodiments, wherein separation comprise centrifuging the sample, the separation can be followed by a washing to remove any extracellular nucleic acid from the filter to improve the efficacy of the separation as will be understood by a skilled person upon reading of the present disclosure. In some embodiments, the washing can be performed by resuspending the cells pelleted from a first centrifugation in a liquid free of the nucleic acid to be quantified, repeating the centrifugation a second time, and then retaining the pellet from the second centrifugation.

In embodiments wherein separation procedure where intact cells can be recovered from a liquid, washing can be performed by reconstituting the cells in a non-lytic, buffer not lethal for the cells to reconstitute the sample, then repeating the same or another separation technique. As a consequence, a series of washing steps is possible, although fewer washes are preferred to reduce time, reagents, and any loss of intact cells from incomplete separation.

In embodiments of same-sample methods and systems herein described separation of a sample comprising a microorganism thus results in a cellular and extracellular fraction as will be understood by a skilled person which are further subjected to quantitative detection of intracellular nucleic and extracellular nucleic acid respectively.

The terms “detect” or “detection” as used herein indicates the determination of the existence, presence or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “qualitative” when it refers, relates to, or involves identification of a quality or kind of the target or signal in terms of relative abundance to another target or signal, which is not quantified. A detection is “quantitative” when it refers, relates to, or involves the measurement of quantity or amount of the target or signal (also referred as quantitation), which includes but is not limited to any analysis designed to determine the amounts or proportions of the target or signal. A quantitative detection in the sense of the disclosure comprises detection performed semi-quantitatively, above/below a certain amount of nucleic acid molecules as will be understood by a skilled person and/or using semiquantitative real time isothermal amplification methods including real time loop-mediated isothermal amplification (LAMP) (see e.g., semi quantitative real-time PCR). For a given detection method and a given nucleic acid input, the output of quantitative or semiquantitative detection method that can be used to calculate a nucleic acid concentration value or nucleic acid concentration ratio (NACR) is a “concentration parameter”.

In methods herein described where the target nucleic acid comprises DNA and/or RNA, quantitative detection of nucleic acid concentration can be performed with various techniques (commonly in combination with reverse transcription for RNA) such as by RNA-seq, DNA-seq, qPCR, digital PCR, and isothermal techniques such as LAMP or digital isothermal, microarrays signals, Nanostring as well high throughput DNA and RNA sequencing as reads per kilobase per million reads (RPKM) or transcripts per million (TPM) for RNA-seq data and additional nucleic acid quantification techniques identifiable to a skilled person. It will be understood that in such methods quantitative detection of expression of a gene is commonly combined with a reverse transcription step to convert the RNA sequence into a cDNA sequence which can be quantified by methods described herein and/or identifiable by a skilled person. Either sequence-specific or sequence-non-specific primers can be used to initiate reverse transcription of a target gene as will be understood by a skilled person.

In some embodiments where the target nucleic acid comprises RNA, detecting nucleic acid concentrations can be performed at the transcription level by performing RNA-seq and calculating RNA concentration values based on the sequence data.

In some embodiments where the target nucleic acid comprises RNA, the RNA concentration values can be detected and provided as transcripts per million (TPM) as will be understood by a person skilled in the art. In particular, to calculate TPM, read counts are first divided by the length of each gene in kilobases, which gives reads per kilobase (RPK). RPKs for all genes are added and the sum is divided by 1,000,000. This gives the “per million” scaling factor. Finally, the RPK value for each gene is divided by the “per million” scaling factor to give TPM.

In embodiments herein described, detection of intracellular nucleic acid is performed, following a lysis treatment of the retentate to provide a lysate comprising the intracellular nucleic acid of any target microorganisms possibly included in the sample as will be understood by a skilled person.

A “lysis treatment” in the sense of the disclosure is a concurrent combined or sequential administration of lytic agents that results in lysis of ≥90%, preferably ≥95%, more preferably ≥97%, and even more preferable ≥99% target cells in a control sample. Depending on the target organism, a lysis treatment can be obtained by exposing the organisms to high and low extremes incubation condition, which will depend on the type and features of the target cells. Exemplary lysis treatment in the sense of the disclosure for some target microorganism comprises high pH, such as pH values greater than 11 for 30 minutes or more, high temperatures such as >90 C for 10 minutes or more. A skilled person will be able to identify the correct conditions for a lysis treatment depending on the taxonomy of the target cell. For example, lytic treatment of Gram-positive cell can be performed with additional enzymatic treatment of the cell wall in combination or in parallel with the above listed conditions. Lytic treatment of a Gram-negative like N. gonorrhoeae can be performed by any one of the conditions above.

Accordingly, lysis treatment of target microorganism in the sense of the disclosure can be performed using lytic agents at conditions directed to result in the lysis of ≥90% or microorganism in the sample. For example, the ionic detergents such as SDS or BAC at concentrations above their critical micelle concentrations (CMC) and/or sonication at powers greater than (Covaris M220 sonication parameters 75 W peak incident power, >15% duty cycle, >200 cycles per burst, and >30 minutes in a volume of 50 uL) for gram negative organism and at higher powers such as 5×, 10×, 100× the power used for gram-negative organisms. Examples of conditional lytic agents suitable to perform a lysis treatment of the disclosure include pHs greater than 8 (see Example 3) and temperatures greater than 90 C for >1 min.

In some embodiments, lysis treatment of target microorganism in the sense of the disclosure can be performed, for example, with a commercial lysis kit such as that provided by Zymo or Qiagen. For gram-negative microorganisms, such kit can include highly denaturing lysis agents containing guanidinium salts in combination with buffers and enzymes to promote complete disruption of all cell envelope and denaturation of cellular proteins alone or in combination with a stressor. A “stressor” is a reagent of a form of energy that acts synergistically with antibiotic to disrupt cell envelope.

A lysis treatment in the sense of the disclosure typically results in conversion of ≥90%, ≥95%, ≥97%, ≥99% of the total intracellular nucleic acids of the target cell to extracellular nucleic acids of the target cell.

Thus in embodiments of same-sample AST a lysis treatment results in making the inaccessible nucleic acid within the microorganism accessible to detecting reagents. The inclusion of this nucleic acid in a same sample cellular fraction distinct from the same-sample extracellular fraction allows the related identification as inaccessible in an accurate fashion as will be understood by a skilled person upon reading of the disclosure.

In some embodiments of same-sample AST methods, the measurement of nucleic acid concentration is performed after extracting the nucleic acids from the extracellular fraction (filtrate) or from the lysed cellular fraction (lysate) of a same sample. Extraction of a nucleic acids is the processing of a sample by mechanical, chemical, thermal, or electrochemical techniques to render nucleic acids in a state amenable for nucleic acid amplification. Extraction is often one step in a protocol. Extraction can include the lysis of cells to release any intracellular nucleic acids that are not accessible to nucleic acid amplification reagents. Extraction can include the inactivation, destruction, or removal of substances that alter the nucleic acid concentration in ways that obscure the effects of phenomena an experiment wishes to measure, such as the degradation of nucleic acids by nuclease enzymes. Extraction can include the destruction or removal of substances or impurities that inhibit the nucleic acid amplification reaction. Lastly, extraction can result in a higher, an equal, or a lower concentration of nucleic acids than was present before the extraction, and still it is considered an extraction. For the quantification of nucleic acids, it is preferred that the extraction preserves information about the in situ extracellular and intracellular nucleic acid concentrations in the antibiotic exposure, although some uncertainty is tolerable. Exemplary extraction techniques include extractions that utilize buffers of known volumes, where the buffer can be Lucigen DNA Extraction Buffer or transport solutions such as Zymo DNA/RNA Shield and guanidinium chloride. Another exemplary extraction technique is mechanical extraction by bead beating. A third exemplary extraction technique would be any nucleic acid extraction system used in existing molecular diagnostics assays such as the NucliSENS easyMAG (bioMérieux, France) and the Magna Pure or Magna Pure LC (Roche Molecular Diagnostics, Pleasanton, Calif.) platforms. The three example extraction techniques just mentioned can be performed in ways that preserve information about original nucleic acid concentrations and can be used for same-sample AST.

In particular, in embodiments of same-sample AST detection of intracellular nucleic acid concentration value is typically performed with methods involving lysis of cellular components of the sample, while detection of extracellular nucleic acid concentration value is performed in an extracellular fraction of the sample separated from the sample.

In methods of the present disclosure nucleic acid concentrations, quantification of nucleic acid amount or concentration by any of the above methods yields one nucleic acid concentration value (NACV). For a sample wherein detection of a nucleic acid concentration value is performed according to a set method, a nucleic acid concentration value is a value obtained by quantitively detecting a target nucleic acid in the sample within the set method. A nucleic acid concentration value in the sense of the disclosure is a value proportional to the true concentration of the target nucleic acid in the sample Any positive number can be used as the proportionality constant, preferably the proportionality constant equal to 1.

In some embodiments, the nucleic acid concentration value is a true concentration. In these embodiments, the nucleic acid concentration value can be detected by a digital quantification method such as digital PCR (dPCR). The concentration of nucleic acids is the ratio of the absolute amount of a nucleic acid in a portion of matter to the volume of that portion of matter. The volume of the portion of matter is usually known and controllable during volumetric manipulation of portions of matter. Thus, the absolute amount of nucleic acids can always be calculated from the concentration, and vice versa. Most instruments, like those using bulk fluorometry, measure concentrations of nucleic acids since the signal measured depends on the volume of the sample analyzed. However, some methods, like all digital amplifications, can be said to measure absolute amounts of nucleic acids (by counting individual molecules).

The concentration of nucleic acids can be measured by performing one of several possible nucleic acid amplification reactions. These nucleic acid amplification reactions include polymerase chain reaction (PCR), reverse transcriptase PCR (RT-PCR), real time quantitative PCR (qPCR), reverse transcriptase quantitative PCR (RT-qPCR), qPCR with dual priming oligonucleotides, digital PCR (dPCR), droplet digital PCR (ddPCR), the preceding qPCR variants performed using with probes or molecular beacons, loop-mediated amplification (LAMP), digital LAMP (dLAMP), rolling circle amplification, helicase-dependent amplification, multiple displacement amplification, recombinase polymerase amplification, nucleic acid sequence-based amplification, and other amplification reactions existing in the literature[13]-[15]. The concentration of nucleic acids can be measured by combining nucleic acid amplification reactions, fluorometric, colorimetric, or electrochemical techniques of nucleic acid quantification with the prior or subsequent recognition of specific sequences by wild-type or modified CRISPR-associated protein nucleases, including CRISPR-associated protein-9 nuclease, CRISPR-associated protein-3 nuclease, CRISPR-associated protein-12a nuclease, CRISPR-associated protein-13a nuclease, and CRISPR-associated protein-14a [16], [17]. The CRISPR-associated proteins can cleave specific sequences, or they can non-specifically cleave nucleic acids after activation, which improves the analytical sensitivity and specificity of the nucleic acid quantification.

Other signals for detecting the occupancy of partitions can be identified by a skilled person. For example, light microscopy, fluorescence microscopy, light spectroscopy, fluorescence spectroscopy, Raman spectroscopy, optical density, electrochemical sensors that detect reduction-oxidation (redox) reactions or redox state, fluorescent dyes metabolized by living cells (e.g. resazurin), pH meters, nano-scale cantilever mass balances, atomic force microscopy, mass spectroscopy, nuclear magnetic resonance imaging, enzyme-linked immunosorbent assays, and other immunoassays have all be described in the literature as modalities for sensing the presence, number, mass, or metabolic activity of bacterial cells aside from nucleic acid amplification [18]-[20]. Any of these measurement modalities as well as and additional techniques can be used to determine the number of total cells in a given sample after partitioning as will be understood by a skilled person.

In some embodiments, the nucleic acid concentration value is not the true concentration but proportionally reflects the amount of nucleic acid in the sample. That is, for a higher amount of true nucleic acid concentration in the sample, a higher nucleic acid concentration value will be obtained.

In some of these embodiments, the nucleic acid concentration value can be a direct measurement from experiments. For example, the nucleic acid concentration can be estimated by detecting a nucleic acid with a digital quantification method such as digital PCR (dPCR), or with correction for amplification efficiency by digital LAMP or digital RPA or other digital isothermal amplification chemistries, or calculated from the number of reads corresponding to the target nucleic acids as measured by many high throughput sequencing methods.

Alternatively, digital methods and other methods could be used to provide a concentration parameter that is proportional to concentration, such as raw concentration or positive counts obtained from digital LAMP or digital RPA or other digital isothermal amplification chemistries, from the number of reads corresponding to the target nucleic acids as measured by many high throughput sequencing methods. In some of the digital methods, correction for Poisson loading of nucleic acid molecules is used to obtain the concentration parameter from the raw data, as would be known to those skilled in the art.

In other embodiments, the nucleic acid concentration value can be obtained by detecting a concentration parameter such as Cq, reaction time, fluorescence intensity, and comparing the detected concentration parameter with a standard calibration curve to obtain the nucleic acid concentration value.

In some embodiments, the nucleic acid concentration value can be obtained by detecting a concentration parameter such as quantification cycle (Cq), threshold cycle (Ct), crossing point (Cp), take-off-point (TOP), reaction time, fluorescence intensity, and comparing the detected concentration parameter with a standard calibration curve to obtain the nucleic acid concentration value.

A Cq value is defined as the number of cycles required for the fluorescent signal to exceed the background fluorescence, also referred to as threshold cycle (Ct), crossing point (Cp), or take-off point (TOP) as will be understood by a person skilled in the art.

In one exemplary embodiment, a nucleic acid concentration value can be obtained from a detected Cq value by using the formula “nucleic acid concentration value”=2\hat{ }(−Cq).

In another exemplary embodiment, a nucleic acid concentration value can be obtained from a concentration parameter such as detected reaction time of an exponential quantification method, such as an isothermal amplification method, by using the formula “nucleic acid concentration value”=n\hat{ }(−reaction time) where n has typically a value larger than 1, and reflects the properties of the detecting reaction. For example, if the isothermal exponential amplification doubles the concentration of the product nucleic acid every 20 seconds, then the relative concentration=2\hat{ }(−reaction time in seconds/20 seconds). For a reaction with inverse linear dependence of reaction time on starting target nucleic acid concentration, “nucleic acid concentration value”=1/(reaction time).

In yet another exemplary embodiment, a nucleic acid concentration value can be obtained from a detected florescence by using the formula relative concentration=n*(fluorescence intensity) where n is a normalization factor determined by constructing a standard calibration curve.

In methods of the present disclosure nucleic acid concentrations, detected in intracellular and extracellular fractions of the same sample provides intracellular concentration value (herein also INACV) and extracellular nucleic acid concentration value (herein also ENACV), respectively, used to perform the AST as will be understood by a skilled person.

In accordance with embodiments herein described, intracellular and extracellular nucleic acid concentration values can be detected in a same sample after exposure of the same sample with antibiotic (herein also test conditions or tested conditions). Intracellular and extracellular nucleic acid concentrations can also be detected in reference samples (such as control samples) or conditions (such as different time of exposure to the antibiotic of the same tested sample).

In embodiments, herein described, the detected intracellular and extracellular nucleic acid concentration values from the sample, are then used to provide an intracellular/extracellular proportion value of the same sample (including same samples which are partitions), reference sample (including reference samples which are partitions, test conditions, and/or reference conditions as will be understood by a skilled person upon reading of the remaining portions of the specification and claims.

The term “intracellular proportion value” or “extracellular proportion value” refers to a proportional value of the intracellular nucleic acids or the extracellular nucleic acids with respect to the total nucleic acid of the same sample comprising the intracellular and extracellular nucleic acids, or a value proportional to, correlated to, or mathematically equivalent to the proportional value. The term “mathematically equivalent” means that there exists a one-to-one correspondence between two sets of numbers, such that knowledge of one number implies knowledge of its corresponding number is guaranteed after a calculation.

Accordingly the term “intracellular/extracellular proportion value” used herein interchangeably with the term “extracellular/intracellular proportion value” (herein also EINAPV) is a measure of a proportion, or value related to a proportion, of extracellular (accessible) and intracellular (inaccessible) nucleic acids of the microorganism in the same sample following the exposure under testing conditions and/or reference conditions. This proportion can be, for example, extracellular to intracellular, intracellular to total (intracellular+extracellular), extracellular to total, the relative difference of extracellular to intracellular, or inverses of these.

Accordingly, an intracellular/extracellular proportion value indicates an increased proportion of extracellular (accessible) and intracellular (inaccessible) nucleic acids of the microorganism in an antibiotic treated sample compared to an untreated sample, and is indicative of increased lysis of cells in the sample, and as a consequence increased dead live cell proportion caused by the antibiotic and therefore susceptibility of the microorganism to the antibiotic as will be understood by a skilled person.

In particular, in same-sample methods and systems herein described, if an intra/extra nucleic acid proportion value increases when the proportion of lysis increases (accessibility increases) with respect to a reference, then the sample cells are considered “susceptible” to the antibiotic if the proportion value is above the reference value and “resistant” when it is not. For cases where the intra/extra nucleic acid proportion value decreases when the proportion of lysis increases (accessibility increases), then the reverse is true (susceptible if below the reference, otherwise resistant). Examples of cases where the proportion value increasing with increasing lysis is E/I, E/(E+I), and rate of lysis (k[t]). Examples where the proportion values decrease with increasing lysis is I/E and I/(E+I).

Accordingly, in embodiments of same-sample methods and systems herein described when the proportion of lysis increases (accessibility increases) with respect to a reference value, susceptibility can be determined if the detected increase is outside the tolerance of the reference value. If instead the comparisons to the reference value is within the tolerance of the reference value the sample can be considered “resistant” as will be understood by a skilled person upon reading of the present disclosure. The tolerance can be a set value or determined by statistical analysis of the data (e.g., measure of dispersion). For example, if the proportion value is within 5% of the reference value, then the sample cells can be considered “resistant”. As used herein, “substantially the same” refers to a tolerance of 5%.

In embodiments of the same-sample methods and systems herein described, the comparison can take the form of a statistical test, as described herein as well as what is known to the skilled person. Those tests can be null hypothesis tests that use the EINAPV and reference value and the dispersion of those two values into the determination of whether the EINAPV differs significantly from the reference. Other forms of comparison as known in the art can also be applicable.

In embodiments of the same-sample methods and systems herein described, the EINAPV is a measure of accessibility of the nucleic acid of the microorganism in the same sample following the exposure under testing conditions and/or reference conditions, allowing live/death and susceptibility/resistance determination in absence and without need of a experiments on a separate sample. In some embodiments, where thresholds are used, embodiments of the same-sample methods and systems herein described can be performed in absence and without need of a further detection and in particular marker detection from the same sample as will be understood by a skilled person upon reading of the disclosure.

The wording “measure” indicates a quantity that is equal, proportional to, or mappable to a reference item, so that there exists a one-to-one function relating the two, or so that there exists a monotonically increasing or decreasing function relating the two.

Accordingly the EINAPV is a measure of the accessibility of the nucleic acid after exposure and can thus serve as the output of each experimental condition of a same-sample AST and the input to the calculation of susceptibility as will be understood by a skilled person. The EINAPV is calculated from at least one ENACV and at least one INACV, possibly in combination with information about the total number of nucleic acid and/or cells. In particular, the total number of cells can be taken into account by normalizing the EINAPV with the total number of cells, making the EINAPV an intensive (vs extensive) measure of antibiotic activity as will be understood by a skilled person.

In embodiments herein described, when two fractions, derived from the same sample are measured by the same method, the proportionality constant connecting nucleic acid concentration value and true concentration is approximately the same and therefore it does not need to be known to calculate a nucleic acid concentration proportion value.

In some embodiments, the EINAPV can be provided by an extracellular proportion value or an intracellular proportion value determined by a combination of the ENACV or INACV, respectively, with the total number of nucleic acid. There are a variety of ways to derive the intracellular or extracellular proportion value as will be understood by a person skilled in the art. For example, the extracellular proportion value can be derived as a percent extracellular or extracellular fraction calculated using the following formula:

PE=FI/(FI+LY), (1)

where PE is the percent extracellular, FI is the filtrate concentration (extracellular nucleic acid concentration value), and LY is the lysate concentration (intracellular nucleic acid concentration value).

The extracellular proportion value can also be derived as any value proportional to or correlated to the percent extracellular, such as a ratio of the extracellular to intracellular nucleic acid concentration values or any value proportional to, correlated to, or mathematically equivalent to such ratio. The intracellular proportion value can be derived as a percent intracellular calculated using the following formula:

PI=LY/(FI+LY), (2)

where PI is the percent intracellular, FI is the filtrate concentration (extracellular nucleic acid concentration value), and LY is the lysate concentration (intracellular nucleic acid concentration value). The intracellular proportion value can also be derived as any value proportional to or positively correlated to the percent intracellular. The intracellular proportion value can also be derived as any value proportional to, correlated to, or mathematically equivalent to the percent intracellular, such as a ratio of the intracellular to extracellular nucleic acid concentration values or any value proportional to, correlated to, or mathematically equivalent to such ratio.

In some embodiments, other quantities that have a one-to-one correspondence to the percent extracellular (and E:I ratio) wherein E indicates extracellular and I indicates intracellular summary statistic include the “percent intracellular”

$$\frac{I}{E + I} ,$$

the “I:E ratio”

$$\frac{I}{E} ,$$

the relative difference (defined in several ways, including

$$\frac{E - I}{E + I} , \frac{I - E}{I + E} , \frac{E - I}{( E + I ) / 2} ,$$

and other arbitrary functions of these definitions), and additional other functions that can be constructed from these statistics by arithmetic operations (multiplication, division, addition, subtraction, exponentiation, logarithm, absolute value, etc.) of these definitions as will be understood by a skilled person.

In some embodiments, the intracellular/extracellular proportion value is a relative difference between the extracellular nucleic acid concentration value and the intracellular nucleic acid concentration value as described in “Summary statistics for determination of antibiotic susceptibility from comparison of detected nucleic acid concentration values” section of the present disclosure.

In some embodiments, the intracellular proportion value and extracellular proportion value can be used to determine a proportion of cell lysis which in turn provides the EINAPV for those embodiments. A proportion of cell lysis is a metric that equals, is correlated to, or is mathematically mappable or transformable to the quantity DEAD/TOT, where DEAD is the number or mass of all cells (or cells that meet a certain criteria) that have lysed so far in a sample, and TOT is the number or mass of all cells (or cells that meet a certain criteria) in a sample.

The “percent extracellular” metric “FI” the filtrate concentration (extracellular nucleic acid concentration value), and LY the lysate concentration (intracellular nucleic acid concentration value) also functions as a proportion of lysis metric because “FI” is proportional to the total mass of lysed cells in the sample “LY” and “FI+LY” is proportional to the total mass of cells “TOT”. Thus, the quantity FI/(FI+LY) is proportional to the proportion of lysed cells in the sample at the time of measurement and therefore is a “proportion of lysis” metric.

In some embodiments, the percent extracellular is the preferred form of the EINAPV when one has a bulk loaded partition with only 1 cycle of separation and detection. Other metrics are discussed elsewhere in this disclosure and can be useful when analyzing a bulk loaded partition with only 1 cycle of separation and detection at the same time as other AST runs with different embodiments.

In same-sample AST method, the intracellular/extracellular proportion value of the same sample is then compared with a reference value indicative of results of the experiments in the sample in absence of the antibiotic treatment of the tested condition, such as reference conditions and/or thresholds. The result of the comparison is indicative of antibiotic susceptibility of the microorganism.

In some embodiments, if a bacterium is susceptible to the antibiotic dose in a test condition, then the extracellular proportion value is expected to increase relative to an extracellular proportion value of a control conditions, or the intracellular proportion value is expected to decrease relative to the intracellular proportion value of control conditions.

In some embodiments when the intracellular/extracellular proportion value is a relative difference between the extracellular nucleic acid concentration, if a bacterium is susceptible to the antibiotic in a test condition, then the relative difference is expected to increase or decrease away from the value of zero. Whether the relative difference increases or decreases depends on how one defines the relative difference. There are several mathematical definitions of a relative difference known to the skilled person as described in “Summary statistics for determination of antibiotic susceptibility from comparison of detected nucleic acid concentration values” below.

In some embodiments, a same-sample methods and systems can be performed of partitions derived for example from a single sample, the partitions being grouped for determination of ENACV, INACV and EINAPV based on the experimental conditions tested within a given test run.

The term test run or run, as used herein indicates a series of exposure, separation, detection and determination of EINAPV possibly followed by AST determination through comparison with a reference value performed with any one of the same-sample methods herein described.

Accordingly, in embodiments of same-sample methods and systems herein described performed on partitions, use of partitions enable multiplex testing of different experimental conditions wherein the ENACV, INACV and related EINAPV for each experimental conditions tested in the run is provided by the ENACV, INACV and EINAPV of the group of partitions under the each condition.

In some embodiments where multiplex testing is performed, same-sample methods and systems can provide a profile intra/extra proportion values, live and dead status of the cells and associated susceptibility/resistance determination, for the specimen and/or sample determined in connection a plurality of test conditions.

In some embodiments an AST run is performed with partitions grouped under only one test condition, to provide a single-condition run which results in a single ENACV, INACV and EINPAV for the one test condition. In those embodiments the EINAPV of the single test condition can then be compared with a reference value such as the EINAPV of another set of partitions control conditions and/or with a threshold as will be understood by a skilled person.

In some embodiments an AST run can be performed with partitions with more than one test condition, each condition applicable to a set of partitions in an AST run, each characterized by a set of independent variables, to provide a multiplex-condition run which results in a multiple ENACV, INACV and EINPAV one for each test condition. In those embodiments the EINAPV of each test condition can be compared with a reference value, such as the EINAPV of another set of partitions under a different set of test conditions, the EINAPV of another set of partitions control conditions and/or with a threshold as will be understood by a skilled person.

In embodiments of same-sample methods and systems herein described performed on partitions, the EINAPV of each set of partitions under a same experimental condition can be a ratio of the ENACV/INACV of the partitions, an extracellular proportion value of the partitions, an intracellular proportion value of the partitions and/or a proportion of lysis of the partitions as will be understood by a skilled person.

Accordingly, in some embodiments of same-sample AST methods, the signal measured from each sample partition, or each subset of nucleic acids from each sample partition, is the concentration in the partition of nucleic acids synthesized by the cells of interest, as will be understood by a skilled person upon reading of the disclosure.

In some embodiments of multiplex condition AST run, the test condition comprise different independent variables (usually antibiotic related independent variables) not comprising different times of exposure. In those embodiments, that the multiplex condition run is a “parallel multiplex” or “multiplex” run.

In some embodiments of multiplex condition AST run, the test condition comprise different independent variables (usually antibiotic related independent variables) comprising different times of exposure, then the AST can be considered a “serial multiplex” AST assay.

Embodiments of same-sample methods and systems herein described performed with partitions as a parallel multiplex assay or as a serial multiplex assay allow one to minimize the impact of the phenomenon and confounding variable of the batch effect on the AST determination. Batch effects arise from slight differences in the execution of an AST protocol, such as fluctuations in the duration of each stage of the protocol; the age of the sample whose partition is analyzed; the age and purity of reagent batches used; or fluctuations in room temperature or sunlight intensity. Many of these batch effects differ far more between AST runs than between sample partitions within an AST run. Thus, experimental conditions that belong to the same AST run share the same batch effect, while those of different runs will not, as will be apparent to the skilled person.

Embodiments of same-sample methods and systems herein described performed with partitions as a parallel multiplex assay or as a serial multiplex assay also allow performing a run with more test conditions and thus to increase the number of queries that a practitioner assesses with the AST assay.

In some embodiments, when the number of cells of the same is known or expected to be low inclusion of more test or control conditions, which will reduce the number of microorganisms loaded into each partition and impact the results. In those embodiments, AST runs with fewer experimental conditions, and possibly only one condition, can be preferred over runs with more conditions depending on the query and the number of cells as will be understood by a skilled person. A skilled person will be able to identify whether a single run or a multiplexed run and the specific type of multiplex run can be applied based on both the clinical needs and on the density (or total number) of cells expected in the type of specimen being assayed.

In some embodiments, a multiplex run can be performed to detect susceptibilities of up to 400 different conditions per AST assay, covering multiple antibiotic compounds and different concentrations of each of the multiple antibiotic compounds. Typical current broth microdilution assays test between 8 and 25 antibiotic compounds per AST assay over 96 conditions in parallel. In some embodiments, the number of conditions is determined in view of the clinical needs, costs of reagents and hardware, and/or the number of microorganisms present in the specimen. More conditions require more reagents for each step, increasing costs. Clinicians do not need to test antibiotics that they will not use, which makes additional costs unnecessary. In some embodiments, about 30 cells are needed per test condition to overcome biological stochasticity, especially within rapid exposure times, assuming that the limit of detection (LOD) of the chosen nucleic acid amplification is not a limiting factor. Therefore, if the number of microorganisms in the specimen is less than the amount required to load all conditions, and their partitions, with the minimum number of cells, then some conditions are excluded.

In some embodiments, minimizing the number of AST runs, by fitting more conditions into fewer, possibly one, multiplex runs can be preferred, due to the elimination of batch effect differences.

In embodiments herein described, wherein same-sample AST methods and systems are performed on partitions, identical experimental conditions which are applied to different subset of partitions are called replicate conditions. In contrast, experimental conditions that are not identical cannot be replicates, and their partitions cannot be reassigned to each other.

In embodiments herein described, wherein same-sample AST methods and systems are performed on partitions in replicate, the choice of how to assign partitions to replicate conditions depends on the goals of the practitioner and on the particular embodiment performed as will be understood by a skilled person.

In some embodiments same-sample methods and system performed on partitions can be performed in bulk or digitally depending on the number of cells comprised in each partition.

In particular in embodiments of accessibility AST of the disclosure performed on digitally-loaded same-sample accessibility AST, the average number of cells in each sample partition when loaded randomly is in the digital range (generally below 3.5 cells per partition).

Accordingly, a digitally-loaded condition is one in which cells are loaded randomly to the condition's partitions, and there is a reliable chance that one or more of the partitions will not receive any cells, because cells are discrete particles and not continuous, divisible entities. The most accurate probability model describing the random loading of cells into partitions is the multinomial distribution, but in the limit of a small ratio between the partition volumes and the volume of the source of cells, the number of cells per partition is Poisson distributed. The chance that a partition receives k cells is found to be

$$P ( k ) = \frac{{λ}^{k} {e}^{- λ}}{k !} ,$$

and the chance that a partition receives 0 cells is thus P(0)=e^{-λ}, where λ is the density of cells in the sample from which the partitions are loaded.

Bulk loading conditions are instead the condition where one has a density higher 3.5 cells per partition, as will be understood by a skilled person, the density of 3.5 cells being a threshold for delineating the digital range, where densities below the threshold are in the digital range and densities above the threshold are not.

In embodiments of same-sample methods and systems of the disclosure performed on partitions, the density of cells per partition is related to the density of cells by the following equation: Density=DensityPerPartition/PartitionVolume, wherein that the density in cells per partition is a function of the partition volume. Controlling either the density of the cells or the volume of the partitions brings the loading in or out of the digital range. Lowering the density or decreasing the volume of the partitions moves one towards digital loading and vice versa. It is preferrable to have a high density of cells when loading but a low density of cells per partition. A high density of cells at loading means that more cells are analyzed and biological stochasticity is overcome, but this density is not controllable by the practitioner when the source of the cells is a clinical specimen. A lower density of cells per partition makes it more likely for partitions containing some cells to be containing only one cell, which makes interpretation of NACVs observed from the partition easier.

In some embodiments of same-sample methods and systems of the disclosure performed on partitions, bulk loading, can be selected in embodiments wherein a high density of cells and a high density of cells per partition at loading is desired. For example, in instances in which the stochasticity of loading cells into partitions is overcome in bulk loading by the central limit theorem, which states that the variance in well loading decreases relative to the mean well loading as the mean increases. Thus, it is preferable to use a smaller number of partitions in bulk loading, compared to a maximum number of partitions in digital-loading. Thus, loadings that are slightly above the digital range fall into a gray zone where the stochasticity of loading is poorly overcome by inference using either Poisson statistics or by the central limit theorem. Such loadings are not preferred, but nonetheless can be analyzed as a bulk loading if they occur.

In embodiments of same-sample methods and systems performed on partitions using digital loading the number of empty wells yields information about the total number of cells at the time of loading. This information is separate from the information about the total number of cells at the end of the exposure found inherently in the collection of ENACVs and INACVs. Furthermore, by detecting single cells, or at least small numbers of cells per partition, digitally-loaded same-sample AST improves the detection of low frequency heterogenous resistance phenomena (heteroresistance and persister cells).

In embodiments of same-sample methods and systems performed on partitions, digitally-loaded same-sample AST, by virtue of monitoring single cells, unlocks a property of accessibility that has advantages over other biological phenomena. The phenomenon of lysis provides a highly binary signal at the cellular level, since in most known bacteria, lysis by beta-lactam antibiotics causes the entire (rather than partial) amount of intracellular nucleic acids in the cell to disperse into solution as extracellular nucleic acids within milliseconds (rather than minutes). Thus, in embodiments of same-sample AST disclosed herein that can detect single cell lysis events, the biological event of lysis does not limit the speed at which lysis events are detected.

In embodiments of same-sample methods and systems performed on partitions digital loading increases the signal to noise ratios of quantification within each partition's NACVs. This increase arises because the same amount of nucleic acids within a single cell is confined to a smaller volume, raising its effective concentration. For example, a loaded partition's nucleic acids would diffuse over twice the volume if it were merged with a nearby empty partition's volume, decreasing the signal by half when nucleic acid concentrations are measured as an intensive property.

In embodiments of same-sample methods and systems performed on partitions, bulk loading allows performance of testing with reduced amounts of reagents needed for quantification, the avoidance of specialized hardware to manipulate myriad small partitions, and simpler (but less powerful) statistical analysis.

In some embodiments of same-sample methods and systems performed on partitions, a digital loading can be achieved by loading a serial dilution of the sample, such that multiple experimental conditions are created, each with a different loading density. Alternatively, is it possible to load the sample into partitions that cover a wide range of volumes [21]. in this case, the partitions of each size are grouped into experimental conditions, as will be understood by a skilled person.

Possible embodiments of same-sample AST are here classified by the number of sample partitions that go through the AST protocol, how these partitions are grouped into experimental conditions, and variations on the timing of separation and extraction.

The embodiments of same-sample AST can be classified along four intersecting characteristics. Firstly, same-sample AST can either be run singly (including multiple runs in series) or as a parallel multiplexed assay. Secondly, same-sample AST can include a concurrent control condition, a temporal control, or a reference information replacing the control condition. Thirdly, same-sample AST can be performed in bulk or in a digitally-loaded architecture. Fourthly, same-sample AST can be performed with one endpoint measurement or as a time-series.

Same-sample methods and systems in accordance with the disclosure can be performed with a digital or a bulk loading as will be understood by a skilled person. For example, an AST run with 1000 partitions loaded with a density in the digital range can be analyzed as a digital loading, or it can be viewed as 1000 bulk loadings with a very high coefficient of variation approaching 1.0. The former approach is preferred over the latter since the former yields more information, An AST run with 1000 partitions loaded with a density above the digital can be analyzed as a failed digital loading or as 1000 bulk loadings. A failed digital loading is considered failed with no wells are empty because the loading density cannot be inferred using Poisson statistics. If only a small (e.g. less than 10%, the exact number depending on the practitioner's tolerance for error in their application) percentage of wells are empty in a digital loading, inference is possible but greatly weakened. In this case, the latter approach of viewing the loading as a bulk loading is preferred, since the former yields no information while the latter will yield a good estimation of the mean and standard deviation of the loading density. A single partition loaded above the digital-range threshold density can be viewed as a failed digital-loading with just one partition, or as a bulk loading of one partition; the latter approach is preferred because the former is not useful. A single partition loaded below the digital-range threshold density is either a bulk loading with a very high coefficient of variance (e.g. starting above 20% and approaching positive infinity as the loading density decreases from 3.5 cells per partition to 0 cells per partition) or a degenerate digital-range partition from which the gained information about the number of cells remains unusably low; no approach is preferred, and more cells are needed for analysis to proceed. The coefficient of variation is the standard deviation of a random variable divided by the mean value of the random variable. The coefficient of variation is a measure of relative noise, as known to the skilled person.

In some embodiments, multiple detection of extracellular nucleic acid concentrations of a same sample are performed in series on extracellular fractions of the sample and of n reconstituted samples obtained by n cycles of i) antibiotic exposure, ii) separation of the sample to obtain the extracellular fraction and a cellular fraction and iii) reconstitution of the sample by adding the culture media to the cellular fraction of the sample.

Accordingly, in these embodiments herein also indicates as same-sample time series, the n reconstituted samples comprise the cellular fraction of the same sample initial sample and extracellular fraction which are separated following antibiotic exposure at the tested conditions of each cycle.

In particular, in embodiments of methods and systems comprising same-sample time series, the n-cycles can be performed in connection with an antibiotic exposure performed at a same or multiple tested conditions, followed by separation of an extracellular fraction and detection of extracellular nucleic acid concentration value therein.

In embodiments of methods and systems comprising same-sample time series, the n-cycles are performed in combination with an n+1 cycle in which the nth reconstituted sample is subjected to antibiotic exposure, separation, detection of extracellular nucleic acid and also by detection of intracellular nucleic acid in the cellular fraction.

In embodiments of methods and systems comprising same-sample time series, the detection of an intracellular nucleic acid concentration of the nth reconstituted sample in connection with a n+1 cycle performed under conclusive same or different test condition, and the related value can be used for calculation of intracellular nucleic acid concentrations of the sample at at least one and typically each of the n-cycles.

In embodiments of methods and systems comprising same-sample time series, intracellular/extracellular nucleic acid proportion values can thus be calculated for the n+1 cycles and used for AST. In particular, in those embodiments, a comparison between intracellular/extracellular nucleic acid proportion values of the n+1 cycles can be performed to determine antibiotic susceptibility and/or increase accuracy of the AST determination when used in combination with comparison with a threshold and/or control conditions as will be understood by a skilled person upon reading of the present disclosure.

For example, in embodiments of methods and systems comprising same-sample time series, where n=2, the method provides three antibiotic-treated extracellular nucleic acid concentration value obtained by detecting a nucleic acid concentration of the extracellular component of the sample and reconstituted sample during the 2 cycles and extracellular nucleic acid concentration value obtained by detecting a nucleic acid concentration of the extracellular component of the n+1 cycle. In those embodiments antibiotic-treated intracellular component obtained during the n+1 (here third cycle) is three times treated with the antibiotic (“thrice-treated intracellular component”), one for each cycle.

In embodiments of methods and systems comprising same-sample time series, during the n+1 cycle an antibiotic-treated intracellular nucleic acid concentration value can be obtained by detecting the nucleic acid concentration of the intracellular component of the trice-treated intracellular component in the presence of a lysis treatment. The antibiotic-treated intracellular nucleic acid concentration value of the sample and reconstituted samples during the first cycles then can be calculated by summing the antibiotic-treated extracellular nucleic acid concentration value of the second cycle with the detected antibiotic-treated intracellular and extracellular nucleic acid concentration values of the third cycle. Similar calculations can be performed to identify the intracellular nucleic acid concentration values of the second cycle, by summing the detected antibiotic-treated intracellular and extracellular nucleic acid concentration values of the third cycle as will be understood by a skilled person.

Thus, in embodiments of methods and systems comprising same-sample time series, a series of intracellular/extracellular nucleic acid proportion values of the sample can be obtained for each cycle of the n+1 cycles using the series of paired secondary antibiotic-treated intracellular and extracellular nucleic acid concentration values as described herein.

In embodiments of methods and systems comprising a same-sample time series, the obtaining n+1 extracellular nucleic acid concentration values and the n-th cycle intracellular nucleic acid concentration value yields a time series, since the n+1 cycles are distributed over time and therefore capture the population dynamics of the tested microorganism. The population dynamics are the changes in number, age, and status of microorganisms in a population over time, or in other words, changes in the size and structure of a population of microorganisms over time.

In embodiments of methods and systems comprising a same-sample time series, the methods for same-sample AST described herein allow obtaining a time series from a single sample, or from multiple samples run in parallel (such as partitions), if the average number of cells in each of the samples is identical and a large number, or from digitally loaded runs, as will be understood by a skilled person upon reading of the present disclosure.

The methods for same-sample AST described herein enable time series measurements from a single sample because they 1) separate the extracellular nucleic acids from the intracellular nucleic acids without destroying the intracellular nature of the intracellular nucleic acids by lysis, or at least the majority of such intracellular nucleic acids, and 2) do not stop the living cells from continuing to respond to antibiotic, such as by killing them.

In embodiments of methods and systems of the disclosure comprising a same-sample series detection, the intracellular/extracellular proportion value can be provided as a and lysis rate proportion of cell lysis, and probability of lysis as will be understood by a skilled person.

In embodiments of methods and systems of the disclosure comprising a same-sample series detection, obtaining a time series of ENACVs, one INACVs, and multiple inferred INACVs, allows the practitioner to detect phenomena that the affect population dynamics of the examined microorganisms in view of the additional information given by a time series enables. Such phenomena include simultaneous growth and antibiotic killing, a lag in antibiotic killing or a lag growth phase, density dependent growth rates, heteroresistance, persister cells, and phenotypic tolerance.

In particular embodiments of methods and systems comprising a same-sample time series, the obtained series of ENACVs and the final INACVs can be used to determine an intra/extra proportion value expressed as rate of lysis as will be understood by a skilled person. In particular, by assuming that the total number of nucleic acids has not changed during the exposure, it is possible to infer and calculate the INACVs for all time points, and then calculate an average relative rate of lysis between each adjacent pair of time points.

The rate of lysis can be used in statistical modeling to address the phenomena that the affect population dynamics of the tested microorganism. For example, if the average relative rates of lysis form a unimodal distribution, one can conclude that the rate of lysis was constant with respect to time during the exposure. If the distribution is bimodal with the earlier average relative rates near 0 percent per unit time, one can conclude that the rate of lysis changed over time, such as if there is an initial time lag in antibiotic killing.

In some embodiments, wherein measurements of intracellular nucleic acid concentration and extracellular nucleic acid concentration are detected in time according to a time series embodiments of the present disclosure the intracellular/extracellular proportion value is provided by a rate of lysis.

The rate of lysis is calculated from the measured extracellular or intracellular nucleic acid concentration values and may be numerically equivalent or different to a literal ratio of those values in time. the rate of lysis can be used as an intracellular/extracellular proportion value in subsequent calculations such as in the calculation of summary statistics (see “Summary statistics for determination of antibiotic susceptibility from comparison of detected nucleic acid concentration values” herein) or the application of statistical tests for calling resistance. A rate of lysis is a metric that equals, is correlated to, or is mathematically transformable to the rate at which cells are lysing from antibiotics within a given window of time. This quantity is sometimes called in the literature a “kill rate”, a “kill rate constant”, a “death rate”, or a “death rate constant”. There are several ways to calculate a rate of lysis, ranging from simple models solvable by algebra to complex models with additional parameters solved by numerical algorithms.

In embodiments wherein rate of lysis is considered, and only 1 cycle of separation is performed, given T units of time elapsed between the start of the antibiotic exposure and the time when intracellular and extracellular nucleic acids were separated and subsequently quantified to yield LY and FI, respectively. Suppose that the mean copy number of nucleic acids per cell, COPYN, is known from the literature or from a prior set of experiments performed by the skilled person. Then the average absolute rate of lysis is found to be

$$\frac{FI}{COPYN * T}$$

in units of cells lysed per unit time. This rate is proportional to both the activity of antibiotic and on the number of initial cells in the sample, the latter being a confounding variable. A better metric would be an average relative rate of lysis. In this particular embodiment, the relative rate of lysis over the whole exposure is

$$\frac{FI}{( FI + LY ) * T}$$

in units of fraction of cells lysed per unit time. This quantity is equal to the “percent extracellular” metric divided by time.

The average relative rate of lysis can be more broadly defined as the fraction of cells lysed per unit time during any duration of time within the antibiotic exposure, not just the whole exposure. This definition applies to any antibiotic exposure in any of the embodiments of same-sample AST. Furthermore, one can define an instantaneous relative rate of lysis to be the limit of the average relative rate of lysis as the duration of time between time points becomes arbitrarily small. In other words, the instantaneous relative rate of lysis is the time derivative of the percent extracellular. The average relative rate of lysis therefore always can serve as an approximation of the instantaneous relative rate of lysis.

The instantaneous relative rate of lysis appears in differential equations describing the live and dead cells in the sample. For example, one can model a population of cells in antibiotics with the following equations:

$$\begin{matrix}\frac{dLive}{dt} = - kLive , & ( 3 ) \\ \frac{dDead}{dt} = kLive , & ( 4 )\end{matrix}$$

with boundary conditions of Dead [t=0]=Dead₀, Live [t=0]=Live₀, and Live₀+Dead₀=FI+LY. Here, Live is either the intracellular nucleic acid concentration value or the number or mass of cells that have lysed in the sample, t is time, dDead/dt is the derivative of Dead with respect to time, k is the rate of lysis, FI is the measured filtrate nucleic acid concentration, LY is the measured lysate nucleic acid concentration, and Dead₀ and Live₀ are the measured Dead and Live at the start of the exposure, as might have been recorded in embodiments where more than 1 cycle of same-sample AST is performed. This ordinary differential equation can be rewritten in closed form as Live=Live₀*e^{-kt} and Dead₀=Dead₀+Live₀*(1−e^{-kt}). For planktonic bacteria species, especially if one washes the cells with a buffer before starting the antibiotic exposure, it is reasonable to assume that little or no extracellular nucleic acids are present before cells are exposed to antibiotics; in such cases, Dead₀=0. The value of k is then found using algebraic rules, or by solving the differential equation using numerical algorithms known to the skilled person, and this calculation can be performed for same-sample AST runs with only 1 cycle of separation and detection. If one has made multiple measurements of Dead by repeating n cycles of same-sample AST (see below), one can use standard fitting algorithms known to the skilled person (such as linear regression) to fit the model equation to the data, yielding a more accurate value of the rate of lysis. The use of differential equations is preferred when the number of cells examined is large. Therefore, differential equations are applicable for interpreting individual bulk-loaded partitions or the ensemble of partitions in a digitally-loaded AST. The use of differential equations is preferred for time series with greater than 1 cycle.

More complicated models and their equations can also be used to define and calculate the rate of lysis. For example, one may define the rate of lysis using the following system of equations:

$$\begin{matrix}\frac{dDead}{dt} = kLive , & ( 5 ) \\ \frac{dLive}{dt} = ( μ - k ) Live , & ( 6 ) \\ Dead [ t = 0 ] = {Dead}_{0} , and & ( 7 ) \\ Live [ t = 0 ] = {Live}_{0} . & ( 8 )\end{matrix}$$

“Dead” is the amount (number or mass) of lysed and dead cells, “Live” is the amount (number or mass) of un-lysed and growing cells, μ is the growth rate of the living cells (also known as the intrinsic growth rate, the growth rate constant, or the Malthusian parameter), t is time, and k is the rate of lysis. This second model also has a closed form solution:

$$Dead ( t ) = {Live}_{0} ( \frac{k}{μ - k} ) ( {e}^{( μ - k ) t} - 1 ) + {Dead}_{0} , Live ( t ) = {Live}_{0} {e}^{( μ - k ) t} . \qquad ( 9 )$$

One can assume a value of μ, since the value of the growth rate constant in rich growth media has been published for most pathogenic bacteria. Assuming a known value for μ and Dead₀ allows the value of k to be estimated by algebra for embodiments which only have 1 cycle of separation and detection. The use of differential equations is preferred when the number of cells examined is large. Therefore, differential equations are preferred for interpreting individual bulk-loaded partitions or the ensemble of partitions in a digitally-loaded AST.

If one has performed a time-series same-sample AST, then the estimation of k can be made even more precisely than by the methods above that apply to each time point (cycle) in the time series. This increased accuracy arises because one becomes able to define a set of equations, one per time point, of the forms described above, and this set of equations is overdetermined. Standard data fitting algorithms and algorithms for numerically solving differential equations, as known to the skilled person and available in commercial and open-source software packages, can be used to find useful values of k and μ in this model without assuming a value of μ or without finding a closed form for the differential equation. The use of differential equations is the preferred analysis for time series with greater than 1 cycle and where one has available either individual bulk-loaded partitions or the ensemble of partitions in a digitally-loaded AST.

In some embodiments, the rate of lysis is not be a constant value during the exposure, but rather a function of time called k[t]. There may also be a rate of cell death not caused by antibiotics, which we call k₀. Then the following equation defines the rate or lysis:

$$\frac{dDead}{dt} = ( k + {k}_{0} ) Live , \frac{dLive}{dt} = ( μ - k - {k}_{0} ) Live .$$

The closed form solution is

$$\begin{matrix}Live [ t ] = {Live}_{0} {e}^{( μ - k ) t} S [ t ] , & ( 10 ) \\ Dead [ t ] = {Live}_{0} [ ( \frac{{k}_{0}}{μ - {k}_{0}} ) ( {e}^{( μ - {k}_{0} ) t} S [ t ] - 1 ) + ( \frac{μ}{μ - {k}_{0}} ) {\int }_{0}^{t} {e}^{( μ - {k}_{0} ) τ} f [ τ ] d τ ] , & ( 11 )\end{matrix}$$

where S[t]=e^{-∫}^{₀}^{^{t}}^{h[t]dt} and f[t]=h[t]S[t]. From this equation, the rate of lysis may be found by algebraic manipulation, possibly aided by numerical approximations of the integrals by standard algorithms known to the skilled user when no closed integral forms are known for the choice of k[t]'s functional form. Alternatively, for embodiments where multiple cycles of same-sample AST are performed, published fitting algorithms such as gradient descent, Bayesian Markov Chain Monte Carlo, expectation maximization, and particle swarm optimization can be used to estimate the values of the constant parameters of the equation, which then yields a parametrized function for k[t] or an empiric description of k[t] in the form of a sequence of values. The approach described in this paragraph is preferred when a lag in antibiotic killing is observed by the practitioner in the results of a time-series AST. The use of differential equations is the preferred analysis for time series with greater than 1 cycle and where one has available either individual bulk-loaded partitions or the ensemble of partitions in a digitally-loaded AST. Differential equations containing more variables enable more phenomena to be detected from time-series AST, but require a higher number of cycles or replicate conditions to be fit unambiguously.

Examples of even more complicated models can be defined by the inclusion of more terms in the equations describing the bacteria population in the presence of antibiotic killing, such as allowing the growth rate to depend on the total number of bacteria (known as density depending population models, logistic growth, logistic population models, Gompertz growth models, and other models known to the skilled person), by including a constant rate of cell death independent of antibiotic concentration, by assuming the existence of persister cells, by assuming heteroresistance, or by including a lag phase where the cell growth rate or rate of lysis differs in an initial interval of time from the start of the antibiotic exposure stage than in the remainder of the antibiotic exposure stage.

In some embodiments of methods and systems comprising a same-sample time series, the intracellular/extracellular proportion value can be a probability of lysis. In some embodiments, the probability of lysis can be calculated from intracellular/extracellular proportion values, and then the resulting probability of lysis then acts itself as an intracellular/extracellular proportion value in subsequent calculations, such as in the calculation of summary statistics (see “Summary statistics for determination of antibiotic susceptibility from comparison of detected nucleic acid concentration values” herein) or the application of statistical tests for calling resistance. A probability of lysis is a metric that equals, is correlated to, or is mathematically mappable or transformable to the probability of a lysis-related event occurring, such as the probability of a given cell lysing before a certain time (often called the “survival probability”), the probability of a given cell lysing within a certain time window given that it has not lysed before the start of that time window (often called the “hazard rate” or “hazard function”), the probability that a population of bacteria has died out by a certain time after the start of the exposure (the “extinction probability”), and the probability that a population of bacteria will eventually go extinct in infinite time (also known as the “extinction probability” or known as “ultimate extinction probability”).

The aforementioned “percent extracellular” metric can function as or be interpreted as a probability of lysis (in addition to being an intracellular/extracellular proportion value, a proportion of lysis, and a rate of lysis). Let P be the probability that a given cell in a population of N cells will lyse by time T, and ignore for now the generation of new healthy cells during this time. Then the expected fraction of cells that have lysed by time T will be equal to P. Assuming that the amount of nucleic acids within each cell is independent of whether they lyse or not, then the extracellular and intracellular nucleic acid concentration values F and Y are directly proportional to the numbers of lysed and unlysed cells, and therefore the percent extracellular defined as F/(F+Y), is also equal to P, or at least serves as the maximum likelihood estimate of P as known to the skilled person. In other words, if 50% of the nucleic acids in a sample are extracellular, and new growth is ignored, then one can estimate that each cell in the sample had a 50% chance of lysing by the time the extracellular and intracellular nucleic acids were separated.

The aforementioned rate of lysis also can function as or be interpreted as a probability of lysis. Specifically, the rate of lysis of a population of cells is equal to the expected fraction of cells that lyse in a given window of time. This is turn is equal to the probability of a given cell lysing in that window of time, and thus is equal to the hazard rate due to antibiotic killing.

In some embodiments, the survival probability is calculated from the percent intracellular. If there is negligible growth of bacteria during the antibiotic exposure, then the survival probability at time T is equal to the percent intracellular at time T.

In some embodiments, where simultaneous growth and antibiotic killing are assumed to occur, the survival probability can be calculated from the hazard rate using the following mathematical identities:

$$S [ t ] = {e}^{- {\int }_{0}^{t} h [ t ] dt} and h [ t ] = - \frac{1}{S [ t ]} \frac{dS [ t ]}{dt} .$$

The hazard rate can either be represented in a parametric form or as an empirically measured function from several cycles of same-sample AST. As previously discussed, the survival probability and hazard rate are the preferred analysis when one has available a time series with greater than 1 cycle and where one has available either individual bulk-loaded partitions or the ensemble of partitions in a digitally-loaded AST.

In some embodiments, the ultimate extinction probability “PUltExtinct” can be calculated as

$$PUltExtinct = {( max \{ \frac{k}{μ} , 1 \} )}^{{N}_{0}} , \qquad ( 12 )$$

where k is the rate of lysis (assumed to be constant and discussed previously), μ is the growth rate constant (discussed previously), and N₀ is the number of cells in the sample at the start of antibiotic exposure. In such a calculation, the cells in the sample are assumed to obey a Galton-Watson branching process model where each cell independently divides into two new cells with probability or dies with probability k. When a bulk loaded partition is available, the value of N₀ can be determined by dividing the total nucleic acid concentration value (FI+LY) by an estimate of the copy number per cell from the literature. For example, for members of the Enterobacteriaceae, the copy number of ribosomes is on the order of 60-70,000 per cell. When a digitally-loaded partition is available, the value of N₀ can be determined using Poisson statistics. The value of k can be estimated as the average relative rate of lysis. Calculating this quantity is preferred when the practitioner has a need to compare same-sample AST with the minimum inhibitory concentration (MIC) obtained by broth microdilution assays.

In some embodiments, the probability of extinction by time t, PExtinct[t], can be calculated as

$$PExtinct [ t ] = {[ \frac{k + {k}_{0}}{μ} ( \frac{{e}^{( μ - ( k + {k}_{0} ) ) t} - 1}{{e}^{( μ - ( k + {k}_{0} ) ) t} - \frac{k + {k}_{0}}{μ}} ) ]}^{{N}_{0}} , \qquad ( 13 )$$

where μ is the intrinsic growth rate constant, k is the rate of lysis or kill rate due to antibiotics, k₀ is the death rate independent of antibiotics, t is time, and N₀ is the number of cells in the sample at the start of antibiotic exposure. This equation arises when one interprets the cells in the same-sample AST to be obeying a mathematical model called the Markov Birth-Death Process with a birth rate of μ and a death rate of k+k₀. The value of k₀ can be measured using concurrent same-sample ASTs containing no antibiotics, in the most preferred approach; in a less preferred approach, k₀ can be assumed to be a value measured by previous experiments for the same pathogen; and in the least preferred but still useful approach, k₀ can be assumed to be 0. When a bulk loaded partition is available, the value of No can be determined by dividing the total nucleic acid concentration value (FI+LY) by an estimate of the copy number per cell from the literature. When a digitally-loaded partition is available, the value of N₀ can be determined using Poisson statistics. The value of k can be estimated as the average relative rate of lysis.

In some embodiments of a same-sample time series, the intracellular/extracellular proportion value is substituted by a probability of lysis. In some embodiments, the probability of lysis can be calculated from intracellular/extracellular proportion values, and then the resulting probability of lysis then acts itself as an intracellular/extracellular proportion value in subsequent calculations, such as in the calculation of summary statistics (see “Summary statistics for determination of antibiotic susceptibility from comparison of detected nucleic acid concentration values”) or the application of statistical tests for calling resistance. A probability of lysis is a metric that equals, is correlated to, or is mathematically mappable or transformable to the probability of a lysis-related event occurring, such as the probability of a given cell lysing before a certain time (often called the “survival probability”), the probability of a given cell lysing within a certain time window given that it has not lysed before the start of that time window (often called the “hazard rate” or “hazard function”), the probability that a population of bacteria has died out by a certain time after the start of the exposure (the “extinction probability”), and the probability that a population of bacteria will eventually go extinct in infinite time (also known as the “extinction probability” or known as “ultimate extinction probability”).

The aforementioned “percent extracellular” metric can function as or be interpreted as a probability of lysis (in addition to being an intracellular/extracellular proportion value, a proportion of lysis, and a rate of lysis). Let P be the probability that a given cell in a population of N cells will lyse by time T, and ignore for now the generation of new healthy cells during this time. Then the expected fraction of cells that have lysed by time T will be equal to P. Assuming that the amount of nucleic acids within each cell is independent of whether they lyse or not, then the extracellular and intracellular nucleic acid concentration values F and Y are directly proportional to the numbers of lysed and unlysed cells, and therefore the percent extracellular defined as F/(F+Y), is also equal to P, or at least serves as the maximum likelihood estimate of P as known to the skilled person. In other words, if 50% of the nucleic acids in a sample are extracellular, and new growth is ignored, then one can estimate that each cell in the sample had a 50% chance of lysing by the time the extracellular and intracellular nucleic acids were separated.

Embodiments of methods and systems comprising a same-sample time series, performing embodiments of methods and systems comprising a same-sample time series, allows one to detect lag time in antibiotic killing as will be understood by a skilled person.

A lag in growth phase occurs when microorganisms enter an environment conducive to growth but do not commence synthesis of nucleic acids or cell division for an initial period of time. During this time, antibiotic kill rate will be reduced. When the microorganisms exit the lag phase and enter a growing phase, antibiotic kill rate will increase. In a time-series same-sample AST, a lag in growth phase will appear the same as a lag in antibiotic killing.

In some embodiments of a same-sample time-series herein described a lag in antibiotic killing is seen when the rate of lysis is low during an initial window of time at the beginning of the antibiotic exposure, then increases to a higher rate for the remainder of the exposure step. If there is no lag in antibiotic killing, and the strain is susceptible, then the ENACV from the first cycle of the time series will have the highest value. Subsequent cycles will yield ENACVs of decreasing value. In the case of a lag in antibiotic killing in a susceptible strain, the first L cycles of ENACVs would be low. The ENACVs would then increase in magnitude, then finally decrease as the population of microorganism goes extinct. Computing the average relative rates of lysis for each cycle of a time series same-sample AST, as described above, would be sufficient to detecting a lag in antibiotic killing. Detecting lags in antibiotic killing is particularly important in diagnostics since stopping an exposure before the lag in killing has elapse will yield a false positive result for resistance (or a false negative result for susceptibility). Detecting a lag for a pairing of microorganism and antibiotic in one AST run informs the use about the minimum exposure duration in future AST runs. In embodiments where methods and systems comprising a same-sample time series The information yielded by time-series ASTs can be used for a quality control workflow in a clinical laboratory to exist to catch these inaccurate results.

Additionally, embodiments of methods and systems comprising a same-sample time series and determination of the rate of lysis allow to address phenomena such as microorganism growth. “Microorganism growth” as used herein indicates proliferation of a microorganisms into two daughter cells When microorganisms grow in a nutrient rich environment, the population grows exponentially until nutrients are depleted. When nutrients are depleted, the population exits the exponential phase and enters the early stationary phase, in which the growth rate slows. When the growth rate has slowed to 0, the population stops growing and enters the stationary phase. The population density at which population growth stops in an environment is called the environment's carrying capacity, and the term density-dependent growth rate describes a growth rate that is a function of population density. For example for E. coli, cells exit the exponential phase at around a density of 90,000,000 cells/mL.

In embodiments of same-sample AST performed where the population remains below the density at which exponential phase ends, the growth rate remains constant. This is the case for most embodiments of same-sample AST performed on clinical samples and clinical isolates and using standard rich broths such as Mueller-Hinton Broth. In those embodiments, the population increases L=L₀e^{μt}, where L is the population at time t and L₀ is the population at time 0, so that when L grows to 90,000,000 cells/mL, the population exits exponential phase and starts to slow down. Typical values for μ lie between 0.017 and 0.034 min⁻¹, and typical exposure times are under 360 minutes. The maximum starting cells L₀ for an exposure of length t is then calculable as L₀=90,000,000e^{μt}.

In embodiments of same-sample AST performed with a high number of starting cells however, such as 90,000,000 cells/mL, or with a growth media that does not contain high nutrients, then it is possible for the growth rate to not be constant during the exposure. This depletion of nutrients can be detected by estimating the density of cells at the end of the exposure by dividing the total nucleic acid concentration value by a likely copy number per cell, then comparing that density to a known density threshold, above which cells would be expected to be nutrient limited. However, if the density threshold for exiting exponential phase is not known, or if the copy number per cells is not known, performing a bulk time-series same-sample AST would enable a user to detect a slowing of the antibiotic-induced rate of lysis in the later cycles, which would suggest a slowing of the growth rate too. It is important to detect the depletion of nutrients because a slow or zero-valued growth rate could appear as a false positive for resistance. Detecting depletion of nutrients will enable users to adjust the starting inoculum of cells for a repeat of the assay.

In embodiments of same-sample AST, simultaneous growth and antibiotic killing occurs when the rate of growth is of a comparable order of magnitude to the rate of lysis and therefore rate of antibiotic killing, or is greater than the rate of lysis. Cells which are not yet killed by antibiotic may be able to continue to synthesize intracellular nucleic acids and possibly to divide into daughter cells. This phenomenon manifests itself in a time series as an increase in the total amount of nucleic acids in the sample over an initial period of time. In embodiments wherein the same-sample AST is performed with an end-point measurement instead of a time-series, the growth of cells during the exposure would appear as a relative increase in the intracellular nucleic acid amount in the sample compared to a sample in which the growth rate is negligible, but this would be indistinguishable from a sample in which cells did not grow but for which the proportion of lysis was less. However, in digitally-loaded, time-series, same-sample AST, the digital loading produces additional information about the initial total number of cells loaded into the experiment's partitions, because the fraction of empty partitions can be used to calculate the density of cells at the time of loading via the formula

$$Density = - ( \frac{1}{V} ) ln ( \frac{\# Empty}{\# Total} ) , \qquad ( 14 )$$

where #Empty is the number of empty partitions, #Total is the number of partitions, and V is the volume of the partitions. Accordingly, the digitally-loaded, time-series, same-sample AST allows one to address the phenomenon of simultaneous growth and antibiotic killing as will be understood by a killed person.

In embodiments, of methods and systems comprising a digitally loaded same-sample time-series herein described the total number of cells at the end of the antibiotic exposure can also be estimated by several means, described below.

In some embodiments of methods and systems comprising a digitally loaded same-sample time-series herein described the total number of cells at the end of the antibiotic exposure can be estimated by summing the ENACVs and final INACV from each partition, then divide by the known copy number per cell to get the number of cells in that partition. In those embodiments, one then sums across all partitions of this number of cells to get the number of cells in the entire sample. Alternatively, one can sum all the ENACVs and final INACV from each partition and across all partitions, then divide the total nucleic acid amount in the sample at the end of the exposure by the copy number per cell. The copy number per cell is known from prior experiments or literature, and it can also be estimated from a given AST run by fitting the final INACVs to a mixture model. The mixture model posits that only integer numbers of cells are allowed to occupy each partition, and if the distribution of final INACVs is multimodal, the modes of the distribution indicate these integer numbers of cells. If the final INACV distribution is not multimodal, then the nucleic acid quantification error is too great to enable inference, and a value from the literature are used.

In some embodiments of a digitally loaded same-sample time-series herein described, in a further method for estimating the total number of cells at the end of the antibiotic, the timepoints of the time series are sufficiently close together (high temporal resolution) so that the lysis of individual cells can be distinguished in the filtrate of a given partition. A lysis event can be detected by the absence (or the background amount) of extracellular nucleic acids in a first time point, an increase of extracellular nucleic acids in a second time point, and a subsequent decrease to the background amount of extracellular nucleic acids in the third and subsequent time points. The number of lysed cells can be estimated by counting the number of lysis events seen in the time series and then using the digital-loading formula to correct for the probability of more than one lysis event being captured in the same time point:

$$\# Lysed = - ( \frac{1}{T} ) ln ( \frac{\# Events}{\# TimePointWindow} ) , \qquad ( 15 )$$

where #Lysed is the estimated number of lysed cells that originated from a given partition, #Events is the number of lysis events observed, #TimePointWindow is a chosen number of time points over which the rate of lysis can be assumed to be nearly constant, and T is the duration of each time point. For example, for typical kill rates of 0.1-0.02 min⁻¹, #TimePointWindow are chosen to cover about 5 minutes in total. Other published algorithms for signal processing, such as those used to detect neuron action potentials in patch-clamp recordings, can also be employed to detect lysis events, especially if the temporal resolution is high [22], [23]

In some embodiments of methods and systems comprising a digitally loaded same-sample time-series herein described the number of cells that lysed in the entire sample is then the sum over all partitions of the number of lysed cells in each partition. A high-resolution time series can be achieved, for example, in a microfluidic chip which creates hundreds of spatially arrayed droplet partitions of a continuously flowing filtrate, yielding a temporal resolution of seconds over an exposure of up to an hour.

Additionally, embodiments of methods and systems comprising a same-sample time series and determination of the rate of lysis allow to address phenomena of heteroresistance, persister cells and antibiotic tolerance. These are three phenotypic phenomena of that would manifest in similar ways in same-sample AST, in both bulk and digitally-loaded embodiments.

“Heteroresistance” refers to a phenotype reported to exist in certain antibiotic and microorganism pairings where an isogenic strain of microorganism contains a subpopulation with increased resistance to that antibiotic, the resistance being non-hereditary or with such decreased fitness that populations immediately revert to a majority susceptible nature when cultured without antibiotics.

The “persister phenotype” refers to antibiotic resistant cells that remain viable and dormant during a long antibiotic exposure, always forming a small fraction of an otherwise susceptible population, with the resistance of these cells being non-hereditary as seen when a culture derived from persister cells is challenged repeatedly to antibiotics.

“Antibiotic tolerance” is a phenotype seen in some microorganisms where a transient ability to survive brief antibiotic exposure is seen, even though the antibiotics are at a concentration above the strain's MIC, but the resistance to the antibiotic is not hereditary.

In embodiments of methods and systems comprising time-series same-sample AST, intra/extra proportion value allows performing AST while addressing of heteroresistance, persister cells and antibiotic tolerance. In particular, in a bulk time-series same-sample AST, these three phenomena of non-genetic resistance would be detected by the absence of extracellular nucleic acids in a series of at least one time point at the end of the exposure coupled with the presence of intracellular nucleic acids at the end of the exposure. For example if a time series were performed with 20 time points, and the ENACVs in order of time first increased, then decreased, and then remained at 0 units starting with the 10-th time point, yet at the 20-th time point the INACV is seen to be 25% of the sum of all 20 ENACVs and the 20-th INACV, then one would suspect that there was a subpopulation of tolerance cells that remains alive while the other subpopulations died out.

In a digitally-loaded time-series same-sample AST, an absence of extracellular nucleic acids across all partitions in a series of at least one time point at the end of the exposure would indicate that all susceptible cells have likely lysed, and so any partitions seen to contain intracellular nucleic acids at the end of the exposure could be interpreted as a resistant subpopulation, the likelihood of a truly resistant subpopulation being increased with the more time points lacking extracellular nucleic acids across all wells. It is important to detect these three phenomena of non-genetic resistance because they would otherwise be interpreted as false positives for resistance. These phenomena can underlie treatment failure during antibiotic therapy, so if any of these three phenomena are detected for a pairing of antibiotic with a patient's microorganism, clinicians may decide against using the identified antibiotics or to use higher doses.

In embodiments of methods and systems comprising time-series same sample detection and in general in any of the embodiments described herein, following detection of the extracellular concentration value and intracellular concentration value and determination of intracellular/extracellular proportion value, the intracellular/extracellular proportion value of the sample is then compared with a reference value indicative of an intracellular/extracellular nucleic acid proportion in the sample in absence of antibiotic treatment, to obtain a treated-reference nucleic acid comparison outcome of the sample.

In particular, in embodiments herein described the comparison can be performed with single intracellular/extracellular proportion value or with plurality of antibiotic treated intracellular/extracellular nucleic acid proportion values of a plurality of samples or sub-samples arranged in a distribution forming a function to provide an antibiotic treated intracellular/extracellular nucleic acid proportion profile of the specimen or of the sample as will be understood by a skilled person.

In embodiments, herein described the reference value can also be a single value or a profile comprising a plurality of reference values, such as for example a reference intracellular/extracellular nucleic acid proportion value of a reference sample corresponding to the antibiotic treated intracellular/extracellular nucleic acid proportion value of the sample and/or threshold values as will be understood by a skilled person upon reading of the present disclosure.

“Reference samples” as used herein indicates samples providing a standard for comparison against an antibiotic treated sample where the factor being tested (here antibiotic treatment) is applied during a testing procedure. Reference samples are used to produce reference nucleic acids.

A part of the treated sample can be used as a reference sample, obtained for example by splitting and processing the treated sample. A second sample treated under the same or different conditions as the first treated sample may also be used as a reference sample.

Reference samples can be control samples, which are samples subjected to the same testing procedure as another corresponding sample, except that the factor being tested is not applied. Reference samples and treated samples can be derived by splitting and manipulating the original sample being tested by the methods herein described. In embodiments herein described the comparison between the intracellular/extracellular proportion value and the reference value, can be performed with various statistical identifiable by a skilled person upon reading of the present disclosure.

The terms “statistical test”, “machine learning technique”, and “machine learning algorithm” used herein refer to any one of a variety of models and algorithms, or combination of such models and algorithms, described in the literature and known to the skilled person which can be employed at any step in the disclosed methods herein requiring one to classify observations from numerical or categorical data; that is, to predict whether observations arose from a certain class of entity[24]-[28]. To perform classifications from data, one may employ statistical tests, which are algorithms that assume an underlying statistical model and give the probability or likelihood of summary statistics. In addition, or as an alternative, one can employ machine learning algorithms, which are algorithms that map data to the classification output, sometimes assuming an underlying statistical model. Example steps in our disclosed inventions that use statistical tests or machine learning techniques include the calling of well loading status during digital sample partitioning, the calling of antibiotic susceptibility by each accessibility AST embodiment, and the creation of thresholds for antibiotic susceptibility calls calculated from prior experiments. Each statistical test or machine learning technique's performance varies depending on the way a particular embodiment of accessibility AST generates its data, and some tests are not appropriate for some situations. Some tests are special cases of a more generalized, more complicated test. Using a more complicated algorithm to analyze a simple data set will be equivalent is not necessary. These unsupervised and supervised machine learning algorithms and statistical tests include: any univariate or multivariate, parametric or non-parametric, one-sided or two-sided, paired or independent, frequentist or Bayesian statistical model and test (t-tests, multiple t-tests, analysis of variance (ANOVA), repeated measures ANOVA, one-way ANOVA, multivariate analysis of variance (MANOVA), analysis of covariance, Pearson's r test, Spearman's r, McNemar test, Friedman test, Durbin test, Fisher's exact test, Boschloo's test, Barnard's test, Chi-square test, the sign test, the exact Z-pooled and Z-unpooled tests, Kruskal-Wallis test, Mann-Whitney U/Wilcoxon rank-sum test, Wilcoxon signed-rank test, Kolmogorov-Smirnov test, bootstrapping, Gaussian and other parametric mixture models, multilevel models, Bayesian hierarchical models); regression analysis (linear regression, multiple regression, gradient descent, ordinary least squares regression, logistic regression, probit regression, generalized linear regression, non-linear regression, mixed effects models, measurement error models, Bayesian regression, ridge regression, LASSO, locally-weighted linear regression, multivariate adaptive regression splines, nonparametric regression); times series analysis (autoregressive models, autoregressive moving average models, autoregressive integrated moving average models, stochastic processes, branching processes, Gaussian processes, survival analysis, Kaplan-Meier estimate, proportional hazards models, Cox proportional hazard model, log-rank test); cluster analysis (k-means clustering, k-medoids clustering, partitioning around medoids clustering, nearest neighbors clustering, hierarchical clustering, agglomerative hierarchical clustering, divisive hierarchical clustering, density-based spatial clustering of applications with noise, stochastic network embeddings); matrix factorization techniques (principle components analysis, non-negative matrix factorization, singular value decomposition, collaborative filtering, spectral clustering), artificial neural networks (including “deep learning”, deep artificial neural networks); other supervised classification algorithms (decision trees, classification and regression trees, random forests, support vector machines), generative or Bayesian probability models (naïve Bayes classifiers, Bayesian/belief/probabilistic graphical networks/models); general adversarial networks, reinforcement learning, and others identifiable to a skilled person.

In embodiments herein described the comparison between intracellular and extracellular proportion value and reference value performed with suitable statistical testes, results in a treated-reference nucleic acid comparison outcome of the sample sub-sample and/or the specimen.

In particular, in some embodiments, the comparison outcome can be a treated-reference nucleic acid comparison value obtained by providing a relative difference between the antibiotic treated intracellular/extracellular nucleic acid proportion profile of the specimen and the reference value or a mathematical equivalent thereof.

In some embodiments, the treated-reference nucleic acid comparison outcome of the specimen is a determination on whether the antibiotic treated intracellular/extracellular nucleic acid proportion profile of the specimen is above or below the reference value.

In some embodiments, herein described, the treated-reference nucleic acid comparison outcome of the specimen pair of test and reference conditions is indicative of resistance or susceptibility of the microorganism to the antibiotic in the test condition.

In embodiments where the test condition contained an antibiotic at a breakpoint concentration, then the overall susceptibility of the microorganism strain will be the same as the susceptibility detected in the test condition as revealed by the treated-reference nucleic acid comparison outcome. Knowing the treated-reference nucleic acid comparison outcomes from additional concentrations of the antibiotic will help confirm this strain-level susceptibility call.

In embodiments where the test condition contained an antibiotic above or below the standardized breakpoint concentration, then the treated-reference nucleic acid comparison outcome reveals the susceptibility of the strain at the examined concentration only. The overall susceptibility of the microorganism strain will require additional treated-reference nucleic acid comparison outcomes from test conditions containing different antibiotic concentrations, preferably including any standardized breakpoint concentrations defined for that pairing of species of microorganism and antibiotic compound. In some embodiments, same-sample AST methods herein described can be performed in high-throughput.

The term “high-throughput” refers to assay designs that enable users to process large numbers of samples in a short amount of time, often with fewer reagents as well, and often utilizing specialized equipment to achieve higher efficiency.

In some embodiments, same-sample AST methods herein described can be parallelized.

The term “parallelized” refers to assay protocols in which multiple same-sample AST assays can be performed simultaneously in a high-throughput fashion.

Measurements or assays that are parallelized can simultaneously test multiple specimens, samples of specimens, or partitions of samples from the same or different patient; test multiple antibiotics against the same clinical specimen; and test multiple different concentrations of each antibiotic. When creating multiple antibiotic exposures from the same clinical sample, one is able to examine multiple different antimicrobial agents and one or more doses of each antimicrobial agents.

For example, parallelized same-sample AST can be used to establish minimal inhibitory concentration (MIC), minimal bactericidal concentration (MBC), and/or other relevant pharmacodynamic parameters that describe effects of antibiotics on bacteria. The definitions of these parameters can be found in the Clinical Laboratory Standards Institute (CLSI) guidelines[4] and European Committee on Antimicrobial Susceptibility Testing (EUCAST) guidelines[ 11]. Finding the MIC, or a range of concentrations in which the true MIC lies, for a certain pairing of microorganism and antimicrobial requires that the practitioner test several different antimicrobial concentrations of the selected antimicrobial to narrow down the range of possible concentrations.

For example, in the standard reference protocol for broth microdilution, the microorganism is inoculated into a 2-fold serial dilution of antimicrobial concentrations. Among the dilutions chosen are the breakpoint concentrations, published in the above guidelines, that delineate whether the microorganisms is considered resistant, intermediate, or susceptible. A parallelized same-sample AST would enable the MIC to be determined in the time necessary for one assay run rather than in the longer time it would take to run each concentration's same-sample AST in series.

To perform same-sample AST in a high-throughput manner, one can employ any of the variety of existing instrumentations, equipment, hardware, machines, devices, consumable products, and other technology used for high-throughput assays, herein referred to as “high-throughput instrumentation” that are employed nowadays in “wet laboratory” settings[9], [10], [13], [29]-[31]. The term “wet laboratories” refer to facilities, spaces, or institutions in which users of the laboratory perform controlled handling or characterization of material substances for the ultimate purpose of information generation, including clinical, forensic, and research laboratories in fields such as but not limited to medicine, veterinary medicine, clinical microbiology, clinical chemistry, laboratory medicine, pathology, analytical chemistry, public health, pharmaceuticals, forensics, law enforcement, bioterrorism and national security, food and beverage, agriculture, natural resource management, basic science including life sciences (biology, chemistry, physics, geosciences, environmental science, material science), engineering, bioengineering, and biotechnology.

High throughput instrumentation includes the use of multiwell vessels such as microtiter plates and filter plates.

A microtiter plate is a type of laboratory vessel, usually consumable but sometimes reusable, comprising multiple individual vessels called “wells”, usually with rigid walls, arranged in a standardized, regular, usually rectangular layout to facilitate easy and rapid repeated or parallel handling, and manufactured from a variety of polymeric plastic or glass materials. Preferred materials for the construction of the plates do not dissolve or react with the intended liquid sample and do not exhibit high binding of any intended analyte chemical species. The user can perform experiments can discern which plates are appropriate with the aqueous solutions that are the intended liquid samples in the same-sample AST disclosed herein. Materials compatible with the disclosed same-sample AST include polystyrene, polyvinyl chloride (vinyl, PVC), polypropylene, polyethylene terephthalate (PET, PETE), polycarbonate, cyclic olefin copolymer, acrylic copolymer, polyacrylonitrile (Barex®), styrene-acrylonitrile resin (SAN), polyethylene, high-density polyethylene (HDPE), polyvinylidene chloride, and polyvinylidene fluoride. Microtiter plates include those made with 12 (3×4), 24 (4×6), 48 (6×8), 96 (8×12), 384 (12×16), and 1536 (32×48) wells, those numbers being common standard layouts in commercially available microtiter plates, but other layouts and numbers of wells are envisioned. The wells may have different shapes, with circular and square prisms being common examples, and the bottom of the wells may be V-shaped, U-shaped, rounded, flat, or any other unspecialized shape, so long as the microtiter plate is being used to hold and keep separate liquid samples during the antibiotic exposure, nucleic acid compartment separation (e.g. filtration or centrifugation), nucleic acid extraction, reverse transcription, and nucleic acid amplification steps of same-sample accessibility AST. Microtiter plates are preferably used when sterile and not containing exogenous substances so as to reduce contamination of any enclosed sample and to prevent incorrect interpretation of assay outputs. Since the well of a microtiter plate is functionally analogous to a single vessel, usually called a tube or test tube, any array on conglomerate of tubes can replace the use of microtiter plates in our protocol. Similarly, some commercial automated broth microdilution AST systems use rigid plastic multiwell cards (e.g. Vitek 2 64-well cards) to house cultures of bacteria; these cards are equivalent in function to microtiter plates. Picotiter plates are another type of laboratory vessel that comprise an array of multiple wells. Picotiter plates are similar to microtiter plates but have a smaller volume and a larger number of wells. It is readily envisioned that same-sample AST can be performed in picotiter plates in a high throughput manner.

A filter plate is a microtiter plate in which the bottom wall of each well contains an outlet that can be reversibly sealed, or which does not need to be sealed due to the slow speed with which contained liquid will leave the outlet when no outside driving force is applied. Before a liquid sample placed into the well can leave the outlet, however, it must pass through a filter membrane spanning the outlet. The driving force that moves the liquid sample through the filter at the desired time can be gravity, centrifugation, positive air pressure, or vacuum suction (negative air pressure). Different choices of materials for filter plate walls and filter membranes are already available commercially. Walls may comprise any rigid polymeric plastic used to make disposable lab plasticware, with preferred materials not dissolving or reacting with the intended liquid sample and not exhibiting high binding of any intended analyte chemical species. Wall materials compatible with the aqueous solutions present in our disclosed method include polystyrene, polyvinyl chloride (vinyl, PVC), polypropylene, polyethylene terephthalate (PET, PETE), polycarbonate, cyclic olefin copolymer, acrylic copolymer, polyacrylonitrile (Barex®), styrene-acrylonitrile resin (SAN), polyethylene, high-density polyethylene (HDPE), polyvinylidene chloride, and polyvinylidene fluoride. Filter membranes may be made of any polymeric material that does not dissolve or react with the intended liquid sample. Filter membrane materials preferably do not exhibit high binding to any intended analyte chemical species, but if binding is detected, coating with a blocking agent mitigates the loss of analyte. Example blocking agents include salmon sperm DNA, yeast tRNA, any nucleic acid not derived from the target microorganisms, bovine serum albumin, and milk powder. Example filter membrane materials compatible with the aqueous solutions used in the disclosed same-sample filtration AST include cellulose nitrate, cellulose acetate, regenerated cellulose, mixed cellulose ester, nitrocellulose, nylon, polyethersulfone (PES, polysulfone), polytetrafluoroethylene (PTFE, Teflon®), polyvinylidene fluoride, polycarbonate, glass fibers, borosilicate glass fibers, quartz fibers, paper, and hardened paper. If the filter membrane is of a material not wettable by the intended liquid sample, the membrane may be coated by detergents. If detergents interfere with downstream applications, they can be removed with a wash step in which the intended liquid (e.g. water) is passed through the filter shortly before use. The use of a filter plate enables the parallel and simultaneous filtration of many samples, thus enabling high throughput assay execution. Filter plates are preferably used when sterile and not containing exogenous substances (except for the use of detergents to coat filter membranes in some cases) so as to reduce contamination of any enclosed sample and to prevent incorrect interpretation of assay outputs. Filter plates are available commercially from several large-scale manufacturers. Individual filter units, tubes, or cartridges are analogous to a single well of a filter plate, so any array or conglomerate of such filter unites, tubes or cartridges can replace the use of a filter plate during the filtration step of the same-sample filtration AST disclosed herein.

High throughput instrumentation also includes the use of manually operated equipment that enables parallel sample processing. Examples of manually operated, parallel processing equipment are the multichannel pipettors and repeating pipettors [30]. Multichannel pipettors, or multichannel micropipettors, are a type of pipettor in which a user can simultaneously draw and expel parallel amounts of liquid from several pipettor tips simultaneously. A pipettor is a handheld volumetric device that is used to draw and expel liquids of known volume into a pipette or pipette tip. Pipettes are narrow, sometimes calibrated tube into which small amounts of liquid are suctioned for transfer or measurement, while pipette tips are disposable and removable pipettes designed to be attached to the ends of some pipettes. Micropipettors are pipettors designed to move microliter-scale amounts of liquid, are ubiquitous pieces of wet laboratory equipment, and usually use air displacement to draw in liquid. Most commercial multichannel pipettors have tips arranged in a straight light with a standard spacing between them that matches commercial plastic ware. Multichannel pipettors with adjustable tip spacing are also commercially available. Repeating pipettors, also known as repeat pipettors or repeater pipettors, are pipettors in which an electronic motor repeatedly dispenses a controlled amount of liquid that is less than the total amount drawn up in the initial drawn. This pipettor design saves time when transferring the same liquid to multiple vessels in series.

High throughput instrumentation also includes the use of laboratory automation systems (LAS). As used herein, “laboratory automation systems” is used to denote those machines that automate physical manipulations which would otherwise be performed manually by humans, usually with motorized moving parts and optionally sensors and computer processors that allow the robot to respond to inputs or to be flexibly programmed by human users. The tasks that laboratory automation systems can automate include specimen identification; specimen delivery; specimen processing; sample introduction and internal transport; sample loading and aspiration; reagent handling and storage; reagent delivery; chemical reaction phase; measurement approaches; and signal processing, data handling, and process control. The manual actions that laboratory automation systems can automate include liquid handling (addition, removal, aliquoting, or transfer of volumes of liquid from one vessel to another); opening and closing of vessel lids or seals (decapping and recapping); liquid mixing (e.g. forceful dispensing, physical stirring, magnetic stirring, vigorous lateral displacement, vortexing); sorting of samples; sample level detection or evaluation of specimen integrity and adequacy; centrifugation; the incubation or thermocycling of vessels at controlled temperatures (thermal regulation, often by air baths, water baths, Peltier tiles, and piezoelectric devices); optical measurements such as fluorescence photometry (fluorometry), reflectance photometry, optical absorbance (turbidimetry, nephelometry), chemiluminescence, bioluminescence, electrochemical measurements, photographic or microscopic imaging, or spectroscopy; and other measurements of physical properties such as temperature, calorimetry, and gas pressure. Laboratory automation systems include devices known as microtiter plate systems, liquid handling robots, automated liquid handling systems, pipetting robots, automated pipetting stations, acoustic droplet ejection systems, acoustic liquid handlers, and plate readers (also known as microplate readers and microplate photometers). Laboratory automation systems may be composed of combinations of the aforementioned machines, and also may combine other motorized and non-motorized laboratory devices to achieve the automation of manual laboratory tasks. These other laboratory devices include plate sealers, incubators/heat blocks/heating elements, shakers, thermocyclers, thermomixers, lamps, cameras, and photometers. Laboratory information systems (LIS) which keep track of specimen identity, maintain databases of assay results, and analyze data from current and prior assays through included software, may be a feature of automated AST systems that perform the same-sample AST method disclosed herein. For example, parallelized measurements may use barcoding and barcode reading equipment for sample identification. In some embodiments, the use of such laboratory information systems provides a beneficial feature, but not necessary for same-sample AST to be performed, and their addition to an automated system performing same-sample AST does not fundamentally change the same-sample AST method.

High throughput instrumentation also includes the use of microfluidic devices. For the purposes of this disclosure, the types of devices known as “lab-on-a-chip” (LOC) devices, Bio-MEMS (biological or biomedical microelectromechanical) devices, or micro total analysis systems (μTAS) are considered to be microfluidic devices. Microfluidic devices are integrated devices that manipulate fluids at microliter scales. A narrower definition of microfluidics states that devices are microfluidic when the behavior of the liquid manipulated is more strongly affected by surface forces than by inertial forces, namely when flow is laminar and the Reynolds number is lower than 2000. For the purposes of high throughput assay design, the broader definition of microfluidics applies, as it is the miniaturization and integration of otherwise manual mechanical actions into the automated device that allows parallelization and high throughput assay performance. Microfluidic devices are generally made of solid materials in which micron-scale patterns have been created by photolithography, micromachining, soft lithography, micromolding, self-assembly, or other microfabrication techniques. Some devices employ continuous fluid flow, wells, valves, mixers, and other components. Others manipulate discrete plugs of fluid within enclosed or partially open (e.g. paper devices) channels or even flat surfaces (electrowetting digital microfluidics).

A subset of microfluidic devices known as droplet microfluidic devices create stable droplet emulsions where the liquid-liquid interface functions as the separation between droplets rather than the walls of solid wells. Properties of the individual droplets can then be measured by droplet reading instruments, such as fluorometers. Since tens of thousands of droplets can be generated quickly, and their properties measured, digital same-sample AST could conceivably be adapted so that the sample partitions become the emulsion droplets.

In general, same-sample methods herein described can be performed with a corresponding system comprising at least one probe specific for a nucleic acid of the target microorganism and reagents for detecting the at least one probe. The at least one probe and reagents are included in the system for simultaneous combined or sequential use in any one of the methods of the present disclosure.

In some embodiments of the system herein described the system comprises primers configured to specifically hybridize with a sequence of nucleic acid from the target organism.

In some embodiments, the systems of the disclosure to be used in connection with methods herein described can further comprise an antibiotic formulated for administration to a sample in combination with the at least one probe.

In some embodiments, the systems of the disclosure, the system further comprises an antibiotic formulated for administration to an individual in an effective amount to treat a microorganism infection in the individual.

In some embodiments, the systems of the disclosure to be used in connection with methods herein described, the reagents comprise DNA extraction, RNA extraction kit and amplification mix. The system can also include one or more antibiotics and/or exposure media with or without the antibiotics. The system can also include reagents required for preparing the sample, such as one or more of buffers e.g. lysis, stabilization, binding, elution buffers for sample preparation, enzyme for removal of DNA e.g. DNase I, and solid phase extraction material for sample preparation, reagents required for quantitative detection such as intercalating dye, reverse-transcription enzyme, polymerase enzyme, nuclease enzyme (e.g. restriction enzymes; CRISPR-associated protein-9 nuclease; CRISPR-associated nucleases as described herein) and reaction buffer. Sample preparation materials and reagents may include reagents for preparation of RNA and DNA from samples, including commercially available reagents for example from Zymo Research, Qiagen or other sample preparations identifiable by a skilled person. The system can also include means for performing DNA or RNA quantification such as one or more of: container to define reaction volume, droplet generator for digital quantification, chip for digital detection, chip or device for multiplexed nucleic acid quantification or semiquantification, and optionally equipment for temperature control and detection, including optical detection, fluorescent detection, electrochemical detection.

In some embodiments, the system can comprise a device combining all aspects required for an antibiotic susceptibility test.

The systems herein disclosed can be provided in the form of kits of parts. In kit of parts for performing any one of the methods herein described the probes and the reagents for the related detection can be included in the kit alone or in the presence of one or more antibiotic, as well as one or more of the high-throughput instrumentation herein described. For example, the kit can comprise a component mixture for preparing a lysis solution that include lysis of the target microorganism including lysis buffers and a mix that can be diluted or reconstituted to make a lysis buffer as will be understood by a person skilled in the art. The kit can also comprise a component mixture for preparing an inactivation solution to inactivate nucleases. The kit can also comprise an amplification reagent compatible with at least one of the lysis solution or inactivation solution herein described.

In a kit of parts, the probes and the reagents for the related detection, antibiotics, and additional reagents identifiable by a skilled person are comprised in the kit independently possibly included in a composition together with suitable vehicle carrier or auxiliary agents. For example, one or more probes can be included in one or more compositions together with reagents for detection also in one or more suitable compositions.

Additional components can include labeled polynucleotides, labeled antibodies, labels, microfluidic chip, reference standards, and additional components identifiable by a skilled person upon reading of the present disclosure.

The terms “label” and “labeled molecule” as used herein refer to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “labeling signal” as used herein indicates the signal emitted from the label that allows detection of the label, including but not limited to radioactivity, fluorescence, chemoluminescence, production of a compound in outcome of an enzymatic reaction and the like.

In embodiments herein described, the components of the kit can be provided, with suitable instructions and other necessary reagents, in order to perform the methods here disclosed. The kit will normally contain the compositions in separate containers. Instructions, for example written or audio instructions, on paper or electronic support such as tapes, CD-ROMs, flash drives, or by indication of a Uniform Resource Locator (URL), which contains a pdf copy of the instructions for carrying out the assay, will usually be included in the kit. The kit can also contain, depending on the particular method used, other packaged reagents and materials (wash buffers and the like).

The methods described herein can be performed by computer or specialized computing machines. For example, the algorithms can be implemented in a system using software, hardware, firmware, or some combination of the above. In some embodiments, the algorithms are implemented on software running on a processor and stored in memory (disc drive, solid state drive, flash drive, etc.). In some embodiments, the system can utilize look-up tables for data retrieval as part of the computations. Look-up tables are arrays of information in memory that relate a set of input values to corresponding pre-determined output values.

A description of exemplary sets of preferred embodiments of the same-sample AST herein described is provided below.

In particular, according to the first aspect a method to detect a nucleic acid of a microorganism in a sample including the microorganism, the method comprising
contacting the sample with an antibiotic to provide an antibiotic-treated sample,
separating the antibiotic-treated sample into an antibiotic-treated extracellular component and an antibiotic-treated cellular component,
detecting a nucleic acid concentration of the antibiotic-treated extracellular component to obtain an antibiotic-treated extracellular nucleic acid concentration value, and
detecting a nucleic acid concentration of the antibiotic-treated cellular component to obtain an antibiotic-treated intracellular nucleic acid concentration value.

In a first set of embodiments of the method according to the first aspect the separating is performed by mechanical separation of the antibiotic treated sample, possibly by filtration and/or centrifugation of the antibiotic treated sample.

In a second set of embodiments the method according to the first aspect, the method further comprises comparing the detected antibiotic treated intracellular nucleic acid (NA) concentration value and the detected antibiotic treated extracellular nucleic acid (NA) concentration value, to provide an antibiotic treated intracellular/extracellular nucleic acid proportion value of the sample.

In some embodiments of the second set of embodiments the method according to the first aspect, the antibiotic treated intracellular/extracellular nucleic acid proportion value of the sample is a ratio of the detected antibiotic treated intracellular concentration value or of the antibiotic treated detected extracellular concentration value and a sum of the detected antibiotic treated intracellular NA concentration value and the detected antibiotic treated extracellular NA concentration value, or a mathematical equivalent thereto.

In some embodiments of the second set of embodiments the method according to the first aspect, the antibiotic treated intracellular/extracellular nucleic acid proportion value of the sample is a relative difference between the detected antibiotic treated intracellular concentration value and the detected antibiotic treated extracellular nucleic acid concentration value or a mathematical equivalent thereto.

In some embodiments of the second set of embodiments the method according to the first aspect, the antibiotic treated intracellular/extracellular nucleic acid proportion value of the sample is a percentage extracellular concentration or an intracellular percentage concentration or a mathematical equivalent thereto.

In some embodiments of the second set of embodiments the method according to the first aspect, the antibiotic treated intracellular/extracellular nucleic acid proportion value of the sample is a probability of lysis.

In some embodiments of the second set of embodiments the method according to the first aspect, the method further comprises determining a proportionality of dead and live microorganism cells in the sample caused by and/or or as a function of, the antibiotic by determining an intra/extra proportion value of the sample to provide a dead/live proportion value of the microorganism cells in the sample

In some embodiments of the second set of embodiments the method according to the first aspect, the method further comprises
comparing the antibiotic treated intracellular/extracellular nucleic acid proportion value of the sample with
a reference value indicative of an intracellular/extracellular nucleic acid proportion in the sample in absence of antibiotic treatment
to obtain a treated-reference nucleic acid comparison outcome of the sample.

In some embodiments of the second set of embodiments the method according to the first aspect directed to obtain a treated reference nucleic acid comparison outcome of the same sample, the treated-reference nucleic acid comparison outcome of the sample is
a treated-reference nucleic acid comparison value obtained by providing a relative difference between the antibiotic treated intracellular/extracellular nucleic acid proportion value of the sample and the reference value or a mathematical equivalent thereof.
   13. The method of claim 11, wherein the treated-reference nucleic acid comparison outcome of the sample is
a determination on whether the antibiotic treated intracellular/extracellular nucleic acid proportion value of the sample is above or below the reference value.

In embodiments of the second set of embodiments the method according to the first aspect directed to obtain a treated reference nucleic acid comparison outcome of the same sample, wherein the treated-reference nucleic acid comparison outcome of the sample is indicative of resistance or susceptibility of the microorganism to the antibiotic.

In some embodiments of the second set of embodiments the method according to the first aspect directed to obtain a treated reference nucleic acid comparison outcome of the same sample, the reference value comprises
a reference intracellular/extracellular nucleic acid proportion value of a reference sample corresponding to the antibiotic treated intracellular/extracellular nucleic acid proportion value of the sample.

In some embodiments of the second set of embodiments the method according to the first aspect directed to obtain a treated reference nucleic acid comparison outcome of the same sample based on a reference intracellular/extracellular nucleic acid proportion value, the reference intracellular/extracellular nucleic acid proportion value is obtained by comparing
a detected reference intracellular nucleic acid concentration value of the reference sample and
a detected reference extracellular nucleic acid concentration value of the reference sample.

In some embodiments of the second set of embodiments the method according to the first aspect directed to obtain a treated reference nucleic acid comparison outcome of the same sample based on a reference intracellular/extracellular nucleic acid proportion value, the reference sample is an antibiotic untreated control sample, and/or second sample treated with antibiotic under different experimental conditions as the antibiotic treated sample.

In some embodiments of the second set of embodiments the method according to the first aspect directed to obtain a treated reference nucleic acid comparison outcome of the same sample, the reference value comprises an extracellular nucleic acid concentration value detected in an untreated extracellular fraction of the sample separated from the sample before the contacting.

In some embodiments of the second set of embodiments the method according to the first aspect directed to obtain a treated reference nucleic acid comparison outcome of the same sample, the reference value is provided by a plurality of reference values arranged in a distribution forming a function to provide a reference profile.

In some embodiments of the second set of embodiments the method according to the first aspect directed to obtain a treated reference nucleic acid comparison outcome of the same sample the method further comprises
determining antibiotic susceptibility when the reference nucleic acid comparison outcome of the sample indicates an increased lysis and an increased dead/live proportion of the microorganism cells in the antibiotic-treated sample compared to a sample treated under reference conditions; or
determining antibiotic resistance when the reference nucleic acid comparison outcome of the sample indicates a substantially same dead/live proportion of the microorganism cells in the antibiotic-treated sample compared to a sample treated under reference conditions.

According to a second aspect, embodiments of the second set of embodiments the method according to the first aspect directed to obtain a treated reference nucleic acid comparison outcome of the same sample are performed with a reference value comprising
a threshold value obtained based on standard deviations of distributions of extracellular and/or intracellular nucleic acid concentrations of the microorganism in absence of antibiotic treatment.

In embodiments according to the second aspect, the reference value can comprise the threshold value obtained from distributions of extracellular and/or intracellular nucleic acid concentrations of the microorganism in presence of background events unrelated to antibiotic treatment.

In embodiments according to the second aspect, the reference value can comprise a threshold value obtained based on standard deviations of a distribution of intracellular/extracellular nucleic acid proportion values of the sample in the absence of antibiotic treatment.

In embodiments according to the second aspect, the reference value can comprise a. threshold value obtained based on standard deviations of a distributions of treated-reference comparison values obtained by
comparing antibiotic treated intracellular/extracellular nucleic acid proportion values of the sample to intracellular/extracellular nucleic acid proportion values of the sample obtained in the absence of antibiotic treatment.

In embodiments according to the second aspect, the reference value can comprise a threshold value obtained from a distribution of antibiotic treated intracellular/extracellular nucleic acid proportion values obtained in the absence of antibiotic treatment in the presence of background events unrelated to antibiotic treatment.

In embodiments according to the second aspect, the reference value can comprise a threshold value obtained from a distributions of treated-reference comparison values obtained by
comparing antibiotic treated intracellular/extracellular nucleic acid proportion value of the sample to intracellular/extracellular nucleic acid proportion values of a reference sample obtained in the absence of antibiotic treatment in the presence of background events unrelated to antibiotic treatment.

In embodiments according to the second aspect, the reference value can comprise a reference intracellular/extracellular nucleic acid proportion value of claim 7 or 8 and a treated-reference comparison values obtained by
comparing antibiotic treated intracellular/extracellular nucleic acid proportion values of the sample to intracellular/extracellular nucleic acid proportion values of the sample obtained in the absence of antibiotic treatment.

In embodiments according to the second aspect, the reference value can comprise a threshold is obtained from the measurement of a reference intracellular/extracellular nucleic acid proportion values of a reference sample

In embodiments according to the second aspect, the reference value can comprise a reference value is provided by a plurality of reference values arranged in a distribution forming a function to provide a reference profile.

In embodiments according to the second aspect, the method can further comprise determining antibiotic susceptibility when the reference nucleic acid comparison outcome of the sample indicates an increased lysis and an increased dead/live proportion of the microorganism cells in the antibiotic-treated sample compared to a sample treated under reference conditions; or determining antibiotic resistance when the reference nucleic acid comparison outcome of the sample indicates a substantially same dead/live proportion of the microorganism cells in the antibiotic-treated sample compared to a sample treated under reference conditions.

According to a third aspect a method is described to detect a nucleic acid of a microorganism in a sample including the microorganism, the method comprising performing n cycles of
contacting a sample with an antibiotic to provide an antibiotic treated sample,
separating the antibiotic treated sample, to obtain an antibiotic treated extracellular fraction of the sample and an antibiotic treated cellular fraction of the sample,
detecting a nucleic acid concentration of the antibiotic treated extracellular fraction, to obtain a detected antibiotic treated extracellular nucleic acid concentration value of the antibiotic treated sample. and
combining the antibiotic treated cellular fraction of the sample with culture media to reconstitute the sample;
   to obtain an nth reconstituted sample, n being an integer equal or higher than 1.

In embodiments of the method according to the third aspect the method can further comprise in an n+1 cycle:
contacting the nth reconstituted sample with an antibiotic to obtain an antibiotic treated nth reconstituted sample
separating the antibiotic treated nth reconstituted sample, to obtain an antibiotic treated extracellular fraction of the nth reconstituted sample and an antibiotic treated cellular fraction of the nth reconstituted sample;
detecting a nucleic acid concentration of the antibiotic treated extracellular fraction of the nth reconstituted sample, to obtain a detected antibiotic treated extracellular nucleic acid concentration value of the nth reconstituted sample and
detecting a nucleic acid concentration of the antibiotic treated cellular fraction of the nth reconstituted sample, to obtain a detected antibiotic treated intracellular nucleic acid concentration value of the antibiotic treated nth reconstituted sample.

In embodiments of the method according to the third aspect, the method further can further comprise
establishing an intracellular nucleic acid concentration of the antibiotic treated sample of each of the n-cycles, to obtain an established antibiotic treated intracellular nucleic acid concentration value of the antibiotic treated sample of each of the n-cycles, by comparing
the detected antibiotic treated extracellular nucleic acid concentration value of each n-cycles,
the detected antibiotic treated extracellular nucleic acid concentration value of the nth reconstituted sample; and
the detected antibiotic treated intracellular nucleic acid concentration value of the nth reconstituted sample.

In embodiments of the method according to the third aspect, the method further can further comprise
comparing the established or detected antibiotic treated intracellular nucleic acid concentration value of the reconstituted sample of each cycle of the n-cycles and the n+1 cycle, with the detected antibiotic treated extracellular nucleic acid concentration value of the reconstituted sample of a same each cycle of the n-cycles and the n+1 cycle,
   to provide an antibiotic treated intracellular/extracellular nucleic acid proportion value of the reconstituted sample of each cycle of the n-cycles and the n+1 cycle, forming
   a plurality of antibiotic treated intracellular/extracellular nucleic acid proportion values of the sample for n+l-cycles.

In methods according to the third aspect directed to provide a plurality of antibiotic treated intracellular/extracellular nucleic acid proportion values of the sample for n+1-cycles, the plurality of antibiotic treated intracellular/extracellular nucleic acid proportion values of the sample for the n+1-cycles can comprise the antibiotic treated intracellular/extracellular nucleic acid proportion values of any one of claims 6 to 8.

In methods according to the third aspect directed to provide a plurality of antibiotic treated intracellular/extracellular nucleic acid proportion values of the sample for n+1-cycles, wherein the plurality of antibiotic treated intracellular/extracellular nucleic acid proportion values of the sample for the n+1 cycles can be arranged in a distribution forming a function to provide an antibiotic treated intracellular/extracellular nucleic acid proportion profile of the sample for the n+l-cycles.

In methods according to the third aspect directed to provide a plurality of antibiotic treated intracellular/extracellular nucleic acid proportion values of the sample for n+1-cycles, the method can further comprise
comparing the antibiotic treated intracellular/extracellular nucleic acid proportion profile of the sample for the n+1-cycles with
a reference value indicative of an intracellular/extracellular nucleic acid proportion in the sample in absence of antibiotic treatment
to obtain a treated-reference nucleic acid comparison outcome of the sample for the n+1 cycles.

In methods according to the third aspect directed to provide a treated-reference nucleic acid comparison outcome of the sample for the n+1 cycles, the treated-reference nucleic acid comparison outcome of the sample for the n+1 cycles can be
a treated-reference nucleic acid comparison value obtained by providing a relative difference between the antibiotic treated intracellular/extracellular nucleic acid proportion profile of the sample for the n+1-cycles and the reference value or a mathematical equivalent thereof.

In methods according to the third aspect directed to provide a treated-reference nucleic acid comparison outcome of the sample for the n+1 cycles, the treated-reference nucleic acid comparison outcome of the sample for the n=1 cycles is
a determination on whether the antibiotic treated intracellular/extracellular nucleic acid proportion profile of the sample for the n+1-cycles is above or below the reference value.

In methods according to the third aspect directed to provide a treated-reference nucleic acid comparison outcome of the sample for the n+1 cycles, the treated-reference nucleic acid comparison outcome of the sample for the n+1-cycles is indicative of resistance or susceptibility of the microorganism to the antibiotic.

In methods according to the third aspect directed to provide a treated-reference nucleic acid comparison outcome of the sample for the n+1 cycles, the reference value is any one of the reference values of any one of the reference value of the method according to the second aspect.

In methods according to the third aspect directed wherein the sample can be a partitioned sample of a plurality of partitioned samples obtained by partitioning a specimen to obtain the plurality of samples.

According to a fourth aspect, in embodiments of the first and second aspect
the sample comprises a plurality of samples of a same specimen, and
the contacting, the separating, the detecting a nucleic acid concentration of the antibiotic-treated extracellular component and the detecting a nucleic acid concentration of the antibiotic-treated cellular component are performed on each sample of the plurality of the samples.
to obtain an antibiotic-treated intracellular nucleic acid concentration value and an antibiotic-treated extracellular nucleic acid concentration value for each sample of the plurality of samples of the specimen.

In embodiments of the method according to the fourth aspect, the contacting can be performed on each sample of the plurality of sample, at a same or different timing, with a same or different antibiotic and/or with a same or different antibiotic amounts.

In embodiments of the method according to the fourth aspect, the method can further comprise
comparing the detected antibiotic treated intracellular concentration value and the detected antibiotic treated extracellular nucleic acid concentration value of each sample of the plurality of samples
   to provide a plurality of an antibiotic treated intracellular/extracellular nucleic acid proportion values of the specimen.

In embodiments of the method according to the fourth aspect direct to obtain a plurality of an antibiotic treated intracellular/extracellular nucleic acid proportion values of the specimen, the plurality of antibiotic treated intracellular/extracellular nucleic acid proportion values of the specimen comprises any one of the antibiotic treated intracellular/extracellular nucleic acid proportion values of the method of the first aspect.

In embodiments of the method according to the fourth aspect direct to obtain a plurality of an antibiotic treated intracellular/extracellular nucleic acid proportion values of the specimen, the plurality of antibiotic treated intracellular/extracellular nucleic acid proportion values of the specimen are used to provide an antibiotic treated intracellular/extracellular nucleic acid proportion profile of the specimen.

In embodiments of the method according to the fourth aspect direct to obtain a plurality of an antibiotic treated intracellular/extracellular nucleic acid proportion profile of the specimen, the method can further comprise
comparing the antibiotic treated intracellular/extracellular nucleic acid proportion profile of the specimen. with
a reference value indicative of an intracellular/extracellular nucleic acid proportion in a sample of the plurality of sample in absence of antibiotic treatment
to obtain a treated-reference nucleic acid comparison outcome of the specimen.

In embodiments of the method according to the fourth aspect direct to obtain a plurality of a treated-reference nucleic acid comparison outcome of the specimen, the treated-reference nucleic acid comparison outcome of the specimen can be
a treated-reference nucleic acid comparison value obtained by providing a relative difference between the antibiotic treated intracellular/extracellular nucleic acid proportion profile of the specimen and the reference value or a mathematical equivalent thereof.

In embodiments of the method according to the fourth aspect direct to obtain a plurality of a treated-reference nucleic acid comparison outcome of the specimen, the treated-reference nucleic acid comparison outcome of the specimen can be
a determination on whether the antibiotic treated intracellular/extracellular nucleic acid proportion profile of the specimen is above or below the reference value.

In embodiments of the method according to the fourth aspect direct to obtain a plurality of treated-reference nucleic acid comparison outcome of the specimen, the treated-reference nucleic acid comparison outcome of the specimen is indicative of resistance or susceptibility of the microorganism to the antibiotic.

In embodiments of the method according to the fourth aspect direct to obtain a plurality of a treated-reference nucleic acid comparison outcome of the specimen and or, the reference value can be any one of the reference values of the second aspect.

In embodiments of the method according to the fourth aspect, the method can further comprise partitioning a specimen to obtain the plurality of samples, and in particular can comprise digital partitioning.

In embodiments of the method according to the fourth aspect comprising digital partitioning, the digital partitioning provides at least one samples of the plurality of samples not having any cells, at least one sample of the plurality of samples with less than 10 cells or less than 5 cells, and/or preferably at least one sample of the plurality of samples having a single cell of the target microorganism.

In embodiments of the method according to the fourth aspect, the plurality of samples is arranged on a multi-well plate.

According to a fifth aspect, in embodiments of the first and second aspect, the method further comprises
splitting the antibiotic-treated sample to obtain a plurality of sub-samples, and in the method according to the fifth aspect
the contacting is performed under at least one set of test condition in a corresponding at least set of subsample,
the separating, the detecting a nucleic acid concentration of the antibiotic-treated extracellular component and the detecting a nucleic acid concentration of the antibiotic-treated cellular component are performed on each sub-sample of the at least one set of subsamples of plurality of sub-samples,
to obtain an antibiotic-treated intracellular nucleic acid concentration value and an antibiotic-treated extracellular nucleic acid concentration value of the at least one set of sub-samples of the plurality of sub-samples

In embodiments of the method according to the fifth aspect, the method can further comprise
comparing the detected antibiotic treated intracellular concentration value and the detected antibiotic treated extracellular nucleic acid concentration value of the at least one set of sub-samples of the plurality of sub-samples
   to provide an antibiotic treated intracellular/extracellular nucleic acid proportion value of each of the at least one set of sub-samples of the plurality of sub-samples

In embodiments of the method according to the fifth aspect directed to provide an antibiotic treated intracellular/extracellular nucleic acid proportion value of each of the at least one set of sub-samples of the plurality of sub-samples, the antibiotic treated intracellular/extracellular nucleic acid proportion value comprises the antibiotic treated intracellular/extracellular nucleic acid proportion values according to anyone of the method according to the first aspect.

In embodiments of the method according to the fifth aspect directed to provide an antibiotic treated intracellular/extracellular nucleic acid proportion value of each of the at least one set of sub-samples of the plurality of sub-samples, antibiotic treated intracellular/extracellular nucleic acid proportion value of each of the at least one set of sub-samples of the plurality of sub-samples are used to provide an antibiotic treated intracellular/extracellular nucleic acid proportion profile of the sample.

In embodiments of the method according to the fifth aspect directed to provide an antibiotic treated intracellular/extracellular nucleic acid proportion profile of the sample, the method can further comprise
comparing the antibiotic treated intracellular/extracellular nucleic acid proportion profile of the sample. with
a reference value indicative of an intracellular/extracellular nucleic acid proportion in the sample in absence of antibiotic treatment
to obtain a treated-reference nucleic acid comparison outcome of the sample.

In embodiments of the method according to the fifth aspect directed to provide a treated-reference nucleic acid comparison outcome of the sample, the treated-reference nucleic acid comparison outcome of the sample can be
a treated-reference nucleic acid comparison value obtained by providing a relative difference between the antibiotic treated intracellular/extracellular nucleic acid proportion profile of the sample and the reference value or a mathematical equivalent thereof.

In embodiments of the method according to the fifth aspect directed to provide a treated-reference nucleic acid comparison outcome of the sample, the treated-reference nucleic acid comparison outcome of the sample can be a determination on whether the antibiotic treated intracellular/extracellular nucleic acid proportion profile of the sample is above or below the reference value.

In embodiments of the method according to the fifth aspect directed to provide a treated-reference nucleic acid comparison outcome of the sample, the treated-reference nucleic acid comparison outcome of the sample is indicative of resistance or susceptibility of the microorganism to the antibiotic.

In embodiments of the method according to the fifth aspect directed to provide a treated-reference nucleic acid comparison outcome of the sample, the reference value can be any one of the reference values of any one of the methods according to a second aspect.

In embodiments of the method according to the fifth aspect, the method can further comprise partitioning a sample to obtain the plurality of sub-samples, and in particular the method can further comprise performing a digital partitioning. In those embodiments the sample or subsample comprises a plurality of digital samples or sub-samples, and the contacting, the separating and the detecting are performed in each digital sample or sub-sample.

In embodiments of the method according to the fifth aspect comprising digital partitioning, the digital partitioning can provide at least one samples of the plurality of samples not having any cells, at least one sample of the plurality of samples with less than 10 cells or less than 5 cells, and/or at least one sample of the plurality of samples having a single cell of the target microorganism.

In embodiments of the method according to the fifth aspect, the plurality of samples can be arranged on a multi-well plate.

In embodiments of the method according to the fifth aspect comprising digital partitioning, the method can further comprise
determining with a well-loading algorithm an integer count of types of digital samples or sub-samples in the plurality of digital samples or sub-samples, each type comprising,
i) digital samples or sub-samples comprising a lysed microorganism,
ii) digital samples or sub-samples comprising an intact microorganism,
iii) digital samples or sub-samples comprising no microorganism, or
iv) digital samples or sub-samples comprising a combination of lysed microorganism and intact microorganism,
the well-loading algorithm being a function of the antibiotic-treated extracellular nucleic acid concentration value and the antibiotic-treated intracellular nucleic acid concentration value of the plurality of digital samples or sub-samples.

In embodiments of the method according to the fifth aspect comprising digital partitioning and using a well loading algorithm the determined integer counts of types of digital sample or sub-sample can be arranged in a contingency table (a cross tabulation), and particularly a confusion matrix.

In embodiments of the method according to the fifth aspect comprising digital partitioning, the method can further comprise
comparing
   the proportion, rate, or probability of cell lysis in the treated condition with
   the proportion, rate, or probability of cell lysis in a reference condition
      by means of a statistical test describing the likelihood of observing the determined integer counts of types of digital sample or sub-sample in the plurality of digital samples or sub-samples,
      the comparison of proportions, rates, or probabilities possibly being implicitly calculated by the statistical test
      to obtain a treated-reference comparison outcome,

In embodiments of the method according to the fifth aspect comprising digital partitioning directed to provide a treated-reference comparison outcome, wherein the statistical test is a two-sample binomial exact test of the determined integer counts of types of digital sample or sub-sample in the plurality of digital samples or sub-samples.

In embodiments of the method according to the fifth aspect comprising digital partitioning directed to provide a treated-reference comparison outcome and using a statistical test the statistical test can be a Pearson's chi-squared test of the determined integer counts of types of digital sample or sub-sample in the plurality of digital samples or sub-samples.

In embodiments of the method according to the fifth aspect comprising digital partitioning directed to provide a treated-reference comparison outcome, the comparison outcome is indicative of antibiotic susceptibility of the microorganism.

In any embodiments of the method according to anyone of the first, second, third, fourth and fifth aspect, the sample can be pretreated to enrich said sample with the target microorganism, and/or to remove human nucleic acid or nucleic of other microorganisms, optionally by size selection.

In any embodiments of the method according to anyone of the first, second, third, fourth and fifth aspect in which the sample is pretreated, removal of human nucleic acid is performed via hybridization to beads or columns with probes specific for human nucleic acid, via selective lysis of human cells and degradation of released human nucleic acid.

In any embodiments of the method according to anyone of the first, second, third, fourth and fifth aspect, the sample comprises can comprise a number of microorganism cells lower than 100, lower than 50, lower than 25, lower than 10, or lower than 5

In any embodiments of the method according to anyone of the first, second, third, fourth and fifth aspect, the sample and/or one or more sub-samples can comprise a single microorganism cell.

In any embodiments of the method according to anyone of the first, second, third, fourth and fifth aspect, the number of cell can be detected through detection of microorganism specific DNA or RNA copies.

In any embodiments of the method according to anyone of the first, second, third, fourth and fifth aspect, contacting the sample with an antibiotic can be performed for up to 90 minutes, up to 45 minutes, up to 30 minutes. up to 15 minutes, or up to 5 minutes.

In any embodiments of the method according to anyone of the first, second, third, fourth and fifth aspect,
the detecting can be performed by digital nucleic acid quantification to obtain a digital nucleic acid quantification concentration value.

In any embodiments of the method according to anyone of the first, second, third, fourth and fifth aspect directed to obtain a digital nucleic acid quantification concentration value, the digital nucleic acid quantification is performed by digital PCR, digital RT-PCR, digital LAMP, digital RT LAMP, digital RPA, or other digital isothermal amplification

In any embodiments of the method according to anyone of the first, second, third, fourth and fifth aspect the nucleic acid can be DNA and the detection can be performed qPCR or by DNA-seq wherein the nucleic acid concentration value is provided based on the sequence data.

In any embodiments of the method according to anyone of the first, second, third, fourth and fifth aspect the nucleic acid can be RNA, and the detection is performed by RT-qPCR or by RNA-seq wherein the nucleic acid concentration value is provided based on the sequence data,

In any embodiments of the method according to anyone of the first, second, third, fourth and fifth aspect, the detecting is performed by contacting a sample with a probe specific for a nucleic acid of the microorganism and or for any nucleic acid complementary to the nucleic acid of the microorganism.

In any embodiments of the method according to anyone of the first, second, third, fourth and fifth aspect the antibiotic is or comprises a beta-lactam and/or a carbapenem.

In any embodiments of the method according to anyone of the first, second, third, fourth and fifth aspect, the contacting can result in the antibiotic disrupting a cell envelope of the microorganism.

In any embodiments of the method according to anyone of the first, second, third, fourth and fifth aspect the microorganism can be Neisseria gonorrhoeae and/or comprise any microorganism belonging to the family Enterobacteriaceae.

In some preferred embodiments, the same-sample methods and systems herein described are performed in specimen with low number of cells. The ability to detect all subsets of nucleic acids in samples partitions obtained from a same specimen is important when the number of cells in the specimen is sufficiently small that the inherent randomness in partitioning the sample is comparable to or outweighs the difference in signals measured and used for susceptibility calling. In the worst case, where only one cell is present in the entire specimen, it becomes impossible to partition the specimen in multiple samples and obtain an even distribution of cells, since the single cell present cannot be split into more than one partitions.

Measuring both subsets of nucleic acids from a same partitioned sample gives one an additional piece of information: an estimate of the total number or mass of cells in the partition. Knowing the total number or mass of cells allows more options in how to process samples to achieve accessibility AST than if one could only measure one of the subsets from a given sample.

Additionally, a same-sample detection of intracellular/inaccessible and extracellular/accessible nucleic acid, in order to quantify the nucleic acids of the sample, allows one to perform such a detection without having any fraction of the sample, removed, altered, destroyed, hidden, or inactivated. In these embodiments, susceptibility is inferred by partitioning a specimen in a plurality of partitions and making one or more measurements from each the partitions separately. One example of a situation in which the specimen is partitioned is when the partitions of a same specimen are exposed to different antibiotic concentrations, one of which could be zero. Another example is to expose specimen partitions to a same antibiotic concentration (e.g. by exposing the specimen and partitioning the specimen into portioned samples after exposure), but to not alter a subset of the nucleic acids in at least one partition. This allows measurement of both the total nucleic acid concentration in the original specimen and the concentration of one subset only of nucleic acids from a same sample If the expected relative difference in the numbers of cells across the partitions is sufficiently small, one may be able to compare differently treated partitions to call susceptibility. The mathematical formula for calculating if the relative difference in the loading of partitions is sufficiently small is discussed later. However, if the number of cells in the entire specimen is too low, then partitioning the specimen introduces too much uncertainty, and the sample partitions cannot be compared to yield a susceptibility call with accuracy acceptable for clinical use.

If one uses filtration or another method that does not remove or alter nucleic acids, then even if one partitions a sample's cells unevenly, deviations in total cell number or amount can be estimated and used during analysis of the measured nucleic acid concentrations. In the extreme case when a sample is partitioned digitally (described in more detail later), and the majority of partitions contain one or zero cells, being able to measure both subsets of nucleic acids is necessary to discern whether partitions were empty or not.

Note that the discussion above pertains to nucleic acid quantification measurements of the sample. Nonetheless, measurement modalities that are not nucleic acid quantification, such as imaging or electrical sensors, can also be used to estimate the cell number or amount in each sample partition, or whether a given partition contained cells or not.

Additionally, to perform same-sample AST, the following steps is typically performed. in presence of a lysis treatment of an antibiotic treated sample targeting the microorganism, preceded by pre-lysis separation of an extracellular fraction of the sample comprising total extracellular nucleic acid, thus separating the total nucleic acids into an intracellular and an extracellular subset, the intracellular nucleic acid maintained in the sample and the extracellular separated in the extracellular fraction. Accordingly, embodiments of same-sample AST methods herein described involve pre-lysis separation of the sample's total nucleic acids into an intracellular and an extracellular subset. In embodiments of same-sample AST by not losing or ignoring any subset of the nucleic acids from any of the sample partitions, one is able to calculate or estimate the total nucleic acid concentration of each partition by summing the intracellular and extracellular nucleic acid concentrations as will be understood by a skilled person.

It is readily conceivable that other methods besides membrane filtration be used to physically separate, or to measure separately in the absence of physical separation, the amount of nucleic acids in each compartment of a partition of the sample. For example, filters not comprising polymer membranes can be constructed. Filters can be made from other materials such as metals, glass, or ceramics. For example, fritted ware can be used for filtering. Fritted ware are laboratory vessels, such as funnels and crucibles, with fritted-glass disks sealed permanently into the lower portion of the unit, and which are used for filtering bacteria from analytical chemical specimens. Filters in microfluidic chips can be constructed by fabricating small holes, or conversely, solid obstacles, in the solid substance of the chip. Physical separation methods that do not use filters include centrifugation, sedimentation, absorption, adsorption, phase separation, size-exclusion chromatography, affinity chromatography, gel electrophoresis, precipitation, crystallization, distillation, evaporation, and other separation techniques common in chemical engineering. All these methods could be employed to physically separate the intracellular and extracellular compartments of a sample (or partition of a sample).

To perform same-sample AST with centrifugation as the method of physical separation, one can expose a clinical specimen to antibiotics, then centrifuge the volume of antibiotic-contacted specimen. Intact cells, being larger in radius than the individual molecules of extracellular nucleic acid, are differentially pelleted at the bottom of the containing vessel, bringing with them the intracellular nucleic acids that are by definition contained within the intact cells. Nucleic acids remaining in the supernatant represent (are highly enriched for) extracellular nucleic acids, while the cells in the pellet represent (are highly enriched for) intracellular nucleic acids. The shape of the vessel in which the cell-containing liquid specimen can be designed to create pellet shapes that are compact and easy to collect, such as by having a high curvature (e.g. a V-shaped bottom). The centrifugation should not be performed at a speed that kills cells or breaches their cell envelopes. The maximum relative centrifugal force depends on the type of cell centrifuged, but in general, for unknown bacteria, the centrifugal force lie below about 10,000×g to ensure that bacteria are not damaged, with forces between 2000×g and 5000×g being commonly used in research. To cushion intact cells against lysing due to any shear stress of the vessel floor on the cells, one can introduce a centrifuge cushion liquid that is denser and immiscible with the aqueous solution so that cells pellet at the liquid-liquid interface between the specimen and the denser cushion liquid. This type of centrifugation is a special case of the technique known as density gradient centrifugation. Fluorocarbons or other dense liquids such as iodixanol are suitable centrifuge cushion liquids.

Accordingly, same-sample AST can be performed in a way that quantifies the quantity of a chemical that the target microorganism produces in a high copy number per cell, or for which amplification of the chemical is inherently easier. For example, one can quantify ribosomal RNAs using the appropriate primers and optimized reverse transcription conditions, as we describe in our example protocols. One can also target small RNAs and transfer RNAs. One can also target high copy number protein targets using ultrasensitive quantification methods such as the Quanterix digital ELISA.

Same-sample AST can be used in combination with various enhancement approaches, as described in our previous patent application. These include sonication, detergents, and other cell envelope stressors that increase an accessibility assay's discrimination of susceptible and resistant strains. Same-sample AST can be performed with all combinations of antimicrobial agents and microorganisms mentioned earlier in this document.

Same-sample AST can be performed in a high-throughput, parallelized fashion. Parallel measurements can simultaneously measure multiple samples from the same or different patient, multiple antibiotic exposures from the same clinical sample, and multiple partitions of the same clinical sample. When creating multiple antibiotic exposures from the same clinical sample, one is able to examine multiple antimicrobial agents and/or multiple doses of the same antimicrobial agents. Modern technology such as microtiter plates, droplet microfluidics, microfluidic devices, and robotics have made high-throughput chemical assays possible. A more detailed description of high-throughput instrumentation can be found in Example 2.

Same-sample AST can be performed using multiplexed measurements, in which multiple different measurements are made simultaneously from the same partition or antibiotic exposure of one or more clinical samples. Multiplexing includes amplifying and independently quantifying multiple nucleic acid sequences in the same reaction volume, sequencing and independently quantifying multiple nucleic acid sequences by nucleic acid sequencing, or making measurements of multiple modalities (e.g. optical measurements, mass measurements, spectroscopic measurements, electrochemical measurements, protein quantification, and nucleic acid amplification).

In some preferred embodiments of same-sample methods and systems herein described, separation comprises performing filtration. Filtration is one method in which both intracellular and extracellular nucleic acids can be recovered from the same sample without intentional loss of any nucleic acids. Because filtration separates intracellular and extracellular nucleic acids without destroying either of them, one can quantify both subsets of nucleic acids from the same partition of a sample.

In same-sample AST experiments herein described, it is demonstrated that filtration by a polymer membrane sufficiently separates nucleic acids in each compartment of the sample. Intact cells are large particles that cannot pass through the filter membrane, and thus intracellular nucleic acids do not pass through the filter membrane. Meanwhile, the extracellular nucleic acids originating from lysed cells are small enough to each pass through the filter membrane.

Filtration is a technique for separating tangible objects by their size. Objects whose minimum dimension is smaller than the filter's pore size will pass through the filter, while objects whose minimum dimension is larger than the pore size will not pass through the filter and will be retained.

A sample of microorganism in liquid media can contain intact cells and lysed cells. Nucleic acids within intact cells, the intracellular nucleic acids, are physically constrained within the boundaries of the cell. Thus, intracellular nucleic acids will be retained filter if the whole cell the nucleic acid resides in is retained on the filter. Nucleic acids originating from cells that have lysed, the extracellular nucleic acids, are freely dissolved in the sample, and they will pass through the filter if their diameter, not their original cell's diameter, is smaller than the filter's pore size.

Cells of microorganism possess a diameter that is larger (by at least 10-100-fold, usually more) than the individual nucleic acid molecules contained within in them. Thus, flowing a sample containing intracellular and extracellular nucleic acids through a filter will separate intracellular and extracellular nucleic acids, so long as the filter's pore size lies between the diameter of a cell of the microorganism and the diameter of a dissolved nucleic acid molecule.

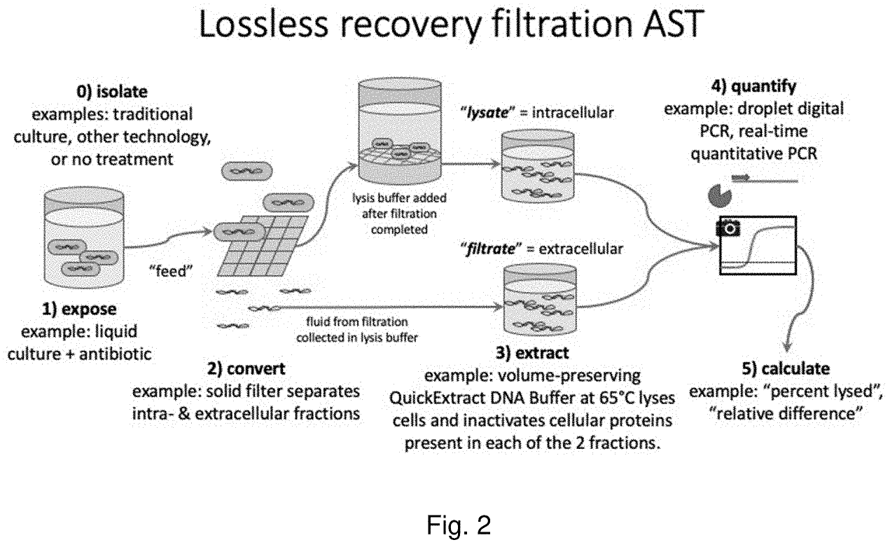

FIG. 2 shows a schematic diagram of an example lossless recovery filtration AST. A typical accessibility AST contains the 6 stages labeled, but specific embodiments may omit any one of the stages.

In some preferred embodiments of same-sample methods and systems herein described, the methods and systems comprise digital sample partitioning. Sample partitioning is defined herein to be the physical splitting of the specimen or sample of the specimen into multiple, separate portions partitions. Digital sample partitioning is defined herein to be a sample partitioning, from a sample with a certain, given density of microorganisms, that includes a sufficiently large number of partitions with a sufficiently small volume such that a sufficient number of partitions do not contain any microorganism. To achieve digital sample partitioning, one can either vary the number and volume of the partitions, or one can dilute the specimen or sample of the specimen such that a sufficient number of partitions do not contain the microorganism. The mathematical formulas for defining and calculating the sufficient number and volume of partitions for a given density of microorganisms are discussed later in the section “Description”. When a sample partitioning is performed in such a way is said to be in the “digital range”, and the sample is analyzed “digitally” using a “digital” method. Otherwise, the sample is said to be analyzed “in bulk” using a “bulk” method.

The same-sample AST of this disclosure can be performed either in bulk or digitally. In an in bulk AST method, one obtains bulk measurements of nucleic acid concentration from the entire sample in order to make a susceptibility call. In addition or in the alternative, when performing a digital same-sample AST, one can perform the measurements on individual partitions, then uses the integer counts of partitions meeting certain criteria to make a susceptibility call.

Digital sample partitioning enables the inference of two kinds of information about a sample: the number (or density) of cells in the sample and individual cells' responses to antimicrobials.

When performing digital sample partitioning, the total number of cells of interest can be estimated by observing the occupancy of the partitions. To perform this estimation, one splits a specimen or sample of the specimen, and thus the cells of interest in the specimen or sample of the specimen, into multiple partitions. For each partition, one or more signals can be measured, separately from the other partitions' signals. A partition's signal reveals whether the given partition is occupied by one or more cells, or whether the partition is not occupied by a cell. (As mentioned above, the signal in some embodiments can be nucleic acid amplification. In other embodiments, it can be one of the other listed modalities.) Counting the number of occupied and unoccupied partitions can be used to make a statistical estimate of the total number of cells in the whole specimen or sample of the specimen.

Specifically, a fraction P of the partitions of a sample of the specimen, ranging between 0 and 1, will not contain any cells. A complementary fraction N of the partitions, equal to 1−P, will each contain one or more cells. The distribution of bacterial cells into the partitions is random and follows the multinomial distribution. The number of trials of the multinomial distribution is the total number of cells in the unpartitioned sample, and the number of categories that each trial can adopt is the number of partitions. For large numbers of identical partitions, the multinomial probability distribution of the number of cells loaded into a single given partition is approximately equal to a Poisson distribution whose “lambda”, or “mean”, parameter is the concentration of cells in the sample. The concentration of cells is defined as the ratio of number of cells in the experiment sample to the volume of the experiment sample. The value of N is related to the concentration of cells of the target microorganism C by the equation C=−ln(N), where ln is the natural logarithm function, or equivalently, N=e^{-C}, where e is the natural logarithm base. Thus, from the observed value of N, one can calculate an estimate of the concentration of pathogen cells in the sample. Multiplying the concentration of cells in the sample C by the total volume of all the partitions yields the estimated total number of cells in all the partitions. Since it is possible for one partition to be loaded with more than one cell, the estimated total number of cells in all partitions is equal or greater than the number of occupied partitions observed.

One is not limited to measuring one signal from each partition. If one uses nucleic acid amplification as the measured signal, then one may further separate the nucleic acids in each partition into intracellular and extracellular subsets, then measure each subset separately. For clarity, in certain embodiments, one makes a first measurement of only lysed cells from a given sample partition, then one makes a measurement of only intact cells from the same partition. From the two measurements, one infers if the partition's cells are lysed, intact, or both (if there was more than one cell in the partition). One then repeats the pair of measurements for each sample partition. The number of partitions with lysed cells and the number of partitions with intact cells gives us an accurate estimate of the number of lysed cells and the number of intact cells in the sample. Note that the sum of these two numbers is the total number of cells, since cells are either lysed or not lysed, so by making two separate measures, one effectively learns the total number of cells without needing to make a third measurement of any kind from those same cells. Poisson statistics can again be used to make this estimation if the partitions are randomly loaded with cells.

The loading status is whether a given antibiotic exposure/sample partition contained a lysed cell, an intact cell, or no cell at the time of filtration. To call loading status, one can employ a variety of unsupervised machine learning algorithms, described herein below as found in the literature or known to the skilled person. It is envisioned that supervised machine learning algorithms can also be used, if one includes appropriate positive and negative controls for the nucleic acid amplification in the given or in prior experiments. These supervised algorithms are also described in section herein below.

### #4 Set of Preferred Embodiments: Summary Statistics for Determination of Antibiotic Susceptibility from comparison of detected nucleic acid concentration values

There are many plausible ways to derive a susceptibility call from the two nucleic acid concentrations measured from each antibiotic exposure (one filtrate and one lysate). In general, in the useful embodiments of accessibility AST, a summary statistic of the nucleic acid concentrations is calculated for each test condition. A “summary statistic” is a calculated numerical value (such as the sample mean) that characterizes some aspect of a sample set of data. The summary statistics of the test conditions are compared to the summary statistics of corresponding control conditions. The control conditions may be performed on the clinical sample at hand, or they may have been performed earlier on other clinical specimens of the same or related bacterial species. If the bacteria in the clinical specimen are susceptible to the dose of antibiotic tested by an antibiotic exposure, then the test condition summary statistics of that antibiotic exposure are expected to be higher, by a statistically significant magnitude, than the statistics resulting if the bacteria were exposed to zero antibiotics. There are many plausible choices for summary statistics, and many algorithms already exist for determining statistical significance by performing hypothesis testing of the summary statistics.

As a skilled practitioner will know, in some hypothesis testing scenarios, one can calculate a statistic that combines test and control summary statistics, then perform hypothesis testing. For example, one can take the difference of a test summary statistic and a control summary statistic, then test the hypothesis that the difference is equal to zero, rather than testing the hypothesis that the two numbers arise from the same distribution. Although difference when expressed in prose, the two approaches mentioned achieve the same end.

In addition to statistical techniques for hypothesis testing, algorithms can be used that make binary calls from data without the explicit calculation of a univariate summary statistic. Often, these algorithms are used when multiple measurements are made from each experimental condition, such as when multiplex nucleic acid quantification is performed during accessibility AST, as will be understood by a skilled person.

In one specific way of deriving a susceptibility call, a summary statistic called “percent extracellular” is calculated. The formula for percent extracellular is X=F/(F+Y), where X is the percent extracellular, F is the filtrate concentration, and Y is the lysate concentration. If a bacterium is susceptible to the antibiotic dose in a test condition, then the percent extracellular is expected to increase relative to the percent extracellular of control conditions.

In another specific way of deriving a susceptibility call, a summary statistic called “relative difference” can be calculated. If a bacterium is susceptible to the antibiotic dose in a test condition, then the relative difference is expected to increase or decrease away from the value of zero. Whether the relative difference increases or decreases depends on how one defines the relative difference. There are several mathematical definitions of a relative difference known to the skilled person. The definitions include the following:
1. Relative difference=(test concentration−control concentration)÷((test concentration+control concentration)÷2);
2. Relative difference=(test concentration−control concentration)÷((abs(test concentration)+abs(control concentration))÷2;
3. Relative difference=(test concentration−control concentration)÷max(test concentration, control concentration);
4. Relative difference=(test concentration−control concentration)÷max(abs(test concentration), abs(control concentration));
5. Relative difference=(test concentration−control concentration)÷min(test concentration, control concentration);
6. Relative difference=(test concentration−control concentration)÷min(abs(test concentration), abs(control concentration)).
7. In addition, any of these formulas may be multiplied or divided by a constant real number, such as in:
   a. Relative difference=(test concentration−control concentration)÷(test concentration+control concentration), where formula “1” has been multiplied by 2.
   b. Relative difference=(control concentration−test concentration)÷(control concentration+test concentration), where formula “1” has been multiplied by −1.

For values compiled beforehand, comparison can be by a univariate threshold or a more complicated statistical hypothesis test. If the comparison is chosen to be multivariate, then multivariate statistical tests and machine learning techniques can be employed, as described herein such as analysis of variance (ANOVA), linear regression, ordinary least squares regression, non-linear regression, logistic regression, probit regression, singular value decomposition, support vector machines, clustering, generative or Bayesian probability models, and principal component analysis.

### EXAMPLES

The same-sample AST and related methods and systems and compositions of the instant disclosure are further exemplified by exemplary protocols for some exemplary preferred embodiments of the same sample AST, such as high-throughput same-sample AST, and digitally loaded, same-sample AST.

In particular, the following examples illustrate exemplary methods and protocols for performing methods directed to detect extracellular/accessible and intracellular/inaccessible nucleic acid in a same sample as well as the determination of the related intracellular/extracellular proportion value, live and dead microorganism cells and/or determination of susceptibility or resistance of the microorganisms.

More particularly efficacy of the exemplary protocols is herewith shown with respect to strains with known susceptibility to the tested antibiotic as a proof of principle concerning the ability of same-sample methods and systems of the disclosure to accurately perform determination of live and dead cells in the sample and/or determination of susceptibility or resistance of the microorganism to the antibiotic, in absence and without the need, of an additional detection in the same sample and/or in a separate sample.

Accordingly, the exposure durations were chosen in view of the goal of providing proof of principle. Any exposure duration within the indicated range can be chosen based on the specific query and context of the test to balance the tradeoff between accuracy of the test and the turnaround time.

A person skilled in the art will appreciate the applicability and the necessary modifications to adapt the features described in detail in the present section, to additional methods and related compositions and systems according to embodiments of the present disclosure.

### Example 1: Same-Sample AST Exemplary Protocol

An exemplary same-sample AST protocol is provided herein below in an outline describing the various sets of operations comprised in the protocol.

1. Providing a Sample,

For the purposes of demonstration, a contrived clinical sample was made by inoculating an Escherichia coli isolate into Brain-Heart Infusion broth. The inoculum was small enough that no detectable difference in the sample's optical density at 600 mm (OD₆₀₀) was detectable by a spectrophotometer with a sensitivity of 0.01 absorbance units. After an incubation at 37° C., the media became turbid with an OD₆₀₀ of 0.26 absorbance units after 2 hours of incubation thus providing a bacteria batch culture.

2 Contacting the Sample with an Antibiotic/Antibiotic Exposure:

To begin the AST protocol, 10 μL of the above bacteria batch culture was added to and mixed with 15 μL of Mueller-Hinton Broth (MHB) growth media containing 1.67 μg/mL of dissolved ertapenem (ETP) antibiotic to create a test condition antibiotic exposure with a final ETP concentration of 1.0 μg/mL.

In parallel, 10 μL of the batch culture was added to and mixed with 15 μL of Mueller-Hinton Broth (MHB) growth media containing no ETP to create a control condition antibiotic exposure corresponding to the 1 μg/mL test condition. The two antibiotic exposures were incubated at 37° C. for 60 minutes.

3. Sample Separation by Filtration:

The entire volume of each antibiotic exposure was transferred to an individual cellulose acetate filter with a 0.2 μm pore size. Fluid that passes through the filter, called the “filtrate”, was collected in a clean microcentrifuge tube. The antibiotic exposures were centrifuged at 2200 relative centrifugal force to speed the passage of the antibiotic exposure through the filter and into the collecting vessel. Filtration was performed for each of the two antibiotic exposures created above to separate extracellular fraction from intracellular fraction of the sample.

With respect to the filtration the filter pore size was chosen to prevent the passage of intact bacterial cells, which are all larger than 0.2 with rare exceptions. The centrifugation speed was chosen to be low enough to prevent cell lysis. Additionally, it is expected that the filtrate will contain all or most of the extracellular nucleic acids present in the antibiotic exposure, but none of the intracellular nucleic acids in the antibiotic exposure.

4. Filter Washing Following Filtration,

50 μL of fresh MHB media was spun through the filters after the first centrifugation (above) to wash away residual extracellular nucleic acids present in the fluid wetting the filters. This wash fluid is not collected with the filtrates. This washing is an optional step. Any type of fluid that does not lyse or degrade cells can be passed through the filter. Examples include other growth medias and buffered solutions of salt compounds found physiologically inside of the bacteria.

5. Extracellular/Accessible Nucleic Acid Extraction from Filtrate,

20 μL of each of the filtrates was added to and mixed with 20 μL of Lucigen DNA Extraction Buffer, heated to 65° C. for 6 minutes, then heated to 98° C. for 4 minutes. The purpose of this step is to prevent chemical degradation of nucleic acids in the filtrate after collection. DNA Extraction Buffer prevents nucleic acid degradation by digesting and inactivating nuclease proteins. Alternative methods to achieve the same end include other RNA stabilization or nucleic extraction reactions or kits. Performance of this extraction step according to this protocol is optional.

6. Cell Lysis to Provide a Lysate Comprising Intracellular/Inaccessible Nucleic Acid:

25 μL of Lucigen DNA Extraction Buffer was placed on top of the filters. The filter membranes and apparatuses were heated to 65° C. for 6 minutes. Then, the filter apparatuses was centrifuged at 2200 RCF and the DNA Extraction Buffer fluid that flowed through the filter was collected in separate, clean microcentrifuge tubes. These collected fluid volumes are termed the “lysate”. The lysates were then heated to 98° C. for 4 minutes.

7. Extraction of Intracellular Nucleic Acid from the Lysate

The purpose of the cell lysis step is to recover the intracellular nucleic acids found in the intact cells retained on the filters. To do so, these intact cells are lysed and their nucleic acids extracted. The lysate is expected to contain all or most of the formerly intracellular, now extracellular nucleic acids.

Alternative ways to extract the intracellular nucleic acids can be performed. For example, the filter membrane can be removed from the filter apparatus using sterile and clean forceps and placed into a volume of DNA Extraction Buffer. This volume of buffer is vortexed vigorously, then heated to 65° C., then heated to 98° C. As a third alternative, intact bacterial cells retained on the filter can be mechanically dislodged (e.g. centrifugation in the opposite direction, stirring), then transferred to a volume of DNA Extraction Buffer, which is then heated to 65° C. and then to 98° C.

8. Reverse Transcription of Extracellular RNA:

Separately, for each of the treated filtrates, 1.5 μL of the treated filtrate were mixed with Lucigen RapiDxFire thermostable reverse transcriptase, deoxyribonucleic acid nucleotides, deionized water, and RapiDxFire thermostable buffer, according to manufacturer's instructions, in a total volume of 3 μL to create a reverse transcription reaction. A primer was also included. This primer's sequence was complementary to the 23S ribosomal RNA in Escherichia coli and specific to the Enterobacteriaceae family. The cDNA product that would be created from this primer contained the primer sites for the future ddPCR reaction occurring later in this AST protocol.

Reverse Transcription of Intracellular RNA

Separately, for each of the treated lysates, another reverse transcription reaction was set up following the same instructions, except 1.5 μL of the lysate was included instead of the 1.5 μL of filtrate. All reverse transcription reactions were heated to 60° C. for 5 minutes to create cDNAs, then heated to 95° C. for 5 minutes to stop the reaction.

With respect to filtrate and lysate a reverse transcription step is optional if one has decided to amplify a DNA molecule found naturally in the cells of interest. However, if the nucleic acid to be quantified in the AST protocol is a ribonucleic acid (RNA) molecule, and the quantification method operates only on deoxyribonucleic acid molecules, then both the filtrate and the lysate can be treated with a reverse transcriptase enzyme to produce complementary DNA molecules (cDNA) prior to nucleic acid quantification. The concentration of cDNA, and thus rRNA, is calculated from the counts of high and low fluorescence droplets.

With respect to reverse transcription of RNA in filtrates and lysates alternative reverse transcription enzymes, protocols, and kits can be used instead of the kit used in this example, as will be understood by a skilled person.

With respect to reverse transcription of RNA in filtrates and lysates alternative primers can be used. Alternative nucleic acid species can be targeted as well, through a choice of primers. As noted earlier in this document, targets with a higher copy number per cell are preferred for accessibility AST.

9. Quantification of Reverse Transcribed RNA Lysates and in Filtrates:

A volume of each of the above reverse transcription reactions was separately added to deionized water and BioRad QX200 ddPCR EvaGreen supermix, according to kit instructions. A pair of PCR primers was also included. These primers' sequences flanked an 80 bp region common to all of the 23S ribosomal RNA in Escherichia coli but specific to the Enterobacteriaceae family. One of the primers was the same primer used in the prior reverse transcription reaction. Droplet digital PCR (ddPCR) was performed on the BioRad QX200 platform according to manufacturer's instructions. The output of the ddPCR run was the nucleic acid concentration in the filtrate and in the lysate of both antibiotic exposures.

With respect to quantification of reverse transcribed RNA lysates and in filtrates alternative nucleic acid quantification methods could have been employed, including all of the methods for nucleic acid quantification enumerated earlier in this document.

10. Determination of Same Sample Nucleic Acid Accessibility Intracellular/Extracellular Proportion Value as a Percent Extracellular NA

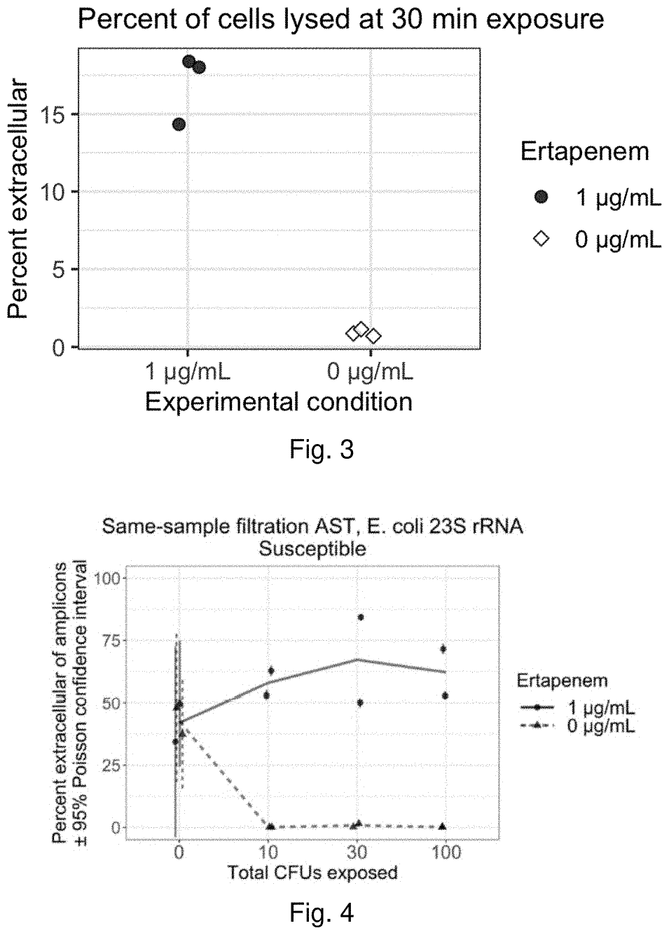

The “percent extracellular” summary statistic was calculated once for the test condition antibiotic exposure and once for the control condition antibiotic exposure wherein an increase in the percent extracellular statistic under test condition compared to the control conditions indicate susceptibility. In this experiment, the entire experiment was reproduced two more times, yielding three replicates as reported in FIG. 3 showing the percent cell lysis at 30 minutes of exposure in view of the percent extracellular nucleic acid concentration detected under test condition (black circle) vs control condition (white diamond).

A susceptibility threshold value was also chosen to be 2 times the sample standard deviation of the control condition percent extracellular values higher than the mean of the control condition percent extracellular values.

In this case, the lower bound of the 95% Poisson confidence interval of all the ddPCR measurements for the test conditions were higher than the susceptibility threshold value, so the isolate was correctly called as susceptible.

Alternative choices and algorithms for choosing for the threshold can be used. For example, if only one experimental replicate was performed, a threshold of 5% could have been used based on prior knowledge that a background rate of 5% lysis has never been observed for Escherichia coli. A list of exemplary appropriate choices is reported in other sections of this document.

### Example 2: Same-sample AST Example Protocol in High Throughput Microtiter Plate

An exemplary same-sample AST protocol in high throughput is provided herein below in an outline describing the various sets of operations comprised in the protocol.

1. Providing a Sample:

For the purposes of demonstration, a series of contrived clinical sample were made by inoculating an Escherichia coli clinical isolate “strain X” into Brain-Heart Infusion broth. The cultures were incubated until the bacteria entered an exponential growth phase. The culture was then diluted to a density of 40 cells/μL.

Preparing an antibiotic loaded plate: A 96 well microtiter plate was prepared with growth media and differing antibiotic amounts as shown in this diagram. Each well contained 15 of Mueller-Hinton Broth (MHB) growth media and antibiotics at the 1.67× the final concentration as shown in the diagram, so that the final concentration of antibiotic after the addition of 10 μL would be the value shown in the diagram. Six distinct beta-lactam antibiotics, including 1 beta-lactam/beta-lactamase inhibitor combinations, were represented, namely ertapenem (ETP), meropenem (MEM), ceftriaxone (CRO), aztreonam (ATM), ampicillin (AMP), ampicillin-sulbactam (SAM). Each antibiotic was tested at 8 concentrations, with 2 replicates for each concentration. One of the concentrations included a 0 μg/mL control condition. The CLSI breakpoint concentrations for each antibiotic were represented as well as reported in the following

TABLE 1
Table 1

| | Col 1 | Col 2 | Col 3 | Col 4 | Col 5 | Col 6 | Col 7 | Col 8 | Col 9 | Col 10 | Col 11 | Col 12 |
|---|---|---|---|---|---|---|---|---|---|---|---|---|
| Row | ETP, | ETP, | MEM, | MEM, | CRO, | CRO, | ATM, | ATM, | AMP, | AMP, | SAM, | SAM, |
| A | 0 | 0 | 0 | 0 | 0 | 0 | 0 | 0 | 0 | 0 | 0 | 0 |
| | μg/mL | μg/mL | μg/mL | μg/mL | μg/mL | μg/mL | μg/mL | μg/mL | μg/mL | μg/mL | μg/mL | μg/mL |
| Row | ETP, | ETP, | MEM, | MEM, | CRO, | CRO, | ATM, | ATM, | AMP, | AMP, | SAM, | SAM, |
| B | 0.125 | 0.125 | 0.125 | 0.125 | 0.5 | 0.5 | 1 | 1 | 1 | 1 | 1/0.5 | 1/0.5 |
| | μg/mL | μg/mL | μg/mL | μg/mL | μg/mL | μg/mL | μg/mL | μg/mL | μg/mL | μg/mL | μg/mL | μg/mL |
| Row | ETP, | ETP, | MEM, | MEM, | CRO, | CRO, | ATM, | ATM, | AMP, | AMP, | SAM, | SAM, |
| C | 0.25 | 0.25 | 0.25 | 0.25 | 1 | 1 | 2 | 2 | 2 | 2 | 2/1 | 2/1 |
| | μg/mL | μg/mL | μg/mL | μg/mL | μg/mL | μg/mL | μg/mL | μg/mL | μg/mL | μg/mL | μg/mL | μg/mL |
| Row | ETP, | ETP, | MEM, | MEM, | CRO, | CRO, | ATM, | ATM, | AMP, | AMP, | SAM, | SAM, |
| D | 0.5 | 0.5 | 0.5 | 0.5 | 2 | 2 | 4 | 4 | 4 | 4 | 4/2 | 4/2 |
| | μg/mL | μg/mL | μg/mL | μg/mL | μg/mL | μg/mL | μg/mL | μg/mL | μg/mL | μg/mL | μg/mL | μg/mL |
| Row | ETP, | ETP, | MEM, | MEM, | CRO, | CRO, | ATM, | ATM, | AMP, | AMP, | SAM, | SAM, |
| E | 1 | 1 | 1 | 1 | 4 | 4 | 8 | 8 | 8 | 8 | 8/4 | 8/4 |
| | μg/mL | μg/mL | μg/mL | μg/mL | μg/mL | μg/mL | μg/mL | μg/mL | μg/mL | μg/mL | μg/mL | μg/mL |
| Row | ETP, | ETP, | MEM, | MEM, | CRO, | CRO, | ATM, | ATM, | AMP, | AMP, | SAM, | SAM, |
| F | 2 | 2 | 2 | 2 | 8 | 8 | 16 | 16 | 16 | 16 | 16/8 | 16/8 |
| | μg/mL | μg/mL | μg/mL | μg/mL | μg/mL | μg/mL | μg/mL | μg/mL | μg/mL | μg/mL | μg/mL | μg/mL |
| Row | ETP, | ETP, | MEM, | MEM, | CRO, | CRO, | ATM, | ATM, | AMP, | AMP, | SAM, | SAM, |
| G | 4 | 4 | 4 | 4 | 16 | 16 | 32 | 32 | 32 | 32 | 32/16 | 32/16 |
| | μg/mL | μg/mL | μg/mL | μg/mL | μg/mL | μg/mL | μg/mL | μg/mL | μg/mL | μg/mL | μg/mL | μg/mL |
| Row | ETP, | ETP, | MEM, | MEM, | CRO, | CRO, | ATM, | ATM, | AMP, | AMP, | SAM, | SAM, |
| H | 8 | 8 | 8 | 8 | 32 | 32 | 64 | 64 | 64 | 64 | 64/32 | 64/32 |
| | μg/mL | μg/mL | μg/mL | μg/mL | μg/mL | μg/mL | μg/mL | μg/mL | μg/mL | μg/mL | μg/mL | μg/mL |

2. Contacting the Sample with the Antibiotic Loaded Plate:

To begin the AST protocol, 10 μL of the above bacteria batch culture was separately added to and mixed with each well's contents to create 96 antibiotic exposures. The antibiotic exposures were then incubated at 37° C. for 60 minutes.

3. Separating the Sample by Filtration and Centrifugation

The entire volume of each antibiotic exposure was transferred to a 96-well filter plate. Each well of the filter plate contained a polyvinylidene fluoride (PVDF) filter membrane with a 0.2 μm pore size. The antibiotic exposures were centrifuged at 2200 relative centrifugal force to speed the passage of the antibiotic exposure through the filter and into 96 collecting vessels. The collected fluid was called the “filtrate.”

In the above filtration plus centrifugation step the filter pore size was chosen to prevent the passage of intact bacterial cells, which are all larger than 0.2 with rare exceptions. The centrifugation speed was chosen to be low enough to prevent cell lysis. In the above filtration plus centrifugation step It is expected that the filtrate will contain all or most of the extracellular/accessible nucleic acids present in the antibiotic exposure, but none of the intracellular/inaccessible nucleic acids in the antibiotic exposure.

4. Filter Washing:

50 μL of fresh MHB media was spun through the filters after the first centrifugation (above) to wash away residual extracellular nucleic acids present in the fluid wetting the filters. This wash fluid is not collected with the filtrates. This is an optional step. Any type of fluid that does not lyse or degrade cells may be passed through the filter. Examples include other growth medias and buffered solutions of salt compounds found physiologically inside of the bacteria.

5. Extracellular DNA Extraction from the Filtrate:

10 μL of each of the filtrates was added to and mixed with 10 μL of Lucigen DNA Extraction Buffer, heated to 65° C. for 6 minutes, then heated to 98° C. for 4 minutes. The purpose of this step is to prevent chemical degradation of nucleic acids in the filtrate after collection. DNA Extraction Buffer prevents nucleic acid degradation by digesting and inactivating nuclease proteins. Alternative methods to achieve the same end include other RNA stabilization or nucleic extraction reactions or kits. This step is optional.

6. Cell Lysis to Provide Lysate Comprising Intracellular/Inaccessible Nucleic Acid

20 μL of Lucigen DNA Extraction Buffer was Placed on Top of the filters. The filter membranes and apparatuses were heated to 65° C. for 6 minutes. Then, the filter apparatuses were centrifuged at 2200 RCF and the DNA Extraction Buffer fluid that flowed through the filter was collected in separate, clean microcentrifuge tubes. These collected fluid volumes are termed the “lysate”. The lysates were then heated to 98° C. for 4 minutes.

7. Extraction of Intracellular Nucleic Acid.

The purpose of providing a cell lysate is to recover the intracellular nucleic acids found in the intact cells retained on the filters. To do so, these intact cells are lysed, and their nucleic acids extracted. The lysate is expected to contain all or most of the formerly intracellular, now extracellular nucleic acids.

Alternative ways to extract the intracellular nucleic acids can be performed. For example, the filter membrane can be removed from the filter apparatus using sterile and clean forceps and placed into a volume of DNA Extraction Buffer. This volume of buffer is vortexed vigorously, then heated to 65° C., then heated to 98° C. As a third alternative, intact bacterial cells retained on the filter can be mechanically dislodged (e.g. centrifugation in the opposite direction, stirring), then transferred to a volume of DNA Extraction Buffer, which is then heated to 65° C. and then to 98° C. The temperatures of 65° C. and 98° C. derive from the manufacturer's instructions for the Lucigen DNA Extraction Buffer kit.

8. Quantification of DNA and RNA Intracellular and Extracellular Nucleic Acid

In this experiment, it was decided to amplify both DNA and RNA targets. Thus, the protocol at this point divides into two branches, one with reverse transcription, and one without.

For the reverse transcription branch, each of the treated filtrates and lysates DNA extractions (192 in total), a 1.5 μL volume was taken and diluted 1000-fold in deionized water. The diluted DNA extractions were used as templates in the Lucigen RapiDxFire thermostable reverse transcription kit, following kit instructions. The primer included was complementary to the 23S ribosomal RNA stranded-ness, specific to the Enterobacteriaceae family, and upstream of the PCR product amplified by the PCR primers of the future PCR stage of this protocol; in fact, the reverse transcription primer was identical to one of the PCR primers.

For the no-reverse transcription branch, no reverse transcription was performed.

A volume of each of the treated filtrates and lysates, with and without reverse transcription, was separately added to deionized water, a pair of PCR primers, and BioRad QX200 ddPCR EvaGreen supermix, according to kit instructions. There were 384 ddPCR reactions in total. The primers' sequences flanked an 80 bp region common to all of the 23S ribosomal RNA genomic loci in Escherichia coli and also specific to the Enterobacteriaceae family. Droplet digital PCR (ddPCR) was performed on the BioRad QX200 platform according to manufacturer's instructions. The output of the ddPCR run was the nucleic acid concentration in the filtrate and in the lysate of both antibiotic exposures.

Alternative nucleic acid quantification methods could have been employed, including all of the methods for nucleic acid quantification enumerated earlier in this document as will be understood by a skilled person.

9. Same-Sample Determination of Nucleic Acid Accessibility and Intracellular/Extracellular Proportion Value as a Percent Extracellular NA

The “percent extracellular” summary statistic was calculated once for the test condition antibiotic exposure and once for the control condition antibiotic exposure. The sample mean and sample standard deviation of the percent extracellular values from all control conditions was calculated. A susceptibility threshold value was chosen to be the control condition sample mean added to three times the control condition sample standard deviation.

Alternative choices and algorithms for choosing for the threshold can be used. A list of appropriate choices was enumerated earlier in this document.

For example, the threshold can be the control condition sample mean added to any multiple of the control condition sample standard deviation. The measurements of control conditions from other runs, from other strains, and even the treated conditions of known resistant strains could be included when calculating the control condition sample mean and sample standard deviation.

For example, other summary statistics besides the standard deviation can be calculated. Or, other machine learning or statistical tests could be used to deterministically calculate. Threshold values can even be arbitrarily drawn, although this is not preferred compared to objectively defined thresholds.

Other summary statistics (a.k.a. “metrics”) can also be calculated. For selecting the univariate threshold values (or functions, if multivariate thresholds are defined) for each of these statistics, the above definition for the percent extracellular statistic can be used, or other methods known to a skilled practitioner can be applied.

Suitable metrics include the relative change in the extracellular nucleic acids for each test and control condition pair. Alternatively, the relative change between each test condition (84 distinct values) and the mean (a single value) of the control conditions can be calculated. Alternatively, the relative chance between the mean of equivalent test conditions (42 distinct values) and the mean of the control conditions (1 distinct value) can be calculated.

Suitable metrics further include the control-to-treated ratio, the treated-to-control ratio, the control-to-treated difference, the control-to-treated difference, and any other metric mentioned in our lab's previous patent application.

Accordingly, the strain is determined to be susceptible at all antibiotic dosages for which the percent lysed is higher than the threshold. Otherwise, the strain is determined to be resistant.

Additionally, or alternatively, one can define additional thresholds and susceptibility categories bordered by such thresholds.

For example, one can determine that statistics lying between 1 and 3 standard deviations of the control condition sample mean belong to an “intermediate resistance” category.

Additional metrics applicable to detection performed according to the above exemplary protocol to determine same sample nucleic acid accessibility and antibiotic susceptibility or resistance can be identified by a skilled person upon reading of the present disclosure.

### Example 3: Same-Sample Filtration AST, for Two Strains at Once, with Multiple Replicate Treated Conditions and Multiple Concurrent Reference Conditions

An exemplary same sample multiplex AST protocol is provided herein below in an outline describing the various sets of operations comprised in the protocol.

1. Providing a Sample:

For the purposes of demonstration, two contrived clinical specimens were made by inoculating Escherichia coli isolates into Brain-Heart Infusion broth, each isolate into a separate tube. Isolate A was susceptible to ertapenem, while isolate B was resistant. The inoculum was small enough that no detectable difference in the sample's optical density at 600 mm (OD₆₀₀) was detectable before and after inoculation by an Ultrospec 10 spectrophotometer with an analytical sensitivity of 0.01 absorbance units. After an incubation at 37° C., the media became turbid with an OD₆₀₀ of 0.5 absorbance units after about 2 hours of incubation. To demonstrate the performance of filtration AST as a function of number of cells analyzed, the cultures were diluted to densities of 4210, 1260, 421, and 0 cells/mL immediately before the introduction of antibiotic to create 8 batch culture dilutions.

2. Contacting the Sample with Antibiotic/Antibiotic Exposure:

To begin the AST protocol, 23.75 μL of the above 8 bacteria batch culture dilutions was twice added to and mixed with 1.25 μL of water containing either 20 μg/mL or 0 μg/mL of dissolved ertapenem (ETP) antibiotic. As a result, 16 treated conditions at 1 μg/mL were created, and 8 untreated reference conditions were created (see Table 2 below). The sixteen antibiotic exposures were incubated at 37° C. for 60 minutes.

TABLE 2
Batch Culture Dilution Treated conditions

| | Antibiotic exposure # | | | | | | | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| ETP concentration | 1 | 1 | 1 | 1 | 1 | 1 | 1 | 1 | 0 | 0 | 0 | 0 | 0 | 0 | 0 | 0 |
| during exposure | | | | | | | | | | | | | | | | |
| (μg/mL) | | | | | | | | | | | | | | | | |
| Average number | 100 | 30 | 10 | 0 | 100 | 30 | 10 | 0 | 100 | 30 | 10 | 0 | 100 | 30 | 10 | 0 |
| of cells exposed | | | | | | | | | | | | | | | | |
| Isolate | A | A | A | A | A | A | A | A | A | A | A | A | A | A | A | A |
| | Antibiotic exposure # | | | | | | | | | | | | | | | |
| | 17 | 18 | 19 | 20 | 21 | 22 | 23 | 24 | 25 | 26 | 27 | 28 | 29 | 30 | 31 | 32 |
| ETP concentration | 1 | 1 | 1 | 1 | 1 | 1 | 1 | 1 | 0 | 0 | 0 | 0 | 0 | 0 | 0 | 0 |
| during exposure | | | | | | | | | | | | | | | | |
| (μg/mL) | | | | | | | | | | | | | | | | |
| Average number | 100 | 30 | 10 | 0 | 100 | 30 | 10 | 0 | 100 | 30 | 10 | 0 | 100 | 30 | 10 | 0 |
| of cells exposed | | | | | | | | | | | | | | | | |
| Isolate | B | B | B | B | B | B | B | B | B | B | B | B | B | B | B | B |

3. Sample Separation by Filtration and Centrifugation:

After 60 minutes of incubation, the entire volume of each antibiotic exposure was transferred to an individual cellulose acetate filter unit with a 0.2 μm pore size. At 65 minutes, the 32 filter units were centrifuged at 4000 rcf for 2 minutes. Fluid that passes through the filter, called the “filtrate”, was collected in a clean microcentrifuge tube. It is expected that the filtrate will contain all or most of the extracellular nucleic acids present in the antibiotic exposure, but none of the intracellular nucleic acids in the antibiotic exposure.

4. Filter Washing

50 μL of fresh MHB media was spun through the filters after the first centrifugation (above) to wash away residual extracellular nucleic acids present in the fluid wetting the filters. This wash fluid is not collected with the filtrates. In this experiment, the wash fluid was collected and analyzed to learn more about the protocol, but this washing step is not necessary for determining susceptibility of the isolate.

5. Cell Lysis to Provide Lysate Comprising Intracellular/Inaccessible Nucleic Acid;

25 μL of Lucigen DNA Extraction Buffer was placed on top of the filters. The filter membranes and apparatuses were heated to 65° C. for 6 minutes in a heat block with a 1.5 mL centrifuge tube adaptors. Then, the filter units were centrifuged at 4000 RCF for 2 minutes. The DNA Extraction Buffer fluid that flowed through the filter was collected in separate, clean microcentrifuge tubes. These collected fluid volumes are termed the “lysate” which comprises intracellular nucleic acid.

6. Lysate Collection:

25 μL of pure water were placed onto the filters of each filter unit, let to sit for 4 minutes, then spun through the filters at 4000 rcf for 2 minutes into the same microcentrifuge tubes containing the lysate, creating a diluted lysate. This step is optional.

The purpose of the step was to ensure the collection of any formerly intracellular, now extracellular nucleic acids that were not collected in the previous step because they remained in the small volume of DNA Extraction Buffer retained on the filter membrane and filter unit.

7. Extraction of Extracellular/Accessible Nucleic Acid:

20 μL of each of the filtrates was added to and mixed with 20 μL of Lucigen DNA Extraction Buffer, heated to 65° C. for 6 minutes, then heated to 98° C. for 4 minutes. The purpose of this step is to prevent chemical degradation of nucleic acids in the filtrate after collection. DNA Extraction Buffer prevents nucleic acid degradation by digesting and inactivating nuclease proteins. Alternative methods to achieve the same end include other RNA stabilization or nucleic extraction reactions or kits. This step is optional.

The diluted lysates and the filtrates were each separately transferred from 1.5 mL collection tubes to PCR tubes (64 in total), then were then heated to 98° C. for 4 minutes to inactivate the DNA Extraction Buffer.

8. Lysates and Filtrates Dilutions:

The diluted lysates and filtrates were separately diluted in water to create template solutions. The lysates and filtrates from exposures with 100 expected cells (1, 5, 9, 13, 17, 21, 25, and 29) were diluted 1:50; those with 30 expected cells were diluted 1:15; those with 10 expected cells were diluted 1:5, and those with 0 expected cells were diluted 1:5.

9. Reverse Transcription of Intracellular and Extracellular RNA

Next, 4.0 μL of each template solution was mixed with 0.1 μL of 3 U/mL Lucigen® RapiDxFire thermostable reverse transcriptase, 0.5 μL of Lucigen® RapiDxFire 10× thermostable buffer, 0.25 μL of 10 mM deoxyribonucleic acid nucleotides, and 0.2 μL of a 10 μM aqueous solution of DNA primer, according to manufacturer's instructions, to create a reverse transcription reaction with a total volume of 5.0 μL.

Lucigen RapiDxFire thermostable reverse transcriptase, deoxyribonucleic acid nucleotides, deionized water, and RapiDxFire thermo stable buffer, according to manufacturer's instructions, in a total volume of 4.98 μL to create a reverse transcription reaction. A primer was also included. This primer had a sequence of 5′-CGTTAGCACCCG(C)^{L}CGTGTGTCTCCCGTG-3′ (SEQ ID NO: 1) and predicted melting temperature of 76° C. The primer contained a locked nucleic acid cytidine, indicated as (C)^{L}. This primer's sequence was complementary to the 23S ribosomal RNA in Escherichia coli and specific to the Enterobacteriaceae family. The cDNA product that was created from this primer contained the primer sites for the future ddPCR reaction occurring later in this AST protocol. All reverse transcription reactions were heated to 69° C. for 5 minutes to create cDNAs, then heated to 95° C. for 5 minutes to stop the reaction and inactivate the reverse transcriptase enzyme.

A reverse transcription step is optional if one has decided to amplify a DNA molecule found naturally in the cells of interest. However, if the nucleic acid to be quantified in the AST protocol is a ribonucleic acid (RNA) molecule, and the quantification method operates only on deoxyribonucleic acid molecules, then both the filtrate and the lysate can be treated with a reverse transcriptase enzyme to produce complementary DNA molecules (cDNA) prior to nucleic acid quantification. The concentration of cDNA, and thus rRNA, is calculated from the counts of high and low fluorescence droplets. Alternative reverse transcription enzymes, protocols, and kits may be used instead of the kit used in this example. Alternative primers may be used. Alternative nucleic acid species can be targeted as well, through a choice of primers. As noted earlier in this document, targets with a higher copy number per cell are preferred for accessibility AST.

10. Digital Quantification of Intracellular and Extracellular 23S Ribosomal RNA:

A 3 μL volume of each of the above reverse transcription reactions was separately added to deionized water and BioRad QX200 ddPCR EvaGreen supermix, according to kit instructions, to make a 20 μL total reaction volume. A pair of PCR primers was also included with the sequences 5′-GGTAGAGCACTGTTTTGGCA-3′ (SEQ ID NO: 2) and 5′-TGTCTCCCGTGATAACTTTCTC-3′ (SEQ ID NO: 3). These primers' sequences flanked an 80 bp region common to all of the 23S ribosomal RNA in Escherichia coli but specific to the Enterobacteriaceae family. Droplet digital PCR (ddPCR) was performed on the BioRad QX200 platform according to manufacturer's instructions.

The output of the ddPCR run was the nucleic acid concentration in the filtrate and in the lysate of both antibiotic exposures. Alternative nucleic acid quantification methods could have been employed, including all of the methods for nucleic acid quantification enumerated earlier in this document. The result of the ddPCRs was 64 expected concentrations of rRNA and a 95% Poisson confidence interval denoting the range over which the same mean concentration would have also appeared 95% of the time if it were to be repeated, with variation due solely to the stochastic loading of template molecules into the droplets.

11. Nucleic Acid Accessibility and Intracellular/Extracellular Proportion Value as a Percent Extracellular Nucleic Acid:

The “percent extracellular” summary statistic was calculated for each of the 32 pairs of concentrations measured from the filtrate and lysate of each antibiotic exposure, using the expected concentration and ignoring the 95% Poisson confidence intervals. These percent extracellular values are extra/intracellular nucleic acid proportion values defined as PE=100×E/(E+I), where E is the filtrate concentration and I is the lysate concentration. Because both the lysate and filtrate concentrations were low for all exposures containing no cells, the percent extracellular values for these conditions were not further evaluated to make susceptibility calls.

The threshold for a low concentration indicating 0 cells in a given condition is determined in two ways depending on how one treats these conditions. If one treats the 0 cell conditions not as derived from actual clinical specimens, but instead as a control condition known to contain no cells (since none of the clinical specimen in this example experiment was actually placed into these exposures), then by definition these conditions are not used when assessing the susceptibility of the isolates and no threshold is needed for comparison. If instead, these 0 cell exposures were treated as having derived from actual clinical specimens to be queried, then the threshold would be derived from statistical analysis of prior experiments performed with 0 cells, such as the mean plus 2 standard deviations of such prior experiments' 0 cell measurements. These prior experiments would be experiments identical to the 0 cell exposures of this current experiment where it is known that 0 cells were added, and any signal seen is the result of measurement noise from the commercial reagents and equipment.

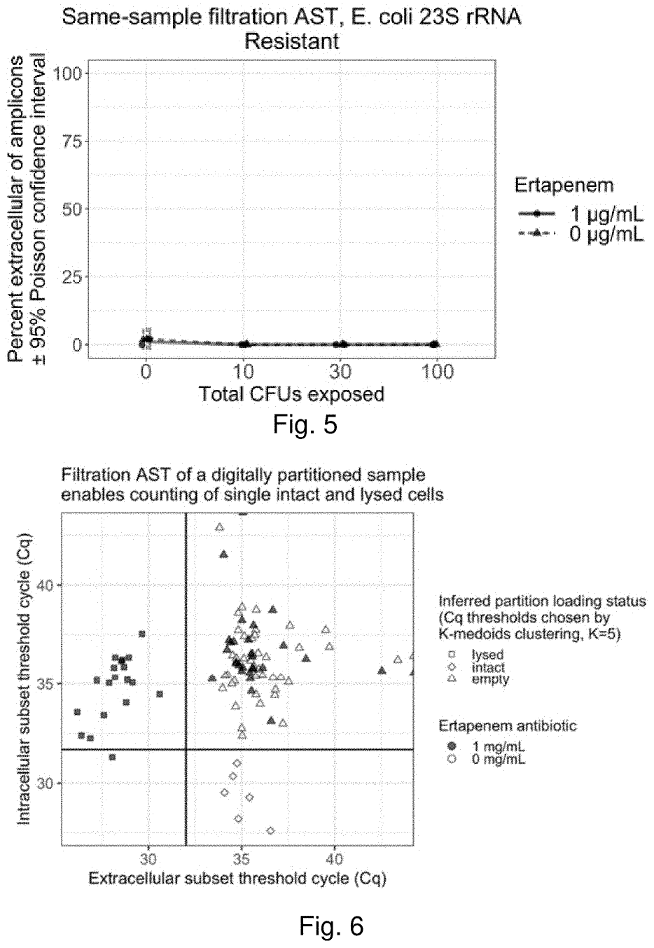

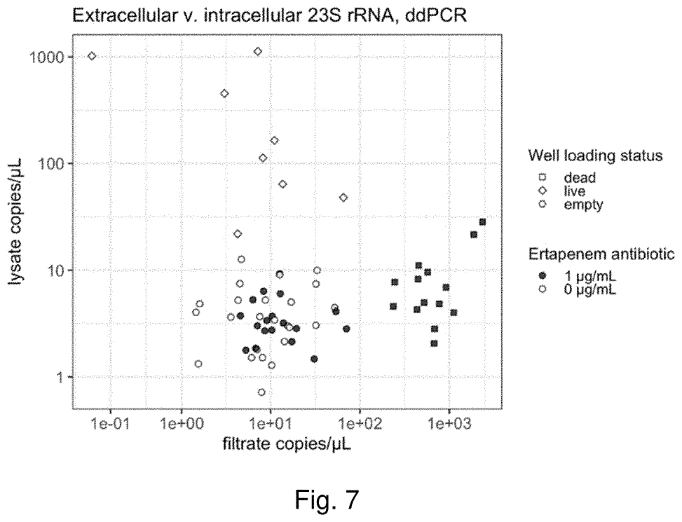

It is possible to calculate the equivalent 95% confidence intervals of the percent extracellular values caused by Poisson loading instead of ignoring this information. To do this, one uses the well-known formula for the propagation of errors. FIGS. 4 and 5 report the percent extracellular (FIG. 4) and intracellular (FIG. 5) of amplicons in an E. coli susceptible sample calculated for each of the 32 pairs of concentrations measured from the filtrate and lysate of each antibiotic exposure, using the expected concentration and ignoring the 95% Poisson confidence intervals. FIGS. 4 and 5 in fact include these derived 95% confidence intervals for the percent extracellular values as the error bars depicted.

12. AST Determination Based on the Percent Extracellular Nucleic Acid as Intracellular/Extracellular Proportion Value

To call susceptibility, a t-test was performed between all isolate A treated (exposures 1 to 8) and isolate A untreated (exposures 9-16) measurements. The null hypothesis of this test was that the treated and untreated measurements arose from the same distribution. A second t-test was performed between all isolate B treated (exposures 17-24) and isolate B untreated (exposures 25-32) measurements, using the same null hypothesis. The test was significant only for isolate A, so isolate A was correctly determined to be susceptible, while isolate B was correctly called as resistant.

Because the treated condition measurements for isolate A have a different variance than the untreated condition measurements for isolate A, one could argue that the t-test may not perform well. In this case, a non-parametric statistical test could be used for hypothesis testing.

Because there were multiple concurrent reference conditions in this experiment, another additional or alternative approach is to define a susceptibility threshold value equal to the mean of each isolate's reference condition fraction extracellular values plus 2 times the sample standard deviation of each isolate's reference condition fraction extracellular values. Optionally, both isolate's reference condition fraction extracellular values can be considered together to calculate the susceptibility threshold. In this case, a majority of the mean measurements for isolate A's treated exposures where there were more than 0 cells were above the threshold, while a majority of isolate B's treated exposures were not above this threshold. Thus, isolate A would have been called as susceptible, and isolate B as resistant.

Alternatively, one can assess the lower bound of the 95% Poisson confidence interval of all the treated ddPCR measurements against a susceptibility threshold value equal to the mean of each isolate's reference condition fraction extracellular values plus 2 times the sample standard deviation of each isolate's reference condition fraction extracellular values. In this case, a majority of isolate A's fraction extracellular values again would pass as susceptible, except for those with no cells, and thus isolate A was called as susceptible. A majority of isolate B's fraction extracellular values were not above isolate B's susceptibility threshold.

Alternative choices for the statistical analysis of the measured concentrations exist that will be known to the skilled practitioner. A list of exemplary appropriate choices are reported in additional sections of the present disclosure.

### Example 4: Filtration AST and Same-Sample Filtration AST in the Same Experiment, with a Spiked Control, in a Microtiter Plate

In the experiment described in this example, there were two possible goals, each with their own interpretation of the same results.

One purpose was to learn about the effect of different filter membrane treatments (washing and heating) on our assay output. This goal is not a question that clinicians using our assay would pursue, but does illustrate optional treatments of the filter membrane that our invention entails.

The other goal of the experiment was that to confirm the susceptibility of the bacteria strain Escherichia coli K12 to demonstrate our assay's validity, even though Escherichia coli K12 susceptibility was already known. This latter goal mimics possible questions pursued by a user of methods and systems of the present disclosure and provide a proof principle of the operability of the same-sample methods and systems of the present disclosure.

The related exemplary same-sample AST protocol is provided herein below in an outline describing the various sets of operations comprised in the protocol.

To prepare for this experiment, two Millipore® 96-well sterile polystyrene MultiScreenHTS® filter plates with 0.22 μm pore size, hydrophilic polyvinylidene fluoride filter membranes (Millipore-Sigma MSGVS2210) were prepared. One plate was then heated to 65° C. for 6 minutes and then at 98° C. for 4 minutes. Certain filters in the plate were washed with nuclease-free pure water as indicated in the table below.

1. Providing a Sample

One contrived clinical specimen was made by inoculating Escherichia coli K12 into Brain-Heart Infusion broth. Escherichia coli K12 is susceptible to ertapenem. The inoculum was small enough that no detectable difference in the sample's optical density at 600 mm (OD₆₀₀) was detectable before and after inoculation by an Ultrospec 10 spectrophotometer with an analytical sensitivity of 0.01 absorbance units. After 2.57 hours of incubation at 37° C., the media became turbid with an OD₆₀₀ of 0.22 absorbance units. To demonstrate the performance of filtration AST as a function of number of cells analyzed, the cultures were diluted to densities of 2,170,000 and 174 cells/mL immediately before the introduction of antibiotic.

2. Contacting the Sample with Antibiotic/Antibiotic Exposure of the Sample

To begin the AST protocol, 23.0 μL of the above 8 bacteria batch culture dilutions was twice added to and mixed with 2.0 μL of water containing either 12.5 μg/mL or 0 μg/mL of dissolved ertapenem (ETP) antibiotic and 50,000 copies/μL of lambda phage DNA. As a result, 16 antibiotic exposure conditions with a total volume of 25 μL were created as indicated in the table below. The 16 conditions were incubated at 37° C. for 60 minutes as outlined in Table 3.

TABLE 3
AST conditions microtiter plate

| Condition # | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| # of cells | 5e4 | 5e4 | 5e4 | 5e4 | 0 | 0 | 4 | 4 | 5e4 | 5e4 | 5e4 | 5e4 | 0 | 0 | 4 | 4 |
| ETP | 0 | 1 | 0 | 1 | 0 | 0 | 0 | 1 | 0 | 1 | 0 | 1 | 0 | 0 | 0 | 1 |
| (μg/mL) | | | | | | | | | | | | | | | | |
| membrane | no | No | yes | yes | no | Yes | yes | yes | no | No | yes | Yes | no | yes | yes | Yes |
| washed | | | | | | | | | | | | | | | | |
| membrane | no | No | no | no | no | no | no | no | yes | yes | yes | Yes | yes | yes | yes | Yes |
| heated | | | | | | | | | | | | | | | | |
| keep feed | yes | Yes | yes | yes | yes | yes | | | yes | yes | yes | Yes | yes | yes | | |
| keep filtrate | yes | Yes | yes | yes | yes | yes | yes | yes | yes | yes | yes | Yes | yes | yes | yes | Yes |
| keep lysate | yes | Yes | yes | yes | | | yes | yes | yes | yes | yes | Yes | | | yes | Yes |
| Reverse | | | | | | | yes | yes | | | | | | | yes | Yes |
| transcription | | | | | | | | | | | | | | | | |

After 60 minutes of incubation, 14 μL of each antibiotic exposure was transferred to its own well of the filter plate. An additional 10 μL of conditions 7, 8, 15, and 16 was transferred to the filter plate. For conditions 1-6 and 9-14, as indicated by the row “keep feed” of the table above, 10 μL of the antibiotic exposure was transferred to a PCR tube containing 10 μL of Lucigen® DNA Extraction Buffer instead of to the filter plate, creating “feed fractions”.

3. Separation of the Sample by in Extracellular/Accessible Nucleic Acid and Intracellular/Inaccessible Nucleic Acid by Filtration and Centrifugation

The filter plate was centrifuged at 2000 rcf for 2 minutes. Fluid that passes through the filter, called the “filtrate fraction”, was collected in a clean 96-well polypropylene microtiter plate. It is expected that the filtrate will contain all or most of the extracellular nucleic acids present in the antibiotic exposure, but none of the intracellular nucleic acids in the antibiotic exposure. While the filter plate was being centrifuged, the feed fractions were vortexed and spun. When the centrifugation of the filter plate was completed, 11 μL of the collected filtrate from conditions 1-6 and 9-14 was separately transferred to and mixed by vortexing with 11 μL of DNA Extraction Buffer to create “filtrate fractions”. 21 μL of the collected filtrate from conditions 7, 8, 15, and 16 was each separately transferred to and mixed by vortexing with 21 μL of DNA Extraction Buffer to create four more filtrate fractions.

4. Cell Lysis to Provide Lysate Comprising Intracellular/Inaccessible Nucleic Acid

20 μL of DNA Extraction Buffer was placed on top of the filters. The filter plate was heated to 65° C. for 6 minutes, shaking at 700 rpm, on a ThermoMixer® flat surface heating block. Then, the filter plate was taped to a clean 96-well polypropylene microtiter plate and centrifuged at 2000 rcf for 2 minutes. The DNA Extraction Buffer fluid that flowed through the filter was collected in the microtiter plate below the filter plate. Next, the microtiter plate was heated to 98° C. for 4 minutes inside a BioRad thermocycler. These collected and heated fluid volumes are termed the “lysate fraction” comprising intracellular nucleic acid.

5. Cold Storage of Feed, Filtrate and Lysate

The feed fractions, filtrate fractions, and lysate fractions were frozen at −80° C. for storage. This cold storage step is optional, as one could proceed to the next step without taking a pause that requires cold storage of one's analytes.

6. Reverse Transcription of Extracellular RNA and Intracellular RNA

3.5 μL of the filtrate and lysate fractions for conditions 7, 8, 15, and 16 were added to 0.1 μL of 3 U/mL Lucigen® RapiDxFire thermostable reverse transcriptase, 0.5 μL of Lucigen® RapiDxFire 10× thermostable buffer, 0.25 μL of 10 mM deoxyribonucleic acid nucleotides, and 0.2 μL of a 10 μM aqueous solution of DNA primer, according to manufacturer's instructions, to create a reverse transcription reaction with a total volume of 5.0 μL. a mixture of Lucigen® RapiDxFire thermostable reverse transcriptase, Lucigen® RapiDxFire thermostable buffer, deoxyribonucleic acid nucleotides, deionized water, and a primer, according to manufacturer's instructions, in a total volume of 5.0 μL to create 8 reverse transcription reaction.

The primer included had a sequence of 5′-TGTCTCCCGTGATAACTTTCTC-3′. (SEQ ID NO; 3) This primer's sequence was complementary to the 23S ribosomal RNA in Escherichia coli and specific to the Enterobacteriaceae family. The cDNA product that was created from this primer contained the primer sites for the future ddPCR reaction occurring later in this AST protocol. The reverse transcription reactions were heated to 60° C. for 5 minutes to create cDNAs, then heated to 95° C. for 5 minutes to stop the reaction and inactivate the reverse transcriptase enzyme. A reverse transcription step was not performed for the other 12 conditions. Alternative reverse transcription enzymes, protocols, and kits may be used instead of the kit used in this example. Alternative primers may be used.

7. Nucleic Acid Quantification in Feed, Filtrate and Lysate:

1.2 μL of the feed fractions, filtrate fractions, and lysate fractions of conditions 1-4 and 9-12 were added, according to kit instructions, to BioRad QX200 ddPCR EvaGreen 2× supermix, deionized water, and a pair of PCR primers for a total volume of 20 For conditions 5, 6, 13, and 14, 5.0 μL of their feed fractions and filtrate fractions were added, according to kit instructions, to BioRad QX200 ddPCR EvaGreen 2× supermix, deionized water, and a pair of PCR primers for a total volume of 20 For conditions 7, 8, 15, and 16, 5.0 μL of their reverse transcription reactions (for their filtrate and lysate fractions) were added, according to kit instructions, to BioRad QX200 ddPCR EvaGreen 2× supermix, deionized water, and a pair of PCR primers for a total volume of 20 The ddPCR reactions only differed in the amount of template replaced by water, which was 3.8 μL added to the first set of ddPCR reactions mentioned versus the other reactions.

The primers used in all ddPCR reactions possessed the following sequences: 5′-GGTAGAGCACTGTTTTGGCA-3′ (SEQ ID NO: 2), 5′-TGTCTCCCGTGATAACTTTCTC-3′(SEQ ID NO: 3). These primers' sequences flanked an 80 bp region common to all of the 23S ribosomal RNA in Escherichia coli but specific to the Enterobacteriaceae family. One of the primers was the same primer used in the prior reverse transcription reaction.

Immediately after setting up the ddPCR reactions, droplet digital PCR (ddPCR) was performed on the BioRad QX200 platform according to manufacturer's instructions. The output of the ddPCR run was the nucleic acid concentration in the filtrate and in the lysate of both antibiotic exposures. Alternative nucleic acid quantification methods could have been employed, including all of the methods for nucleic acid quantification enumerated earlier in this document.

The result of the ddPCRs was 74 expected concentrations of rRNA and a 95% Poisson confidence interval denoting the range over which the same mean concentration would have also appeared 95% of the time if it were to be repeated, with variation due solely to the stochastic loading of template molecules into the droplets.

### Example 5: Digital Same-Sample Filtration AST with One Treated Condition and One Concurrent Reference Condition

An exemplary digital same-sample AST protocol is provided herein below in an outline describing the various sets of operations comprised in the protocol.

1. Providing a Sample:

For the purposes of demonstration, a contrived clinical specimen was created by inoculating an Escherichia coli isolate into Brain-Heart Infusion broth. The inoculum was small enough that no detectable difference in the sample's optical density at 600 mm (OD₆₀₀) was detectable by a spectrophotometer with a sensitivity of 0.01 absorbance units. After an incubation at 37° C., the media became turbid with an OD₆₀₀ of 0.18 absorbance units after 3 hours of incubation.

2. Digital Partitioning of the Sample:

In this experiment, the number and volume of sample partitions was restricted, for logistical reasons, to 96 partitions 10 μL, in volume, specifically the wells of a 96-well plate. A goal was chosen of having a >98% chance of ending up with at least 48 empty partitions. When all partitions are the same volume, the following formula relates the expected number of empty partitions, the partition volume, and the density of cells.

$${\sum }_{k = n}^{N} ( \begin{matrix}N \\ k\end{matrix} ) {{e}^{- D V k} ( 1 - {e}^{- D V} )}^{N - k} > t \qquad ( 13 )$$

N is the total number of partitions, n is the number of empty partitions, V is the partition volume, D is the density of cells, and t is a threshold probability chosen by the practitioner. Thus, in order to achieve digital sample partitioning with the available 96-well plate, the contrived clinical specimen was diluted to a cell density of 0.0375 cells/μL. Each 10 μL sample of the specimen would then contain 0.375 cells on average.

Although the density of bacterial cells in the clinical specimen is not known, in clinical scenarios a plausible range of densities is known, and so the partition number and volumes can always be chosen so that it is highly likely for a desired number of partitions to not receive any bacterial cells, with random chance being the reason different partitions differ in the number of cells loaded. Clinical specimens with high densities of cells can also be diluted to increase the maximum allowed volume of the partitions or decrease the minimum required number of partitions.

3. Antibiotic Exposure of the Digitally Partitioned Sample:

To begin the AST protocol, the contrived clinical specimen was physically split into the 96 partitions by transferring 10 μL of the sample, in 96 separate transfers (actually 12 transfers with a multichannel pipette), to 96 wells of a microtiter plate. Each well contained 15 μL of Mueller-Hinton Broth (MHB) growth media. Half of the wells (48) contained 0 μg/mL of dissolved ETP antibiotic and served as reference condition antibiotic exposures. The other half of the wells contained 1.67 μg/mL of ETP (for a final concentration of 1.0 μg/mL) and served as 48 treated condition antibiotic exposures. The 96 antibiotic exposures were incubated at 37° C. for 70 minutes.

4. Digitally Partitioned Sample Separation by Filtration and Centrifugation

The entire volume of each antibiotic exposure was transferred to a Millipore® 96-well sterile polystyrene MultiScreenHTS® filter plate (Millipore-Sigma MSGVS2210). Each well of the filter plate contained a hydrophilic polyvinylidene fluoride (PVDF) filter membrane with a 0.22 μm pore size. The antibiotic exposures were centrifuged at 2200 relative centrifugal force to speed the passage of the antibiotic exposure through the filter and into 96 collecting vessels. The collected fluid was called the “filtrate.”

The filter pore size was chosen to prevent the passage of intact bacterial cells, which are all larger than 0.2 μm, with rare exceptions. The centrifugation speed was chosen to be low enough to prevent cell lysis.

It is expected that the filtrate will contain all or most of the extracellular nucleic acids present in the antibiotic exposure, but none of the intracellular nucleic acids in the antibiotic exposure.

5. Lysate Collection:

50 μL of fresh MHB media was spun through the filters after the first centrifugation (above) to wash away residual extracellular nucleic acids present in the fluid wetting the filters. This wash fluid is not collected with the filtrates.

This is an optional step. Any type of fluid that does not lyse or degrade cells may be passed through the filter. Examples include other growth medias and buffered solutions of salt compounds found physiologically inside of the bacteria. Solutions that are hypoosmotic to the cell interior, such as pure water, increase the osmotic pressure across the cell wall and will lyse cells without rigid cell walls. Bacteria have rigid cell walls and some are adapted to survive sudden increases in osmotic pressure. Bacteria whose cell walls are damaged by antibiotic but have not yet lysed may be induced to lyse by sudden exposure to a hypoosmotic solution.

If the wash solution is collected, accurate susceptibility calling is possible by treated the wash solution as a second filtrate. If not, inaccuracy is introduced into the number of intact cells and the number of total cells in the sample.

6. Extracellular Nucleic Acid Extraction

10 μL of each of the filtrates was added to and mixed with 10 μL of Lucigen DNA Extraction Buffer, heated to 65° C. for 6 minutes, then heated to 98° C. for 4 minutes.

The purpose of this step is to prevent chemical degradation of nucleic acids in the filtrate after collection. DNA Extraction Buffer prevents nucleic acid degradation by digesting and inactivating nuclease proteins. Alternative methods to achieve the same end include other RNA stabilization or nucleic extraction reactions or kits. This step is optional.

7. Cell Lysis to Provide a Lysate Comprising Intracellular/Inaccessible Nucleic Acid

20 μL of DNA Extraction Buffer was placed on top of the filters. The filter plate was heated to 65° C. for 6 minutes, shaking at 700 rpm, on a ThermoMixer® flat surface heating block. Then, the filter plate was taped to a clean 96-well polypropylene microtiter plate and centrifuged at 2000 rcf for 2 minutes. The DNA Extraction Buffer fluid that flowed through the filter was collected in the microtiter plate below the filter plate. Next, the microtiter plate was heated to 98° C. for 4 minutes inside a BioRad thermocycler. These collected and heated fluid volumes are termed the “lysates” which comprise intracellular nucleic acid.

In particular, the purpose of this step is to recover the intracellular nucleic acids found in the intact cells retained on the filters. To do so, these intact cells are lysed and their nucleic acids extracted. The lysate is expected to contain all or most of the formerly intracellular, now extracellular nucleic acids.

Alternative ways to extract the intracellular nucleic acids can be performed. For example, the filter membrane can be removed from the filter apparatus using sterile and clean forceps and placed into a volume of DNA Extraction Buffer. This volume of buffer is vortexed vigorously, then heated to 65° C., then heated to 98° C.

As a third alternative, intact bacterial cells retained on the filter can be mechanically dislodged (e.g. centrifugation in the opposite direction, stirring), then transferred to a volume of DNA Extraction Buffer, which is then heated to 65° C. and then to 98° C.

The temperatures of 65° C. and 98° C. derive from the manufacturer's instructions for the Lucigen DNA Extraction Buffer kit.

8. Reverse Transcription of Intracellular and Extracellular RNA:

Separately, for each of the 96 extracted filtrates and for each of the 96 extracted lysates, 1.00 μL of the extracted filtrate was mixed with 0.02 μL of 3 U/mL Lucigen® RapiDxFire thermostable reverse transcriptase, 0.2 μL of Lucigen® RapiDxFire 10× thermostable buffer, 0.1 μL of 10 mM deoxyribonucleic acid nucleotides, 0.6 μL of deionized water, and 0.08 μL of a 10 μM aqueous solution of DNA primer, according to manufacturer's instructions, to create a reverse transcription reaction with a total volume of 2.0 μL.

The reagents except for the templates were first mixed together to form a 192 μL master mix; they were not individually added to each of the 192 reverse transcription reactions. The DNA primer included had a sequence of 5′-TGTCTCCCGTGATAACTTTCTC-3′ (SEQ ID NO:3). The primer's sequence was complementary to the 23S ribosomal RNA in Escherichia coli and specific to the Enterobacteriaceae family. The cDNA product that would be created from this primer contained the primer sites for the future ddPCR reaction occurring later in this AST protocol. All 192 reverse transcription reactions were heated to 60° C. for 5 minutes to create cDNAs, then heated to 95° C. for 5 minutes to stop the reaction and inactivate the reverse transcriptase enzyme.

A reverse transcription step is optional if one has decided to amplify a DNA molecule found naturally in the cells of interest. However, if the nucleic acid to be quantified in the AST protocol is a ribonucleic acid (RNA) molecule, and the quantification method operates only on deoxyribonucleic acid molecules, then both the filtrate and the lysate can be treated with a reverse transcriptase enzyme to produce complementary DNA molecules (cDNA) prior to nucleic acid quantification. The concentration of cDNA, and thus rRNA, is calculated from the counts of high and low fluorescence droplets.

Alternative reverse transcription enzymes, protocols, and kits may be used instead of the kit used in this example.

Alternative primers can be used as will be understood by a skilled person. Alternative nucleic acid species can be targeted as well, through a choice of primers. As noted earlier in this document, targets with a higher copy number per cell are preferred for accessibility AST.

9. Quantification of Extracellular and Intracellular Nucleic Acid

A 10 μL volume of each of the above reverse transcription reactions was separately added, according to kit instructions, to 2.5 μL of BioRad SsoFast qPCR EvaGreen 2× supermix, 1.30 μL nuclease-free water, and 0.2 μL of a pair of DNA PCR primers at 10 μM each, to create a 5 μL qPCR reaction.

The DNA primers' sequences flanked an 80 bp region common to all of the 23S ribosomal RNA in Escherichia coli but specific to the Enterobacteriaceae family. One of the primers was the same primer used in the prior reverse transcription reaction. Real time qPCR of the qPCR reactions was performed on the BioRad CFX96 platform according to manufacturer's instructions. The real time qPCR protocol comprised 45 cycles of 30 seconds of denaturing at 95° C. and 60 seconds of annealing and extension at 60° C. The output of the qPCR run was the threshold cycles, which reflect nucleic acid concentration, of the filtrate and in the lysate of both antibiotic exposures.

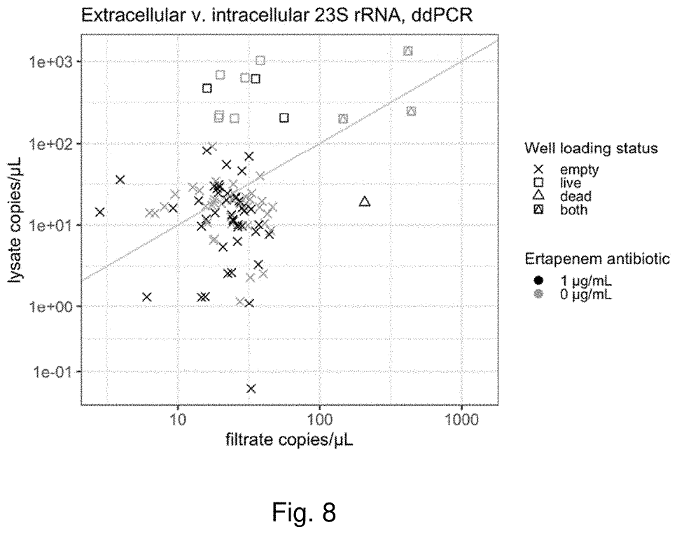

The results shown in FIG. 6 illustrated as a cluster analysis presented to also report the loading status of the 96 sample partitions, under treated conditions (black markings) and test conditions (white markings). In particular, the cluster analysis shown in FIG. 6 indicate that in that of the 96 partitions, 19 lysed cells (square) and 6 included intact cells (diamond) were detected, while 71 partitions contained no cells (circles). No partitions were inferred to contain both intact and lysed cells. All detected & antibiotic-treated cells underwent lysis (100% extracellular), while all detected & untreated cells remained intact (0% extracellular), indicating that the strain was susceptible.

Alternative nucleic acid quantification methods could have been employed, including all of the methods for nucleic acid quantification enumerated earlier in this document.

Indeed, a different method, ddPCR, was performed in this experiment to demonstrate the flexibility in the detection methods of the same sample approach and is discussed below. From the 96 pairs of filtrate and lysate nucleic acid concentrations measured, the loading status of the 96 antibiotic exposures were estimated using K-medoids clustering with 5 clusters. The cluster with the lowest filtrate threshold cycle (and highest filtrate cDNA concentration) was determined to represent wells with lysed cells. The cluster with the lowest lysate threshold cycle (and highest lysate cDNA concentration) was determined to represent wells with intact cells. The other clusters were called as empty wells.

Alternative choices for the well loading status algorithm can be used. A non-exhaustive list of appropriate choices is described in other sections of the present disclosure.

The number of wells in each of the experimental conditions (treated and reference) possessing each loading status were counted. From the counts, the fraction of cells that lysed was calculated in each experimental condition. Fisher's exact test was performed to test the hypothesis that the rate of lysis is the same in both experimental conditions. Since the chance of the data arising from the same rate of lysis was much smaller than 0.05, the strain was correctly called as susceptible.

Other statistical hypothesis testing methods can be used, including Barnard's test, Boschloo's test, the Chi-square test, the T-test, and other frequentist tests. Bayesian statistical models of varying complexity could also be defined and applied to the data. For some of these tests to apply, one can use data from prior runs that replicate this experiment. These data can be obtained in prior repetitions of this protocol, or in repetitions of this protocol performed at the same time (e.g. in a high throughput set up).

According to the above settings, in parallel to the qPCR quantification, ddPCR was performed. A volume of each of the above reverse transcription reactions was separately added to water and BioRad QX200 ddPCR EvaGreen supermix, according to kit instructions. A pair of PCR primers was also included. These primers' sequences flanked an 80 bp region common to all of the 23S ribosomal RNA in Escherichia coli but specific to the Enterobacteriaceae family. One of the primers was the same primer used in the prior reverse transcription reaction. Droplet digital PCR (ddPCR) was performed on the BioRad QX200 platform according to manufacturer's instructions. The output of the ddPCR run was the nucleic acid concentration in the filtrate and in the lysate of both antibiotic exposures.

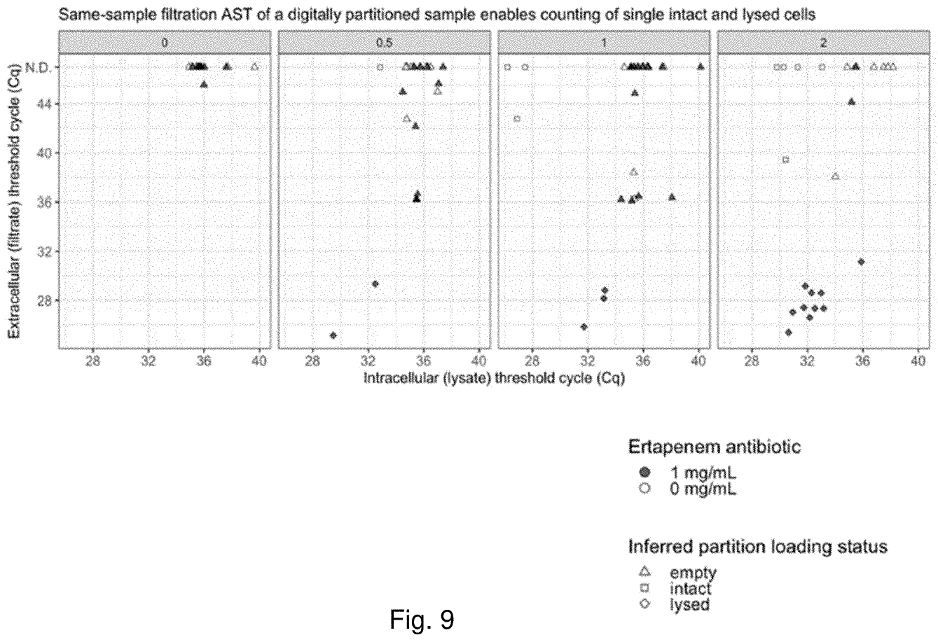

The results are illustrated in the cluster analysis shown in FIG. 7 performed on 56 of the 96 sample partitions/antibiotic exposures analyzed by qPCR (not all 96 partitions from FIG. 6 were analyzed). In particular, the cluster analysis shown in FIG. 7 shows that 9 lysed cells and 4 intact cells were detected and 43 partitions contained no cells. No partitions were inferred to contain both intact and lysed cells. All detected & antibiotic-treated cells underwent lysis (100% extracellular), while all detected & untreated cells remained intact (0% extracellular), indicating that the strain was susceptible.

Similar to the qPCR data, the loading status of each antibiotic exposure/sample partition was determined by k-medoids clustering, the number of lysed and intact cells in each experimental condition counted, and the susceptibility call made by Fisher's exact test. The strain was correctly determined to be susceptible.

Further alternative or additional nucleic acid quantification methods can have been employed, including all of the methods for nucleic acid quantification indicated in different sections of the present disclosure, as will be understood by a skilled person.

### Example 6: Digital Same-Sample Filtration AST with One Treated Condition and One Concurrent Reference Condition

An exemplary digital same-sample AST protocol is provided herein below in an outline describing the various sets of operations comprised in the protocol.

1. Providing a Sample

For the purposes of demonstration, a contrived clinical sample was created by inoculating a carbapenem-resistant Escherichia coli isolate into Brain-Heart Infusion broth. The inoculum was small enough that no detectable difference in the sample's optical density at 600 mm (OD₆₀₀) was detectable by a spectrophotometer with a sensitivity of 0.01 absorbance units. The OD₆₀₀ of the culture measured every 30 minutes after the culture was incubated at 37° C. to calculate the doubling time of the strain. The media became turbid with an OD₆₀₀ of 0.28 absorbance units after 3.1 hours of incubation.

2. Sample Partitioning

In this experiment, the number and volume of sample partitions was restricted, for logistical reasons, to 96 partitions 10 μL, in volume, specifically the wells of a 96-well plate. A goal was chosen of having a >98% chance of ending up with at least 48 empty partitions. When all partitions are the same volume, the following formula relates the expected number of empty partitions, the partition volume, and the density of cells.

$${\sum }_{k = n}^{N} ( \begin{matrix}N \\ k\end{matrix} ) {{e}^{- D V k} ( 1 - {e}^{- D V} )}^{N - k} > t \qquad ( 16 )$$

N is the total number of partitions, n is the number of empty partitions, V is the partition volume, D is the density of cells, and t is a threshold probability chosen by the practitioner. Thus, in order to achieve digital sample partitioning with the available 96-well plate, the contrived clinical specimen was diluted to a cell density of 0.0375 cells/μL. Each 10 μL sample of the specimen would then contain 0.375 cells on average.

Although the density of bacterial cells in the clinical specimen is not known, in clinical scenarios a plausible range of densities is known, and so the partition number and volumes can always be chosen so that it is highly likely for a desired number of partitions to not receive any bacterial cells, with random chance being the reason different partitions differ in the number of cells loaded. Clinical specimens with high densities of cells can also be diluted to increase the maximum allowed volume of the partitions or decrease the minimum required number of partitions.

3. Antibiotic Exposure of the Sample Partitions

To begin the AST protocol, the contrived clinical sample was physically split into the 96 partitions by transferring 10 μL of the sample, in 96 separate transfers (actually 12 transfers with a multichannel pipette), to 96 wells of a microtiter plate. Each well contained 15 μL of Mueller-Hinton Broth (MHB) growth media. Half of the wells (48) contained 0 μg/mL of dissolved ETP antibiotic and served as reference condition antibiotic exposures. The other half of the wells contained 1.67 μg/mL of ETP (for a final concentration of 1.0 μg/mL) and served as 48 treated condition antibiotic exposures.

The 96 antibiotic exposures were incubated at 37° C. for 40 minutes.

4. Separation of Sample Partitions by Filtration and Centrifugation

The entire volume of each antibiotic exposure was transferred to a Millipore® 96-well sterile polystyrene MultiScreenHTS® filter plate (Millipore-Sigma MSGVS2210). Each well of the filter plate contained a hydrophilic polyvinylidene fluoride (PVDF) filter membrane with a 0.22 μm pore size. The antibiotic exposures were centrifuged at 2200 relative centrifugal force to speed the passage of the antibiotic exposure through the filter and into 96 collecting vessels. The collected fluid was called the “filtrate.” It is expected that the filtrate will contain all or most of the extracellular nucleic acids present in the antibiotic exposure, but none of the intracellular nucleic acids in the antibiotic exposure.

The filter pore size was chosen to prevent the passage of intact bacterial cells, which are all larger than 0.22 with rare exceptions.

The centrifugation speed was chosen to be low enough to prevent cell lysis, as will be understood by a skilled person upon reading of the present disclosure.

The exposure duration was chosen to be 40 minutes because this experiment had a secondary goal of validating the effect of the exposure duration. Any exposure duration longer than 30 minutes and up to 240 minutes could have been chosen for a different clinical context, such as weighing the accuracy of the test more than the turnaround time. Any exposure shorter than 30 minutes could also have been chosen if the turnaround time was deemed more important than the accuracy of the test.

5. Filter Washing

50 μL of fresh MHB media was spun through the filters after the first centrifugation (above) to wash away residual extracellular nucleic acids present in the fluid wetting the filters. This wash fluid is not collected with the filtrates.

This is an optional step. Any type of fluid that does not lyse or degrade cells may be passed through the filter. Examples include other growth medias and buffered solutions of salt compounds found physiologically inside of the bacteria.

Solutions that are hypoosmotic to the cell interior, such as pure water, increase the osmotic pressure across the cell wall and will lyse cells without rigid cell walls. Bacteria have rigid cell walls and some are adapted to survive sudden increases in osmotic pressure. Bacteria whose cell walls are damaged by antibiotic but have not yet lysed may be induced to lyse by sudden exposure to a hypoosmotic solution. If the wash solution is collected, accurate susceptibility calling is possible by treated the wash solution as a second filtrate. If not, inaccuracy is introduced into the number of intact cells and the number of total cells in the sample.

6. Extraction of Extracellular Nucleic Acid from the Filtrate

10 μL of each of the filtrates was added to and mixed with 10 μL of Lucigen DNA Extraction Buffer, heated to 65° C. for 6 minutes, then heated to 98° C. for 4 minutes to create extracted filtrates.

The purpose of this step is to prevent chemical degradation of nucleic acids in the filtrate after collection. DNA Extraction Buffer prevents nucleic acid degradation by digesting and inactivating nuclease proteins.

Alternative methods to achieve the same end include other RNA stabilization or nucleic extraction reactions or kits. This step is optional. 8. Cell Lysis to Provide Extracted Lysate Comprising Intracellular Nucleic Acid

20 μL of DNA Extraction Buffer was placed into all of the wells of the filter plate, on top of the filters. The filter plate was heated to 65° C. for 6 minutes, shaking at 700 rpm, on a ThermoMixer® flat surface heating block. Then, the filter plate was taped to a clean 96-well polypropylene microtiter plate and centrifuged at 2000 rcf for 2 minutes. The DNA Extraction Buffer fluid that flowed through the filter was collected in the microtiter plate below the filter plate. Next, the microtiter plate was heated to 98° C. for 4 minutes inside a BioRad thermocycler. These collected and heated fluid volumes are termed the “extracted lysate”.

The purpose of this step is to recover the intracellular nucleic acids found in the intact cells retained on the filters. To do so, these intact cells are lysed and their nucleic acids extracted. The lysate is expected to contain all or most of the formerly intracellular, now extracellular nucleic acids.

Alternative ways to extract the intracellular nucleic acids can be performed. For example, the filter membrane can be removed from the filter apparatus using sterile and clean forceps and placed into a volume of DNA Extraction Buffer. This volume of buffer is vortexed vigorously, then heated to 65° C., then heated to 98° C.

As a third alternative exemplary method, intact bacterial cells retained on the filter can be mechanically dislodged (e.g. centrifugation in the opposite direction, stirring), then transferred to a volume of DNA Extraction Buffer, which is then heated to 65° C. and then to 98° C. The temperatures of 65° C. and 98° C. derive from the manufacturer's instructions for the Lucigen DNA Extraction Buffer kit.

9. Reverse Transcription of 23 SRNA in Filtrates and Lysates

Separately, for each of the 96 extracted filtrates and for each of the 96 extracted lysates, 1.00 μL of the extracted filtrate was mixed with 0.02 μL of 3 U/mL Lucigen® RapiDxFire thermostable reverse transcriptase, 0.2 μL of Lucigen® RapiDxFire 10× thermostable buffer, 0.1 μL of 10 mM deoxyribonucleic acid nucleotides, 0.6 μL of deionized water, and 0.08 μL of a 10 μM aqueous solution of DNA primer, according to manufacturer's instructions, to create a reverse transcription reaction with a total volume of 2.0 μL. The reagents except for the templates were first mixed together to form a 192 μL master mix; they were not individually added to each of the 192 reverse transcription reactions. The DNA primer included had a sequence of 5′-TGTCTCCCGTGATAACTTTCTC-3′ (SEQ ID NO:3). The primer's sequence was complementary to the 23S ribosomal RNA in Escherichia coli and specific to the Enterobacteriaceae family. The cDNA product that would be created from this primer contained the primer sites for the future ddPCR reaction occurring later in this AST protocol. All 192 reverse transcription reactions were heated to 60° C. for 5 minutes to create cDNAs, then heated to 95° C. for 5 minutes to stop the reaction and inactivate the reverse transcriptase enzyme.

A reverse transcription step is optional if one has decided to amplify a DNA molecule found naturally in the cells of interest. However, if the nucleic acid to be quantified in the AST protocol is a ribonucleic acid (RNA) molecule, and the quantification method operates only on deoxyribonucleic acid molecules, then both the filtrate and the lysate can be treated with a reverse transcriptase enzyme to produce complementary DNA molecules (cDNA) prior to nucleic acid quantification. The concentration of cDNA, and thus rRNA, is calculated from the counts of high and low fluorescence droplets.

Alternative reverse transcription enzymes, protocols, and kits may be used instead of the kit used in this example as will be understood by a skilled person. Alternative primers can also be used as will also be understood by a skilled person. Alternative nucleic acid species can be targeted as well, through a choice of primers. As noted earlier in this document, targets with a higher copy number per cell are preferred for accessibility AST.

10. Nucleic Acid Quantification of in Filtrate and Lysates

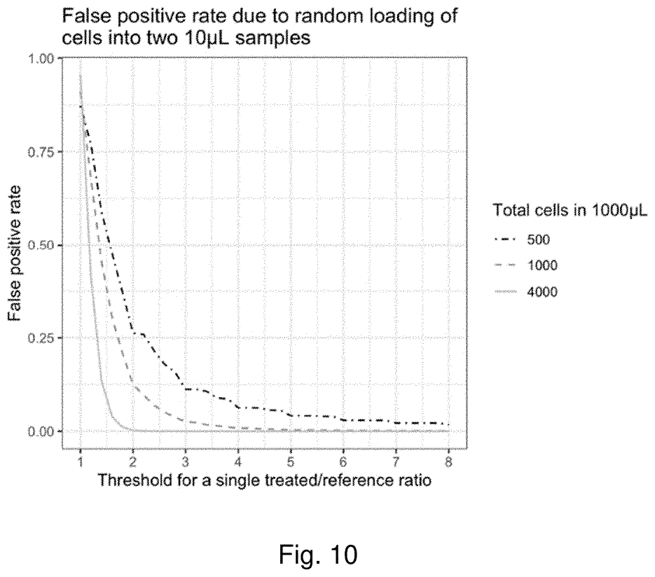

A 1.5 μL volume of each of the above 192 reverse transcription reactions was separately added to 5.4 μL water, 0.6 μL of 10 μM PCR forward and reverse primers, and 7.5 μL of BioRad SsoFast QX200 ddPCR EvaGreen supermix to make a 15 μL ddPCR reaction, using micropipettors and multichannel pipettes. The pair of PCR primers possessed the following sequences: 5′-GGTAGAGCACTGTTTTGGCA-3′ (SEQ ID NO: 2), 5′-TGTCTCCCGTGATAACTTTCTC-3′ (SEQ ID NO: 3). These primers' sequences flanked an 80 bp region common to all of the 23S ribosomal RNA in Escherichia coli but specific to the Enterobacteriaceae family. One of the primers was the same primer used in the prior reverse transcription reaction. Digital droplet PCR was performed on the BioRad QX200 platform according to manufacturer's instructions. The output of the 192 ddPCR reactions was 96 pairs of absolute nucleic acid concentrations. 48 pairs came from the treated condition and 48 pairs came from the reference condition. Each pair comprised an ENACV from the filtrate of a sample and an INACV from the lysate of the same sample. The results are shown in FIG. 8.

In particular, FIG. 8 shows a comparison of an extracellular nucleic acid concentration value (ENACV) from the filtrate of the sample partitions and an intracellular nucleic acid concentration value (INACV) from the lysate of the same sample partitions in terms of copies/ul, the ENCV and the INACV are reported for each pair of the 48 pairs from the treated condition (black symbols) (white symbols) and 48 pairs from the reference condition.

Alternative nucleic acid quantification methods could have been employed, including all of the methods for nucleic acid quantification enumerated as indicated in the present disclosure.

11. Well Loading Status Algorithm (Intermediary Step Towards Estimating Single Cell Statuses)

From the 96 pairs of filtrate and lysate nucleic acid concentrations measured, the loading status of the 96 antibiotic exposures were estimated using a well loading status algorithm. The well loading status algorithm used involved a combination of two thresholds. was K-medoids clustering with 5 clusters. The cluster with the highest ENACV was determined to represent wells with killed cells. The cluster with the highest INACV was determined to represent wells with intact cells. The remaining clusters were called as empty wells.

The number of wells in each of the experimental conditions (treated and reference) possessing each loading status were counted. The results are found in Table 3 below, as well as being shown in FIG. 8 in particular as shown by the illustration of FIG. 8, and Table 3 44 of the 48 treated partitions were determined to be empty, 1 was determined to have EINACV only, 3 partitions were determined to have 3 INACV only and none was determined to have both.

TABLE 3
Number of cells in treated and reference samples

| | | Extracellular | Intracellular | |
|---|---|---|---|---|
| | Empty | only | only | Both |
| Treated (1.0 μg) | 44 | 1 | 3 | 0 |
| Reference (0.0 μg) | 39 | 0 | 6 | 3 |

No prior reference condition data was used in this algorithm because the algorithm performed adequately without such data.

Alternative choices for the well loading status algorithm can be used. A non-exhaustive list of appropriate choices is indicated in a different section of the present disclosure.

12. Estimation of Single Cell Responses to Antibiotic

The next step of the analysis involves estimating the number of killed and intact cells from the tallies of samples in each loading status. From the counts, the density C of cells was estimated to be

$$C = - ln ( \frac{44 + 39}{96} ) = 0.146 \frac{cell}{sample} ,$$

indicating that there were most likely 13 cells in all the samples at the time of sample partitioning. Because 3 wells were placed the “Both” category, it was estimated that 3 additional cells arose at minimum by the time filtration was performed, so that each “Both” well contained one killed and one intact cell. Thus, the treated condition contained 1 killed cell and 3 intact cells, while the reference condition contained 3 killed cells and 9 intact cells at the time of filtration.

An alternative analysis is possible if one assumes that all cells were undergoing exponential division during the antibiotic exposure. Since the doubling time of the strain (measured in step 1) was approximately 30 minutes, each cell in a sample with some intracellular nucleic acid would have divided at least once by the time the 40 minute exposure ended. Thus, an estimate of 1+2(3)+2(6)+2(3)=25 cells in total is made. The treated condition would contain 1 killed cell and 2(3)=6 intact cells, while the reference condition would contain 3 killed cells and 1(3)+2(6)=15 intact cells.

The estimate of how many cells exist in each sample at the end of the exposure is most preferably modeled by a branching process probability model of the antibiotic exposures. The parameters in this model are fitted from prior experiments with the same species of bacteria.

13. Calculation of the EINAPV

The next step in the analysis involves calculating an extracellular/intracellular nucleic acid proportion value (EINAPV) for each experimental condition. For example, the fraction extracellular served as the EINAPV. The treated EINAPV was 1/(1+3)=1/4, while the reference EINAPV was 3/(3+9)=1/4.

Using an alternative estimate of the total number of cells, the treated EINAPV would be 1/(1+6)=1/7 and the reference EINAPV would be 3/(3+15)=1/6.

14. Susceptibility Call. Determination of Susceptibility Using the Extracellular/Intracellular Nucleic Acid Proportion Values (EINAPV) from Each Experimental Condition.

Finally, the statistical significance of treated EINAPV was assessed. Specifically, the treated EINAPV was compared to the null hypothesis that its value could have arisen from the reference condition distribution due to chance alone. There are several comparable ways to test statistical significance, each making different assumptions and yielding similar results.

The results of this determination is shown in FIG. 8, where the empty wells are indicated with an x, the wells including live cells are indicated with a square, the wells including dead cells are indicated with a triangle and the wells that include both live and dead cells are indicated with a square including a triangle.

Because there is one treated condition and one concurrent reference condition, because a digital same-sample AST was performed, and because the digital same-sample AST yields integer counts of killed and intact cells, it becomes possible to apply several statistical tests defined for contingency tables. For example, Fisher's exact test can be performed to test the hypothesis that the rate of lysis is the same in both experimental conditions. Fisher's exact test on the above 2×2 contingency table of counts of cells in each state yields a p-value of 1. Using the counts from the alternative analysis in Fisher's exact test also yields a p-value of 1. Since the p-value was greater than the traditional 0.05 significant threshold chosen a priori, the strain was correctly called as resistant. Other statistical hypothesis testing methods for contingency tables could be used, including Barnard's test, Boschloo's test, and Pearson's chi-square test.

Bayesian statistical models of varying complexity could also be defined and applied to the data. For some of these tests to apply, one may need data from prior runs that replicate this experiment. These data could be obtained in prior repetitions of this protocol, or in repetitions of this protocol performed at the same time (e.g. in a high throughput set up).

Alternatively, because there is only one treated EINAPV and one concurrent reference EINAPV, one could gather or create prior reference condition data from untreated reference conditions as a sampling of a reference distribution similar to the true concurrent reference distribution. These data comprise the EINAPVs obtained when a same-sample AST protocol, preferably this same protocol, is performed up to this step on samples known to contain the same or closely related species of microorganism (such as positive clinical specimens, or less preferably contrived clinical specimens spiked with the microorganism) and in which the microorganisms were not contacted with the antibiotic (contacted only with the vehicle of the antibiotic (e.g. pure water) and not the antibiotic compound itself, or less preferably where no contacting is performed). It is desired to include microorganisms in the prior reference condition data that are as similar to the currently tested microorganism as possible, with a tradeoff occurring between a larger number of prior reference condition data and the similarity of the microorganism in the included prior data. One would then compare the concurrent reference EINAPV to the prior reference condition data.

If the concurrent reference EINAPV is not significantly different from the prior reference condition data, then the prior reference condition data is a good approximation of the true reference condition distribution. The significance is found by comparing the treated condition EINAPV to a threshold as described herein.

Otherwise, one uses the single concurrent reference condition as the best approximation of the true reference condition distribution, or one needs to repeat the assay to obtain more reference and/or more treated replicates. In the former, a susceptible call is made so long as the treated EINAPV indicates more lysis than does the concurrent reference EINAPV. This simple comparison method has a relatively high rate of assay error. In this experiment, using the simpler estimate for the numbers of killed and intact cells, the treated fraction extracellular of ¼ is equal to the reference fraction extracellular of ¼, so the strain would be correctly called as resistant. In this experiment, using the alternative estimate for the numbers of killed and intact cells, the treated fraction extracellular of 1/7 is less than the reference fraction extracellular of ⅙, so the strain would again be correctly called as resistant.

### Example 7: Digital Same-Sample Filtration AST with One Treated Condition and One Concurrent Reference Condition

In the experiment described in this example, there were two possible goals, each with their own interpretation of the same results. One purpose was to validate the stochastic loading of cells as a function of cell density. This goal is not a question that clinicians using our assay would pursue, but it does illustrate optional treatments of the filter membrane that our invention entails. The other goal of the experiment would be to confirm the susceptibility of the bacteria strain Escherichia coli K12 to demonstrate our assay's validity, even though we already knew Escherichia coli K12 is susceptible. This latter goal mimics the questions pursued by future users of this invention.

The related exemplary digital same-sample AST protocol is provided herein below in an outline describing the various sets of operations comprised in the protocol.

1. Providing a Sample

For the purposes of demonstration, a contrived clinical sample was created by inoculating Escherichia coli K12 into Brain-Heart Infusion broth. The inoculum was small enough that no detectable difference in the sample's optical density at 600 mm (OD₆₀₀) was detectable by a spectrophotometer with a sensitivity of 0.01 absorbance units. After an incubation at 37° C., the media became turbid with an OD₆₀₀ of 0.22 absorbance units after 2.25 hours of incubation.

The above sample was provided with a known susceptible bacterial strain to provide a proof of principle. Samples can be provided from specimen to perform testing for microorganisms whose susceptibility/resistance to the antibiotic is unknown without inoculation modifying the above procedure in a manner identifiable by a skilled person upon reading of the present disclosure

2. Partitioning of the Sample to Obtain Targeted 0, 0.5, 1, and 2 Cells Per Partition

In this experiment, the number and volume of sample partitions was restricted, for logistical reasons, to 96 partitions 10 μL in volume, specifically the wells of a 96-well plate. A goal was chosen of having a >98% chance of ending up with at least 48 empty partitions. When all partitions are the same volume, the following formula relates the expected number of empty partitions, the partition volume, and the density of cells.

$${\sum }_{k = n}^{N} ( \begin{matrix}N \\ k\end{matrix} ) {{e}^{- D V k} ( 1 - {e}^{- D V} )}^{N - k} > t \qquad ( 17 )$$

N is the total number of partitions, n is the number of empty partitions, V is the partition volume, D is the density of cells, and t is a threshold probability chosen by the practitioner. Thus, in order to achieve digital sample partitioning with the available 96-well plate, each 10 μL sample of the specimen practically contains between 0.02 and 2.5 cells on average. In this experiment, densities of 0, 0.5, 1, and 2 cells per sample were targeted. Due to systematic bias in the conversion of OD600 to cell density, the actual densities achieved were closer to 0, 0.09375, 0.1875, and 0.375 cells per sample. The specimen itself was diluted to a density of 37.5 CFU/mL (0.0375 cells/μL), 18.75 CFU/mL, and 9.375 CFU/mL to achieve the target sample densities. For the 0 cell samples, the specimen comprised sterile media.

Although the density of bacterial cells in the clinical specimen is not known, in clinical scenarios a plausible range of densities is known, and so the partition number and volumes can always be chosen so that it is highly likely for a desired number of partitions to not receive any bacterial cells, with random chance being the reason different partitions differ in the number of cells loaded. Clinical specimens with high densities of cells can also be diluted to increase the maximum allowed volume of the partitions or decrease the minimum required number of partitions.

3. Contacting the Sample Partitions with Antibiotic (Testing Conditions) or Culture Medium (Reference Conditions)

To begin the AST protocol, the 4 contrived clinical specimen dilutions were physically split into the 24 partitions each by transferring 10 μL of the sample, in 24 separate transfers (actually 3 transfers with a multichannel pipette of each dilution), to wells of a 96-well microtiter plate. Each well contained 15 μL of Mueller-Hinton Broth (MHB) growth media. Half of the wells (48) contained 0 μg/mL of dissolved ETP antibiotic and served as reference condition antibiotic exposures. The other half of the wells contained 1.67 μg/mL of ETP (for a final concentration of 1.0 μg/mL) and served as 48 treated condition antibiotic exposures. Thus, in total there were 8 groups of 12 replicate wells: a treated and an untreated group of wells for each of the four specimen dilutions. In actuality, however, due to operator error, 4 treated 0 cell conditions and 4 untreated 0 cell conditions were converted into the corresponding 0.1875 cell conditions.

The 96 antibiotic exposures were incubated at 37° C. for 60 minutes.

4, Sample Separation of the Partitions by Filtration and Centrifugation

The entire volume of each antibiotic exposure was transferred to a Millipore® 96-well sterile polystyrene MultiScreenHTS® filter plate (Millipore-Sigma MSGVS2210). Each well of the filter plate contained a hydrophilic polyvinylidene fluoride (PVDF) filter membrane with a 0.22 μm pore size. A 96-well polypropylene microtiter plate was affixed to the bottom of the filter plate. At approximately 78 minutes after the start of the exposure, the antibiotic exposures were centrifuged at 2200 relative centrifugal force to speed the passage of the antibiotic exposure through the filter and into 96-well microtiter plate. The collected fluid was called the “filtrate.” It is expected that the filtrate will contain all or most of the extracellular nucleic acids present in the antibiotic exposure, but none of the intracellular nucleic acids in the antibiotic exposure.

The filter pore size was chosen to prevent the passage of intact bacterial cells, which are all larger than 0.22 μm, with rare exceptions.

The centrifugation speed was chosen to be low enough to prevent cell lysis, as described herein.

Any exposure duration longer than 30 minutes and up to 240 minutes can performed instead of the 78-minute exposure actually chosen, and the exposure duration would have remained in the preferred range. Exposures shorter than 30 minutes with the exemplified protocol also enables accurate susceptibility calls in absence of an additional detection in the same sample and/or in a separate sample as will be understood by a skilled person upon reading of the present disclosure,

For clinical applications, the susceptibility can still be correctly called even though manual operation may introduce uncertainty into whether the growth rate of the cells (which is affected by temperature of the media) was constant throughout the entire antibiotic exposure, so long as the temperature did not kill the cells by exiting the range of 4° C. to 40° C., preferably remaining in the range of 25° C. to 37° C., and so long as the duration of uncertain temperature does not exceed or become comparable in length (e.g. ≥50%) to the duration of known temperature. In this experiment, the use of a stopwatch helped minimize uncertainty in the length of the exposure duration, and the 18 minutes of cooler room temperature antibiotic exposure was 30% of the intended 60-minute exposure.

5. Filter Washing

As soon as possible (e.g. 0-10 minutes) after the centrifugation of the filter plate, 50 of fresh MHB media was spun through the filters after the first centrifugation (above) to wash away residual extracellular nucleic acids present in the small amount (about 3 μL) of fluid wetting the filters. This wash fluid is not collected with the filtrates.

This is an optional step. Any type of fluid that does not lyse or degrade cells may be passed through the filter. Examples include other growth medias, phosphate buffered saline, and other buffered solutions of salt compounds found physiologically inside of the bacteria.

Solutions that are hypoosmotic to the cell interior, such as pure water, increase the osmotic pressure across the cell wall and will lyse cells without rigid cell walls. Bacteria have rigid cell walls, and some are adapted to survive sudden increases in osmotic pressure. Bacteria whose cell walls are damaged by antibiotic but have not yet lysed may be induced to lyse by sudden exposure to a hypoosmotic solution. If the wash solution is collected, accurate susceptibility calling is possible by treated the wash solution as a second filtrate. If not, inaccuracy is introduced into the number of intact cells and the number of total cells in the sample.

6. Filtrate Preservation by Freezing

The filtrates were frozen at −80° C. to prevent hypothetical rRNA degradation. Lucigen DNA Extraction Buffer was not used to “extract” the filtrate DNA. The use of an extraction buffer would have diluted the filtrate nucleic acids. If the reverse transcription reaction are performed immediately after the filtrates were created, then freezing can be omitted as will be understood by a skilled person.

7 Cell Lysis to Provide Extracted Lysate Comprising Intracellular Nucleic Acid

20 μL of DNA Extraction Buffer was placed into all of the wells of the filter plate, on top of the filters. The filter plate was heated to 65° C. for 6 minutes, shaking at 700 rpm, on a ThermoMixer® flat surface heating block. Then, the filter plate was taped to a clean 96-well polypropylene microtiter plate and centrifuged at 2200 rcf for 2 minutes. The DNA Extraction Buffer fluid that flowed through the filter was collected in the microtiter plate below the filter plate. Next, the microtiter plate was heated to 98° C. for 4 minutes inside a BioRad thermocycler. These collected and heated fluid volumes are termed the “extracted lysate”.

The purpose of this step is to recover the intracellular nucleic acids found in the intact cells retained on the filters. To do so, these intact cells are lysed and their nucleic acids extracted. The lysate is expected to contain all or most of the formerly intracellular, now extracellular nucleic acids.

Alternative ways to extract the intracellular nucleic acids can be performed. For example, the filter membrane can be removed from the filter apparatus using sterile and clean forceps and placed into a volume of DNA Extraction Buffer. This volume of buffer is vortexed vigorously, then heated to 65° C., then heated to 98° C.

As a third alternative, intact bacterial cells retained on the filter can be mechanically dislodged (e.g. centrifugation in the opposite direction, stirring), then transferred to a volume of DNA Extraction Buffer, which is then heated to 65° C. and then to 98° C.

The temperatures of 65° C. and 98° C. derive from the manufacturer's instructions for the Lucigen DNA Extraction Buffer kit.

8. Preservation of the Extracted Lysates by Freezing

In this experiment, the extracted lysates were frozen at −80° C. to pause the experiment. Freezing extracted nucleic acids can be omitted if one immediately continues to the next step in the protocol (the reverse transcription step).

9. Reverse Transcription of RNA in Filtrates and Lysates

Separately, for each of the 96 extracted filtrates and for each of the 96 extracted lysates, 1.00 μL of the extracted filtrate was mixed with 0.02 μL of 3 U/mL Lucigen® RapiDxFire thermostable reverse transcriptase, 0.2 μL of Lucigen® RapiDxFire 10× thermostable buffer, 0.1 μL of 10 mM deoxyribonucleic acid nucleotides, 0.6 μL of deionized water, and 0.08 μL of a 10 μM aqueous solution of DNA primer, according to manufacturer's instructions, to create a reverse transcription reaction with a total volume of 2.0 μL. The reagents except for the templates were first mixed together to form a 192 μL master mix; they were not individually added to each of the 192 reverse transcription reactions. The DNA primer included had a sequence of 5′-TGTCTCCCGTGATAACTTTCTC-3′ (SEQ ID NO: 3). The primer's sequence was complementary to the 23S ribosomal RNA in Escherichia coli and specific to the Enterobacteriaceae family. The cDNA product that would be created from this primer contained the primer sites for the future ddPCR reaction occurring later in this AST protocol. All 192 reverse transcription reactions were heated to 60° C. for 5 minutes to create cDNAs, then heated to 95° C. for 5 minutes to stop the reaction and inactivate the reverse transcriptase enzyme.

A reverse transcription step is optional if one has decided to amplify a DNA molecule found naturally in the cells of interest. However, if the nucleic acid to be quantified in the AST protocol is a ribonucleic acid (RNA) molecule, and the quantification method operates only on deoxyribonucleic acid molecules, then both the filtrate and the lysate can be treated with a reverse transcriptase enzyme to produce complementary DNA molecules (cDNA) prior to nucleic acid quantification. The concentration of cDNA, and thus rRNA, is calculated from the counts of high and low fluorescence droplets.

Alternative reverse transcription enzymes, protocols, and kits may be used instead of the kit used in this example.

Alternative primers may be used. Alternative nucleic acid species can be targeted as well, through a choice of primers. As noted earlier in this document, targets with a higher copy number per cell are preferred for accessibility AST.

10. Quantification of Extracellular and Intracellular Nucleic Acid in Filtrates and Lysate of Each Partition

A 1 μL volume of each of the above reverse transcription reactions was separately added, according to kit instructions, to 2.5 μL of BioRad SsoFast qPCR EvaGreen 2× supermix, 1.30 μL nuclease-free water, and 0.2 μL of a pair of DNA PCR primers at 10 μM each, to create a 5 μL qPCR reaction. The pair of PCR primers possessed the following sequences: 5′-GGTAGAGCACTGTTTTGGCA-3′ (SEQ ID NO: 2), 5′-TGTCTCCCGTGATAACTTTCTC-3′(SEQ ID NO: 3). The DNA primers' sequences flanked an 80 bp region common to all of the 23S ribosomal RNA in Escherichia coli but specific to the Enterobacteriaceae family. One of the primers was the same primer used in the prior reverse transcription reaction. Real time qPCR of the qPCR reactions was performed on the BioRad CFX96 platform according to manufacturer's instructions. The real time qPCR protocol comprised 45 cycles of 30 seconds of denaturing at 95° C. and 60 seconds of annealing and extension at 60° C.

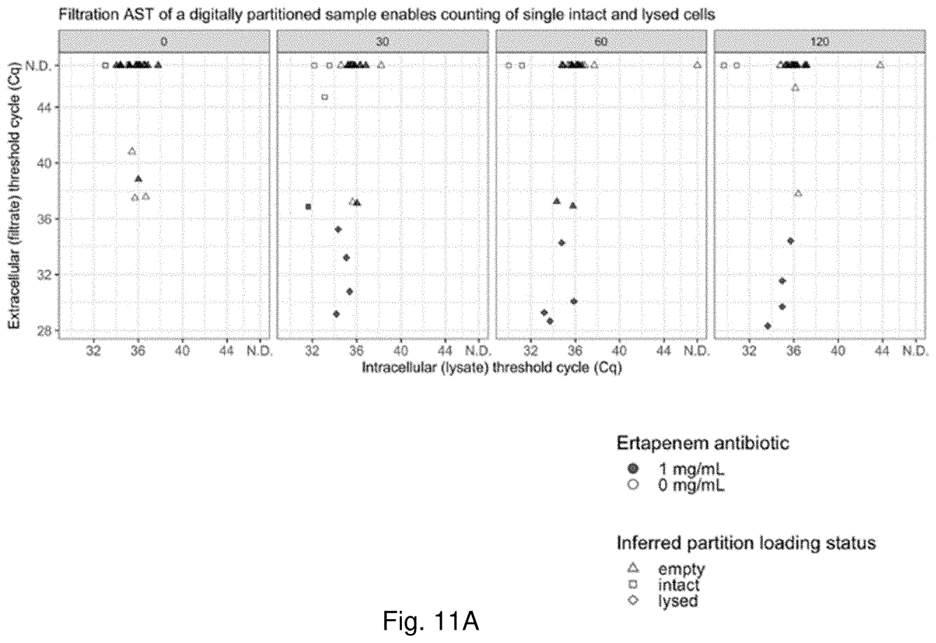

The output of the qPCR run was the threshold cycles, which reflect nucleic acid concentration, of the filtrate and in the lysate of both antibiotic exposures. The outputted threshold cycles are plotted in FIG. 9 which shows extracellular threshold cycles (Cq) and intracellular threshold cycles (Cq) for samples having a cell density of 0, 0.5, 1, and 2

Alternative nucleic acid quantification methods could have been employed, including digital droplet PCR and all of the methods for nucleic acid quantification enumerated earlier in this document.

11. Detection of the Extracellular Nucleic Acid Concentration Value (ENACV) and Intracellular Nucleic Acid Concentration Value (INACV) for Each Partition

From the 96 pairs of filtrate and lysate nucleic acid concentrations measured, the loading status of the 96 antibiotic exposures were estimated using a well loading status algorithm. The well loading status algorithm used was K-medoids clustering with 4 clusters. The cluster with the highest ENACV was determined to represent wells with killed cells. The cluster with the highest INACV was determined to represent wells with intact cells. The remaining clusters were called as empty wells.

The number of wells in each of the experimental conditions (treated and reference) possessing each loading status were counted. The results are found in the Table 4 below, as well as being shown in FIG. 9.

TABLE 4
ENACV and INACV of treated and reference partitions

| Batch culture | | | Extracellular | Intracellular | | |
|---|---|---|---|---|---|---|
| dilution | | Empty | only | only | Both | TOTAL |
| Treated (1.0 μg ETP/mL) | | | | | | |
| 0 | cells | 8 | 0 | 0 | 0 | 8 |
| 0.09375 | cells | 10 | 2 | 0 | 0 | 12 |
| 0.1875 | cells | 13 | 3 | 0 | 0 | 16 |
| 0.375 | cells | 2 | 10 | 0 | 0 | 12 |
| Reference (0.0 μg ETP/mL) | | | | | | |
| 0 | cells | 8 | 0 | 0 | 0 | 8 |
| 0.09375 | cells | 11 | 0 | 1 | 0 | 12 |
| 0.1875 | cells | 13 | 0 | 3 | 0 | 16 |
| 0.375 | cells | 7 | 0 | 5 | 0 | 12 |

No zero-cell reference condition data was used in this algorithm because the experiment already included zero-cell controls, and the algorithm performed adequately without such data.

Alternative choices for the well loading status algorithm can be used. A non-exhaustive list of appropriate choices was indicated in other sections of the present disclosure as will be understood by a skilled person.

12. Determination of Live and Death Cells Based on the ENACV and INACV

The next step of the analysis involves estimating the number of killed and intact cells from the tallies of samples in each loading status. The ratios of empty samples to total samples for each of the dilutions were 16/16, 21/24, 26/32, and 9/24, in order of increasing target density. The most likely densities estimated by the equation

$$Density = - ln ( \frac{\# empty}{\# total} )$$

were 0, 0.1335, 0.2076, and 0.9808 cells per sample. The expected number of cells in all samples of a given density is given by Density*(#total samples). Meanwhile, the probability of a sample (prior to any observation of that sample's nucleic acids) being loaded with X cells, given that the density is “D”, follows the Poisson distribution parameterized by D and is found by

$$Poisson ( X ; D ) = \frac{{D}^{X} {e}^{- D}}{X !} . \qquad ( 18 )$$

The probability of a sample being loaded with X cells, given that the density is “D”, and given that the sample is observed to be non-empty, is calculated (using the definition of a conditional probability) to be

$$\frac{Poisson ( X ; D )}{1 - Poisson ( 0 ; D )} = \frac{\frac{{D}^{X} {e}^{- D}}{X !}}{1 - \frac{{D}^{0} {e}^{- D}}{0 !}} = \frac{{D}^{X} {e}^{- D}}{X ! ( 1 - {e}^{- D} )} . \qquad ( 19 )$$

Given this model of cell loading, the expected number of cells in any group of N non-empty samples is equal to

$$\frac{ND}{1 - {e}^{- D}} .$$

For any group or IN empty samples, the expected number of cells is equal to 0. (Side note, one can also calculate that each non-empty sample in the densest batch culture dilution has a

$$\frac{1 - Poisson ( 0 ; D ) - Poisson ( 1 ; D )}{1 - Poisson ( 0 ; D )} = 0.411$$

chance of containing >1 cell.) Using the last two equations, one can calculate the expected number of killed and intact cells in each of the conditions as shown in the table below.

In the present set of experiments, due to the apparently complete killing of cells by antibiotic over the chosen exposure duration, no samples were observed to contain both extracellular and intracellular nucleic acids, and so it is irrelevant how one assumes the number of cells in such samples to be allocated.

As an example, the expected number of killed cells in the treated samples from the “0.375 cell/sample” batch culture dilution is

$$\frac{10 * 0.9808}{1 - exp ( - 0.9808 )} = 15.69$$

because there were 10 samples observed to contain only extracellular nucleic acids, and the density of this batch culture was observed to be 0.9808 cells/sample (a bit higher from the target of 0.375 cells/sample). The expected number of intact cells is 0 because no samples were observed to have intracellular nucleic acids, and it is expected that there are 0 cells in the empty wells.
The results are of the determination are reported in Table 5 below

TABLE 5
live and dead determination for the partitions based on cell number

| | | |
|---|---|---|
| Batch culture dilution | Intact | Killed |

Treated (1.0 μg ETP/mL)

| | | | |
|---|---|---|---|
| 0 | cells | 0 | 0 |
| 0.09375 | cells | 0 | 2.14 |
| 0.1875 | cells | 0 | 3.32 |
| 0.375 | cells | 0 | 15.69 |

Reference (0.0 μg ETP/mL)

| | | | |
|---|---|---|---|
| 0 | cells | 0 | 0 |
| 0.09375 | cells | 1.07 | 0 |
| 0.1875 | cells | 3.32 | 0 |
| 0.375 | cells | 7.85 | 0 |

The above analysis assumes that the number of cells present is due only to loading of the samples and not due to further changes of bacterial populations within each sample. Indeed, the pattern of empty vs non-empty samples in a digital AST is accurately modeled (where modeling is the interpreting of results given a set of assumptions) as a function of sample loading only, but the expected number of cells in those samples after minutes of incubation have passed is more accurately modeled when one predicts the population within each sample over time (a.k.a. population dynamics).

It is expected that live cells will continue to divide to create more cells and synthesize more nucleic acids, while cells that are killed stop producing more cells and stop synthesizing nucleic acids. The population dynamics of a single sample can be modeled by any of the population models used in the biology literature for bacteria, cells, and living organisms in general. Example population models include ordinary differential equations such as but not limited to the exponential growth equation, the logistic growth equation, and the Gompertz equation, and any variation of these models as will be known to the skilled practitioner. Other example population models may use branching stochastic processes and stochastic differential equations, such as Galton-Watson processes, multi-type Galton-Watson processes, continuous time Markov chain processes (simulated using the Gillespie algorithm), the Bellman-Harris process, and any variation of these models as will be known to the skilled practitioner.

The estimation of the four actual cell densities of the batch culture dilutions could be refined through use of a Bayesian model. Stochasticity introduces some imprecision when estimating the actual cell densities from the fraction of samples that were empty. A Bayesian model would enable one to incorporate (e.g. as a prior probability) the information that the batch culture dilutions' densities were known multiples of each other together with the observed fraction of samples that were empty, potentially yielding more accurate estimations.

13. Determination of Susceptibility Using an Extracellular/Intracellular Nucleic Acid Proportion Value (EINAPV) for Each Test Condition and Reference Condition

The next step in the analysis involves calculating an extracellular/intracellular nucleic acid proportion value (EINAPV) for each experimental condition. For example, the fraction extracellular,

$$\frac{\# killed}{\# intact + \# killed} ,$$

can serve as the EINAPV in this experiment.

Finally, the statistical significance of treated EINAPV was assessed. Specifically, the treated EINAPV was compared to the null hypothesis that its value could have arisen from the reference condition distribution due to chance alone. There are several comparable ways to test statistical significance, each making different assumptions and yielding similar results.

In a first possible route of analysis, one can treat the results of this experiment as a digital same-sample AST with 1 treated condition and 1 untreated reference condition by ignoring any possible effect of inoculating density (e.g. an inoculum effect), considering all 48 treated samples as part of one treated condition, and considering all 48 untreated references samples as part of one reference condition.

It is possible to group the different batch culture dilutions because they are all of the same strain, because the exposure duration was the same for all conditions, and because the number of cells in each digitally-loaded non-empty sample in this experiment is likely the same or within 2-fold of each other with high probability, suggesting that inoculum effects cannot be important.

Because a digital same-sample AST with one treated condition and one concurrent reference condition was performed and yielded integer counts of killed and intact cells, it becomes possible to apply several statistical tests defined for 2×2 contingency tables. For clarity, these statistical test do not use the treated or reference EINAPV, but instead directly use the two ENACVs and two INACVs used to calculate the EINAPVs.

The most reasonable model is a binomial test. In the binomial exact test, it is assumed that every cell has an identical chance of lysing, l, during the exposure. The most likely value for l, called \hat{l}, is the observed ratio of lysed vs total cells for all cells assumed to share the same value of l. In other words,

\hat{l}=(x_{RE}+x_{TE})÷(x_{RI}+x_{RE}+x_{TI}+x_{TE}), (20)

where x_{TE} is the number of lysed treated cells, x_{TI} is the number of intact treated cells, x_{RE} is the number of lysed untreated cells, and x_{RI} is the number of intact untreated cells. In this experiment, we estimate that 2.14+3.32+15.69=21.25 cells lysed, which we round to 21 lysis events, while 1.07+3.32+7.85=12.24 cells did not lyse, which we round to 12 no-lysis events.

There are 21+12=33 events in total in the most likely situation, and so \hat{l}=21/(21+12)≈0.64. (In the average situation, we would not round the number of cells, and \hat{l}=21.25/(21.2 5+12.24)≈0.63.) The one-sided p-value of the binomial exact test for one set of samples is found by the equation

$$Binomial Probability ( X \leq {x}_{RE} ; n = {x}_{RI} + {x}_{RE} , p = \hat{l} ) * Binomial Probability ( X \geq {x}_{TE} ; n = {x}_{TI} + {x}_{TE} , p = \hat{l} ) = {\sum }_{X = 0}^{{x}_{RE}} [ ( \begin{matrix}{x}_{RI} + {x}_{RE} \\ X\end{matrix} ) {( \hat{l} )}^{X} {( 1 - \hat{l} )}^{{x}_{RI} + {x}_{RE} - X} ] [ 1 - {\sum }_{X = 0}^{{x}_{TE} - 1} [ ( \begin{matrix}{x}_{TI} + {x}_{TE} \\ X\end{matrix} ) {( \hat{l} )}^{X} {( 1 - \hat{l} )}^{{x}_{TI} + {x}_{TE} - X} ] ] = ( \begin{matrix}12 \\ 0\end{matrix} ) {( 0.64 )}^{0} {( 1 - 0.64 )}^{12} ( \begin{matrix}21 \\ 21\end{matrix} ) {( 0.64 )}^{21} {( 1 - 0.64 )}^{0} = 4.03 \times {10}^{- 10} . \qquad ( 21 )$$

This p-value is less than a reasonable significance threshold of 0.001, so the strain is susceptible. Any other choice than 21/33 for the probability of lysis per event would have resulted in a smaller p-value.

Alternatively, other statistical hypothesis testing methods for contingency tables could be used, including Fisher's exact test, Barnard's test, Boschloo's test, Berger's test, Pearson's chi-square test, the G-test, McNemar's test, the Wald statistic, the Kolmogorov-Smirnov test, simple and multiple logistic regression models, and simple and multiple probit regression models.

In a second possible route of analysis, one could view this experiment as a digital same-sample AST with 4 treated conditions and 4 untreated reference conditions. The 0-cell conditions are removed from further analysis because of the lack of non-empty samples. The 3 remaining untreated condition EINAPVs have values of

$$\begin{matrix}\frac{\# killed}{\# intact + \# killed} = \frac{0}{0 + 1.07} = 0 , & ( 22 ) \\ \frac{\# killed}{\# intact + \# killed} = \frac{0}{0 + 3.32} = 0 , and & ( 23 ) \\ \frac{\# killed}{\# intact + \# killed} = \frac{0}{0 + 7.85} = 0 , & ( 24 )\end{matrix}$$

and they are used as a proxy for the reference condition distribution. Meanwhile, the 3 treated condition EINAPVs are found to have values of 1, 1, and 1. Applying an unpaired or paired t-test to these two sets of 3 numbers all yield significant p-values below 0.001. Applying a Z-test to the data also yields significant p-values below 0.001. Equivalent to a Z-test, one can calculate a threshold value from the untreated reference condition EINAPVs as the mean+3 standard deviations, or 0+3*0=0; then more than 95% (where 95% serves as a 0.05 significance threshold) of the three treated values lie above this threshold.

In a third possible route of analysis, one can treat the results of this experiment as a digital same-sample AST with 1 treated condition and 1 untreated reference condition by ignoring any possible effect of inoculating density (e.g., an inoculum effect), considering all 48 treated samples as part of one treated condition, and considering all 48 untreated references samples as part of one reference condition.

It is possible to group the different batch culture dilutions because they are all of the same strain, because the exposure duration was the same for all conditions, and because the number of cells in each digitally-loaded non-empty sample in this experiment is likely the same or within 2-fold of each other with high probability, suggesting that inoculum effects cannot be important. The one treated EINAPV is equal to

$$\begin{matrix}\frac{\# killed}{\# intact + \# killed} = \frac{( 0 + 2.14 + 3.32 + 15.69 )}{( 0 + 0 + 0 + 0 ) + ( 0 + 2.14 + 3.32 + 15.69 )} = 1 , & ( 25 ) \\ and the one untreated reference EINAPV is equal to & \\ \frac{\# killed}{\# intact + \# killed} = \frac{( 0 + 0 + 0 + 0 )}{( 0 + 0 + 0 + 0 ) + ( 0 + 1.07 + 3.32 + 7.85 )} = 0. & ( 26 )\end{matrix}$$

We can compare these two numbers to each other using the following algorithm.

Obtain additional “non-concurrent” reference EINAPVs. One can gather the non-concurrent reference EINAPVs from existing experiments or from new experiments one performs. It is preferred but not necessary to attempt to obtain non-concurrent reference EINAPVs; one could at any time skipped to the 4^{th} step of this algorithm where an a priori threshold value (APTV) is used without the use of non-concurrent reference EINAPVs.

The non-concurrent reference data comprise the EINAPVs obtained when a same-sample AST protocol, preferably this same protocol, is performed up to this step on samples known to contain the same or closely related species of microorganism (such as positive clinical specimens, or less preferably contrived clinical specimens spiked with the microorganism) and in which the microorganisms were not contacted with the antibiotic (contacted only with the vehicle of the antibiotic (e.g. pure water) and not the antibiotic compound itself, or less preferably where no contacting is performed). It is desired to include microorganisms in the prior reference condition data that are as similar to the currently tested microorganism as possible, with a tradeoff occurring between a larger number of prior reference condition data and the similarity of the microorganism in the included prior data.

Use a statistical test such as the Z-test to compare the non-concurrent reference EINAPVs to the one concurrent reference EINAPV and calculate the likelihood of the one concurrent reference EINAPV arising from the distribution of non-concurrent reference EINAPVs. If the test shows that the likelihood is higher than a chosen significance threshold, then the non-concurrent reference condition data is a good approximation of the true reference condition distribution, and one can then perform a second Z-test to compare the treated EINAPV and the non-concurrent reference EINAPVs to determine susceptibility.

As a weaker but similar alternative, one can also perform a statistical test to compare a concurrent treated-reference proportion quantity (TRPQ) to the distribution of non-concurrent reference treated-reference proportion quantities to determine susceptibility. The concurrent TRPQ is a function, such as the relative difference or the ratio, of the concurrent treated EINAPV and the concurrent untreated reference EINAPV. The non-concurrent reference TRPQ is formed by repeated application of the function to two reference EINAPVs, one acting as a treated EINAPV even though it is an untreated EINAPV.

The significance threshold is an arbitrary value chosen by practitioners to meet their specific needs, as will be understood by a skilled person. The choice of threshold involves a trade-off between the assay's diagnostic sensitivity and specificity. Thresholds of 0.05 or lower are commonly used in the literature.

If the one concurrent reference EINAPV is significantly unlikely to have arisen from the non-concurrent reference EINAPVs, then more relevant non-concurrent reference EINAPVs are obtained and the algorithm repeated.

If non-concurrent EINAPV data from experiments more similar to the current assay cannot be obtained, then one chooses an a priori threshold value (APTV) and then compare the concurrent TRPQ to this APTV. The concurrent TRPQ is a function, such as the relative difference or the ratio, of the concurrent treated EINAPV and the concurrent untreated reference EINAPV. The APTV is a value that corresponds to a certain false positive rate that results from a given guessed cell density, under the assumption that unequal random partitioning of cells during sampling of the clinical specimen is the only cause for any reference extracellular NACV being higher than the treated extracellular NACV.

The APTV can range from 1 to infinity and reflects one's beliefs prior to the experiment or the collection of non-concurrent reference data, which 1 being the most generous possible threshold that is useful, and APTVs between 1 and 8 being preferred. If one makes the assumption that unequal random partitioning of cells during sampling of the clinical specimen is the only cause for any reference extracellular NACV being higher than the treated extracellular NACV, and that one knows the cell density of the specimen and the volumes of the treated and reference samples, then the number of cells in each sample will be multinomially distributed with probability parameters equal to the proportions of the sample volumes to the total specimen volume.

Any choice of the APTV will then correspond to a certain guess of the cell density and to a certain percentile of false positive cases that would result given the guessed cell density. For example, for two untreated samples X and Y, each 10 μL, taken from a 1000 μL specimen with 4000 CFU/mL, the chance that the number of cells in sample X is 2 or more times that of sample Y (or vice versa) was calculated to be about 0.002754 using the statistical software R. Therefore, if an assay's treated-reference ratio is greater than or equal to an APTV of 2.0, then there is only a 0.275% chance of a “Susceptible” call being incorrect. If the specimen density were actually lower than 4000 CFU/mL, and the chosen APTV remains at 2.0, then the chance of an incorrect “Susceptible” call increases.

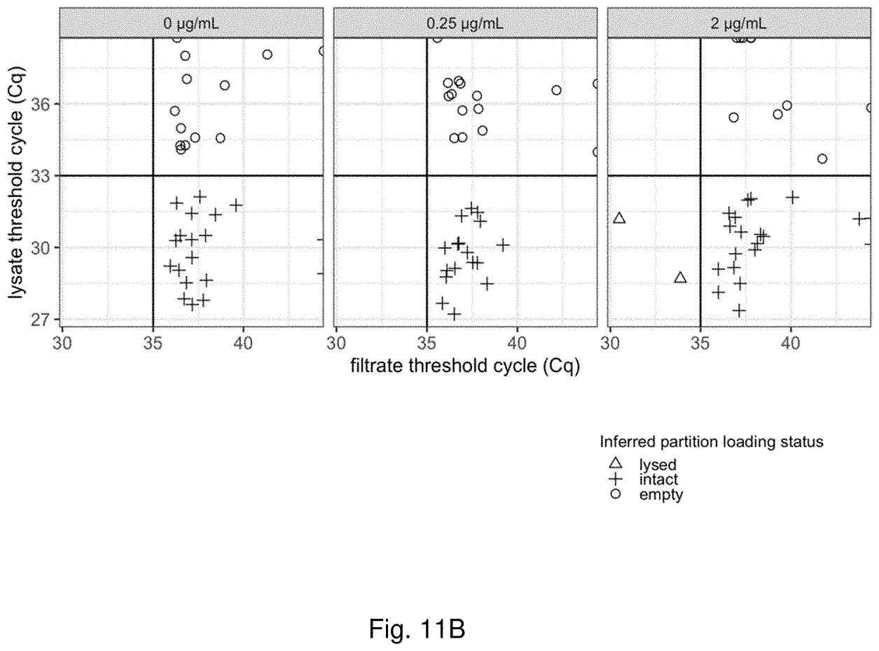

A graph of the false positive rate (where a “susceptible” call is considered positive) as a function of APTV and three cell densities is shown in FIG. 10 The choice of the APTV value is necessarily subjective, reflects a trade-off between assay diagnostic sensitivity and diagnostic specificity, and is chosen by the user to fit their specific clinical needs.

In this experiment, there was an intended 0.09375 cells/sample in the most dilute batch culture dilution. We assume that all 96 samples were loaded at this cell density (a situation true when volume of the batch culture dilution is high and the batch culture dilution contains a large number of cells approaching infinity). With an APTV of 3.0, the chance of a false positive due to differential loading of cells is. With an APTV of 4.6, the chance of a false positive

In actuality, 56 of the samples were loaded with a higher cell density than 0.09375 cells/sample. We would expect the variation due to stochastic loading to be smaller than if the 0.09375 cells/sample density were used to load all 96 samples, so our chosen APTV is overly conservative. Nonetheless, since our measured TRPQ is higher than the APTV, we can confidently predict that the E. coli strain tested is susceptible.

In a fourth possible route of analysis, Bayesian statistical models of varying complexity could also be defined and applied to the data. For some of these tests to apply, one may need data from prior runs that replicate this experiment. These data could be obtained in prior repetitions of this protocol, or in repetitions of this protocol performed at the same time (e.g. in a high throughput set up).

### Example 8: Digital Same-Sample Filtration AST with Multiple Non-Replicate Treated Conditions and Multiple Non-Replicate Concurrent Reference Conditions

In the experiment described in this example, there were two possible goals, each with their own interpretation of the same results.

One purpose was to measure the lag time in antibiotic killing. This goal instead provides data to which models of bacteria population dynamics that be fitted. This goal is not a question that clinicians using the assay of the present disclosure will necessarily pursue, but if a clinician decides that a parameter of bacteria population dynamics is to be used to determine susceptibility, then in further examples a preferred method is demonstrated for measuring population dynamics that has a lower limit of detection for the same number of cells analyzed. An assay with a lower limit of detection can be called more efficient, sensitive, or informative than one with a higher limit of detection.

The other purpose of the set of experiments of the present example was to confirm the susceptibility of the bacteria strain Escherichia coli K12 to demonstrate our assay's validity, even though we already knew Escherichia coli K12 is susceptible. This latter goal mimics the questions pursued by future users of this invention.

The exemplary digital multiplex same-sample AST protocol used in this set of experiments provided herein below in an outline describing the various sets of operations comprised in the protocol.

1. Providing a Sample

For the purposes of demonstration, a contrived clinical sample was created by inoculating Escherichia coli K12 into Brain-Heart Infusion broth. The inoculum was small enough that no detectable difference in the sample's optical density at 600 mm (OD₆₀₀) was detectable by a spectrophotometer with a sensitivity of 0.01 absorbance units. After an incubation at 37° C., the media became turbid with an OD₆₀₀ of 0.34 absorbance units after 3.08 hours of incubation.

The above sample was provided with a known susceptible bacterial strain to provide a proof of principle. Samples can be provided from specimen to perform testing for microorganisms whose susceptibility/resistance to the antibiotic is unknown without inoculation modifying the above procedure in a manner identifiable by a skilled person upon reading of the present disclosure

2. Sample Partitioning Planning

In this experiment, the number and volume of sample partitions was restricted, for logistical reasons, to 96 partitions 10 μL in volume, specifically the wells of a 96-well plate. A goal was chosen of having a >98% chance of ending up with at least 48 empty partitions. When all partitions are the same volume, the following formula relates the expected number of empty partitions, the partition volume, and the density of cells.

$${\sum }_{k = n}^{N} ( \begin{matrix}N \\ k\end{matrix} ) {{e}^{- DVk} ( 1 - {e}^{- DV} )}^{N - k} > t \qquad ( 27 )$$

N is the total number of partitions, n is the number of empty partitions, V is the partition volume, D is the density of cells, and t is a threshold probability chosen by the practitioner. In order to achieve digital sample partitioning with the available 96-well plate, each 10 μL sample of the specimen needed to contain between 0.02 and 2.5 cells on average. In this experiment, a density of 1 cells per sample were targeted. Due to systematic bias in the conversion of OD600 to cell density, the actual density achieved in the exposure was closer to 0.281 cells per sample. The batch culture specimen itself was diluted to a density of 28.1 CFU/mL (0.0281 cells/μL) to achieve the target cells/sample density.

Although the density of bacterial cells in a typical clinical specimen is not known precisely, in clinical scenarios a plausible range of densities is known, and so the partition number and volumes can always be chosen so that it is highly likely for a desired number of partitions to not receive any bacterial cells, with random chance being the reason different partitions differ in the number of cells loaded. Clinical specimens with high densities of cells can also be diluted to increase the maximum allowed volume of the partitions or decrease the minimum required number of partitions. It is also possible for devices to prepare partitions of varying volumes so that a wider range of cell loading densities falls within the digital range for some of the prepared partitions [21].

3. Contacting the Sample Partitions with Antibiotic (Test Conditions) or Culture Media (Reference Conditions) for Different Exposure Times

To begin the AST protocol, the contrived clinical specimen (containing 28.1 CFU/mL) was physically partitioned into the 96 samples by transferring 10 μL of the specimen, in 96 separate transfers (actually 8 transfers with a multichannel pipette), to each well of a separable 96-well microtiter plate. Then, the entire plate was sealed with a RNase/DNase-free plastic, adhesive sealing membrane.

Each well of the 96-well microtiter plate contained 15 μL of Mueller-Hinton Broth (MHB) growth media, placed there before the specimen was partitioned. Half of the wells (48) contained 0 μg/mL of dissolved ETP antibiotic and served as reference condition antibiotic exposures. The other half of the wells contained 1.67 μg/mL of ETP (for a final concentration of 1.0 μg/mL) and served as 48 treated condition antibiotic exposures. The separable plate comprised 4 detachable sections bearing 3 columns and 8 rows of wells.

The treated and reference conditions were arranged so that each of the four groups of wells contained 12 treated and 12 reference conditions. In total, there would eventually be eight experimental conditions: 4 exposure durations of 0, 30, 60, and 120 min, each with 12 treated and 12 reference conditions.

Immediately after the specimen had been partitioned, one section (24 wells in total) of the separable 96-well plate was detached to serve as the 0-minute set of conditions. The rest of the separable 96-well plate was incubated at 37° C. Then, the following step was repeated for each time point of 0, 30, 60, and 120 minutes.

4. Sample Separation for Each Partitions by Filtration and Centrifugation

The entire volume of each antibiotic exposure was transferred to a Millipore® 96-well sterile polystyrene MultiScreenHTS® filter plate (Millipore-Sigma MSGVS2210). Each well of the filter plate contained a hydrophilic polyvinylidene fluoride (PVDF) filter membrane with a 0.22 μm pore size. A 96-well polypropylene microtiter plate was affixed to the bottom of the filter plate. The filter plate was promptly centrifuged at 2200 relative centrifugal force for 3 minutes to speed the passage of the antibiotic exposure sample through the filter and into 96-well microtiter plate. The collected fluid was called the “filtrate.”

It is expected that the filtrate will contain all or most of the extracellular nucleic acids present in the antibiotic exposure, but none of the intracellular nucleic acids in the antibiotic exposure. The filtrates were transferred to new containers and then frozen at −80° C. to prevent hypothetical rRNA degradation. Lucigen DNA Extraction Buffer was not used to “extract” the filtrate DNA. The use of an extraction buffer would have diluted the filtrate nucleic acids. If the reverse transcription reaction were to be performed immediately after the filtrates were created, then freezing would be unnecessary.

The filter pore size was chosen to prevent the passage of intact bacterial cells, which are all larger than 0.22 with rare exceptions.

The centrifugation speed was chosen to be low enough to prevent cell lysis, as would be understood by a skilled person upon reading of the present disclosure.

Any exposure duration, with a reasonable range being up to 24 hours, could have been chosen instead the exposure durations actually chosen. Because this AST experiment was performed manually, the time between first antibiotic exposure and the separation of extracellular and intracellular nucleic acids by filtration did not fall exactly on the target time points of 0, 30, 60, and 120 minutes. In this experiment, a stopwatch was used to record the time of every action taken by the experiment operator starting with the dilution of the batch culture/contrived clinical specimen. The use of a stopwatch helped minimize uncertainty in the length of the exposure duration. For example, the 0-minute time point actually represented exposures between 8.45 to 8.72 minutes. For clinical applications, the susceptibility can still be correctly called even though manual operation may introduce uncertainty into the control of the environment experienced by the bacterial cells during the protocol, so long as the uncertainty is not comparable in magnitude to the corresponding aspect of the protocol.

5. Filter Washing

The washing of the filter membranes, performed in the next step, could instead be performed at each time point immediately after separation by filtration. It would be preferred to wash the filters as soon as possible after the initial separation by filtration. In actuality, in this experiment, the washing of the filters was delayed until all the samples had been filtered. Thus, for the 0-min samples, about 120 minutes elapsed between the filtration and the washing.

After all 96 samples had been filtered at the target time point, a new 96-well plate is affixed to the bottom of the filter plate, and 25 μL of fresh MHB media was centrifuged (2200 rcf, 3 min) through the filters after the first centrifugation (above) to wash away residual extracellular nucleic acids present in the small amount (about 3 μL) of fluid wetting the filters. This wash fluid was collected separately and not analyzed, it could have been analyzed by nucleic acid quantification or it could be combined with the filtrate (with the downside of diluting the filtrate signal).

Any type of fluid that does not lyse or degrade cells may be passed through the filter. Examples include other growth medias, phosphate buffered saline, and other buffered solutions of salt compounds found physiologically inside of the bacteria.

Any volume of wash fluid that covers the entire filter membrane could have been used, namely from about 15 μL up to 250 μL. 250 μL is the maximum capacity of the MultiScreenHTS filter plate's wells.

Solutions that are hypoosmotic to the cell interior, such as pure water, increase the osmotic pressure across the cell wall and will lyse cells without rigid cell walls. Bacteria have rigid cell walls and some are adapted to survive sudden increases in osmotic pressure. Bacteria whose cell walls are damaged by antibiotic but have not yet lysed may be induced to lyse by sudden exposure to a hypoosmotic solution. If the wash solution is collected, accurate susceptibility calling is possible by treated the wash solution as a second filtrate. If not, inaccuracy is introduced into the number of intact cells and the number of total cells in the sample.

5. Cell Lysis to Provide Extracted Lysate Comprising Intracellular Nucleic Acid

Next, 20 μL of DNA Extraction Buffer was placed into all of the wells of the filter plate, on top of the filters. The filter plate was heated to 65° C. for 6 minutes, without shaking, on a ThermoMixer® flat surface heating block. Then, the filter plate was taped to a clean 96-well polypropylene microtiter plate and centrifuged at 2200 rcf for 3 minutes. The DNA Extraction Buffer fluid that flowed through the filter was collected in the microtiter plate below the filter plate. Next, the microtiter plate was heated to 98° C. for 4 minutes inside a BioRad thermocycler. These collected and heated fluid volumes are termed the “extracted lysate”.

The purpose of this step is to recover the intracellular nucleic acids found in the intact cells retained on the filters. To do so, these intact cells are lysed and their nucleic acids extracted. The lysate is expected to contain all or most of the formerly intracellular, now extracellular nucleic acids.

Alternative ways to extract the intracellular nucleic acids can be performed. For example, the filter membrane can be removed from the filter apparatus using sterile and clean forceps and placed into a volume of DNA Extraction Buffer. This volume of buffer is vortexed vigorously, then heated to 65° C., then heated to 98° C.

As a third alternative, intact bacterial cells retained on the filter can be mechanically dislodged (e.g. centrifugation in the opposite direction, stirring), then transferred to a volume of DNA Extraction Buffer, which is then heated to 65° C. and then to 98° C.

Additional lytic reagents such as lysozyme can be added to the DNA Extraction Buffer to increase lysis efficiency.

The temperatures of 65° C. and 98° C. derive from the manufacturer's instructions for the Lucigen DNA Extraction Buffer kit.

6. Preservation of the Extracted Lysates by Freezing

In this experiment, the extracted lysates were frozen at −80° C. to pause the experiment. Freezing extracted nucleic acids is not necessary if one immediately continues to the next step in the protocol (the reverse transcription step).

7. Reverse Transcription of RNA in Filtrates and Lysates

Separately, for each of the 96 extracted filtrates and for each of the 96 extracted lysates, 2.10 μL of the extracted filtrate was mixed with 0.024 μL of 3 U/mL Lucigen® RapiDxFire thermostable reverse transcriptase, 0.300 μL of Lucigen® RapiDxFire 10× thermostable buffer, 0.150 μL of 10 mM deoxyribonucleic acid nucleotides, 0.306 μL of deionized water, and 0.120 μL of a 10 μM aqueous solution of DNA primer, according to manufacturer's instructions, to create a reverse transcription reaction with a total volume of 3.0 μL. The reagents except for the templates were first mixed together to form a (3.0-2.1)*192=172.8 μL master mix; they were not individually added to each of the 192 reverse transcription reactions. The DNA primer included had a sequence of 5′-TGTCTCCCGTGATAACTTTCTC-3′ (SEQ ID NO: 3). The primer's sequence was complementary to the 23S ribosomal RNA in Escherichia coli and specific to the Enterobacteriaceae family. The cDNA product that would be created from this primer contained the primer sites for the future ddPCR reaction occurring later in this AST protocol. All 192 reverse transcription reactions were heated to 60° C. for 10 minutes to create cDNAs, then heated to 95° C. for 5 minutes to stop the reaction and inactivate the reverse transcriptase enzyme.

A reverse transcription step is optional if one has decided to amplify a DNA molecule found naturally in the cells of interest. However, if the nucleic acid to be quantified in the AST protocol is a ribonucleic acid (RNA) molecule, and the quantification method operates only on deoxyribonucleic acid molecules, then both the filtrate and the lysate can be treated with a reverse transcriptase enzyme to produce complementary DNA molecules (cDNA) prior to nucleic acid quantification. The concentration of cDNA, and thus rRNA, is calculated from the counts of high and low fluorescence droplets.

Alternative reverse transcription enzymes, protocols, and kits may be used instead of the kit used in this example.

Alternative primers may be used. Alternative nucleic acid species can be targeted as well, through a choice of primers. As noted earlier in this document, targets with a higher copy number per cell are preferred for accessibility AST.

8. Quantification of Extracellular and Intracellular Nucleic Acid in Filtrates and Lysate of Each Partitions

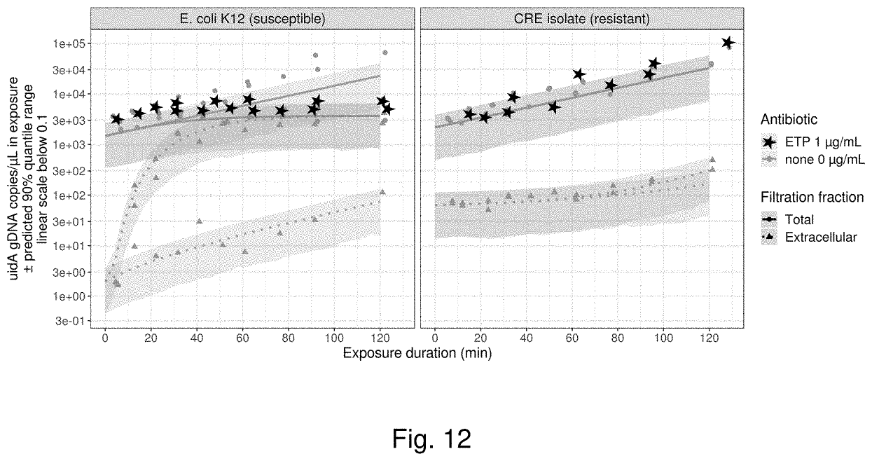

A 1 μL volume of each of the above reverse transcription reactions was separately added, according to kit instructions, to 3.0 μL of BioRad SsoFast qPCR EvaGreen 2× supermix, 1.76 μL nuclease-free water, and 0.24 μL of a pair of DNA PCR primers at 10 μM each, to create a 6 μL qPCR reaction. The pair of PCR primers possessed the following sequences: 5′-GGTAGAGCACTGTTTTGGCA-3′ (SEQ ID NO:2), 5′-TGTCTCCCGTGATAACTTTCTC-3′(SEQ ID NO: 3). The DNA primers' sequences flanked an 80 bp region common to all of the 23S ribosomal RNA in Escherichia coli but specific to the Enterobacteriaceae family. One of the primers was the same primer used in the prior reverse transcription reaction. Real time qPCR of the qPCR reactions was performed on the BioRad CFX96 platform according to manufacturer's instructions. The real time qPCR protocol comprised 46 cycles of 30 seconds of denaturing at 95° C. and 60 seconds of annealing and extension at 60° C. The output of the qPCR run was the threshold cycles, which reflect nucleic acid concentration, of the filtrate and in the lysate of both antibiotic exposures. The outputted threshold cycles are plotted in FIG. 11A which shows extracellular threshold cycles (Cq) and intracellular threshold cycles (Cq) for samples having an antibiotic exposure durations of 0, 30, 60, and 120 min.

Alternative nucleic acid quantification methods could have been employed, including digital droplet PCR and all of the methods for nucleic acid quantification enumerated earlier in this document.

9. Detection of the Extracellular Nucleic Acid Concentration Value (ENACV) and Intracellular Nucleic Acid Concentration Value (INACV) for Each Partition

From the 96 pairs of filtrate and lysate nucleic acid concentrations measured, the loading status of the 96 antibiotic exposures were estimated using a well loading status algorithm. The resulting tally of the number of wells in each of the experimental conditions (treated and reference) possessing each loading status are found in Table 6 below.

TABLE 6
ENACV and INACV of treated and reference partitions

| | | | | | |
|---|---|---|---|---|---|
| Exposure | | Extracellular | Intracellular | | |
| duration | Empty | only | only | Both | TOTAL |

Treated (1.0 μg ETP/mL)

| | | | | | | |
|---|---|---|---|---|---|---|
| 0 | min | 12 | 0 | 0 | 0 | 12 |
| 30 | min | 7 | 4 | 1 | 0 | 12 |
| 60 | min | 8 | 4 | 0 | 0 | 12 |
| 120 | min | 8 | 4 | 0 | 0 | 12 |

Reference (0.0 μg ETP/mL)

| | | | | | | |
|---|---|---|---|---|---|---|
| 0 | min | 11 | 0 | 1 | 0 | 12 |
| 30 | min | 9 | 0 | 3 | 0 | 12 |
| 60 | min | 10 | 0 | 2 | 0 | 12 |
| 120 | min | 10 | 0 | 2 | 0 | 12 |

The following “manual” well loading status algorithm was used: 1) for each of the filtrate and lysate fraction, calculate the PCR per cycle efficiency by a) finding all fluorescence measurements for any of the 96 wells between 20 and 200 absorbance units, b) calculating the ratios of fluorescence measurements for each pair of adjacent cycles from the same well of the same fraction, and then 3) taking the average of all the ratios from the same fraction; 2) calculate an amount of nucleic acid using the equation A=E^{-C}^{_{q}}, where E is the PCR per cycle efficiency for the relevant fraction, of each of the 192 Cq's; 3) plotting the amount of filtrate nucleic acid against the amount of lysate nucleic acids, as depicted in FIG. 11A; 4) deciding manually where there is a distinct break between the points plotted near the value of 35. The distinct break serves as the background Cq threshold, and one background Cq threshold each is drawn for the filtrate and for the lysate fractions. The background Cq thresholds were chosen to be close to 35 because a Cq of 35 is known to be the lowest limit of detection for most commercial qPCR kits and corresponds the Cq of non-specific primer amplification. The true background Cq threshold for a given qPCR reaction depends on PCR per cycle efficiency, and the PCR per cycle efficiency is influenced by undefined, potentially inhibitory substances in the template. In this experiment, background Cq thresholds of 33 Cq for the filtrate and 36 Cq for the lysate were chosen, partly based on this experiments data and partly because the same values were found by k-means clustering for a previous experiment with an essentially identical protocol.

The less subjective method of multivariate Gaussian mixture modeling with the Cq values (not the amounts values) was attempted but the software package used did not handle censored data, and the fit was unsuccessful. “Censored data” are data that are not specifically measured due to the dynamic range of one's measurement device, but which are known to reside in a certain interval of values. The censored data in this experiment comprised the Cqs from wells where there was no Cq measured due to lack of amplification within 46 cycles. The true Cq is known to be greater than 46 but is not specifically measured, making the data “censored”.

K-means clustering with k=3, 4, 5, 6, 7, and 8 was also attempted but failed due to the censored data in the filtrate data.

When the above automated algorithms failed to yield plausible results, additional non-concurrent zero-cell data could have been obtained from either existing or new experiments. This non-concurrent zero-cell data would comprise nucleic acid Cq values (from each fraction) measured from samples known to contain zero bacterial cells and processed by the identical protocol as above. The combination of this non-concurrent zero-cell data and the concurrent data would enable the use of supervised machine learning algorithms, and it would improve the performance of unsupervised machine learning algorithms. (The above Gaussian mixture modeling and K-means clustering algorithms are unsupervised algorithms.) Non-concurrent zero-cell data was not used in this experiment for brevity, because the practitioner found that the informed manual threshold selection performed adequately without such data.

If the addition of non-concurrent zero-cell data was omitted for practicality or was insufficient, and if one wishes to analyze data in a completely automated way, the algorithm could have simply rejected this qPCR measurement as flawed due to inhibition of the filtrate qPCR reaction. The remedy would be to run a larger qPCR reaction volume for the same volume of template, as any inhibitory compounds in the template is diluted relative to the qPCR reagents. However, a skilled person who is trained in statistics and understands the mechanism of qPCR or who has seen prior experiments would still be able to interpret the flawed data, as was done in this experiment.

Alternative choices for the well loading status algorithm can be used and may perform better than the algorithm used in this experiment. A non-exhaustive list of appropriate choices is described in a further section of the present disclosure and identifiable by a skilled person.

11. Determination of Susceptibility Using an Extracellular/Intracellular Nucleic Acid Proportion Value (EINAPV) for Test Condition and Reference Conditions

Finally, the susceptibility of the strain is called. There are several possible ways to perform statistical analysis that calls susceptibility. For bulk assays, the present disclosure describes how to calculate EINAPVs and TRPQs, possibly adjusted using population dynamics models, and compare them to a combination of concurrent reference values, non-concurrent reference values, and APTVs. For digitally-partitioned assays, the empty samples do not benefit the analysis of EINAPVs and TRPQs if treated as possibly non-empty. Instead, one analyzes the sample loading (the “sample loading” being the results of the well loading status algorithm) with or without consideration of the NACVs that were inputted into the well loading status algorithm. The sample loading, with a model of population dynamics that takes exposure duration into account (or which can be trivially simple and not model population growth over time), can be used to estimate the true number of lysed and intact cells, either as a distribution over the possible sample loadings (possibly as one component of a more comprehensive probabilistic model of the digital AST NACVs), or as a single most likely sample loading. From the estimated true numbers of intact and lysed cells, one can apply statistical tests to the cell counts to call susceptibility via null hypothesis testing, one can perform statistical inference other than null hypothesis testing such as Bayesian modeling, or one can further calculate derived quantities such as EINAPVs and TRPQs which can be compared to combinations of concurrent reference values, non-concurrent reference values, and APTVs as done with bulk assays. Bulk time-series ASTs are analyzed as separate bulk AST assays, possibly with a 0-minute time point serving as a proxy for accumulated antibiotic-independent extracellular nucleic acid, but with the additional step, whenever possible, of fitting the measured NACVs to population dynamics models used to calculate certain EINAPV functions, with or without non-concurrent treated and reference data. Digital time-series ASTs are analyzed as separate digital AST assays, possibly with a 0-minute time point serving as a proxy for accumulated antibiotic-independent extracellular nucleic acid, and with the additional step, whenever possible, of fitting the results of the well loading status algorithm to population dynamics models used to estimate the true number of lysed and intact cells, with or without non-concurrent treated and reference data.

In this experiment, susceptibility was called by performing statistical tests for integer data on an approximation of the most likely sample loadings. First, we estimate the number of killed and intact cells from the tallies of samples in each loading status. The most likely density per sample, for digitally-loaded samples of equal volume, can be estimated by the equation

$$Density = - ln ( \frac{\# empty samples}{\# total samples} ) . \qquad ( 28 )$$

Since there were 75 empty samples out of 96 total samples of equal volume, the most likely density is 0.247 cells per sample, or 24.7 CFU/mL in the diluted batch culture. This density is indeed close to the intended target of 0.281 cells per sample or 28.1 CFU/mL, namely 87.8% of the intended target value.

With 96 samples, the expected number of cells examined is 23.7 cells, implying that the most likely number of cells analyzed is 24, and that it is most likely (but by no means necessary) that 3 of the 21 non-empty samples contained more than one cells. It would be sufficient to use maximum likelihood estimation for the sample loadings to choose the most likely sample loading (an example of estimation of a multivariate discrete parameter), but all non-empty samples are equally likely to contain an additional cell, however, so there is no unique arrangement of sample loadings that maximizes the likelihood of observing 75 empty samples.

In this example, it was possible to elect to simplify the subsequent analysis by ignoring the possibility of more than one cell per sample. With this new assumption, the most likely sample loading for this experiment becomes the loading depicted in the following Table 7.

TABLE 7
Determination of live and death cells
based on the ENACV and INACV

| | | | |
|---|---|---|---|
| Exposure duration | Intact | Killed | Total |

Reference (0.0 μg ETP/mL)

| | | | | |
|---|---|---|---|---|
| 0 | min | 1 | 0 | 1 |
| 30 | min | 3 | 0 | 3 |
| 60 | min | 2 | 0 | 2 |
| 120 | min | 2 | 0 | 2 |

Treated (1.0 μg ETP/mL)

| | | | | |
|---|---|---|---|---|
| 0 | min | 0 | 0 | 0 |
| 30 | min | 1 | 4 | 5 |
| 60 | min | 0 | 4 | 4 |
| 120 | min | 0 | 4 | 4 |

A unique maximum likelihood sample loading would be possible to identify if bacteria population dynamics were assumed to occur, and that the concentration of nucleic acids in a sample is a function of exposure duration, susceptibility, and the starting number of cells. After correction for nucleic acid synthesis during the exposure and after assuming a certain susceptibility, the assumption is made that samples with a higher nucleic acid concentration in either the filtrate or the lysate were more likely to have contained more than one cell. The population dynamics of a single sample can be modeled by any of the population models used in the biology literature for bacteria, cells, and living organisms in general.

Example population models include ordinary differential equations such as but not limited to the exponential growth equation, the logistic growth equation, and the Gompertz equation, and any variation of these models as will be known to the skilled practitioner. Other example population models may use branching stochastic processes and stochastic differential equations, such as Galton-Watson processes, multi-type Galton-Watson processes, continuous time Markov chain processes (simulated using the Gillespie algorithm), the Bellman-Harris process, and any variation of these models as will be known to the skilled practitioner.

Unlike maximum likelihood estimation, a Bayesian probabilistic model that includes a prior distribution would be able to calculate the posterior probability of strain susceptibility marginalized over all possible sample loadings. A Bayesian model that includes bacterial population dynamics could interpret the nucleic acid concentrations of each sample instead of only the binary well loading status call.

Next, for each of the four sets of exposure durations, a binomial exact test was performed to test the hypothesis that each cell in the treated and the reference conditions had the same probability of lysing. A significance threshold of 0.005 was chosen a priori. In the binomial exact test, we assume that every cell has an identical chance of lysing, l, during the exposure. The most likely value for l, called \hat{l}, is the observed ratio of lysed vs total cells for all cells assumed to share the same value of l. In other words,

\hat{l}=(x_{RE}+x_{TE})÷(x_{RI}+x_{RE}+x_{TI}+x_{TE}), (29)

where x_{TE} is the number of lysed treated cells, x_{TI} is the number of intact treated cells, x_{RE} is the number of lysed untreated cells, and x_{RI} is the number of intact untreated cells. In this experiment, we assume that the value of l is different for each exposure duration, so the four values of \ddot{l} are (0+0)÷(1+0+0+0)=0, (0+4)÷(3+0+1+4)=0.5, (0+4)÷(2+0+0+4)=0.67, and (0+4)÷(2+0+0+4)=0.67, for the 0, 30, 60, and 120-min exposures respectively. In addition, we treat the 4 sets of samples as independent experiments, since our one AST assay with 4 non-replicate pairs of treated & reference conditions can be seen as four separate digital AST assays, each with 1 pair of treated & reference conditions. The one-sided p-value of the binomial exact test for one set of samples is found by the equation

$$Binomial Probability ( X \leq {x}_{RE} ; n = {x}_{RI} + {x}_{RE} , p = \hat{l} ) * Binomial Probability ( X \geq {x}_{TE} ; n = {x}_{TI} + {x}_{TE} , p = \hat{l} ) = {\sum }_{X = 0}^{{x}_{RE}} [ ( \begin{matrix}{x}_{RI} + {x}_{RE} \\ X\end{matrix} ) {( \hat{l} )}^{X} {( 1 - \hat{l} )}^{{x}_{RI} + {x}_{RE} - X} ] [ 1 - {\sum }_{X = 0}^{{x}_{TE} - 1} [ ( \begin{matrix}{x}_{TI} + {x}_{TE} \\ X\end{matrix} ) {( \hat{l} )}^{X} {( 1 - \hat{l} )}^{{x}_{TI} + {x}_{TE} - X} ] ] . \qquad ( 30 )$$

The four p-values, in order of increasing exposure duration, were found to be 1.0, 0.023, 0.022, and 0.022. In the assumed model, since all four sets of samples were independent experiments, and because our binomial p-values are all true probabilities, the overall p-value for the test is the product of the four calculated p-values. This overall p-value is 0.000011, which is less than our significance threshold of 0.005. Thus, the strain is correctly called as susceptible.

Alternatively, other statistical hypothesis testing methods for contingency tables could be used, including Fisher's exact test, Barnard's test, Boschloo's test, Berger's test, Pearson's chi-square test with or without Yate's continuity correction, the G-test, McNemar's test, the Wald statistic, the Kolmogorov-Smirnov test, simple and multiple logistic regression models, and simple and multiple probit regression models.

Other routes of analysis were possible, as mentioned in step 12. For example, many simple population dynamics imply that the chance of lysis increases over the exposure durations examined in this experiment, but this information was not used to constrain the four values of calculated above.

Furthermore, one could have further calculated the treated and untreated EINAPVs of each of the four time points, and then, from those eight EINAPVs, calculate four TRPQs. These eight EINPAVs or four TRPQs could be compared to population dynamic models to obtain a p-value (a form of regression). They could also be compared, without the use of population dynamics, to combinations of concurrent reference values, non-concurrent reference values, and APTVs, as done with bulk assays.

### Example 9: Same-Sample Filtration AST with One Treated Condition and No Concurrent Reference Conditions

A clinical specimen comprising bodily fluid, processed bodily fluids is obtained using standard collection techniques. For example, human cells in the specimen may be lysed by saponin treatment, and growth medium added to the sample to keep cells viable during transport; the specimen may also be briefly incubated with growth media; or the microorganisms in the cells can be enriched by mechanical, chemical, or electrical apparatuses. Alternatively, the clinical specimen may comprise a pure culture of microorganisms obtained from a clinical specimen using standard isolation techniques.

One sample of the clinical specimen is taken and contacted with an antibiotic of interest (ABX) to create a treated antibiotic exposure condition. The antibiotic and sample of the clinical specimen are incubated together for a desired duration of time (the “exposure duration”).

The concentration of the antibiotic is chosen according to the desires of clinicians, with any one of the relevant CLSI breakpoint concentrations being the preferred choice.

A minimal exposure duration or an exposure duration that maximizes assay confidence can be found by the prior compiling of assay results from a sampling of pathogenic microorganisms, or a rough approximation such as for 30 minutes or 60 minute can be employed.

For an AST to be useful with only one treated condition and species-specific primers for amplification, it is assumed at this point that the microorganism has already been identified using standard identification assays so that the correct primers are used. Otherwise, approaches using universal primers, multiplexed primers, high-resolution melting curves, or sequencing could be used.

The one treated antibiotic exposure condition is subjected to a physical separation, such as filtration or centrifugation, and both the extracellular and intracellular nucleic acid fractions are separately collected and extracted in a way that preserves information about the in situ extracellular and intracellular nucleic acid concentrations in the antibiotic exposure. Suitable extractions are identifiable by a skilled person upon reading of the present disclosure.

Nucleic acid amplification (with or without prior reverse transcription) is used to quantify both the extracellular and intracellular nucleic acid fractions, yielding one treated extracellular nucleic acid concentration value (ENACV) and one treated intracellular nucleic acid concentration value (INACV). The decision to include reverse transcription prior to nucleic acid amplification is described a further section of the present disclosure. Alternative methods for obtaining nucleic acid concentration values are described herein and would be identifiable by a skilled person upon reading of the present disclosure.

The treated ENACV and the treated INACV are entered into the well loading status algorithm. If the one sample (which is also the one antibiotic exposure condition present) is found to be empty, then the assay is inconclusive as no microorganisms of interest were present in the clinical specimen. Either the assay is repeated with a new clinical specimen, or the lack of infection is suspected. If the sample is found to be not empty, then proceed to the next step of analysis.

Since there is only one treated condition and no concurrent reference conditions, the following algorithm is a suitable choice for the well loading status algorithm. Prior reference condition data are gathered (or created) to serve as a sampling of a reference distribution similar to the true concurrent reference distribution. These data are the ENACVs and INACVs obtained when this protocol (including the same choices of nucleic acid separation, extraction, and quantification) is performed up to this step on samples known to contain no microorganism, such as pure water or sterilized body fluids donated from healthy volunteers. In this set of experiment a goal was to compare the multiple prior reference condition data's ENACVs and INACVs with the one pair of treated ENACV and INACVs. The treated ENACV is a single scalar number, so we define a background ENACV threshold equal to the 99^{th}-percentile of the prior reference condition ENACVs. The treated INACV is a single scalar number, so we define a background INACV threshold equal to the 99^{th}-percentile of the prior reference condition INACVs. The sample is considered empty if the treated ENACV and the treated INACV are both below their respective background thresholds. If the empirical 99^{th}-percentile is difficult to calculate due to the lack of sufficient prior reference NACVs, one can estimate the 99^{th}-percentile by assuming the prior reference NACVs follow a certain probability distribution. For example, if one assumes the NACVs are normally distributed, then the 99^{th}-percentile is the sample mean of the prior reference NACVs plus a multiple (Φ⁻¹(0.99)=2.33) of the sample standard deviation of the NACVs, where ⁻¹ is the inverse cumulative distribution function of the standard normal distribution. The values of Φ⁻¹ can be found in a published standard normal table. The exact percentile (e.g. 99% here) used for the background cutoff can be varied subjectively, with a resulting tradeoff between the sensitivity and specificity of the well loading status algorithm.

Adequate performance of the well loading status algorithm occurs with thresholds between 90% and 99%. If the user desire to find the optimal percentile, the users can employ model selection algorithms as described in the machine learning literature.

The following algorithm is another nearly equivalent choice. Prior reference condition data is gathered comprising pair of ENACVs and INACVs. A multivariate Gaussian distribution is fitted to the prior reference condition data using the sample mean (a vector)

$$\hat{μ} = \frac{1}{m} {\sum }_{i = 1}^{m} {x}^{( i )} \qquad ( 31 )$$

and the unbiased sample covariance matrix

$$\hat{\sum } = \frac{1}{m - 1} {\sum }_{i = 1}^{m} ( {x}^{( i )} - \hat{μ} ) {( {x}^{( i )} - \hat{μ} )}^{T} . \qquad ( 32 )$$

Then, the well is considered empty when the likelihood of observing the treated ENACV and INACV pair of values, or a pair with a greater ENACV or a greater INACV value, is less than a subjectively chosen significance threshold probability, such as 0.01 or 0.05. The choice of the significance threshold controls a tradeoff between the algorithm's sensitivity and specificity. Most commercial or open-source statistical software can perform the above fitting and likelihood calculation. When there is no correlation between ENACVs and INACVs of empty samples, then this algorithm is equivalent to the above algorithm when NACVs are assumed to be normally distributed.

If the sample is called as not empty, an extra/intracellular nucleic acid proportion value (EINAPV) such as the fraction extracellular is calculated from the TENACV and the TINACV. Suitable formulas for the EINAPV are described elsewhere.

Since there is no concurrent reference condition, since none of the samples were found to be empty of cells, one can treat this assay as a bulk assay and evaluate the statistical significance of the one available treated EINAPV versus a null hypothesis that the strain is not responsive to antibiotic. If the EINAPV is statistically significant from the null hypothesis predictions, then the strain is considered susceptible. Otherwise, it is considered resistant.

The following is a suitable algorithm to determine the statistical significance of one treated EINAPV. First, gather or create prior reference condition data from untreated reference conditions as a sampling of a reference distribution similar to the true concurrent reference distribution. These data are the EINAPVs obtained when a same-sample AST protocol, preferably the same protocol, is performed up to this step (with the same choices of nucleic acid separation, extraction, and quantification) on samples known to contain the same or closely related species of microorganism (such as positive clinical specimens, or less preferably contrived clinical specimens spiked with the microorganism) and in which the microorganisms were not contacted with the antibiotic (contacted only with the vehicle of the antibiotic (e.g. pure water) and not the antibiotic compound itself, or less preferably where no contacting is performed). It is desired to include microorganisms in the prior reference condition data that are as similar to the currently tested microorganism as possible, with a tradeoff occurring between a larger number of prior reference condition data and the similarity of the microorganism in the included prior data. Keeping organisms within the same taxonomical species is a suitable criteria. Keeping organisms within the same taxonomical genus can be a suitable criteria for certain genera as well. Since there is only one tested EINAPV, one can compare it to a threshold value equal to the 99^{th}-percentile of the prior reference condition EINAPVs. The 99^{th}-percentile can be found empirically by ranking the prior reference condition EINAPVs (as can be done with commercial or open-source statistical software) or by fitting an assumed probability distribution to the prior reference condition data. As before, the percentile value used as a threshold can be varied subjectively in a tradeoff between AST assay sensitivity and specificity. Equivalently to using a 99^{th}-percentile threshold, one can calculate the likelihood of obtaining the tested EINAPV or a more extreme value given a distribution whose parameters are estimated from the prior reference condition data, and conclude a significant deviation if the likelihood is less than an a priori chosen significance threshold like 0.01, with the choice of the threshold being up to the user's subjective needs for AST assay sensitivity and specificity.

### Example 10: Same-Sample Filtration AST with Multiple Replicate Treated Conditions and No Concurrent Reference Conditions

An exemplary multiplex same-sample AST protocol is with multiple replicate treated conditions and no concurrent reference condition provided herein below in an outline describing the various sets of operations comprised in the protocol.

1. Clinical Specimen

A clinical specimen comprising bodily fluid, processed bodily fluids is obtained using standard collection techniques. For example, human cells in the specimen may be lysed by saponin treatment, and growth medium added to the sample to keep cells viable during transport; the specimen may also be briefly incubated with growth media; or the microorganisms in the cells can be enriched by mechanical, chemical, or electrical apparatuses. Alternatively, the clinical specimen may comprise a pure culture of microorganisms obtained from a clinical specimen using standard isolation techniques.

2. Sample Partitioning

Multiple samples of the clinical specimen, where N is the number of samples, are taken and contacted with one concentration of an antibiotic of interest (ABX) to create N treated antibiotic exposure conditions. The antibiotic exposure conditions are incubated together for a desired duration of time (the “exposure duration”).

3. Antibiotic Exposure

The concentration of the antibiotic is chosen according to the desires of clinicians, with any one of the relevant CLSI breakpoint concentrations being a preferred choice.

A minimal exposure duration or an exposure duration that maximizes assay confidence can be found by the prior compiling of assay results from a sampling of pathogenic microorganisms, or a rough approximation such as for 30 minutes or 60 minute can be employed.

The case of multiple antibiotic concentrations and/or multiple antibiotics is considered in another example as will be understood by a skilled person

4. Separation and Extraction

The N treated antibiotic exposure conditions are each subjected to a physical separation, such as filtration or centrifugation, and both the extracellular and intracellular nucleic acid fractions are separately collected and extracted in a way that preserves information about the in situ extracellular and intracellular nucleic acid concentrations in the antibiotic exposure. Suitable extractions are identifiable by a skilled person upon reading of the present disclosure.

5. Quantification

Nucleic acid amplification (with or without prior reverse transcription) is used to quantify both the N extracellular and the N intracellular nucleic acid fractions of the treated conditions, yielding N treated extracellular nucleic acid concentration values (ENACV) and N treated intracellular nucleic acid concentration values (INACV). The decision to include reverse transcription prior to nucleic acid amplification can be performed as described in other section of the present disclosure. Alternative methods for obtaining nucleic acid concentration values are also identifiable by a skilled person upon reading of the present disclosure.

6. Well Loading Status Algorithm

The N pairs of treated ENACVs and the treated INACVs are entered into the well loading status algorithm. For multiple treated conditions and no concurrent reference conditions, there are several preferred well loading status algorithms which yield comparable results in most cases and which are derived from the machine learning literature.

A first algorithm is equivalent to testing multiple hypotheses, one for each treated sample, where the null hypothesis is that the sample is empty and that its ENACV and INACV arise from the reference condition distribution. The reference condition distribution is estimated by fitting an assumed distribution to reference condition data gathered or created prior. This prior reference condition data comprises ENACVs and INACVs obtained when this protocol is performed up to this step (with the same choices of nucleic acid separation, extraction, and quantification) on samples known to contain no microorganism, such as pure water or sterilized body fluids donated from healthy volunteers. There is an infinite variety of probability distributions one can assume, but a normal (a.k.a. Gaussian) or a log-normal distribution are preferred choices. A choice between whether one's data is normally or log-normally distributed can be made by performing statistical tests for normality on the data and the log-transformed data, then choosing the distribution with a higher likelihood. Example tests for normality include the Shapiro-Wilk test, the Kolmogorov-Smirnov test, and visual inspection of Q-Q plots. Once a distribution is chosen and fitted, then each sample's likelihood can be calculated using the probability density function of the fitted distribution. If a sample's likelihood is below a significance threshold, then it is considered not to be empty.

A second algorithm is equivalent to fitting a mixture model. A mixture model is a statistical model in which the data are assumed to arise from one of several subpopulations but it is unknown which subpopulation gave rise to each datum. Each subpopulation arises as a random variable with a simple parameterized form, and the probability that a given datum arises from a given subpopulation is multinomially distributed. The mixture model can be fitted using the expectation-maximization algorithm, and implementations of mixture model fitting can be found in many commercial or open-source statistical software. The relevant output of the mixture model is the most likely assignment of each datum to a subpopulation. One can assume a fixed number of subpopulations, or one can allow the number of subpopulations to be chosen by the expectation-maximization algorithm. In the well loading status algorithm, we assume that the log-transformed data is distributed according to a multivariate Gaussian mixture model with between 1 and 4 subpopulations, possibly more if there are outlier data.

The fitting of mixture models is an example of a clustering technique and an example of an unsupervised machine learning algorithm. A list of known unsupervised machine learning algorithms is found in a further section of the present disclosure. All of these algorithms can be used singularly or in combination as the well loading status algorithm.

If the well loading status algorithm returns the result that none of the samples are empty, then one can proceed as if one has performed N bulk same-sample ASTs and continue with this example. If 1 or more but fewer than N^{-k} of the samples are empty, where k is a subjective threshold between 0% and 100%, preferably between 50% and 80%, more preferably between 55% and 65%, and most preferably equal to 60%, then the specimen was close to being digitally partitioned but not enough partitions were used to enable accurate estimation of the total number of cells in all of the samples. In this case, assuming a number Z of the samples were empty, one can proceed with this example as if the Z empty samples were 0 cell references and not treated conditions, and as if one has performed multiple bulk same-sample ASTs with the rest of the N−Z partitions. One can also flag the results as having less accurate results due to a low density of microorganism. If the number of empty samples is greater than N^{-k} but less than N (at least one sample is not empty), then the sample is digitally partitioned, and one instead follows the protocol in example #12 for digital same-sample AST with one treated condition and no concurrent reference conditions. If all N of the partitions are empty, then either the assay is repeated with a new clinical specimen, or the lack of infection is suspected.

7. Calculation of the EINAPV

If the sample is called as not empty, an extra/intracellular nucleic acid proportion value (EINAPV) such as the fraction extracellular is calculated for each of the non-empty samples from the ENACV and the INACV from that sample. Suitable formulas for the EINAPV are identifiable by a skilled person upon reading of the present disclosure.

8. Susceptibility Determination

To call susceptibility, one next evaluates the statistical significance of the many treated EINAPVs versus a null hypothesis that the strain is not responsive to antibiotic. If the EINAPVs are together statistically significant from the null hypothesis predictions, then the strain is considered susceptible. Otherwise, it is considered resistant.

The following is a suitable algorithm to determine the statistical significance of multiple treated EINAPVs. First, gather or create prior reference condition data from untreated reference conditions as a sampling of a reference distribution similar to the true concurrent reference distribution. These data are the EINAPVs obtained when a same-sample AST protocol, preferably the same protocol, (including the same choices of nucleic acid separation, extraction, and quantification) is performed up to this step on samples known to contain the same or closely related species of microorganism (such as positive clinical specimens, or less preferably contrived clinical specimens spiked with the microorganism) and in which the microorganisms were not contacted with the antibiotic (contacted only with the vehicle of the antibiotic (e.g. pure water) and not the antibiotic compound itself, or less preferably where no contacting is performed). It is desired to include microorganisms in the prior reference condition data that are as similar to the currently tested microorganism as possible, with a tradeoff occurring between a larger number of prior reference condition data and the similarity of the microorganism in the included prior data. Keeping organisms within the same taxonomical species is a suitable criteria. Keeping organisms within the same taxonomical genus can be a suitable criteria for certain genera as well. Since there is more than one tested EINAPV, and these multiple values serve as a sampling of the distribution of treated EINAPVs, one performs a statistical test that compares whether the distribution of treated EINAPVs is the same as the true concurrent reference distribution. Suitable statistical tests are identifiable by a skilled person upon reading of the present disclosure and include the two independent sample t-test, the Wilcoxon-Mann-Whitney test, and the two sample Kolmogorov-Smirnov test. The choice of the significance threshold is up to the user's subjective needs when balancing the tradeoff between AST assay sensitivity and specificity.

### Example 11: Digital Same-Sample Filtration AST with One Treated Condition and No Concurrent Reference Conditions

An exemplary digital same-sample AST protocol in is provided herein below in an outline describing the various sets of operations comprised in the protocol.

1. Clinical Specimen

A clinical specimen comprising bodily fluid or processed bodily fluids is obtained using standard collection techniques, identifiable by a skilled person upon reading of the present disclosure. Alternatively, the clinical specimen may comprise a culture of microorganisms obtained from bodily fluid using standard isolation techniques.

2. Sample Partitioning

A large number of samples or sample partitions of the clinical specimen, where N is the number of samples, are taken and contacted with one concentration of an antibiotic of interest (ABX) to create N treated antibiotic exposure conditions. The antibiotic exposure conditions are incubated together for a desired duration of time (the “exposure duration”).

To achieve digital loading, the number of samples N is maximized to the extent that is technically feasible, such as at least 100, more preferably at least 1000, more preferably at least 10,000, more preferably at least 100,000, and most preferably at least 1,000,000. When all partitions are the same volume, the following formula relates the expected number of empty partitions, the partition volume, and the density of cells

$${\sum }_{k = n}^{N} ( \begin{matrix}N \\ k\end{matrix} ) {{e}^{- D V k} ( 1 - {e}^{- D V} )}^{N - k} > t \qquad ( 33 )$$

N is the total number of partitions, n is the number of empty partitions, V is the partition volume, D is the density of cells, and t is a threshold probability chosen by the practitioner. Theoretically, the choice of n can be as low as 1 and as high as N−1; however, in practice, the choice of n is preferably at least 20% to 60% of N, more preferably at least 40% of N. The choice of t is preferably greater than 0.05, and more preferably greater than 0.5. In practice, either the minimum volume V that allows for growth of the microorganism is the limiting variable, or the maximum number N of partitions is limited by device size and reagent cost. The density of cells is usually not limiting when the clinical specimen is a bodily fluid, but it may be limiting when the clinical specimen is a cultured isolate. If the density of cells is too high, one can dilute the clinical specimen until the left side of the above equation is greater than the threshold probability t. Although the density of bacterial cells in the clinical sample is not known, in clinical scenarios a plausible range of densities is known, and so the partition number and volumes can always be chosen so that it is highly likely for a desired number of partitions to not receive any bacterial cells.

3. Antibiotic Exposure

The concentration of the antibiotic is chosen according to the desires of clinicians, with any one of the relevant CLSI breakpoint concentrations being a preferred choice.

A minimal exposure duration or an exposure duration that maximizes assay confidence can be found by the prior compiling of assay results from a sampling of pathogenic microorganisms, or a rough approximation such as for 30 minutes or 60 minute can be employed.

4. Separation and Extraction

The N treated antibiotic exposure conditions are each subjected to a physical separation, such as filtration or centrifugation, and both the extracellular and intracellular nucleic acid fractions are separately collected and extracted in a way that preserves information about the in situ extracellular and intracellular nucleic acid concentrations in the antibiotic exposure. Suitable extractions are identifiable by a skilled person upon reading of the present disclosure.

5. Quantification

Nucleic acid amplification (with or without prior reverse transcription) is used to quantify both the N extracellular and the N intracellular nucleic acid fractions of the treated conditions, yielding N treated extracellular nucleic acid concentration values (ENACV) and N treated intracellular nucleic acid concentration values (INACV). The decision to include reverse transcription prior to nucleic acid amplification is described in a further section of the present disclosure. Alternative methods for obtaining nucleic acid concentration values are also identifiable by a skilled person upon reading of the present disclosure.

6. Well Loading Status Algorithm

The N pairs of treated ENACVs and the treated INACVs are entered into the well loading status algorithm. There are a large number of samples which are being treated as one treated condition in this example, but as multiple replicate treated conditions in example #11. There are no concurrent reference conditions. There are thus several preferred well loading status algorithms which yield comparable results in most cases and which are derived from the machine learning literature.

A first suitable algorithm is equivalent to testing multiple hypotheses, one for each NACV of each treated sample, where the null hypothesis is that the sample is empty and that its ENACV and INACV arise from a reference distribution. The reference distribution is estimated by fitting an assumed distribution to reference condition data gathered or created prior. This prior reference condition data comprises ENACVs and INACVs obtained when this protocol is performed up to this step (with the same choices of nucleic acid separation, extraction, and quantification) on samples known to contain no microorganism, such as pure water or sterilized body fluids donated from healthy volunteers. There is an infinite variety of probability distributions one can assume, but a multivariate normal (a.k.a. Gaussian) or a multivariate log-normal distribution are preferred choices. A choice between whether one's data is normally or log-normally distributed can be made by performing statistical tests for normality on the data and the log-transformed data, then choosing the distribution with a higher likelihood. Once a distribution is chosen and fitted, then each sample's likelihood can be calculated using the probability density function of the fitted distribution. If a sample's likelihood reaches below a significance threshold, then it is considered not to be empty. If the sample is not empty, then it will need to be called as containing only extracellular nucleic acids from antibiotic-killed cells, as only containing intracellular nucleic acids in intact cells, or as containing both extracellular and intracellular nucleic acids. This can be done by examining the marginal distributions of the reference distribution and comparing each NACV with a significance threshold, or by other machine learning algorithms.

A second suitable algorithm is equivalent to fitting a mixture model. A mixture model is a statistical model in which the data are assumed to arise from one of several subpopulations but it is unknown which subpopulation gave rise to each datum. Each subpopulation arises as a random variable with a simple parameterized form, and the probability that a given datum arises from a given subpopulation is multinomially distributed. The mixture model can be fitted using the expectation-maximization algorithm, and implementations of mixture model fitting can be found in many commercial or open-source statistical software. The relevant output of the mixture model is the most likely assignment of each datum to a subpopulation. One can assume a fixed number of subpopulations, or one can allow the number of subpopulations to be chosen by the expectation-maximization algorithm. In the well loading status algorithm, we assume that the log-transformed data is distributed according to a multivariate Gaussian mixture model with between 1 and 4 subpopulations, possibly more if there are outlier data. The subpopulation or cluster R that contains the reference condition data is considered to represent empty samples. Any cluster with a mean ENACV or INACV less than the mean ENACV or INACV or cluster R is also called as empty. The other clusters are considered to represent non-empty samples. If the sample is not empty, then it will need to be called as containing only extracellular nucleic acids from antibiotic-killed cells, as only containing intracellular nucleic acids in intact cells, or as containing both extracellular and intracellular nucleic acids. To annotate (interpret, label, classify) these non-empty clusters, one can use the marginal distributions of the cluster R. If a non-empty cluster X has a mean ENACV that is not above the 99^{th}-percentile of cluster R's mean ENACV, then cluster X contains only intracellular nucleic acid. If a non-empty cluster X has a mean INACV that is not above the 99^{th}-percentile of cluster R's mean INACV, then cluster X contains only extracellular nucleic acid. If a non-empty cluster X doesn't satisfy the preceding two criteria, then it contains both extracellular and intracellular nucleic acids.

The fitting of mixture models is an example of a clustering technique and an example of an unsupervised machine learning algorithm. A list of known unsupervised machine learning algorithms is identifiable by a skilled person upon reading of the present disclosure. All of these algorithms can be used singularly or in combination as the well loading status algorithm.

If the well loading status algorithm returns the result that none of the samples are empty, then one did not achieve digital loading. One proceeds as if one has performed N bulk same-sample ASTs and continue with the protocol in example #11. If 1 or more but fewer than N^{-k} of the samples are empty, where k is a subjective threshold between 0% and 100%, preferably between 50% and 80%, more preferably between 55% and 65%, and most preferably equal to 60%, then the specimen was close to being digitally partitioned and in a way that most loaded partitions contain only 1 cell, but not enough partitions were used to enable accurate estimation of the total number of cells in all of the samples.

In particular in the experimental setting of this example, a more stringent criteria for digital-loading has been applied. This is because a goal of the experimenter is not only to determine the initial cell density but also to reduce the uncertainty linking single cell responses to well status by loading single cells in the majority of wells.

In this case, assuming a number Z of the samples were empty, one proceeds with the protocol in example #11 as if the Z empty samples were 0 cell references and not treated conditions, and as if one has performed multiple bulk same-sample ASTs with the rest of the N−Z partitions. One also flags the results as having less accurate results due to a low density of microorganism. If the number of empty samples is greater than N^{-k} but less than N (at least one sample is not empty), then the sample is digitally partitioned, and one continues with this example. If all N of the partitions are empty, then either the assay is repeated with a new clinical specimen, or the lack of infection is suspected.

6. Determination of Single Cell States (Latter Portion of the Well Loading Status Algorithm)

If the samples were digitally loaded, one next estimates the number of killed cells K and the number of intact cells L present in all of the samples. First, tally up the number of empty samples Z, the number of samples E with only extracellular nucleic acids, the number of samples I with only intracellular nucleic acids, and the number of samples B with both extracellular and intracellular nucleic acids. Let C be the most probable concentration (or density) of microorganism in the specimen at the time of sample partitioning. C can be calculated by the following equation:

$$C = - ln ( \frac{Z}{Z + E + I + B} ) , \qquad ( 34 )$$

where ln(x) is the natural logarithm of x. If one makes additional assumptions, additional terms can be added to the equation to make it more precise, such as by assuming that all samples with both extracellular and intracellular nucleic acids have more than 1 cell. The simplest calculation of K and L uses the following equations:

$$K = \frac{E + B}{D} and \qquad ( 35 )$$

$$L = \frac{I + B}{D} . \qquad ( 36 )$$

This equation assumes that no additional division of cells has occurred during the antibiotic exposure, and that any sample with both extracellular and intracellular nucleic acids contains at most 2 cells.

7. Determination of the EINAPV

Once one has obtained the estimates of K and L, calculate a proportion value from K and L. Suitable formulas for the EINAPV are described in additional sections of the present disclosure. If the formulas are given in terms of ENACVs and INACVs, one substitutes the ENACV with K and substitute the INACV with L. The one resulting proportion value can still be called a treated EINAPV.

8. Susceptibility Call

To call susceptibility, one next evaluates the statistical significance of the one treated EINAPV versus a null hypothesis that the strain is not responsive to antibiotic. If the EINAPV is statistically significant from the null hypothesis predictions, then the strain is considered susceptible. Otherwise, it is considered resistant.

The following is a suitable algorithm to determine the statistical significance of one treated EINAPV. First, gather or create prior reference condition data from untreated reference conditions as a sampling of a reference distribution similar to the true concurrent reference distribution. These data are the EINAPVs obtained when a same-sample AST protocol, preferably the same protocol, is performed up to this step (with the same choices of nucleic acid separation, extraction, and quantification) on samples known to contain the same or closely related species of microorganism (such as positive clinical specimens, or less preferably contrived clinical specimens spiked with the microorganism) and in which the microorganisms were not contacted with the antibiotic (contacted only with the vehicle of the antibiotic (e.g. pure water) and not the antibiotic compound itself, or less preferably where no contacting is performed). It is desired to include microorganisms in the prior reference condition data that are as similar to the currently tested microorganism as possible, with a tradeoff occurring between a larger number of prior reference condition data and the similarity of the microorganism in the included prior data. Keeping organisms within the same taxonomical species is a suitable criteria. Keeping organisms within the same taxonomical genus can be a suitable criteria for certain genera as well. Since there is only one tested EINAPV, one compares it to a threshold value equal to the 99^{th}-percentile of the prior reference condition EINAPVs. The 99^{th}-percentile can be found empirically by ranking the prior reference condition EINAPVs (as can be done with commercial or open-source statistical software) or by fitting an assumed probability distribution to the prior reference condition data. As before, the percentile value used as a threshold can be varied subjectively in a tradeoff between AST assay sensitivity and specificity. Equivalently to using a 99^{th}-percentile threshold, one can calculate the likelihood of obtaining the tested EINAPV or a more extreme value given a distribution whose parameters are estimated from the prior reference condition data, and conclude a significant deviation if the likelihood is less than an a priori chosen significance threshold like 0.01, with the choice of the threshold being up to the user's subjective needs for AST assay sensitivity and specificity.

### Example 12: Time-Series Same-Sample Filtration AST with One Treated Condition and No Concurrent Reference Conditions, and with One 0-Minute Time Points

An exemplary time series same-sample AST protocol is provided herein below in an outline describing the various sets of operations comprised in the protocol.

1. First Separation Prior to Antibiotic Contact

A clinical specimen comprising bodily fluid, processed bodily fluids is obtained using standard collection techniques. For example, human cells in the specimen may be lysed by saponin treatment, and growth medium added to the sample to keep cells viable during transport; the specimen may also be briefly incubated with growth media; or the microorganisms in the cells can be enriched by mechanical, chemical, or electrical apparatuses. Alternatively, the clinical specimen may comprise a pure culture of microorganisms obtained from a clinical specimen using standard isolation techniques.

One sample of the clinical specimen is obtained by partitioning part of the specimen into a clean vessel. Since a 0-minute time point is planned, this clean vessel is a filtration device such as a commercial centrifuge-based or vacuum-based filter cartridge whose outlet can be reversibly closed so that bacteria can be cultured in growth media in the filter cartridge for extended amounts of time. The sample is passed through the filter, and the collected liquid (the 0-minute filtrate) is extracted and stored.

Any method of physical separation of intact cells and extracellular nucleic acids which does not destroy the viability of the intact cells can be used for a time-series same-sample AST. Centrifugation is one such physical separation technique. The pellet of intact cells created by a centrifugation can be resuspended in growth media after the supernatant is removed.

An exemplary filter cartridge is the Corning Costar Spin-X 2 mL filter unit used in other examples. Although the filter unit does not have a lid on its outlet, liquid passes through the filter at a slow rate when the unit is not centrifuged, and any liquid that does pass through can be collected in a clean tube and included in the filtrate of the next centrifugation without disturbing the assay results.

2. Antibiotic Exposure and Time-Series Separations

The sample is contacted with an antibiotic of interest in new growth media to create a treated antibiotic exposure condition. The concentration of the antibiotic is chosen according to the desires of clinician, with any one of the relevant CLSI breakpoint concentrations being the preferred choice.

At any number “T” of chosen time points, such as at 15, 30, 45, 60, 75, 90, 105, and 120 minutes, the sample is separated by filtration and the filtrate is collected. New growth media with the same antibiotic is added to the filtration device's vessel and the incubation of bacteria and antibiotics resumes. The time of each action is recorded (e.g. by stopwatch, video recording, or timer component of an automated system). The filtrate is chemically stabilized via an extraction protocol (e.g., Lucigen DNA Extraction Buffer) and proper storage (e.g. freezers). Each filtrate is expected to contain all extracellular nucleic acids that have accumulated in the time since the previous filtration.

Inclusion of more exposure durations will increase the accuracy of the subsequent statistical analysis. Inclusion of more exposure durations will increase the cost of reagents in subsequent steps (extraction protocol, reverse transcription, nucleic acid amplification). The exposure durations can be chosen so at least one of the time points is expected, according to any population dynamics models assumed for the strain of interest, to capture extracellular nucleic acids if the strain were to be susceptible. Generally, this means that one time point is after a minimum exposure duration of about 10 to 60 minutes.

If the filtration vessel used allows continuous filtration, then there is no time where the cells are not incubated in optimal conditions, and many more time points can be collected. For example, one can construct a continuous flow microfluidic device where intact bacteria are entrapped in a chamber. The “mother machine” microfluidic device is one such design. Growth media and antibiotics flow past the bacteria and are then partitioned into discrete, ordered volumes. The ordered volumes can an ordered array of droplets separated by marker droplets of a different volume, as shown in [32], or microfabricated wells. It is also possible to use an unordered collection of droplets in an oil emulsion, where the droplets are uniquely barcoded by fluorescent dyes [33], by nucleic acid barcodes on polymer beads [34], or other method known to the skilled user. Each volume then undergoes nucleic acid quantification. The nucleic acid quantification method could be a real-time reverse-transcription isothermal LAMP reaction monitored by fluorescence imaging of the droplet array.

3. End of Antibiotic Exposure and Extraction of Remaining Intracellular Nucleic Acids

After the last of the chosen filtrations is performed, the nucleic acids within the remaining intact cells are extracted. In this embodiment, DNA extraction buffer is added to the filter cartridge and the cartridge is heated to 65° C. for at least 6 minutes. The extraction buffer is then collected by centrifugation to create the lysate extraction.

4. Quantification of Nucleic Acid Concentration Values

Nucleic acid amplification (with or without prior reverse transcription) is used to quantify all of the extracellular fractions and the one intracellular nucleic acid fractions, yielding T treated extracellular nucleic acid concentration values (ENACVs) and 1 treated intracellular nucleic acid concentration value (INACVs). The decision to include reverse transcription prior to nucleic acid amplification is described in a further section of the present disclosure. Suitable methods for obtaining nucleic acid concentration values, such as ddPCR or qPCR, would be identifiable by a skilled person upon reading of the present disclosure.

For an AST to be useful with only one treated condition and species-specific primers for amplification, it is assumed at this point that the microorganism has already been identified using standard identification assays so that the correct primers are used. Otherwise, approaches using universal primers, multiplexed primers, high-resolution melting curves, or sequencing could be used.

5. Calculation of EINAPV and Susceptibility Call

Finally, one uses any one of a variety of mathematical models to call susceptibility. In general, a population model is fitted to the time series data to calculate a parameter such as a kill rate or a maximum EINAPV, and then the parameter is compared to non-concurrent reference values of that parameter or to a table compiled beforehand of which parameter values are considered susceptible or resistant. Four models are described below.

In a first model, one can calculate the final fraction f_{T} of the total population lysed by the last time point, which is a type of EINAPV. This EINAPV is s equal to

$${f}_{T} = \frac{{\sum }_{i = 0}^{T} {E}_{i}}{{I}_{T} + {\sum }_{i = 0}^{T} {E}_{i}} , \qquad ( 37 )$$

or the sum of all ENACVs divided by the sum of all ENACVs and the final INACV. Then, one can compare the f_{T} EINAPV to non-concurrent reference data or to an arbitrary a priori threshold value as discussed in other sections of the present disclosure.

In a second model, one ignores any growth of the cell population occurring after the antibiotic exposure begins. Under such a model of population dynamics (in which the population happens to not be “dynamic”), the fraction extracellular of the population at each time point can be calculated because the total nucleic acid remains constant at all time points. Let E₀, E₁₅, . . . , Eₜ, E_{T=120} be the T measured ENACVs, and let I_{T=120} be the final INACV. The total nucleic acids in the sample is I_{T}+Σᵢ₌₀^{T}Eᵢ. Since any nucleic acids remaining at time t must have been intracellular at time of the previous filtration, the amount of intracellular nucleic acids at time t, Iₜ, is equal to Iₜ=I_{T}+Σᵢ₌ₜ₊₁^{T}Eᵢ. For example, I_{T-1=105min}=I_{T}+Σ_{i=T}^{T}Eᵢ=I_{T=120min}+E_{T=120min}, and I_{T-2=90min}=I_{T}+Σ_{i=T-1}^{T}Eᵢ=I_{T-1=105min}+E_{T-1=105min}. The fraction fₜ of the total population lysed by time t is equal to

$${f}_{t} = \frac{{\sum }_{i = 0}^{t} {E}_{i}}{{I}_{T} + {\sum }_{i = 0}^{T} {E}_{i}} , \qquad ( 38 )$$

while the kill rate k(t) at between time t−1 and t is

$$k ( t ) = \frac{{E}_{t}}{{I}_{t} + {E}_{t}} = \frac{{E}_{t}}{{I}_{T} + {\sum }_{i = t}^{T} {E}_{i}} . \qquad ( 39 )$$

The kill rate is a measure of antibiotic susceptibility and can be compared to kill rates measured from non-concurrent control experiments, such as a table of background lysis rates for the relevant organism or a table that shows kill rate as a function of strain MIC and antibiotic concentration. In the former, if and only if the kill rate is significantly higher than the background lysis rate, then the strain is susceptible. In the latter, the kill rate is mapped to an MIC value, which indicates a susceptible, intermediate, or resistant phenotype according to CLSI or equivalent guidelines.

In a third model, growth of the cell population is assumed to occur during the exposure. The population growth (and death due to antibiotics) is assumed to obey the following ordinary differential equations:

$$\frac{dI [ t ]}{dt} = ( μ - k - h [ t ] ) * I [ t ] , \frac{dE [ t ]}{dt} = ( k + h [ t ] ) * I [ t ] , h [ t ; α , β ] = \frac{{β}^{α} {t}^{α - 1} {e}^{- β t}}{Γ [ α ] Q [ α , β t ]} , \qquad ( 40 )$$

where I[t] is the amount of intracellular nucleic acids (and thus live cells) at time t, E[t] is the amount of extracellular nucleic acids (and thus the accumulated dead cells) at time t, μ is the growth rate of the bacterial species, k is the background lysis rate, α is a parameter unique to each antibiotic representing the effective number of damage events caused by antibiotics needed to lyse a cell, β is the rate at which those antibiotic damage events occur, Γ[α] is the gamma function evaluated at α, and Q[α, βt] is the regularized upper incomplete gamma function with a shape parameter of α evaluated at βt. The microorganism species-specific values of μ and k can be measured in non-concurrent experiments that include an untreated reference condition, and the results compiled in a table for known microorganism species. Similarly, values of α for common antibiotics, possibly for pairs of antibiotics and species of microorganisms, are measured in untreated conditions and compiled into a table. The observed ENACV and INACV data are fitted to the model using a Bayesian probabilistic model that assumes normal and/or log-normal measurement error when ddPCR is used to measure the nucleic acid concentrations. The non-concurrent values for μ and k are incorporated into the Bayesian model as priors for the values of μ and k. When the model is fit, an estimate of the value of the kill rate β is obtained. If the kill rate is significantly higher than the value of μ, then the strain is susceptible. Alternatively, the kill rate can be compared to a table of background lysis rates for the relevant organism or a table that shows kill rate as a function of strain MIC and antibiotic concentration. In the former, if and only if the kill rate is significantly higher than the background lysis rate, then the strain is susceptible. In the latter, the kill rate is mapped to an MIC value, which indicates a susceptible, intermediate, or resistant phenotype according to CLSI or equivalent guidelines.

### Example 13: Digital Same-Sample Filtration AST with Multiple Non-Replicate Treated Conditions and One Concurrent Reference Condition

An exemplary time series same-sample AST protocol is provided herein below in an outline describing the various sets of operations comprised in the protocol.

In the experiment described in this example, the goal was to measure the susceptibility of an isolate to two different concentrations of beta-lactam antibiotic at 30 minutes of exposure.

A 96-well microtiter plate was prepared for the experiment by placing Mueller-Hinton Broth (MHB) growth media and one of three different concentrations of ertapenem (ETP) sodium salt into each well. 32 of the wells contained 0 μg/mL of ETP antibiotic and served as reference condition antibiotic exposures; pure water was added instead of ETP dissolved in pure water. Another 32 of the wells contained 0.417 μg/mL of ETP (for a final concentration of 0.25 μg/mL). The last 32 wells contained 3.33 μg/mL of ETP (for a final concentration of 2.0 μg/mL). The 64 wells containing some ertapenem constituted the treated condition antibiotic exposures.

1. Providing a Sample

For the purposes of demonstration, a contrived clinical sample was created by inoculating Escherichia coli K12 into Brain-Heart Infusion broth. The inoculum was small enough that no detectable difference in the sample's optical density at 600 mm (OD₆₀₀) was detectable by a spectrophotometer with a sensitivity of 0.01 absorbance units. After an incubation at 37° C., the media became turbid with an OD₆₀₀ of 0.334 absorbance units after 3.5 hours of incubation.

In this experiment, the number and volume of sample partitions was restricted, for logistical reasons, to 96 partitions 25 μL in volume, specifically the wells of a 96-well plate. A target loading density of 0.5 cells per partition was set. The contrived clinical sample was diluted by a factor of 9.97239*10\hat{ }−7 via a three-step serial dilution to an estimated density of 20 CFU/mL.

In the 2 minutes before the start of the AST protocol, 10 μL of the diluted contrived clinical specimen (containing 20 CFU/mL) was added to each well of an empty sterile 96-well plate. This plate was sealed with an RNase/DNase-free sealing foil and placed in a 37° C. incubator overnight. Since 26 of the wells were found the next morning to be empty, while the others contained a pellet of bacteria, an accurate estimate of the density of cells at the start of the exposure was

$$\frac{1}{10 µL} \times - ln ( \frac{26}{96} ) = 0.1 \; 31 CFU / u L$$

or 131 CFU/mL, rather than the target of 20 CFU/mL. This estimation of the true density of cells is not necessary for determining susceptibility, but was included in this example as a quality control.

2. Antibiotic Exposure

To begin the AST protocol, the diluted contrived clinical specimen was physically partitioned into the 96 samples by transferring 10 μL of the specimen, in 96 separate transfers (actually 8 transfers with a multichannel pipette), to each well of the 96-well microtiter plate containing ETP and MHB. Then, the entire plate was sealed with a RNase/DNase-free plastic, adhesive sealing membrane and placed on a thermal block set to 37° C. and shaking at 700 rpm.

3. Sample Separation by Filtration and Centrifugation

After 8 minutes of incubation at 37° C., the exposures were taken back to the lab bench. The entire volume of each antibiotic exposure was transferred to a Millipore® 96-well sterile polystyrene MultiScreenHTS® filter plate (Millipore-Sigma MSGVS2210). Each well of the filter plate contained a hydrophilic polyvinylidene fluoride (PVDF) filter membrane with a 0.22 pore size. A 96-well polypropylene microtiter plate was affixed to the bottom of the filter plate. The filter plate was promptly centrifuged at 2200 relative centrifugal force for 5.0 minutes to speed the passage of the antibiotic exposure sample through the filter and into 96-well microtiter plate. The collected fluid was called the “filtrate.” In total, between 19.0 and 21.2 minutes elapsed between the first addition of cells to the exposure volume and the start of the centrifugation. The total time elapsed differed slightly among wells because cells could not be manually added to all wells at the exact same time.

4. Extraction of Extracellular/Accessible Nucleic Acid

8 μL of each filtrate was transferred to new containers containing 6 μL of Lucigen DNA Extraction Buffer, vortexed, centrifuged briefly to collect liquid at the bottom of the tube, and then heated for 6 minutes at 65° C. and then 4 minutes at 98° C.

The filter pore size was chosen to prevent the passage of intact bacterial cells, which are all larger than 0.22 with rare exceptions.

The centrifugation speed was chosen to be low enough to prevent cell lysis, as would be understood by a skilled person upon reading of the present disclosure.

Any exposure duration, with a reasonable range being up to 24 hours, could have been chosen instead the exposure durations actually chosen.

5. Filter Washing

After the creation of the filtrates, 30 μL of fresh MHB media was added to all filters. The filters at this time possessed intact cells on their surface, whose nucleic acids remained intracellular. The purpose of this new volume of MHB was to wash away residual extracellular nucleic acids that might be confused for intracellular nucleic acids when the lysate was collected. The 30 μL of fresh MHB was centrifuged for 5 minutes at 2200 rcf into a clean 96-well plate, then discarded.

6. Cell Lysis and Extraction of Intracellular/Inaccessible Nucleic Acid

Next, 20 μL of DNA Extraction Buffer was placed into all of the wells of the filter plate, on top of the filters. The filter plate was heated to 65° C. for 6 minutes, without shaking, on a ThermoMixer® flat surface heating block. Intracellular nucleic acids were released from lysed cells into the DNA Extraction Buffer fluid. Then, the filter plate was taped to a clean 96-well polypropylene microtiter plate and centrifuged at 2200 rcf for 5 minutes. The DEB fluid that flowed through the filter was collected in the microtiter plate below the filter plate.

Next, that microtiter plate containing collected DEB was heated to 98° C. for 4 minutes inside a BioRad thermocycler. These collected and heated fluid volumes are termed the “extracted lysate”. The purpose of this step is to recover the intracellular nucleic acids found in the intact cells retained on the filters. To do so, these intact cells are lysed and their nucleic acids extracted. The lysate is expected to contain all or most of the formerly intracellular, now extracellular nucleic acids. In this experiment, the extracted lysates were frozen at −80° C. to pause the experiment. Freezing extracted nucleic acids is not necessary if one immediately continues to the next step in the protocol (the reverse transcription step).

Alternative ways to extract the intracellular nucleic acids can be performed. For example, the filter membrane can be removed from the filter apparatus using sterile and clean forceps and placed into a volume of DNA Extraction Buffer. This volume of buffer is vortexed vigorously, then heated to 65° C., then heated to 98° C.

As a third alternative, intact bacterial cells retained on the filter can be mechanically dislodged (e.g. centrifugation in the opposite direction, stirring), then transferred to a volume of DNA Extraction Buffer, which is then heated to 65° C. and then to 98° C.

Additional lytic reagents such as lysozyme can be added to the DNA Extraction Buffer to increase lysis efficiency.

The temperatures of 65° C. and 98° C. derive from the manufacturer's instructions for the Lucigen DNA Extraction Buffer kit.

7. Reverse Transcription of Nucleic Acid in the Filtrates and Lysates

Separately, for each of the 96 extracted filtrates and for each of the 96 extracted lysates, 1.50 μL of the nucleic acid was mixed in a 4 μL reaction containing 0.024 U/mL Lucigen® RapiDxFire thermostable reverse transcriptase, Lucigen® RapiDxFire thermostable buffer, 0.5 mM deoxyribonucleic acid nucleotides, deionized water, and 0.4 μM aqueous solution of DNA primer, according to manufacturer's instructions. The DNA primer included had a sequence of 5′-TGTCTCCCGTGATAACTTTCTC-3′ (SEQ ID NO: 3). The primer's sequence was complementary to the 23S ribosomal RNA in Escherichia coli and specific to the Enterobacteriaceae family. The cDNA product that would be created from this primer contained the primer sites for the future ddPCR reaction occurring later in this AST protocol. All 192 reverse transcription reactions were heated to 75° C. for 15 seconds to denature rRNA, 60° C. for 10 minutes to create cDNAs, then heated to 95° C. for 5 minutes to stop the reaction and inactivate the reverse transcriptase enzyme.

A reverse transcription step is optional if one has decided to amplify a DNA molecule found naturally in the cells of interest. However, if the nucleic acid to be quantified in the AST protocol is a ribonucleic acid (RNA) molecule, and the quantification method operates only on deoxyribonucleic acid molecules, then both the filtrate and the lysate can be treated with a reverse transcriptase enzyme to produce complementary DNA molecules (cDNA) prior to nucleic acid quantification. The concentration of cDNA, and thus rRNA, is calculated from the counts of high and low fluorescence droplets.

Alternative reverse transcription enzymes, protocols, and kits may be used instead of the kit used in this example.

Alternative primers may be used. Alternative nucleic acid species can be targeted as well, through a choice of primers. As noted earlier in this document, targets with a higher copy number per cell are preferred for accessibility AST.

8. Quantification of Extracellular/Accessible Nucleic Acid and Intracellular/Inaccessible nucleic acid

A 1 μL volume of each of the above reverse transcription reactions was separately added, according to kit instructions, to 3.5 μL of BioRad SsoFast qPCR EvaGreen 2× supermix, 2.22 μL nuclease-free water, and 0.28 μL of a pair of DNA PCR primers at 10 μM each, to create a 6 μL qPCR reaction. The pair of PCR primers possessed the following sequences: 5′-GGTAGAGCACTGTTTTGGCA-3′ (SEQ ID NO: 2), 5′-TGTCTCCCGTGATAACTTTCTC-3′(SEQ ID NO: 3). The DNA primers' sequences flanked an 80 bp region common to all of the 23S ribosomal RNA in Escherichia coli but specific to the Enterobacteriaceae family. One of the primers was the same primer used in the prior reverse transcription reaction. Real time qPCR of the qPCR reactions was performed on the BioRad CFX96 platform according to manufacturer's instructions. The real time qPCR protocol comprised 50 cycles of 30 seconds of denaturing at 95° C. and 60 seconds of annealing and extension at 60° C. A melt curve between 55° C. and 95° C. with a ramp rate of 0.5° C./s was also performed. The output of the qPCR run was the threshold cycles, which reflect nucleic acid concentration, of the filtrate and in the lysate of both antibiotic exposures. The outputted threshold cycles are plotted in FIG. 11B.

9. Well Loading Status

From the 96 pairs of filtrate and lysate nucleic acid concentrations measured, the loading status of the 96 antibiotic exposures were estimated using a well loading status algorithm. The resulting tally of the number of wells in each of the experimental conditions (treated and reference) possessing each loading status are found in the Table 8 below.

TABLE 8

| | | Extracellular | Intracellular | | |
|---|---|---|---|---|---|
| Antibiotic dosage | Empty | only | only | Both | TOTAL |
| Reference (0.0 μg | 13 | 0 | 19 | 0 | 32 |
| ETP/mL) | | | | | |
| Treated (0.25 μg | 15 | 0 | 17 | 0 | 32 |
| ETP/mL) | | | | | |
| Treated (2.00 μg | 10 | 0 | 20 | 2 | 32 |
| ETP/mL) | | | | | |

The well loading status algorithm used comprised manually select a threshold of 35 and 33 for the filtrate and lysate Cq values. Manual selection was possible because of the clean bimodal distribution of the Cqs along each axis, and the prior knowledge that any Cq greater than or equal to 35 is typically due to non-specific amplification and is to be considered as failing to detect template molecules.

Alternative choices for the well loading status algorithm can be used and may perform better than the algorithm used in this experiment. A non-exhaustive list of appropriate choices is described in a further section of the present disclosure and identifiable by a skilled person.

10. Live/Dead Determination and Susceptibility Determination

Finally, the susceptibility of the strain is called. First, the number of killed and intact cells from the tallies of samples in each loading status was estimated. The most likely density per sample, for digitally-loaded samples of equal volume, can be estimated by the equation

$$Density = - ln ( \frac{\# empty samples}{\# total samples} ) . \qquad ( 41 )$$

Since there were 38 empty partitions out of 96 total partition of equal volume, the most likely density is 0.927 cells per sample, or 92.7 CFU/mL in the diluted batch culture. With 96 samples, assuming each well's number of cells is independently Poisson distributed with a mean of 0.927, and given that 58 wells are non-empty, the most likely loading of wells is that 36 wells contain 1 cell, 16 wells contain 2 cells, and 6 wells contain more than 2 cells.

The expected number of cells in the plate in total is 89. We will assume that the wells with both lysed and unlysed cells were loaded with more than one cell, especially since the exposure duration was short. To facility a manual computation in this example, we assume that the expected excess of 89−58=31 cells is distributed evenly among the observed non-empty wells, except that 2 are assigned to the wells with both intra- and extracellular nucleic acids. This means that the wells with intracellular nucleic acids only now contain an extra 0.517 cells. With these new assumptions, the most likely number of intact and lysed cells is depicted in the following Table 9

TABLE 9

| Antibiotic dosage | Intact | Lysed | TOTAL |
|---|---|---|---|
| Reference (0.0 μg ETP/mL) | 29 | 0 | 29 |
| Treated (0.25 μg ETP/mL) | 26 | 0 | 26 |
| Treated (2.00 μg ETP/mL) | 32 | 2 | 34 |

A unique maximum likelihood sample loading would be possible to identify if bacteria population dynamics were assumed to occur, and that the concentration of nucleic acids in a sample is a function of exposure duration, susceptibility, and the starting number of cells. After correction for nucleic acid synthesis during the exposure and after assuming a certain susceptibility, the assumption is made that samples with a higher nucleic acid concentration in either the filtrate or the lysate were more likely to have contained more than one cell. The population dynamics of a single sample can be modeled by any of the population models used in the biology literature for bacteria, cells, and living organisms in general. Example population models include ordinary differential equations such as but not limited to the exponential growth equation, the logistic growth equation, and the Gompertz equation, and any variation of these models as will be known to the skilled practitioner. Other example population models may use branching stochastic processes and stochastic differential equations, such as Galton-Watson processes, multi-type Galton-Watson processes, continuous time Markov chain processes (simulated using the Gillespie algorithm), the Bellman-Harris process, and any variation of these models as will be known to the skilled practitioner.

Unlike maximum likelihood estimation, a Bayesian probabilistic model that includes a prior distribution would be able to calculate the posterior probability of strain susceptibility marginalized over all possible sample loadings. A Bayesian model that includes bacterial population dynamics could interpret the nucleic acid concentrations of each sample instead of only the binary well loading status call.

Next, for each of the four sets of exposure durations, a binomial exact test was performed to test the hypothesis that each cell in the treated and the reference conditions had the same probability of lysing. A significance threshold of 0.05 was chosen a priori. In the binomial exact test, we assume that every cell has an identical chance of lysing, l, during the exposure. The most likely value for l, called \hat{l}, is the observed ratio of lysed vs total cells for all cells assumed to share the same value of l. In other words, \hat{l}=(x_{RE}+x_{TE})÷(x_{RE}+x_{RE}+x_{TI}+x_{TE}), where x_{TE} is the number of lysed treated cells, x_{TI} is the number of intact treated cells, x_{RE} is the number of lysed untreated cells, and x_{RI} is the number of intact untreated cells. In this experiment, 1=2/89=0.0225. The one-sided p-value of the binomial exact test for one set of samples is found by the equation

$$BinomialProbability ( X \leq {x}_{R E} ; n = {x}_{R I} + {x}_{R E} , p = \hat{l} ) * BinomialProbability ( X \geq {x}_{T E} ; n = {x}_{T I} + {x}_{T E} , p = \hat{l} ) = ( {\sum }_{X = 0}^{{x}_{R E}} [ ( \begin{matrix}{x}_{RI} + {x}_{R E} \\ X\end{matrix} ) {( \hat{l} )}^{X} {( 1 - \hat{l} )}^{{x}_{RI} + {x}_{R E} - X} ] ) [ 1 - {\sum }_{X = 0}^{{x}_{T E - 1}} [ ( \begin{matrix}{x}_{TI} + {x}_{T E} \\ X\end{matrix} ) {( \hat{l} )}^{X} {( 1 - \hat{l} )}^{{x}_{TI} + {x}_{T E} - X} ] ] . \qquad ( 42 )$$

For this experiment, the p-value for the 0.25 μg/mL condition is (1−2/89)²⁹(1)=0.517, and the p-value for the 2.0 μg/mL condition is (1−2/89)²⁹[1−[(1−2/89)³⁴+34(2/89)¹(1−2/89)³³]]=0.092. In both conditions, we would be unable to conclude, with the typical 5% error tolerance we selected a priori, that the strain is susceptible. However, in the 2.0 μg/mL case, we can construct an alternative hypothesis that the strain is susceptible with a proportion of lysis equal to 2/34, while the background rate of lysis in the reference condition is 0/29. In the null hypothesis, the likelihood of the observed data is

$$BinomialProbability ( X = {x}_{R E} ; n = {x}_{R I} {x}_{R E} , p = \frac{2}{89} ) * BinomialProbability ( X = {x}_{T E} ; n = {x}_{T I} {x}_{T E} , p = \frac{2}{89} ) = 0.51730 \; 67 * 0.1368922 = 0.0 708. \qquad ( 43 )$$

In the alternative hypothesis, the likelihood is

$$BinomialProbability ( X = {x}_{R E} ; n = {x}_{R I} {x}_{R E} , p = \frac{0}{29} ) * BinomialProbability ( X = {x}_{T E} ; n = {x}_{T I} {x}_{T E} , p = \frac{2}{34} ) = 1 * 0.27895 \; 81 = 0.279 . \qquad ( 44 )$$

Thus, the likelihood ratio of the strain being susceptible is 0.279/0.0708=3.94, which in a non-clinical scenario would lead us to interpret that the strain is more likely to be susceptible, but that more exposure time or more cells are needed to yield a conclusive assay result.

### Example 14: Bulk Filtration Reflects Bacterial Population Dynamics at Short Time Scales

An exemplary bulk filtration was performed in replicates of bulk accessibility AST following the protocol described in US2019/0194726, US2021/0301326, and WO2019/075624 incorporated herein by reference in their entirety.

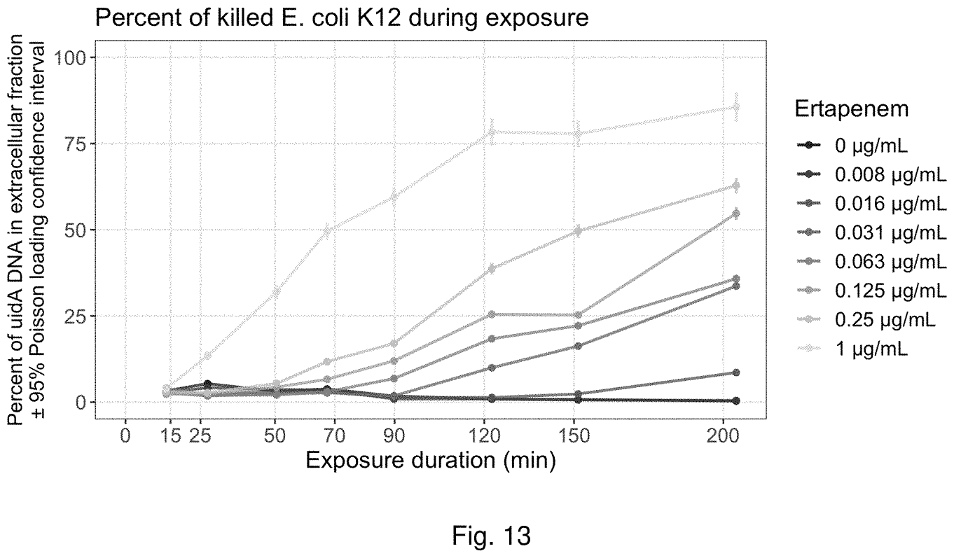

The results shown FIG. 12 illustrate the extracellular and total genomic DNA as a function of time. The same population dynamic phenomena shown in FIG. 12 will occur in an exposure in same-sample AST performed in bulk.

In the illustration of FIG. 12, one can see that before the treated, susceptible population has died out from antibiotic, there is continued growth of the total nucleic acid biomass in the exposure due to continued growth of living cells. One can also see that a lag in antibiotic killing is present in the first 20 minutes, although this concave lag is rendered less noticeable because the logarithmic-scale y axis reduced the concavity of the treated, extracellular curve. One can also see that background lysis occurs at a rate of about 0.01% per minute. In these bulk AST runs, approximately 9375 cells were loaded per partition, with between 8 to 10 partitions per run. Genomic DNA was measured by primers for the uidA gene.

### Example 15: Bulk Filtration AST Reveals Pharmacodynamics at Short Time Scales

An exemplary bulk filtration was performed in replicates of bulk accessibility AST following the protocol described in US2019/0194726, US2021/0301326, and WO2019/075624 incorporated herein by reference in their entirety.

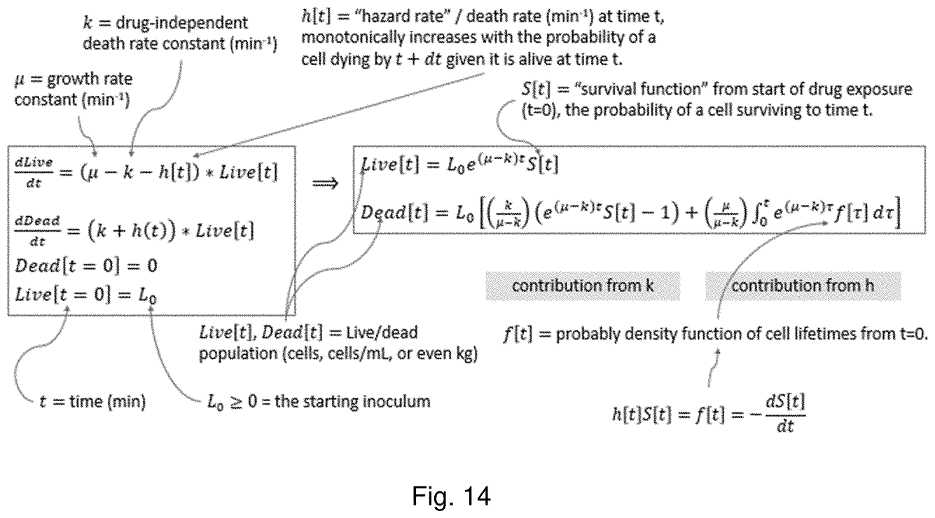

The results shown in FIG. 13 illustrate the percent of total DNA that is extracellular as a function of time and antibiotic concentration. The same population dynamic phenomena shown in FIG. 13 will occur in an exposure in same-sample AST performed in bulk.

In the results shown in FIG. 13 one can see that the rate of antibiotic killing increases monotonically with antibiotic concentration. One can see that the apparent lag in antibiotic killing also shortens as antibiotic concentrations increase. In these bulk AST runs, approximately 50000 cells were loaded into each of 48 partitions of this multiplexed AST run. Genomic DNA was measured by primers for the uidA gene.

### Example 16: Rate of Lysis Over Time

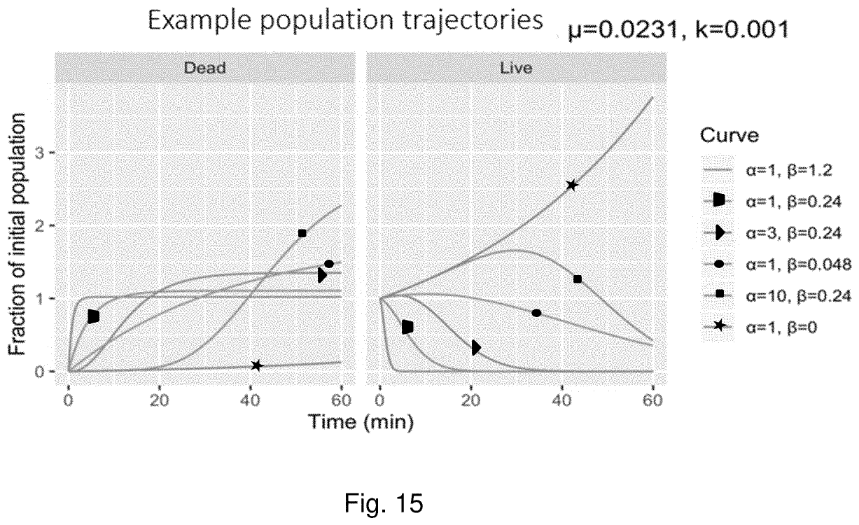

As explained above, in some cases the rate of lysis is a function of time h[t]. In these cases, a compartment model of in vitro antibiotic exposure can be used to determine Live[t] and Dead[t], as shown in FIG. 14.

The system of equations provide a closed form solution for Live[t] and Dead[t] for any choice of h[t]. If a parametric form for h[t] is chosen, then by fitting data to this system of equations, one can estimate its parameters at the same time, namely the growth rate (μ), the starting number of cells (L₀), background death rate k, and parameters in the expression for h[t]. Preferred choices for h[t] are described below.

The hazard rate h[t] for the equations can take on a simplified form for a single-hit model (where the effective number of damaging “events” (α) to occur for a cell to die and lyse is equal to 1−the drug being tested) such that

h[t]=β (45)

where β is the rate at which the damaging effects (as independent, random Poisson distributed processes) occur (“kill rate”).

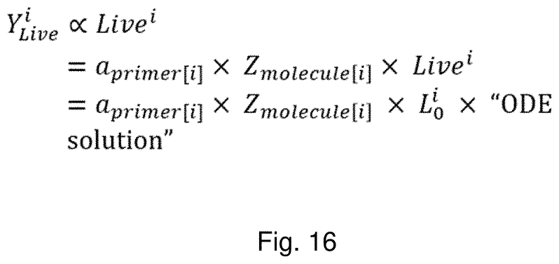

For more complicated systems where there are multiple types of damaging events (α>1), the hazard rate can be modelled as

$$h [ t ] = \frac{{β}^{α} {t}^{( α - 1 )} {e}^{- β t}}{Γ [ α ] Q [ α , β t ]} \qquad ( 46 )$$

where Q[x] is the regularized upper incomplete gamma function (Γ[x]). FIG. 15 shows example population trajectories for a growth rate of μ=0.0231 min⁻¹ and a background (non-drug) lysis rate of k=0.001 min⁻¹. Curves are given for various values of α and β. For example, the curve for α=1 and β=0 shows a rapid, unending growth with negligible deaths over time. This is as expected, as the only cause of deaths is the background lysis, which is minimal.

This would produce a very small hazard rate which, for the single-hit model, would be effectively 0. In contrast, the curves for α=1 and β=1.2 shows a rapid extinction of the entire population, giving a high hazard rate (1.2 for the single-hit model). A more complicated example is shown for α=10 and β=0.24, where the population thrives (h[t]≈0) for about half an hour before declining (h[t] increases). Since this has many events (10), the multiple-hit model is more appropriate as it will approximate the changing hazard rate over time.

The kill rate β can be approximated by the Hill equation

$$β = \frac{{{β}_{max} [ A b x ]}^{γ}}{{[ A b x ]}^{γ} + E {C}_{50}^{γ}} \qquad ( 47 )$$

where βₘₐₓ is the maximum kill rate at saturation (min⁻¹), [Abx] is the concentration of antibiotics (μg/mL), γ is the Hill coefficient (controls the shape of the curve), and EC₅₀ is the effective concentration of the antibiotics (m/mL) that cause a rate of lysis equal to 50% of the maximum rate of lysis. EC₅₀ can be seen as a measure of cell susceptibility, with EC₅₀ being higher in value but correlated with the customary minimum inhibitory concentration (MIC). This allows the modelling of the rate of lysis (killing) as a function of antibiotic concentration.

### Example 17: Linking Nucleic Acid Concentrations to Numbers or Biomass of Cells

When the nucleic acid quantified is genomic DNA or ribosomal RNA, the copy number of the nucleic acid is proportional to the size of the cell and the growth rate of the cell, but not to any other biological state of the cell (unlike mRNA, which is also subject to gene expression regulation.) The difference in copy number between individual cells within the same growth phase is usually less than 2-fold, which is not a great amount. Furthermore, the copy numbers of these nucleic acid species in lysed cells is also drawn from roughly the same distribution as the copy numbers in live cells. Therefore, when one is examining a large collection of unsynchronized cells in the same growth phase, the total amount of genomic DNA or ribosomal RNA in that collection is primarily a function of the biomass of the cells only, or equivalently, to the number of cells. For nucleic acid species like mRNA where the amount of that nucleic acid is not only a function of the number of cells more complex functions are used.

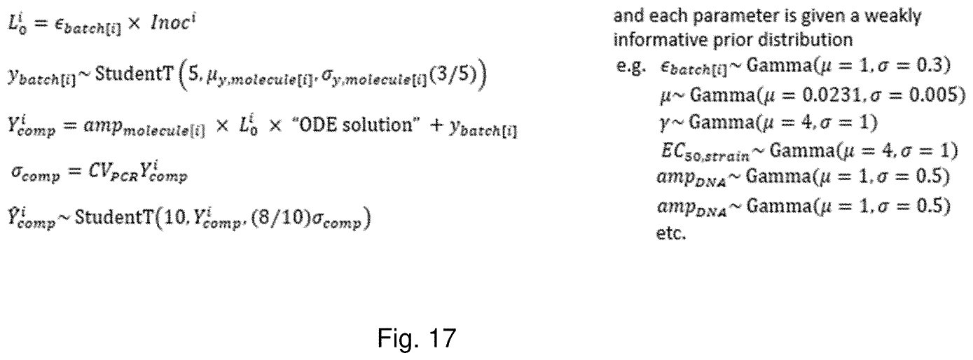

FIG. 16 shows the choice of function to link the cell population to the nucleic acid quantity for all the ASTs in this description that quantified genomic DNA or ribosomal RNA, with extracellular amount (and corresponding concentration values) proportional to lysed cell populations and intracellular amounts (and corresponding concentration values) proportional to living cell populations. The term “aₚᵣᵢₘₑᵣ” refers to the amplification efficiency of a given primer and enzyme combination and “Z_{molecule}” is the copy number per cell of a given nucleic acid species (both for experiment number “i”). Unless one can distinguish between these two variables, such as when one uses more than one primer set per nucleic acid species, then these parameters are only visible when combined as an overall amplification efficiency amp_{molecule}. If one has used more than one primer pair to amplify the same nucleic acid species, then even though the primer pairs may differ in efficiency due to different secondary structure and melting temperature as known to the skilled user, the resulting amplifications will be correlated due to the fact that the maximum number of molecules that can result, Z_{molecule}, is the same in all quantification reactions. In other words, by changing the sequence of the primer pairs, one creates a new independent variable that enables amp molecule to be split into aₚᵣᵢₘₑᵣ and Z_{molecule}. The term Y_{comp}ⁱ is the nucleic acid quantity in compartment “comp” (Live, Dead, or Total) for the i-th experiment, and L₀ⁱ is the initial inoculum of experiment i.

### Example 18: Correcting Batch Effects

As in any instance of molecular biology, non-biological factors can influence the results of the experiments. Examples include changes in lab conditions, time of day, the personnel carrying out the experiments, changes of instruments, etc.

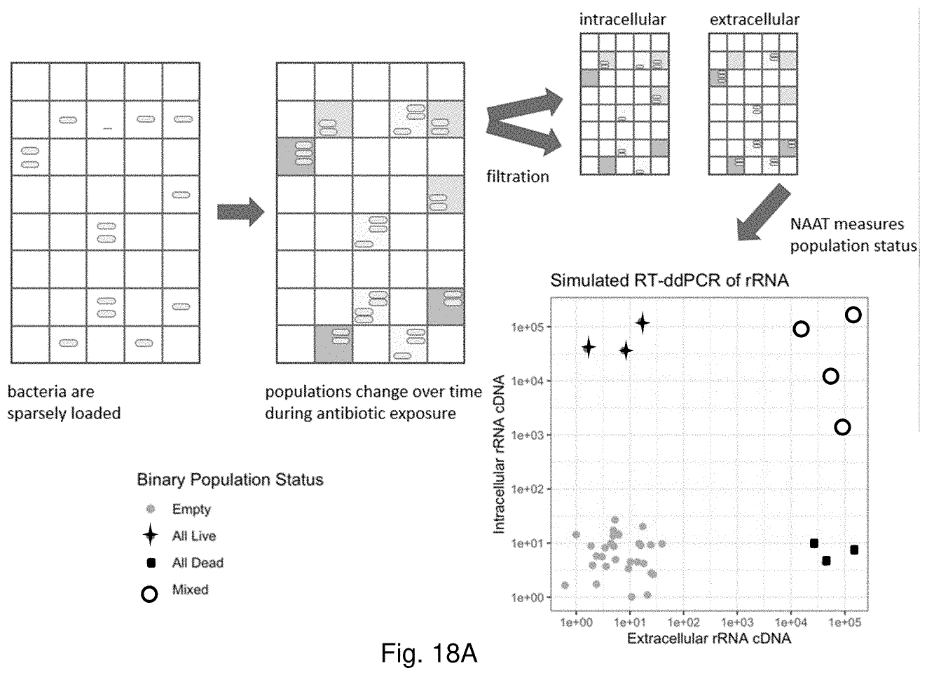

These are known as “batch effects”. An example of dealing with these batch effects is shown in FIG. 17. In the AST runs of the examples in this description, the initial starting numbers of cells in the contrived clinical specimens were controlled by performing a serial dilution immediately after measuring the optical density of an exponential phase culture of bacteria. It was deduced from the data and from control experiments that the starting number of cells loaded into the ASTs was the largest source of batch error across these AST runs.

The batch error likely arose due to slightly different average cell sizes of the batch culture at different optical densities, noise in the optical density measurements, fluctuations in the time needed to execute the serial dilution, the temperature of the media during the serial dilution, and small volumetric errors during the serial dilution. Since these effects manifest through the starting number of cells, the batch effects were modeled as deviations from the intended starting number of cells. The influence of other batch effects through other variables was assumed to be negligible and not included to simplify the model. In this example, a hierarchical Bayesian statistical error model is used.

The term L₀ⁱ is the true initial inoculum of experiment i, Inocⁱ is the intended/target average inoculum of experiment i, imperfectly controlled by the operator's serial dilution. The term ϵ_{barch} is the error in the initial starting inoculum of a given batch culture (in this example, each day's experiment was derived from 1 batch culture). y_{batch[i]} is the background nucleic acid contamination of batch i μ_{y,molecule[i]} is the average background nucleic acid across all batches,

σ_{y,molecule[i]} is proportional to the standard deviation of the background nucleic acid across all batches, CV_{PCR} is the coefficient of variation of the PCR measurement error that is proportional to analyte concentration, Y_{comp}ⁱ is the nucleic acid quantity in compartment “comp” (Live, Dead, or Total) for the i-th experiment, and Ŷ_{comp}ⁱ is the observed nucleic acid quantity in compartment “comp” (Live, Dead, or Total) for the i-th experiment.

There are a few ways to estimate Ŷ_{comp}ⁱ. For example, one can use

$${\hat{Y}}^{i} ∼ Normal ( {Y}^{i} , \sum C {\sum }^{T} ) , C = [ \begin{matrix}1 & ρ \\ ρ & 1\end{matrix} ] , \sum = [ \begin{matrix}{σ}_{Total} & 0 \\ 0 & {σ}_{D e a d}\end{matrix} ] , {σ}_{c o m p} = \sqrt{{( C {V}_{P C R} {Y}_{c o m p}^{i} )}^{2} + {σ}_{primer}^{2}} \qquad ( 48 )$$

$${\hat{Y}}^{i} ∼ Normal ( {Y}^{i} , [ \begin{matrix}{σ}_{Total}^{2} & {σ}_{Total} {σ}_{D e a d} ρ \\ {σ}_{Total} {σ}_{D e a d} ρ & {σ}_{Dead}^{2}\end{matrix} ] ) , \qquad ( 49 )$$

$${σ}_{comp}^{2} = {( C {V}_{PCR} {Y}_{c o m p}^{i} )}^{2} + {σ}_{primer}^{2} .$$

### Example 19: Hamiltonian Monte Carlo Fitting Algorithm

When fitting the curves of extracellular and/or intracellular nucleic acid measurements herein, one way to fit the curves is by a Hamiltonian Monte Carlo algorithm, which uses a Markov chain Monte Carlo method to obtain a sequence of random samples which converge to being distributed according to a target probability distribution for which direct sampling is difficult.

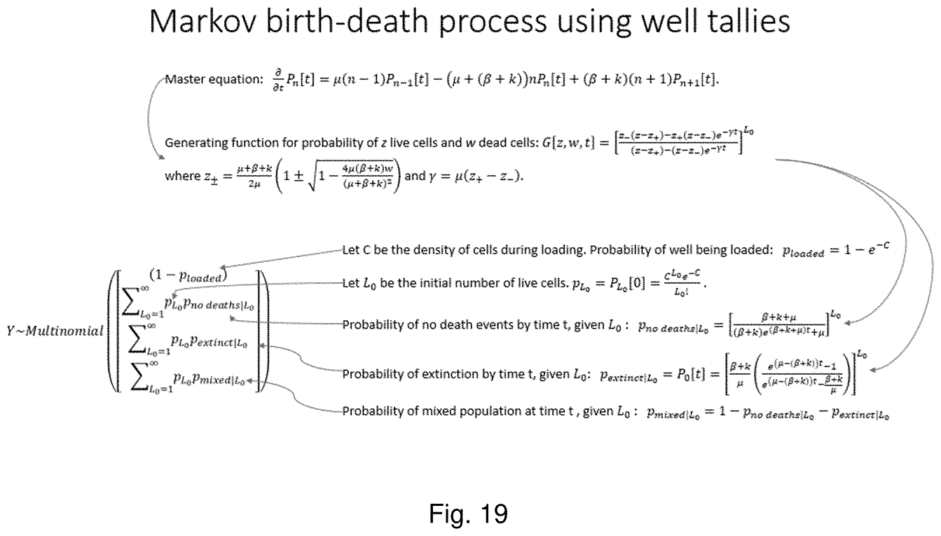

FIG. 19 shows some example values for the parameters as given for the various equations herein. This can be implemented in software and will return the joint posterior distribution of the parameters.

### Example 20 Digitally Loaded Filtration AST Enables Estimation of Single-Cell Responses

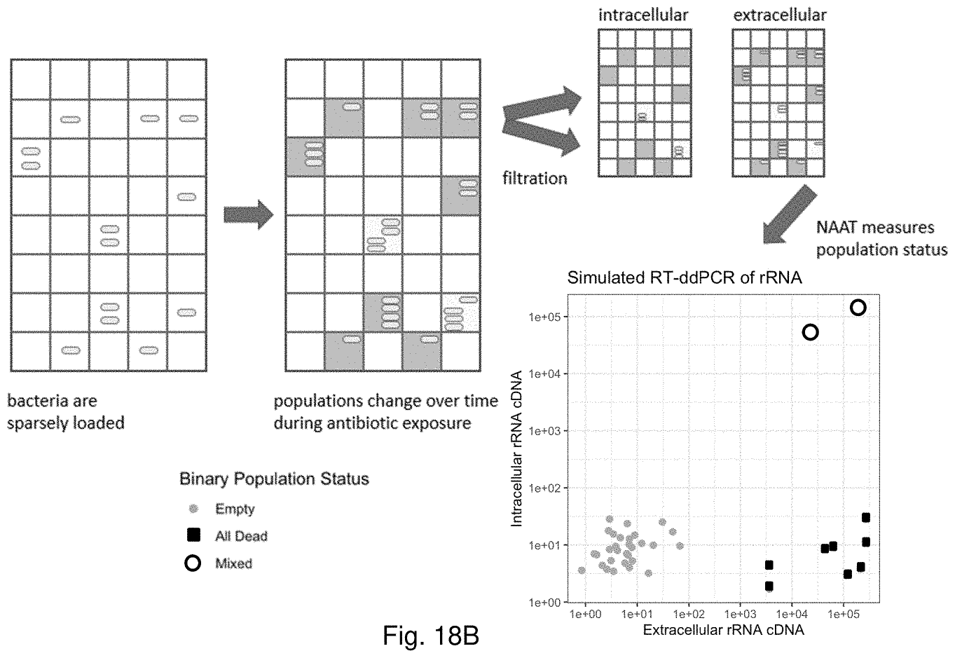

The two diagrams of FIG. 18A and FIG. 18B show a schematic of how digitally-loaded same-sample AST can be performed, using simulated data. Cells are digitally-loaded into a large array of partitions, 40 being present here. Within each partition, the population of cells changes over time, simultaneously dividing and dying with a certain probability per window of time. At the end of the exposure, some populations have gone extinct, some have had lysis events but still contain some intact cells, and some having had no lysis events occur to their cells yet. Each partition is subjected to a separation, extraction, and quantification, yielding 40 ENACVs and 40 INACVs in 40 pairs.

The 40 pairs of ENACVs and INACVs are plotted in the 2-dimensional ENACV vs INACV space shown by the square graph, and a well loading status algorithm is used to classify each partition into one of four categories, here labeled as “Binary Population Statuses”. From the tallies of each of the 40 population statuses, one can estimate the rate of lysis, which serves as the EINAPV for this condition, as well as the strain resistance metric. This is illustrated by the fact that FIGS. 18A and 18B differ only in the value of the lysis rate parameter used to simulate the data. The overall proportion of “all dead” to “all live” and of “mixed” to “all live” partition populations is higher in FIG. 18B due to its rate of lysis parameter being 3 times the growth rate, while it is only 2 times the growth rate in FIG. 18A.

As an aside, if the noise in the ENACV and INACV measurements is small enough, it may be possible for the well loading status algorithm to estimate the integer number of intact and lysed cells in each partition, instead of the less-informative, categorical well loading status. The responses of single cells to antibiotics would be more accurately assessed if the number, rather than the binary presence, of cells were estimated.

### Example 21 Markov Birth-Death Process Using Well Tallies

FIG. 19 shows the derivation of a mathematical expression which is the likelihood of observing the observed tally of well loading statuses given values of parameters, and assuming that the population behaves according to a Markov birth-death process. This likelihood expression is used in Bayesian statistics to calculate what are the most probable values of model parameters, especially the antibiotic kill rate. The statuses of sample partitions are obtained when one performs a digitally-loaded same-sample AST. Within the derivation, one derives an expression for the probability of a discrete population going extinct by time t. This probability can serve as an EINAPV alternative to the rate of lysis. A probability of going extinct by time t is positively correlated with the rate of lysis. The probability of going extinct is closer in meaning to the current convention of measuring susceptibilities by the minimum inhibitory concentration.

### Example 22: Model Fitting Algorithm and Results

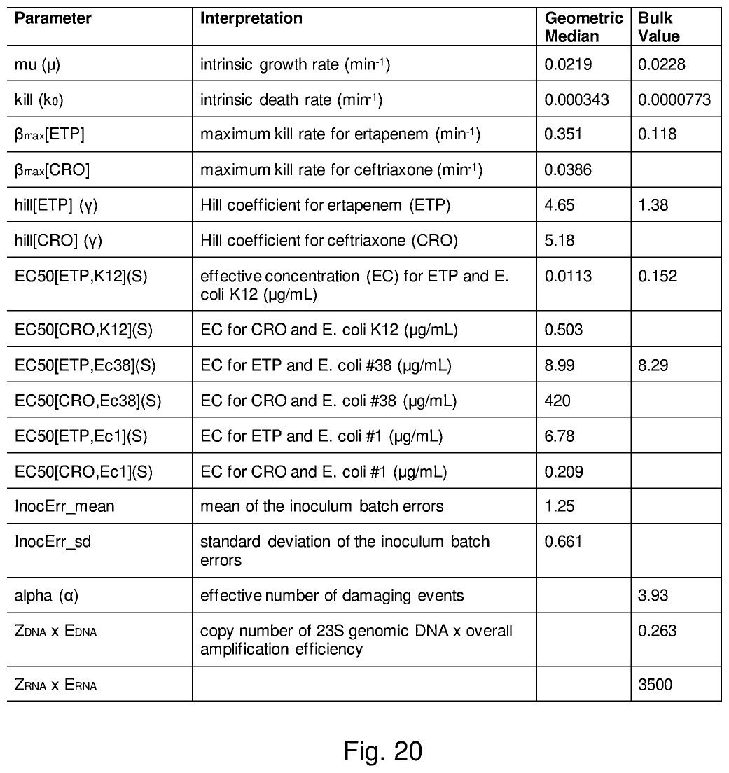

Two Bayesian models were fitted on AST results using the software Stan using weakly informative priors. Model A was fitted to bulk loaded accessibility AST, and Model B on digitally-loaded same-sample AST. The equations for Model A were those in FIGS. 14, 16, and 17. The equations for Model B were those in FIG. 19. The resulting parameters are shown in FIG. 20, where the column “geometric mean” contains the results from model B, and the column “bulk value” contains the results from Model A. The parameter values are usable whenever the parameters are needed to enable analysis of a particular embodiment of same-sample AST, but the parameters cannot be estimated from the data of that AST run, with the skilled person understanding that obtaining parameter values from more data, from data from microorganisms more closely related taxonomically, and from data from experimental conditions more similar to the practitioner's query is always preferred.

### Example 23: Modeling-Driven Workflow for Same-Sample Methods

The following considerations can be taken into account in identify the settings of same-sample AST of the disclosure:

In order to identify the best settings and the proper intracellular/extracellular proportion value to address a query using same-sample AST, one can—
1. Identify important variables and phenomena, such as:
   1. Entities/volumes created by each physical manipulation
   2. Dynamics of the size and structure of the population of microorganisms.
   3. Function linking observed nucleic acids to bacterial populations
   4. Random effects model to encompass all remaining variables.
2. Relate variables in a system of equations and assess if solvable.
   1. Discrete stochastic models needed for low cell numbers.
   2. If not solvable, modify assay to obtain missing measurements.
3. Fit model to experimental data from prototype
   1. Perform experiments, guided by solvability of model.
   2. Choose fitting algorithm to “solve” system of equations, run algorithm, assess fits by cross-validation.
4. Repeat above steps, modifying types of measurements & model components to improve model fit.

In case the test is set to be performed in clinical settings and
5. Evaluate potential barriers to clinical adoption. Repeat from start.
   1. Does assay exactly fulfill the target clinical need?
   2. Sensitivity and specificity adequate for application
   3. Measure the complete sample-to-answer assay time
   4. Amenable to automation/integration
      1. can fit in workflow
      2. robust to operator and conditions
   5. Cost, regulatory needs/POC.
6. Use assay to perform diagnostic testing in field trials
   1. “Using assay” means solving model for each specimen.
   2. Solving model for given specimens may require accumulating a database of parameter values from surveys of specimens.
   3. Mechanistic algorithms may be augmented by mechanism-agnostic ML algorithms.

In addition to the above,
7. Repeat above steps, modifying types of measurements & model components to improve model fit/assay accuracy.

Additional, exemplary embodiments features, objects, and advantages of the present disclosure will be apparent to a skilled person from the claims and the instant disclosure in its entirety,

In summary provided herein is an antibiotic susceptibility and related compositions, methods and systems based on nucleic acid detection based on detected intracellular and extracellular nucleic acid from a same sample, which allows determination of antibiotic susceptibility of microorganisms as well as the diagnosis and/or treatment of related infections in individuals.

The examples set forth above are provided to give those of ordinary skill in the art a complete disclosure and description of how to make and use the embodiments of the compounds, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosures of each document cited (including webpages patents, patent applications, journal articles, abstracts, laboratory manuals, books, or other disclosures) in the Summary, Description, Examples, and Appendix are hereby incorporated herein by reference. All references cited in this disclosure, including references cited in any one of the Appendices, are incorporated by reference to the same extent as if each reference had been incorporated by reference in its entirety individually. However, if any inconsistency arises between a cited reference and the present disclosure, the present disclosure takes precedence.

Further, the computer readable form of the sequence listing of the ASCII text file P2569-US-Sequence-Listing_ST25 is incorporated herein by reference in its entirety.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible sub-combinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims.

### REFERENCES

[1] R. F. Ismagilov, E. S. Savela, N. Schoepp, and E. J. Liaw, “Antibiotic susceptibility of microorganisms and related compositions, methods and systems,” US20190194726A1, Jun. 27, 2019 Accessed: Jan. 9, 2020. [Online]. Available: https://patents.google.com/patent/US20190194726A1/en?oq=US2019%2f0194726
[2] R. F. Ismagilov, E. S. Savela, N. Schoepp, and E. J. Liaw, “Antibiotic susceptibility of microorganisms and related compositions, methods and systems,” WO2019075264A1, Apr. 18, 2019 Accessed: Feb. 27, 2022. [Online]. Available: https://patents.google.com/patent/WO2019075264A1/en?oq=US2019%2f0194726
[3] R. F. Ismagilov, N. G. Schoepp, E. S. Savela, E. J. Liaw, A. Winnett, and M. M. Cooper, “Antibiotic susceptibility of microorganisms and related methods and systems,” US20210301326A1, Sep. 30, 2021 Accessed: Feb. 27, 2022. [Online]. Available: https://patents.google.com/patent/US20210301326A1/en?oq=US2021%2f0301326
[4] M. P. Weinstein and Clinical and Laboratory Standards Institute, Performance standards for antimicrobial susceptibility testing. 2021.
[5] “EUCAST: Clinical breakpoints and dosing of antibiotics.” https://www.eucast.org/clinical_breakpoints/ (accessed Feb. 27, 2022).
[6] W. C. Winn and E. W. Koneman, Koneman's color atlas and textbook of diagnostic microbiology. Philadelphia: Lippincott Williams & Wilkins, 2006.
[7] S. P. Lapage, P. H. A. Sneath, E. F. Lessel, V. B. D. Skerman, H. P. R. Seeliger, and W. A. Clark, Eds., International Code of Nomenclature of Bacteria: Bacteriological Code, 1990 Revision. Washington (D.C.): ASM Press, 1992. Accessed: Feb. 27, 2022. [Online]. Available: http://www.ncbi.nlm.nih.gov/books/NBK8817/[8]
[8] L. K. Logan and R. A. Weinstein, “The Epidemiology of Carbapenem-Resistant Enterobacteriaceae: The Impact and Evolution of a Global Menace,” J Infect Dis, vol. 215, no. suppl_1, pp. S28-S36, Febuary 2017, doi: 10.1093/infdis/jiw282.
[9] K. C. Carroll et al., Eds., Manual of clinical microbiology, 12th edition. Washington, D.C.: ASM Press, 2019.
[10] A. L. Truant, Y.-W. Tang, K. B. Waites, C. Bébéar, and R. P. Rennie, Eds., Manual of Commercial Methods in Clinical Microbiology: International Edition. Hoboken, N.J.: John Wiley & Sons, Inc, 2016. doi: 10.1002/9781119021872.
[11] The European Committee on Antimicrobial Susceptibility Testing, “Breakpoint tables for interpretation of MICs and zone diameters. Version 12.0, 2022.” The European Committee on Antimicrobial Susceptibility Testing, 2022. Accessed: Feb. 27, 2022. [Online]. Available: https://www.eucast.org/fileadmin/src/media/PDFs/EUCAST_files/Breakpoint_tables/v_12.0_Breakpoint_Tables.pdf
[12] E. S. Savela et al., “Surfactant-enhanced DNA accessibility to nuclease accelerates phenotypic β-lactam antibiotic susceptibility testing of Neisseria gonorrhoeae,” PLOS Biology, vol. 18, no. 3, p. e3000651, March 2020, doi: 10.1371/journal.pbio.3000651.
[13] C. A. Burtis, E. R. Ashwood, D. E. Bruns, and N. W. Tietz, Eds., Tietz textbook of clinical chemistry and molecular diagnostics, 5th ed. St. Louis, Mo.: Saunders, 2013.
[14] G. A. Obande and K. K. Banga Singh, “Current and Future Perspectives on Isothermal Nucleic Acid Amplification Technologies for Diagnosing Infections,” Infect Drug Resist, vol. 13, pp. 455-483, February 2020, doi: 10.2147/IDR.S217571.
[15] T. N. A. Nguyen, V. Anton-Le Berre, A.-L. Bailuls, and T. V. A. Nguyen, “Molecular Diagnosis of Drug-Resistant Tuberculosis; A Literature Review,” Frontiers in Microbiology, vol. 10, 2019, Accessed: Feb. 28, 2022. [Online]. Available: https://www.frontiersin.org/article/10.3389/fmicb.2019.00794
[16] M. M. Kaminski, O. O. Abudayyeh, J. S. Gootenberg, F. Zhang, and J. J. Collins, “CRISPR-based diagnostics,” Nat Biomed Eng, vol. 5, no. 7, pp. 643-656, July 2021, doi: 10.1038/s41551-021-00760-7.
[17] S. Kim, S. Ji, and H. R. Koh, “CRISPR as a Diagnostic Tool,” Biomolecules, vol. 11, no. 8, p. 1162, August 2021, doi: 10.3390/biom11081162.
[18] Y. Li, X. Yang, and W. Zhao, “Emerging microtechnologies and automated systems for rapid bacterial identification and antibiotic susceptibility testing,” SLAS TECHNOLOGY. Translating Life Sciences Innovation, vol. 22, no. 6, pp. 585-608, 2017.
[19] H. Leonard, R. Colodner, S. Halachmi, and E. Segal, “Recent Advances in the Race to Design a Rapid Diagnostic Test for Antimicrobial Resistance,” ACS Sens, vol. 3, no. 11, pp. 2202-2217, November 2018, doi: 10.1021/acssensors.8b00900.
[20] A. van Belkum, C.-A. D. Burnham, J. W. A. Rossen, F. Mallard, O. Rochas, and W. M. Dunne, “Innovative and rapid antimicrobial susceptibility testing systems,” Nat Rev Microbiol, vol. 18, no. 5, Art. no. 5, May 2020, doi: 10.1038/s41579-020-0327-x.
[21] J. E. Kreutz, T. Munson, T. Huynh, F. Shen, W. Du, and R. F. Ismagilov, “Theoretical Design and Analysis of Multivolume Digital Assays with Wide Dynamic Range Validated Experimentally with Microfluidic Digital PCR,” Anal. Chem, vol. 83, no. 21, pp. 8158-8168, November 2011, doi: 10.1021/ac201658s.
[22] S. Guzman, A. Schlogl, and C. Schmidt-Hieber, “Stimfit: quantifying electrophysiological data with Python,” Frontiers in Neuroinformatics, vol. 8, 2014, Accessed: Feb. 18, 2022. [Online]. Available: https://www.frontiersin.org/article/10.3389/fninf.2014.00016
[23] M. Sekerli, C. A. Del Negro, R. H. Lee, and R. J. Butera, “Estimating action potential thresholds from neuronal time-series: new metrics and evaluation of methodologies,” IEEE Transactions on Biomedical Engineering, vol. 51, no. 9, pp. 1665-1672, September 2004, doi: 10.1109/TBME.2004.827531.
[24] D. Altman, D. Machin, T. Bryant, and M. Gardner, Statistics with confidence: confidence intervals and statistical guidelines. John Wiley & Sons, 2013.
[25] A. Gelman, H. S. Stern, J. B. Carlin, D. B. Dunson, A. Vehtari, and D. B. Rubin, Bayesian data analysis. Chapman and Hall/CRC, 2013.
[26] Stephen. Marsland, Machine learning: an algorithmic perspective, 2nd ed. Boca Raton, Fla.: Chapman and Hall/CRC, 2015.
[27] D. C. Montgomery, Design and Analysis of Experiments. John Wiley & Sons, Incorporated, 2017. [Online]. Available: https://books.google.com/books?id=Py7bDgAAQBAJ
[28] S. E. White and B. Dawson, Eds., Basic & clinical biostatistics, Fifth edition. New York: McGraw-Hill, 2020.
[29] L. A. Seidman, C. J. Moore, and J. Mowery, Basic laboratory methods for biotechnology: textbook and laboratory reference, Third edition. Boca Raton: CRC Press, 2022.
[30] D. A. Skoog, Fundamentals of analytical chemistry, 9th Ed. Belmont, Calif.: Cengage—Brooks/Cole, 2012.
[31] Joseph. Sambrook, E. F. Fritsch, and Tom. Maniatis, Molecular cloning: a laboratory manual. 1 1. Cold Spring Harbor: Cold Spring Harbor Laboratory Press, 2012.
[32] J. Q. Boedicker, L. Li, T. R. Kline, and R. F. Ismagilov, “Detecting bacteria and determining their susceptibility to antibiotics by stochastic confinement in nanoliter droplets using plug-based microfluidics,” Lab on a Chip, vol. 8, no. 8, pp. 1265-1272, 2008, doi: 10.1039/B804911D.
[33] Y. Hori, C. Kantak, R. M. Murray, and A. R. Abate, “Cell-free extract based optimization of biomolecular circuits with droplet microfluidics,” Lab Chip, vol. 17, no. 18, pp. 3037-3042, September 2017, doi: 10.1039/C7LC00552K.
[34] P. Shahi, S. C. Kim, J. R. Haliburton, Z. J. Gartner, and A. R. Abate, “Abseq: Ultrahigh-throughput single cell protein profiling with droplet microfluidic barcoding,” Scientific Reports, vol. 7, no. 1, Art. no. 1, March 2017, doi: 10.1038/srep44447.

1. A method to detect a nucleic acid of a microorganism in a sample including the microorganism, the method comprising
contacting the sample with an antibiotic to provide an antibiotic-treated sample,
separating the antibiotic-treated sample into an antibiotic-treated extracellular component and an antibiotic-treated cellular component,
detecting a nucleic acid concentration of the antibiotic-treated extracellular component to obtain an antibiotic-treated extracellular nucleic acid concentration value, and
detecting a nucleic acid concentration of the antibiotic-treated cellular component to obtain an antibiotic-treated intracellular nucleic acid concentration value.

2. The method of claim 1, wherein the mechanical separation is performed by filtration and/or centrifugation of the antibiotic treated sample.

3. The method of claim 1, further comprising
comparing the detected antibiotic treated intracellular nucleic acid (NA) concentration value and the detected antibiotic treated extracellular nucleic acid (NA) concentration value
to provide an antibiotic treated intracellular/extracellular nucleic acid proportion value of the sample.

4. The method of claim 3, wherein the antibiotic treated intracellular/extracellular nucleic acid proportion value of the sample is
a ratio of the detected antibiotic treated intracellular concentration value or of the antibiotic treated detected extracellular concentration value and a sum of the detected antibiotic treated intracellular NA concentration value and the detected antibiotic treated extracellular NA concentration value,
or a mathematical equivalent thereto.

5. The method of claim 3, wherein the antibiotic treated intracellular/extracellular nucleic acid proportion value of the sample is a percentage extracellular concentration or an intracellular percentage concentration or a mathematical equivalent thereto.

6. The method of claim 3, wherein the antibiotic treated intracellular/extracellular nucleic acid proportion value of the sample is a probability of lysis.

7. The method of claim 3, wherein the method further comprises
determining a proportionality of dead and live microorganism cells in the sample caused by and/or or as a function of, the antibiotic by determining an intra/extra proportion value of the sample to provide a dead/live proportion value of the microorganism cells in the sample.

8. The method of claim 3, further comprising
comparing the antibiotic treated intracellular/extracellular nucleic acid proportion value of the sample with
a reference value indicative of an intracellular/extracellular nucleic acid proportion in the sample in absence of antibiotic treatment
to obtain a treated-reference nucleic acid comparison outcome of the sample.

9. The method of claim 8, wherein the reference value comprises
a reference intracellular/extracellular nucleic acid proportion value of a reference sample corresponding to the antibiotic treated intracellular/extracellular nucleic acid proportion value of the sample.

10. The method of claim 9, wherein the reference sample is an antibiotic untreated control sample.

11. The method of claim 8, wherein the reference value comprises
a threshold value obtained based on standard deviations of distributions of extracellular and/or intracellular nucleic acid concentrations of the microorganism in absence of antibiotic treatment.

12. The method of claim 8, wherein the reference value is provided by a plurality of reference values arranged in a distribution forming a function to provide a reference profile.

13. The method of claim 8; wherein the method further comprises
determining antibiotic susceptibility when the treated-reference nucleic acid comparison outcome of the sample indicates an increased lysis and an increased dead/live proportion of the microorganism cells in the antibiotic-treated sample compared to a sample treated under reference conditions; or
determining antibiotic resistance when the treated-reference nucleic acid comparison outcome of the sample indicates a substantially same dead/live proportion of the microorganism cells in the antibiotic-treated sample compared to a sample treated under reference conditions.

14. The method of claim 1, wherein the method further comprises
splitting the antibiotic-treated sample to obtain a plurality of sub-samples before the contacting,
and wherein
the contacting is performed under at least one set of test condition in a corresponding at least set of sub-sample,
the separating, the detecting a nucleic acid concentration of the antibiotic-treated extracellular component and the detecting a nucleic acid concentration of the antibiotic-treated cellular component are performed on each sub-sample of the at least one set of sub-samples of plurality of sub-samples,
to obtain an antibiotic-treated intracellular nucleic acid concentration value and an antibiotic-treated extracellular nucleic acid concentration value for the at least one set of sub-samples of the plurality of sub-samples.

15. The method of claim 14, wherein splitting the sample is performed by digital partitioning.

16. The method of claim 15, wherein the digital partitioning provides at least one samples of the plurality of samples not having any cells, at least one sample of the plurality of samples with less than 10 cells or less than 5 cells, and/or at least one sample of the plurality of samples having a single cell of the target microorganism.

17. The method of claim 14, further comprising
comparing the detected antibiotic treated intracellular concentration value and the detected antibiotic treated extracellular nucleic acid concentration value of the at least one set of sub-samples of the plurality of sub-samples of the plurality of sub-samples
to provide an antibiotic treated intracellular/extracellular nucleic acid proportion value for each of the at least one set of sub-samples of the plurality of sub-samples.

18. The method of claim 17, wherein the plurality of antibiotic treated intracellular/extracellular nucleic acid proportion values of the sample are used to provide an antibiotic treated intracellular/extracellular nucleic acid proportion profile of the sample.

19. The method of claim 18, further comprising
comparing the antibiotic treated intracellular/extracellular nucleic acid proportion profile of the sample with
a reference value indicative of an intracellular/extracellular nucleic acid proportion in the sample in absence of antibiotic treatment
to obtain a treated-reference nucleic acid comparison outcome of the sample.

20. The method of claim 14; wherein the method further comprises
determining antibiotic susceptibility when the treated-reference nucleic acid comparison outcome of the sample indicates an increased lysis and an increased dead/live proportion of the microorganism cells in the antibiotic-treated sample compared to a sample treated under reference conditions; or
determining antibiotic resistance when treated-reference nucleic acid comparison outcome of the sample indicates a substantially same dead/live proportion of the microorganism cells in the antibiotic-treated sample compared to a sample treated under reference conditions.

21. The method of claim 1, wherein the sample is pretreated to enrich said sample with the target microorganism, and/or to remove human nucleic acid or nucleic of other microorganisms, optionally by size selection.

22. The method of claim 1, wherein the sample comprises a number of microorganism cells lower than 100, lower than 50, lower than 25, lower than 10, or lower than 5.

23. The method of claim 1, wherein the sample and/or one or more sub-samples comprises a single microorganism cell.

24. The method of claim 1, wherein contacting the sample with an antibiotic is performed for up to 90 minutes, up to 45 minutes, up to 30 minutes, up to 15 minutes, or up to 5 minutes.

25. The method of claim 1, wherein the detecting is performed by digital nucleic acid quantification to obtain a digital nucleic acid quantification concentration value.

26. The method of claim 25, wherein the digital nucleic acid quantification is performed by digital PCR, digital RT-PCR, digital LAMP, digital RT LAMP, digital RPA, or other digital isothermal amplification.

27. The method of claim 1, wherein the nucleic acid is DNA and the detection is performed by qPCR or by DNA-seq wherein the nucleic acid concentration value is provided based on the sequence data.

28. The method of claim 1, wherein the nucleic acid is RNA, and the detection is performed by RT-qPCR or by RNA-seq wherein the nucleic acid concentration value is provided based on the sequence data.

29. The method of any one of claim 1, wherein the antibiotic is or comprises a beta-lactam and or a carbapenem.

30. The method of any one of claim 1, wherein the microorganism is Neisseria gonorrhoeae and/or the microorganism belongs to the family Enterobacteriaceae.


SEQUENCE LISTING (3 sequences)

SEQ ID NO: 1 - DNA, 28 bp, Artificial Sequence
  synthetic polynucleotide
  misc_feature (13)..(13): locked nuleic acid
cgttagcacccgccgtgtgtctcccgtg

SEQ ID NO: 2 - DNA, 20 bp, Artificial Sequence
  synthetic polynucleotide
ggtagagcactgttttggca

SEQ ID NO: 3 - DNA, 22 bp, Artificial Sequence
  synthetic polynucleotide
tgtctcccgtgataactttctc